(19) United States
(12) Patent Application Publication
(10) Pub. No.: US 2024/0166669 A1
(43) Pub. Date: May 23, 2024

(54) QUINAZOLINE AMINE DERIVATIVES AS KRAS INHIBITORS

(71) Applicant: Nikang Therapeutics, Inc., Wilmington, DE (US)

(72) Inventors: Jiping Fu, Danville, CA (US); Yan Lou, Wilimington, DE (US)

(21) Appl. No.: 18/549,007

(22) PCT Filed: Mar. 3, 2022
(86) PCT No.: PCT/US22/18760

§ 371 Date: Sep. 5, 2023

Related U.S. Application Data
(60) Provisional application No. 63/221,743, filed on Jul. 14, 2021.
(60) Provisional application No. 63/170,948, filed on Apr. 5, 2021.
(60) Provisional application No. 63/158,867, filed on Mar. 9, 2021.
(60) Provisional application No. 63/157,624, filed on Mar. 5, 2021.

(51) Int. Cl. C07D 519/00

(52) U.S. Cl. CPC C07D 519/00

(57) ABSTRACT

The present disclosure provides certain quinazoline derivatives that inhibit certain K-Ras proteins and are therefore useful for the treatment of cancers mediated by such proteins. Also provided are pharmaceutical compositions containing such compounds and processes for preparing such compounds.

### CROSS REFERENCE TO RELATED APPLICATIONS

This application claims priority to, and the benefit of, U.S. Provisional Application Nos. 63/157,624, filed Mar. 5, 2021; 63/158,867, filed Mar. 9, 2021; 63/170,948, filed Apr. 5, 2021; and 63/221,743, filed Jul. 14, 2021, the contents of each of which are incorporated by reference in their entireties.

### FIELD OF THE DISCLOSURE

The present disclosure provides certain quinazoline amine derivatives compounds that inhibit certain K-Ras proteins and are therefore useful for the treatment of cancers mediated by such proteins. Also provided are pharmaceutical compositions containing such compounds and processes for preparing such compounds.

### BACKGROUND

Kirsten Rat Sarcoma 2 Viral Oncogene Homolog (KRAS) gene is a prevalent oncogene that encodes a small GTPase transductor protein called K-Ras. K-Ras can serve as a molecular switch by cycling between active GTP-bound and inactive GDP-bound forms (see Science 2001; 294:1299-304.). K-Ras signaling is activated by RAS guanine nucleotide exchange factors (GEFs), e.g., Son of Sevenless homologue (SOS) protein, that facilitate the GDP to GTP exchange of K-Ras (see Curr Biol 2005; 15:563-74.). The interaction between K-Ras and GTPase-activating proteins (GAPs) such as p120GAP and neurofibromin, potentiates K-Ras intrinsic GTPase activity and accelerates GTP hydrolysis and diminishing K-Ras signaling (see Curr. Biol. 2005; 15:563-74.).

K-Ras plays a crucial role in the regulation of cell proliferation, differentiation and survival by signaling through several major downstream pathways, including the MAPK, the PI3K and the Ral-GEFs pathways (see Lung Cancer 2018; 124: 53-64), among them the MAPK 30 pathway is the best characterized (see Mol. Cell Biol. 1995; 15:6443-6453.). K-Ras-GTP binds to and activates RAF kinases, which phosphorylates MEK and subsequently phosphorylates ERK. Phospho-ERK can further activate downstream cytosolic proteins, and which then translocate to the nucleus to drive the expression of diverse genes, propagating the growth signal.

PI3K pathway is also involved in RAS-mediated tumorigenesis (see Cell 2007; 129:957-968.). Upon activation by K-Ras-GTP, PI3K phosphorylates PIP2 to form PIP3, activates PDK1 and then phosphorylates AKT. pAKT yields phosphorylation of several physiological substrates, e.g., mTOR, FOXO and NF-κB that promote metabolism, cell-cycle progression, resistance to apoptosis, cell survival and migration. The Ral-GEFs signaling pathway plays a key role in RAS-mediated oncogenesis as well (see Proc. Natg. Acad. Sci. U.S.A 1994; 91:11089-11093.). The K-Ras effector, RALGDS, stimulates the RAS family RAL-A/B small GTPases for the subsequent signaling cascades. RALGDS can also promote the JNK pathway to stimulate transcription of pro-survival and cell-cycle progression genes for cell proliferation and survival.

KRAS gene is the most frequently mutated oncogene in human cancer. KRAS mutations are associated with poor clinical outcome and found at high frequency in pancreatic cancer (˜90%), colorectal cancer (˜44%) and non-small-cell lung cancer (NSCLC) (˜29%) (see Cancer Discov. 2021; 11:1-16). KRAS mutations are also present in breast cancer, liver cancer, biliary tract malignancies, endometrial cancer, cervical cancer, bladder cancer and myeloid leukemia. The most common KRAS mutations are observed at residues G12 (77%), G13 (10%), and Q61 (6%), and the most predominant KRAS variant in human malignancies is G12D (35%), followed by G12V (29%), G12C (21%), G12A (7%), G12R (5%), and G12S (3%) (see Cancer Discov. 2021; 11:1-16). These mutations perturbate GTP hydrolysis of K-Ras by interfering with GAP binding/stimulation and/or reducing K-Ras intrinsic GTPase activity, resulting in constitutive activation of the protein and K-Ras signaling.

Targeting KRAS signaling has been a long pursuit in drug discovery. Among KRAS mutants, K-Ras G12C offers special opportunity, because it harbors a non-native cysteine residue, which can act as nucleophile and therefore can be targeted by covalent attachment. Several such covalent inhibitors, including AMG510, MRTX849, JNJ-74699157 and LY349944631, are in clinical trials for treating cancer patients with KRAS G12C mutation (see ACS Cent. Sci. 2020; 6:1753-1761). These compounds occupy a dynamic pocket in the switch II region of K-Ras thereby irreversibly locking K-Ras G12C in inactive GDP-bound state. Since KRAS mutations, including G12C, enrich predominantly active-state protein in cancer cells, sufficient residual GTPase activity and nucleotide cycling are required for effective inhibition of K-Ras by inactive state-selective drugs (see Cell 2020; 183(4):850-859). Currently, there are no molecules in clinical trial that can inhibit K-Ras G12C by binding to its active GTP form or both GTP and GDP forms. Inhibitors of active form of K-Ras should be more effective at suppressing cell growth and survival, as well as less susceptible to adaptive resistance than inhibitors binding to its inactive form. Compared to K-Ras G12C mutant, other prevalent K-Ras mutants, such as G12D, do not contain non-native cysteine residue and cycle through inactive state at extremely low rate, thus making non-G12C mutant-specific drug discovery more challenging.

Given the role of K-Ras mutants in human malignancy, there is still unmet medical need for development of new treatments for cancer patients with KRAS mutations. The present disclosure fulfills this and related needs.

### SUMMARY

In a first embodiment of the first aspect, provided is a compound of Formula (IIA1′):

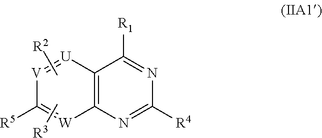

wherein:

U, V, and W are CH; or one or two of U, V, and W are N and the other of U, V, and W are CH;

R¹ is a ring of formula:

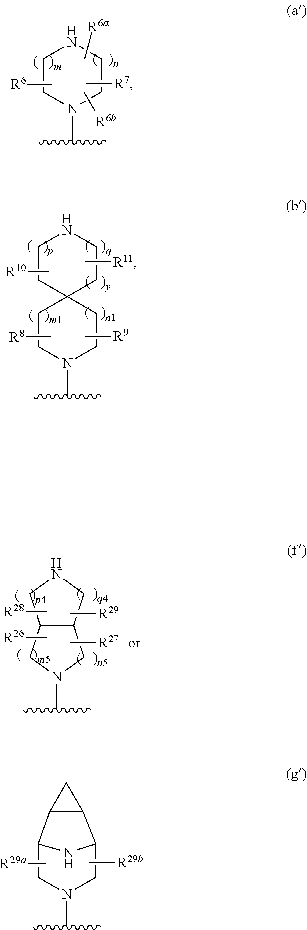

where:

one of m and n is 0, 1, or 2, and the other of m and n is 0, 1, 2, or 3;

m1, n1, m5 and n5 are independently 0, 1, or 2, provided one of m5 and n5 is at least 1;

p, q, p4 and q4 are independently 0, 1, or 2, y is 0 or 1, provided one of p4 and q4 is at least 1;

R⁶, R⁸, R¹⁰, R²⁶, and R²⁸ are independently hydrogen, deuterium, alkyl, alkoxy, halo, haloalkyl, hydroxy, hydroxylalkyl, alkoxyalkyl, cyano, or cyanomethyl, provided R⁶, R¹⁰, and R²⁸ are not attached to the ring —NH—;

R⁷, R⁹, R¹¹, R²⁷, and R²⁹ are independently hydrogen, deuterium, alkyl, alkoxy, halo, haloalkyl, hydroxy, hydroxylalkyl, or alkoxyalkyl, provided R⁷, R¹¹, and R²⁹ are not attached to the ring —NH—; or

when R⁶ and R⁷ are attached to the carbon atoms of the ring that are opposite or diagonal to each other, then R⁶ and R⁷ can combine to form —(CH₂)_{z}— where (z is 1, 2, or 3), or —CH═CH—;

R^{6a} is hydrogen, deuterium, alkyl, alkylidenyl, alkoxy, halo, haloalkyl, hydroxy, hydroxyalkyl, alkoxyalkyl, cyano, or cyanomethyl, provided R^{6a} is not attached to the ring —NH—;

R^{6b} is hydrogen or alkyl, provided R^{6b} is not attached to the ring —NH—, or

when R^{6a} and R^{6b} are attached to the same carbon of ring (a′), they can combine to form cycloalkylene;

R^{29a} and R^{29b} are independently hydrogen, alkyl, hydroxv, cyano, or cyanomethyl provided R^{29a} and R^{29b} are not attached to the ring —NH—;

R² is hydrogen, deuterium, alkyl, halo, haloalkyl, alkoxy, hydroxy, or cyano, provided that R² is absent when two of U, V, and W are N;

R³ is hydrogen, deuterium, alkyl, halo, haloalkyl, alkoxy, cycloalkyloxy, hydroxy, or cyano;

R⁴ is:

(i) —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, heterocyclyl fused bicyclic heterocyclyl, heterocyclyl fused bicyclic heterocyclylalkyl, tricyclic heterocyclyl, tricyclic heterocyclylalkyl, fused tricyclic heterocyclyl, fused tricyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, heterocyclyl fused bicyclic heterocyclyl, by itself of as part of heterocyclyl fused bicyclic heterocyclylalkyl, tricyclic heterocyclyl, by itself or as part of tricyclic heterocyclylalkyl, fused tricyclic heterocyclyl, by itself or as part of fused tricyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, alkenyl, haloalkenyl, cycloalkyl, cycloalkyloxy, cycloalkylalkyl, bridged cycloalkyl, halo, haloalkyl, haloalkoxy, hydroxy, alkoxy, alkylidenyl, haloalkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; or

(ii) —CR³³═CR³⁴R³⁵ where R³³ and R³⁴ are independently selected from hydrogen, deuterium, alkyl, halo, and haloalkyl; and

R³⁵ is hydrogen, deuterium, alkyl, halo, haloalkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, or spiro heterocyclyl; or

R³⁴ and R³⁵ together with the carbon atom to which are attached form cycloalkyl, bridged cycloalkyl, fused cycloalkyl, spiro cycloalkyl, heterocyclyl, bridged heterocyclyl, fused heterocyclyl, or spiro heterocyclyl, wherein:

(a) the groups alkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, and spiro heterocyclyl of R³⁵; and (b) the groups cycloalkyl, bridged cycloalkyl, fused cycloalkyl, spiro cycloalkyl, heterocyclyl, bridged heterocyclyl, fused heterocyclyl, and spiro heterocyclyl formed by R³⁴ and R³⁵ together, are independently substituted with R^{h}, R, and RJ independently selected from hydrogen, alkyl, halo, haloalkyl, hydroxyalkyl, alkoxyalkyl, cyano, cycloalkyl, bridged cycloalkyl, optionally substituted heterocyclyl, alkylsulfonylalkyl, dialkyl(oxo)phosphinyl, dialkyl(oxo)phosphinylalkyl, —O(alk)_{z1}R^{k}, —O(alk)OR^{l}, —S(O)R^{m}, —S(O)₂Rⁿ, —NR^{p}C(O)R^{o}, —NR^{r}S₂R^{q}, —OC(O)NR^{s}R^{t}, —C(O)NR^{u}R^{v}, —S(O)₂NR^{w}R^{x}, and —NR^{y}R^{z}, where z1 is 0 or 1, alk is alkylene, and R^{k}, R^{l}, R^{m}, Rⁿ, R^{o}, R^{p}, R^{q}, R^{r}, R^{s}, R^{t}, R^{u}, R^{v}, R^{w}, R^{x}, R^{y}, and R^{z} are independently selected from hydrogen, alkyl, cycloalkyl, cycloalkylalkyl, halo, hydroxyalkyl, alkoxyalkyl, and aminoalkyl; and

R⁵ is -Q-R³⁶ where Q is bond, alkylene, or —C(═O)—; and R³⁶ is hydrogen, cycloalkyl, fused cycloalkyl, aryl, aralkyl, heteroaryl, or heteroaralkyl, wherein aryl, aryl in aralkyl, heteroaryl, and heteroaryl in heteroaralkyl are independently substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl; or

a pharmaceutically acceptable salt thereof.

In a second embodiment of first aspect, provided is a compound of Formula (IIA′):

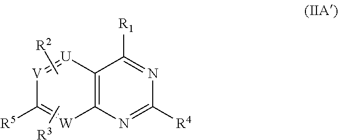

wherein:

U, V, and W are CH; or one or two of U, V, and W are N and the other of U, V, and W are CH;

R¹ is a ring of formula:

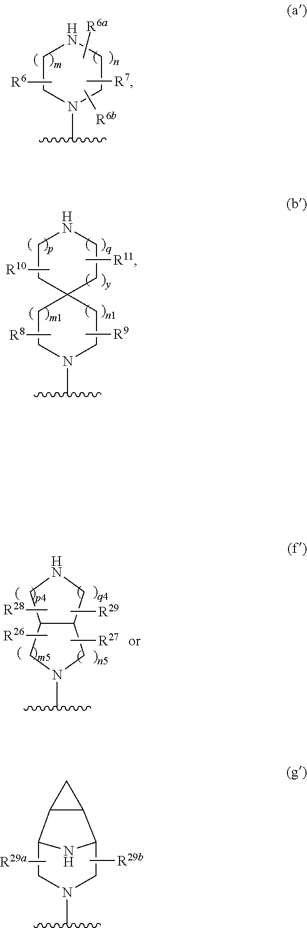

where:

one of m and n is 0, 1, or 2, and the other of m and n is 0, 1, 2, or 3, provided m+n is not more than 8;

m1, n1, m5 and n5 are independently 0, 1, or 2, provided one of m5 and n5 is at least 1;

p, q, p4 and q4 are independently 0, 1, or 2, y is 0 or 1; provided that one of p4 and q4 is at least 1;

R⁶, R⁸, R¹⁰, R²⁶, and R²⁸ are independently hydrogen, deuterium, alkyl, alkoxy, halo, haloalkyl, hydroxy, hydroxylalkyl, alkoxyalkyl, cyano, or cyanomethyl, provided R⁶, R¹⁰, and R²⁸ are not attached to the ring —NH—;

R⁷, R⁹, R¹¹, R²⁷, and R²⁹ are independently hydrogen, deuterium, alkyl, alkoxy, halo, haloalkyl, hydroxy, hydroxylalkyl, or alkoxyalkyl, provided R⁷, R¹¹, and R²⁹ are not attached to the ring —NH—; or

when R⁶ and R⁷, R⁸ and R⁹, and R¹⁰ and R¹¹ are attached to the carbon atoms of the ring that are opposite or diagonal to each other, then R⁶ and R⁷ can combine to form —(CH₂)_{z}— where (z is 1, 2, or 3), or —CH═CH—;

R^{6a} is hydrogen, deuterium, alkyl, alkylidienyl, alkoxy, halo, haloalkyl, hydroxy, hydroxyalkyl, alkoxyalkyl, cyano, or cyanomethyl, provided R^{6a} is not attached to the ring —NH—;

R^{6b} is hydrogen or alkyl, provided R^{6b} is not attached to the ring —NH—; or

when R^{6a} and R^{6b} are attached to the same carbon of ring (a′), they can combine to form alkylidienyl or cycloalkylene;

R^{29a} and R^{29b} are independently hydrogen, alkyl, hydroxy, cyano, or cyanomethyl, provided R^{29a} and R^{29b} are not attached to the ring —NH—;

R² is hydrogen, deuterium, alkyl, halo, haloalkyl, alkoxy, hydroxy, or cyano, provided that R² is absent when two of U, V, and W are N;

R³ is hydrogen, deuterium, alkyl, halo, haloalkyl, alkoxy, cycloalkyloxy, hydroxy, or cyano;

R⁴ is:

(i) —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, heterocyclyl fused bicyclic heterocyclyl, heterocyclyl fused bicyclic heterocyclylalkyl, tricyclic heterocyclyl, tricyclic heterocyclylalkyl, fused tricyclic heterocyclyl, fused tricyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, heterocyclyl fused bicyclic heterocyclyl, by itself of as part of heterocyclyl fused bicyclic heterocyclylalkyl, tricyclic heterocyclyl, by itself or as part of tricyclic heterocyclylalkyl, fused tricyclic heterocyclyl, by itself or as part of fused tricyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, alkenyl, haloalkenyl, cycloalkyl, cycloalkyloxy, cycloalkylalkyl, bridged cycloalkyl, halo, haloalkyl, haloalkoxy, hydroxy, alkoxy, alkylidienyl, haloalkylidienyl, alkoxyalkylidienyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl;

(ii) —CR³³═CR³⁴R³⁵ where R³³ and R³⁴ are independently selected from hydrogen, deuterium, alkyl, halo, and haloalkyl; and

R³⁵ is hydrogen, deuterium, alkyl, halo, haloalkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, or spiro heterocyclyl; or

R³⁴ and R³⁵ together with the carbon atom to which are attached form cycloalkyl, bridged cycloalkyl, fused cycloalkyl, spiro cycloalkyl, heterocyclyl, bridged heterocyclyl, fused heterocyclyl, or spiro heterocyclyl, wherein:

(a) the groups alkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, and spiro heterocyclyl of R³⁵; and (b) the groups cycloalkyl, bridged cycloalkyl, fused cycloalkyl, spiro cycloalkyl, heterocyclyl, bridged heterocyclyl, fused heterocyclyl, and spiro heterocyclyl formed by R³⁴ and R³⁵ together, are independently substituted with R^{h}, Rⁱ, and R^{j} independently selected from hydrogen, alkyl, halo, haloalkyl, hydroxyalkyl, alkoxyalkyl, cyano, cycloalkyl, bridged cycloalkyl, optionally substituted heterocyclyl, alkylsulfonylalkyl, dialkyl(oxo)phosphinyl, dialkyl(oxo)phosphinylalkyl, —O(alk)_{z1}R^{k}, —O(alk)OR^{l}, —S(O)R^{m}, —S(O)₂Rⁿ, —NR^{p}C(O)R^{o}, —NR^{r}SO₂R^{q}, —OC(O)NR^{s}R^{t}, —C(O)NR^{u}R^{v}, —S(O)₂NR^{w}R^{x}, and —NR^{y}R^{z}, where z1 is 0 or 1, alk is alkylene, and R^{k}, R^{l}, R^{m}, Rⁿ, R^{o}, R^{p}, R^{q}, R^{r}, R^{s}, R^{t}, R^{u}, R^{v}, R^{w}, R^{x}, R^{y}, and R^{z} are independently selected from hydrogen, alkyl, cycloalkyl, cycloalkylalkyl, halo, hydroxyalkyl, alkoxyalkyl, and aminoalkyl; and

R⁵ is -Q-R³⁶ where Q is bond, alkylene, or —C(═O)—; and R³⁶ is hydrogen, cycloalkyl, fused cycloalkyl, aryl, aralkyl, heteroaryl, or heteroaralkyl wherein aryl, aryl in aralkyl, heteroaryl, and heteroaryl in heteroaralkyl are independently substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl; or

a pharmaceutically acceptable salt thereof.

In a second aspect, provided is a compound of Formula (II′):

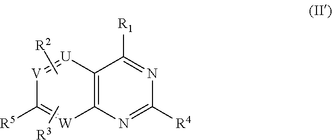

wherein:

U, V, and W are CH; or one or two of U, V, and W are N and the other of U, V, and W are CH;

R¹ is a ring of formula:

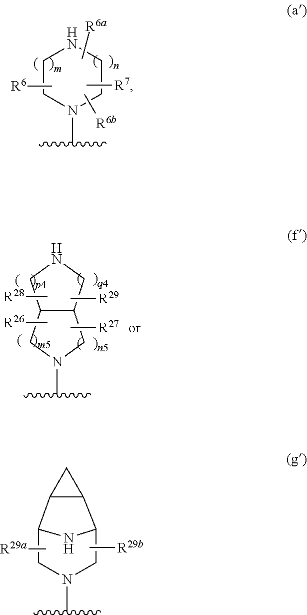

where:

m, m5, n, and n5 are independently 0, 1, or 2, provided at least one of m5 and n5 is 1;

p⁴ and q4 are independently 0, 1, or 2, provided at least one of p4 and q4 is 1;

R⁶, R²⁶, and R²⁸ are independently hydrogen, deuterium, alkyl, alkoxy, halo, haloalkyl, hydroxy, hydroxylalkyl, alkoxyalkyl, cyano, or cyanomethyl, provided R⁶ and R²⁸ are not attached to the ring —NH—;

R⁷, R²⁷, and R²⁹ are independently hydrogen, deuterium, alkyl, alkoxy, halo, haloalkyl, hydroxy, hydroxylalkyl, or alkoxyalkyl, provided R⁷ and R²⁹ are not attached to the ring —NH—; or

when R⁶ and R⁷ are attached to the carbon atoms of the ring that are opposite or diagonal to each other, then R⁶ and R⁷ can combine to form —(CH₂)_{z}— where (z is 1, 2, or 3), or —CH═CH—;

R^{6a} is hydrogen, deuterium, alkyl, alkylidenyl, alkoxy, halo, haloalkyl, hydroxy, hydroxylalkyl, alkoxyalkyl, cyano, or cyanomethyl, provided R^{6a} is not attached to the ring —NH—;

R^{6b} is hydrogen or alkyl, provided R^{6b} is not attached to the ring —NH—; or

when R^{6a} and R^{6b} are attached to the same carbon of ring (a′), they can combine to form alkylidenyl or cycloalkylene;

R^{29a} and R^{29b} are independently hydrogen, alkyl, hydroxy, cyano, or cyanomethyl, provided R^{29a} and R^{29b} are not attached to the ring —NH—;

R² is hydrogen, deuterium, alkyl, halo, haloalkyl, alkoxy, hydroxy, or cyano, provided that R² is absent when two of U, V, and W are N;

R³ is hydrogen, deuterium, alkyl, halo, haloalkyl, alkoxy, cycloalkyloxy, hydroxy, or cyano;

R⁴ is:

(i) —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, tricyclic heterocyclyl, tricyclic heterocyclylalkyl, fused tricyclic heterocyclyl, fused tricyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, tricyclic heterocyclyl, by itself or as part of tricyclic heterocyclylalkyl, fused tricyclic heterocyclyl, by itself or as part of fused tricyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, alkenyl, haloalkenyl, cycloalkyl, cycloalkyloxy, cycloalkylalkyl, bridged cycloalkyl, halo, haloalkyl, haloalkoxy, hydroxy, alkoxy, alkylidenyl, haloalkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; or

(ii) —CR³³═CR³⁴R³⁵ where R³³ and R³⁴ are independently selected from hydrogen, deuterium, alkyl, halo, and haloalkyl; and

R³⁵ is hydrogen, deuterium, alkyl, halo, haloalkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, or spiro heterocyclyl; or

R³⁴ and R³⁵ together with the carbon atom to which are attached form cycloalkyl, bridged cycloalkyl, fused cycloalkyl, spiro cycloalkyl, heterocyclyl, bridged heterocyclyl, fused heterocyclyl, or spiro heterocyclyl, wherein:

(a) the groups alkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, and spiro heterocyclyl of R³⁵; and (b) the groups cycloalkyl, bridged cycloalkyl, fused cycloalkyl, spiro cycloalkyl, heterocyclyl, bridged heterocyclyl, fused heterocyclyl, and spiro heterocyclyl formed by R³⁴ and R³⁵ together, are independently substituted with R^{h}, Rⁱ, and R^{j} independently selected from hydrogen, alkyl, halo, haloalkyl, hydroxyalkyl, alkoxyalkyl, cyano, cycloalkyl, bridged cycloalkyl, optionally substituted heterocyclyl, alkylsulfonylalkyl, dialkyl(oxo)phosphinyl, dialkyl(oxo)phosphinvlalkyl, —O(alk)_{z1}R^{k}, —O(alk)OR^{l}, —S(O)R^{m}, —S(O)₂Rⁿ, —NR^{p}C(O)R^{o}, —NR^{r}SO₂R^{q}, —OC(O)NR^{s}R^{t}, —C(O)NR^{u}R^{v}, —S(O)₂NR^{w}R^{x}, and —NR^{y}R^{z}, where z1 is 0 or 1, alk is alkylene, and R^{k}, R^{l}, R^{m}, Rⁿ, R^{o}, R^{p}, R^{q}, R^{r}, R^{s}, R^{t}, R^{u}, R^{v}, R^{w}, R^{x}, R^{y}, and R^{z} are independently selected from hydrogen, alkyl, cycloalkyl, cycloalkylalkyl, halo, hydroxyalkyl, alkoxyalkyl, and aminoalkyl; and

R⁵ is -Q-R³⁶ where Q is bond, alkylene, or —C(═O)—; and R³⁶ is hydrogen, cycloalkyl, fused cycloalkyl, aryl, aralkyl, heteroaryl, or heteroaralkyl, wherein aryl, aryl in aralkyl, heteroaryl, and heteroaryl in heteroaralkyl are independently substituted with R^{aa}, R^{bb}, R^{cc} and Rad wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl; or

a pharmaceutically acceptable salt thereof.

In a third aspect, provided is a compound of Formula (II):

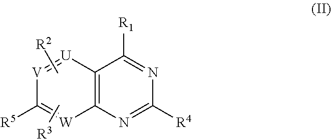

wherein:

U, V, and W are CH; or one or two of U, V, and W are N and the other of U, V, and W are CH;

R¹ is a ring of formula:

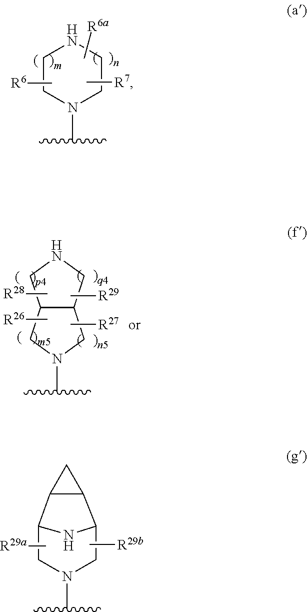

where:

m, m5, n, and n5 are independently 0, 1, or 2, provided at least one of m5 and n5 is 1;

p4 and q4 are independently 0, 1, or 2, provided at least one of p4 and q4 is 1;

R⁶, R^{6a}, R²⁶, and R²⁸ are independently hydrogen, deuterium, alkyl, alkoxy, halo, haloalkyl, hydroxy, hydroxylalkyl, alkoxyalkyl, cyano, or cyanomethyl, provided R⁶, R^{6a}, and R²⁸ are not attached to the ring —NH—;

R⁷, R²⁷, and R²⁹ are independently hydrogen, deuterium, alkyl, alkoxy, halo, haloalkyl, hydroxy, hydroxylalkyl, or alkoxyalkyl, provided R⁷ and R²⁹ are not attached to the ring —NH—; or

when R⁶ and R⁷ are attached to the carbon atoms of the ring that are opposite or diagonal to each other, then R⁶ and R⁷ can combine to form —(CH₂)_{z}— where (z is 1, 2, or 3), or —CH═CH—;

R^{29a} and R^{29b} are independently hydrogen, alkyl, hydroxy, cyano, or cyanomethyl, provided R^{29a} and R^{29b} are not attached to the ring —NH—;

R² is hydrogen, deuterium, alkyl, halo, haloalkyl, alkoxy, hydroxy, or cyano, provided that R² is absent when two of U, V, and W are N;

R³ is hydrogen, deuterium, alkyl, halo, haloalkyl, alkoxy, cycloalkyloxy, hydroxy, or cyano;

R⁴ is:

(i) —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; or

(ii) —CR³³═CR³⁴R³⁵ where R³³ and R³⁴ are independently selected from hydrogen, deuterium, alkyl, halo, and haloalkyl; and

R³⁵ is hydrogen, deuterium, alkyl, halo, haloalkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, or spiro heterocyclyl; or

R³⁴ and R³⁵ together with the carbon atom to which are attached form cycloalkyl, bridged cycloalkyl, fused cycloalkyl, spiro cycloalkyl, heterocyclyl, bridged heterocyclyl, fused heterocyclyl, or spiro heterocyclyl, wherein:

(a) the groups alkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, and spiro heterocyclyl of R³⁵; and (b) the groups cycloalkyl, bridged cycloalkyl, fused cycloalkyl, spiro cycloalkyl, heterocyclyl, bridged heterocyclyl, fused heterocyclyl, and spiro heterocyclyl formed by R³⁴ and R³⁵ together, are independently substituted with R^{h}, Rⁱ, and R^{j} independently selected from hydrogen, alkyl, halo, haloalkyl, hydroxyalkyl, alkoxyalkyl, cyano, cycloalkyl, bridged cycloalkyl, optionally substituted heterocyclyl, alkylsulfonylalkyl, dialkyl(oxo)phosphinyl, dialkyl(oxo)phosphinylalkyl, —O(alk)_{z1}R^{k}, —O(alk)OR^{l}, —S(O)R^{m}, —S(O)₂Rⁿ, —NR^{p}C(O)R^{o}, —NR^{r}SO₂R^{q}, —OC(O)NR^{s}R^{t}, —C(O)NR^{u}R^{v}, —S(O)₂NR^{w}R^{x}, and —NR^{y}R^{z}, where z1 is 0 or 1, alk is alkylene, and R^{k}, R^{l}, R^{m}, Rⁿ, R^{o}, R^{p}, R^{q}, R^{r}, R^{s}, R^{t}, R^{u}, R^{v}, R^{w}, R^{x}, R^{y}, and R^{z} are independently selected from hydrogen, alkyl, cycloalkyl, cycloalkylalkyl, halo, hydroxyalkyl, alkoxyalkyl, and aminoalkyl; and

R⁵ is -Q-R³⁶ where Q is bond, alkylene, or —C(═O)—; and R³⁶ is hydrogen, cycloalkyl, fused cycloalkyl, aryl, aralkyl, heteroaryl, or heteroaralkyl, wherein aryl, aryl in aralkyl, heteroaryl, and heteroaryl in heteroaralkyl are independently substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl; or

a pharmaceutically acceptable salt thereof.

Compound of Formula (IIA′), (II′) and (II) are a subset of compounds of Formula (IIA1′).

In a fourth aspect, provided is a pharmaceutical composition comprising a compound of Formula (IIA1′), (IIA′), (II′), or (II) (or any of the embodiments thereof described herein), or a pharmaceutically acceptable salt thereof, and a pharmaceutically acceptable excipient.

In a fifth aspect, provided is a method of inhibiting K-Ras, in particular K-Ras G12D, in a cell, comprising contacting the cell with a compound of Formula (IIA1′), (IIA′), (II′) or (II) (or any of the embodiments thereof described herein). In one embodiment of the fifth aspect, the contacting is in vitro. In another embodiment of the fifth aspect, the contacting is in vivo.

In a sixth aspect, provided is a method of inhibiting cell proliferation in vitro or in vivo, comprising contacting a cell with a compound of Formula (IIA1′), (IIA′), (II′), or (II) (or any of the embodiments thereof described herein) or a pharmaceutical composition thereof as disclosed herein. In one embodiment of the sixth aspect, the contacting is in vitro. In another embodiment of the sixth aspect, the contacting is in vivo.

In a seventh aspect, provided is a method of treating cancer in a patient, preferably the patient is in need of such treatment, which method comprises administering to the patient, preferably a patient in need of such treatment, a therapeutically effective amount of a compound of Formula (IIA1′), (IIA′), (II′), or (II) (or any of the embodiments thereof described herein), or a pharmaceutically acceptable salt thereof; or a pharmaceutical composition thereof as disclosed herein.

In an eighth aspect, provided is a method of treating cancer associated with K-Ras, in particular K-Ras G12D, in a patient, preferably the patient is in need of such treatment, which method comprises administering to the patient, preferably a patient in need of such treatment, a therapeutically effective amount of a compound of Formula (IIA1′), (IIA′), (II′) or (II) (or any of the embodiments thereof described herein), or a pharmaceutically acceptable salt thereof, or a pharmaceutical composition thereof as disclosed herein.

In a ninth aspect, provided is a compound of Formula (IIA1′), (IIA′), (II′), or (II), (or any embodiments thereof described herein), or a pharmaceutically acceptable salt thereof, or a pharmaceutical composition thereof as disclosed herein, for use as a medicament. In one embodiment, the medicament is useful for the treatment of cancer.

In a tenth aspect, provided is a compound of Formula (IIA1′), (IIA′), (II′), or (II), (or any embodiments thereof described herein), or a pharmaceutically acceptable salt thereof, or a pharmaceutical composition thereof as disclosed herein, for use as a therapy.

In an eleventh aspect, provided is a compound of Formula (IIA1′), (IIA′), (II′), or (II), (or any embodiments thereof described herein), or a pharmaceutically acceptable salt thereof, or a pharmaceutical composition thereof as disclosed herein, for use in the treatment of cancer.

In a twelfth aspect, provided is a compound of Formula (IIA1′), (IIA′), (II′) or (II), (or any embodiments thereof described herein), or a pharmaceutically acceptable salt thereof, or a pharmaceutical composition thereof as disclosed herein, for use in the treatment of cancers associated with KRas, in particular cancers associated with K-Ras G12D.

In a thirteenth aspect, provided is a compound of Formula (IIA1′), (IIA′), (II′) or (II), (or any embodiments thereof described herein), or a pharmaceutically acceptable salt thereof, or a pharmaceutical composition thereof as disclosed herein, for use in inhibiting K-Ras, in particular K-Ras G12D.

In any of the aforementioned aspects involving the treatment of cancer, are further embodiments comprising administering the compound of Formula (IIA1′), (IIA′), (II′), or (II) (or any embodiments thereof disclosed herein), or a pharmaceutically acceptable salt thereof, in combination with at least one additional anticancer agent. When combination therapy is used, the agents can be administered simultaneously or sequentially.

### DETAILED DESCRIPTION

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkenyl” means a linear monovalent hydrocarbon radical of two to six carbon atoms or a branched monovalent hydrocarbon radical of three to six carbon atoms containing a double bond e.g., ethenyl, propenyl, 2-propenyl, butenyl, pentenyl, and the like.

“Alkynyl” means a linear monovalent hydrocarbon radical of two to six carbon atoms or a branched monovalent hydrocarbon radical of three to six carbon atoms containing a triple bond e.g., ethynyl, propynyl, 2-propynyl, butynyl, and the like.

“Alkylamino” means a —NHR radical where R is alkyl as defined above, e.g., methylamino, ethylamino, and the like.

“Alkylsulfonyl” means a —SO₂R radical where R is alkyl as defined above, e.g., methylsulfonyl, ethylsulfonyl, and the like.

“Alkylsulfonylalkyl” means a -(alkylene)-SO₂R radical where R is alkyl as defined above, e.g., methylsulfonylmethyl, ethylsulfonylmethyl, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, such as one or two alkoxy groups, as defined above, e.g., 2-methoxyethyl, 1-, 2-, or 3-methoxypropyl, 2-ethoxyethyl, and the like.

“Alkoxyalkyloxy” means a —OR radical where R is alkoxyalkyl as defined above. Examples include, but are not limited to, 2-methoxyethyloxy, 1-, 2-, or 3-methoxypropyloxy, 2-ethoxyethyloxy, and the like.

“Alkylidenyl” means refers to a group of formula R═ where R is alkyl as defined above. Examples include, but are not limited to, methylidenyl (H₂C═), ethylidenyl (CH₃CH═), propylidenyl (═C(CH₃)₂), hexylidenyl (CH₃(CH₂)₄CH═), and the like. For example, in the compound below:

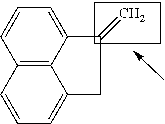

the alkylidene group, methylidenyl, is enclosed by the box which is indicated by the arrow.

“Alkoxyalkylidenyl” means refers to a group of formula ═R where R is alkoxyalkyl as defined above. Examples include, but are not limited to, methoxethylidenyl (CH₃OCH₂CH═), methoxyethylidenyl (═C(CH₃)OCH₃), ethoxyethylidenyl (C₂H₅OCH₂CH═), and the like. For example, in the compound below:

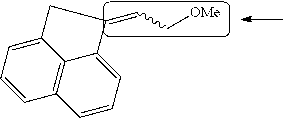 [SMILES: COCC=C1Cc2cccc3cccc1c23]

the alkoxyalkylidenyl group, methoxethylidenyl, is enclosed by the box which is indicated by the arrow.

“Alkoxycarbonyl” means a —C(O)OR radical where R is alkyl as defined above, e.g., methoxycarbonyl, ethoxycarbonyl, and the like.

“Acyl” means a —C(O)R radical where R is alkyl, haloalkyl, cycloalkyl, optionally substituted phenyl, optionally substituted heteroaryl, or optionally substituted heterocyclyl, as defined herein, e.g., methylcarbonyl, ethylcarbonyl, benzoyl, trifluoromethylcarbonyl, cyclopropylcarbonyl, and the like. When R is alkyl, acyl is also referred to herein as alkylcarbonyl.

“Amino” means a —NH₂ radical.

“Aminoalkyl” means a linear monovalent hydrocarbon radical of one to six carbon atoms or a branched monovalent hydrocarbon radical of three to six carbons substituted with —NR′R″ where R′ and R″ are independently hydrogen, alkyl, cycloalkyl, cycloalkylalkyl (wherein cycloalkyl and cycloalkyl ring in cycloalkylalkyl is optionally substituted with one, two, or three substituents independently selected from alkyl, hydroxyalkyl, haloalkyl, halo, hydroxy, alkoxy, —NH₂, alkylamino, dialkylamino, and cyano), hydroxyalkyl, alkoxyalkyl, alkylcarbonyl, optionally substituted aryl, optionally substituted heteroaryl, optionally substituted heteroaralkyl, optionally substituted heterocyclyl, or optionally substituted heterocyclylalkyl, each as defined herein, e.g., aminomethyl, aminoethyl, methylaminomethyl, and the like.

“Aryl” means a monovalent monocyclic or bicyclic aromatic hydrocarbon radical of 6 to 10 ring atoms e.g., phenyl or naphthyl.

“Aralkyl” means a -(alkylene)-R radical where R is aryl as defined above. Examples include, but are not limited to, benzyl, phenethyl, and the like.

“Bicyclic heterocyclyl” means a saturated monovalent fused bicyclic ring of 8 to 12 ring atoms in which one or two ring atoms are heteroatom independently selected from N, O, and S(O)ₙ, where n is an integer from 0 to 2, the remaining ring atoms being C, unless stated otherwise. Additionally, one or two ring carbon atoms in the heterocyclyl ring can optionally be replaced by a —CO— group. More specifically the term bicyclic heterocyclyl includes, but is not limited to, hexahydro-1H-pyrrolizinyl, and the like.

“Bicyclic heterocyclylalkyl” means a -(alkylene)-R radical where R is bicyclic heterocyclyl as defined above. Examples include, but are not limited to, hexahydro-1H-pyrrolizinylmethyl, hexahydro-1H-pyrrolizinyl-7a-methyl, hexahy dro-1H-pyrrolizinylethyl, hexahydro-1H-pyrrolizinyl-7a-ethyl and the like.

“Bridged cycloalkyl” means a saturated monocyclic ring having 5 to 7 ring carbon ring atoms in which two non-adjacent ring atoms are linked by a (CRR′)ₙ group where n is 1 to 3 and R and R′ are independently H or methyl (also may be referred to herein as “bridging” group). Unless otherwise stated, bridged cycloalkyl is optionally substituted with one or two substituents independently selected from alkyl, halo, alkoxy, hydroxy, and cyano. Examples include, but are not limited to, bicyclo[1.1.1]pentane, bicyclo[2.1.1]hexane, bicyclo[2.2.2]-octane, and the like.

“Bridged heterocyclyl” means a saturated monocyclic ring having 5 to 7 ring carbon ring atoms in which two non-adjacent ring atoms are linked by a (CRR′)n1 group where n1 is 1 to 3 and R and R′ are independently H or methyl (also may be referred to herein as “bridging” group) and further wherein one or two ring carbon atoms, including an atom in the bridging group, is replaced by a heteroatom selected from N, O, and S(O)n, where n is an integer from 0 to 2. Unless otherwise stated, bridged heterocyclyl is optionally substituted with one or two substituents independently selected from alkyl, halo, alkoxy, hydroxy, and cyano. Examples include, but are not limited to, 2-azabicyclo[2.2.2]octyl, quinuclidin-4-yl, 7-oxabicyclo-[2.2.1]heptane, 1-azabicyclo[2.2.1]heptane, 2-azabicyclo[2.2.1]heptane, 3λ²-azabicyclo[3.1.0]-hexane, and the like.

“Bridged heterocyclylalkyl” means a-(alkylene)-R radical where R is bridged heterocyclyl as defined above. Examples include, but are not limited to, 2-azabicyclo[2.2.2]-octylmethyl, 3λ²-azabicyclo[3.1.0]hexylethyl, and the like.

“Cycloalkyl” means a monocyclic saturated monovalent hydrocarbon radical of three to ten carbon atoms. Examples include, but are not limited to, cyclopropyl, cyclobutyl, cyclopentyl, cyclohexyl, and the like.

“Cycloalkylene” means a monocyclic saturated divalent hydrocarbon radical of three to ten carbon atoms. Examples include, but are not limited to, 1,1-cyclopropylene, 1,1-cyclobutylene, 1,1-cyclopentylene, and the like.

“Cycloalkylalkyl” means a -(alkylene)-R radical where R is cycloalkyl as defined above. Examples include, but are not limited to, cyclopropylmethyl cyclobutylethyl, cyclopentylmethyl, cyclohexylmethyl, and the like.

“Cycloalkyloxy” means a —OR radical where R is cycloalkyl as defined above. Examples include, but are not limited to, cyclopropyloxy, cyclobutyloxy, cyclopentyloxy, cyclohexyloxy, and the like.

“Cyanoalkyl” means a linear monovalent hydrocarbon radical of one to six carbon atoms or a branched monovalent hydrocarbon radical of three to six carbons substituted with cyano e.g., cyanomethyl, cyanoethyl, and the like.

“Cyanoalkynyl” means an alkynyl radical as defined above where one of the hydrogen atoms in the alkynyl chain is replace by a cyano. Examples include, but are not limited to, —C≡C(CN), —CH₂C≡C(CN), and the like.

“Carboxy” means —COOH.

“Deuterium” mean refers to ²H or D.

“Dialkylamino” means a —NRR′ radical where R and R′ are independently alkyl as defined above, e.g., dimethylamino, methylethylamino, and the like.

“Dialkylaminocarbonylalkyl” means a -(alkylene)-CONR′R″ radical where R′ and R″ are independently alkyl as defined herein, e.g., dimethylaminocarbonylmethyl, dimethylaminocarbonylethyl, and the like.

“Dialkyl(oxo)phosphinyl” means a —P(═O)RR′ radical where R and R′ are independently alkyl as defined above, e.g., dimethyl(oxo)phosphinyl, diethyl(oxo)phosphinyl, and the like.

“Dialkyl(oxo)phosphinylalkyl” means a -(alkylene)-P(═O)RR′ radical where R and R′ are independently alkyl as defined above radical where R is alkyl as defined above, e.g., dimethyl(oxo)phosphinylmethyl, diethyl(oxo)phosphinylethyl, and the like.

“Fused bicyclic heterocyclyl” means a saturated monovalent fused bicyclic ring of 8 to 10 ring atoms in which one or two ring atoms are heteroatoms independently selected from N, O, and S(O)ₙ, where n is an integer from 0 to 2, the remaining ring atoms being C, unless stated otherwise, and where two adjacent ring atoms of the bicyclic ring are fused to two adjacent ring atoms of phenyl or a five or six membered heteroaryl, each as defined herein, unless stated otherwise. More specifically the term fused bicyclic heterocyclyl includes, but is not limited to, 2,3-dihydro-TH-pyrrolo[2,1-a]isoindol-9b(5H)-yl, 2,3-dihydro-1H-pyrrolo[1,2-a]-indol-9a(9H)-yl, 1,5,6,8-tetrahydropyrrolo[3,2-a]pyrrolizin-3b(4H)-yl, and the like.

“Fused bicyclic heterocyclylalkyl” means a -(alkylene)-R radical where R is fused bicyclic heterocyclyl as defined above. Examples include, but are not limited to, 2,3-dihydro-1H-pyrrolo[2,1-a]isoindol-9b(5H)-ylmethyl, 2,3-dihydro-1H-pyrrolo[1,2-a]indol-9a(9H)-ylmethyl, and the like.

“Fused cycloalkyl” as used herein, means cycloalkyl as defined above where two adjacent ring atoms of the cycloalkyl ring are fused to two adjacent ring atoms of phenyl or a five or six membered heteroaryl, each as defined herein, unless stated otherwise. The fused cycloalkyl can be attached at any atom of the ring. Non limiting examples of the fused cycloalkyl include bicyclo[4.1.0]hepta-1,3,5-triene, bicyclo[4.2.0]octa-1,3,5-triene, and the like.

“Fused heterocyclyl” as used herein, means a saturated monovalent monocyclic ring of 4 to 7 ring atoms having from one to three heteroatoms independently selected from N, O, and S and the remaining ring atoms being carbon, and further wherein two adjacent ring atoms of the monocyclic ring are fused to two adjacent ring atoms of a cycloalkyl, phenyl or a five or six membered heteroaryl, each as defined herein, unless stated otherwise. The nitrogen and sulfur atoms are optionally oxidized, and the nitrogen atom(s) are optionally quaternized and one or two carbon atoms of the fused ring atoms in the saturated monocyclic ring includes the two common ring vertices shared with the fused phenyl or five or six membered heteroaryl. The fused heterocyclyl can be attached at any atom of the ring. Non limiting examples of the fused heterocyclyl include 2,3-dihydrobenzo[b][1,4]-dioxinyl, 2-oxabicyclo[3.1.0]hexanyl, indolinyl, and the like.

“Fused heterocyclylalkyl” as used herein, means a -(alkylene)-R radical where R is fused heterocyclyl, as defined herein.

“Fused tricyclic heterocyclyl” means a saturated monovalent fused tricyclic ring of 9 to 16 ring atoms, preferably 10 to 14 ring atoms, in which one or two ring atoms are heteroatoms independently selected from N, O, and S(O)ₙ, where n is an integer from 0 to 2, the remaining ring atoms being C, unless stated otherwise, and where two adjacent ring atoms of the tricyclic ring (preferably two adjacent ring atoms of a ring other than the central ring of the tricyclic ring) are fused to two adjacent ring atoms of cycloalkyl, phenyl or a five or six membered heteroaryl, each as defined herein, unless stated otherwise. The term fused tricyclic heterocyclyl includes, but is not limited to,

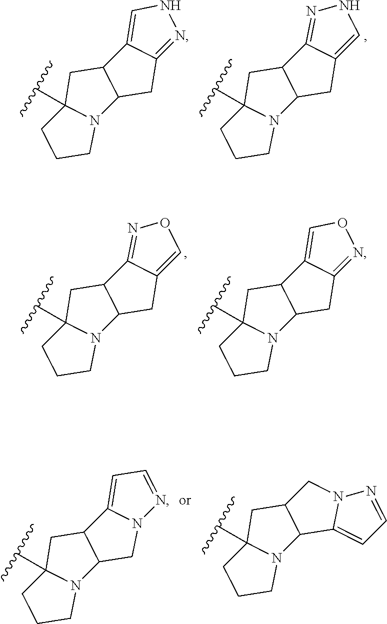 [SMILES: *C12CCCN1C1Cc3n[nH]cc3C1C2 ; *C12CCCN1C1Cc3c[nH]nc3C1C2 ; *C12CCCN1C1Cc3conc3C1C2 ; *C12CCCN1C1Cc3nocc3C1C2 ; *C12CCCN1C1Cn3nccc3C1C2 ; *C12CCCN1C1c3ccnn3CC1C2]

and the like.

“Fused tricyclic heterocyclylalkyl” means a -(alkylene)-R radical where R is fused tricyclic heterocyclyl as defined above. Examples include, but are not limited to,

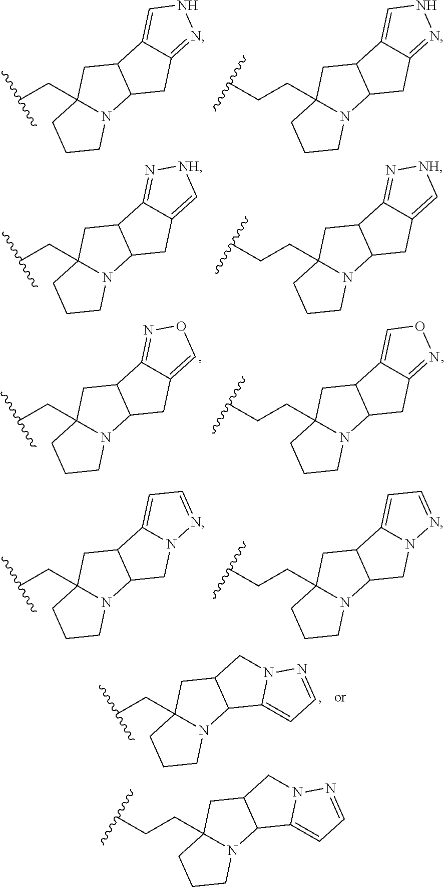

and the like.

“Halo” means fluoro, chloro, bromo, or iodo, preferably fluoro or chloro.

“Haloalkyl” means alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkenyl” means alkenyl radical as defined above, which is substituted with one or more halogen atoms, e.g., one to five halogen atoms, such as fluorine or chlorine, including those substituted with different halogens, e.g., —CH═CHF, 1-fluoroethylidenyl (═CFCH₃), —CH═CF₂, and the like. When the alkenyl is substituted with only fluoro, it can be referred to in this Application as fluoroalkenyl.

“Haloalkylidenyl” means refers to a group of formula ═R where R is haloalkyl as defined above. Examples include, but are not limited to, difluoromethylidenyl (═CF₂), fluoroethylidenyl (═CHCHF₂), and the like. For example, in the compound below:

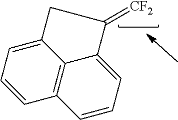

the group pointed to by the arrow is the haloalkylidenyl group, difluoromethylidenyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present, they both are not on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heteroalkyl” mean alkyl radical as defined above wherein one or two carbon atoms are replaced by O, NR (R is H or alkyl), or S, provided the heteroalkyl group is attached to the remainder of the molecule via a carbon atom, e.g., methoxymethyl, methylethylaminoethyl, and the like.

“Heteroaryl” means a monovalent monocyclic or fused bicyclic aromatic radical of 5 to 10 ring atoms, unless otherwise stated, where one or more, (in one embodiment, one, two, or three), ring atoms are heteroatom selected from N, O, and S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5- or 6 ring atoms it is also referred to herein as 5-or 6-membered heteroaryl.

“Heteroaralkyl” means a -(alkylene)-R radical where R is heteroaryl as defined above, e.g., pyridinylmethyl, and the like. When the heteroaryl ring in heteroaralkyl contains 5- or 6 ring atoms it is also referred to herein as 5-or 6-membered heteroaralkyl.

“Heterocyclyl” means a saturated or unsaturated monovalent monocyclic group of 4 to 8 ring atoms in which one or two ring atoms are heteroatom independently selected from N, O, and S(O)ₙ, where n is an integer from 0 to 2, the remaining ring atoms being C, unless stated otherwise. Additionally, one or two ring carbon atoms in the heterocyclyl ring can optionally be replaced by a —CO— group. More specifically the term heterocyclyl includes, but is not limited to, pyrrolidinyl, piperidinyl, homopiperidinyl, 2-oxopyrrolidinyl, 2-oxopiperidinyl, morpholinyl, piperazinyl, tetrahydro-pyranyl, thiomorpholinyl, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group.

“Heterocyclvlalkyl” or “heterocycloalkyl” means a -(alkylene)-R radical where R is heterocyclyl ring as defined above e.g., tetravdrofuranylmethyl, piperazinylmethyl, morpholinylethyl, and the like.

“Heterocyclyl fused bicyclic heterocyclyl” means a bicyclic heterocyclyl as defined herein (preferably a bicyclic heterocyclyl of 8 to 10 ring atoms) where two adjacent ring atoms of the bicyclic heterocyclyl are fused to two adjacent ring atoms of a hetereocyclyl ring as defined herein, provided the heterocyclyl ring contains at least two heteroatoms independently selected from N, O, and S(O)ₙ, where n is an integer from 0 to 2. The term heterocyclyl fused bicyclic heterocyclyl includes, but is not limited to,

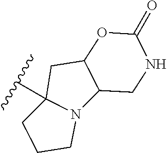 [SMILES: *C12CCCN1C1CNC(=O)OC1C2]

and the like.

“Heterocyclyl fused bicyclic heterocyclyl” means a -(alkylene)-R radical where R is heterocyclyl fused bicyclic heterocyclyl as defined above.

“Oxo,” as used herein, alone or in combination, refers to ═(O).

“Optionally substituted aryl” means aryl as defined above, that is optionally substituted with one, two, or three substituents independently selected from alkyl, hydroxyl, cycloalkyl, carboxy, alkoxycarbonyl, hydroxy, alkoxy, alkylsulfonyl, amino, alkylamino, dialkylamino, halo, haloalkyl, haloalkoxy, and cyano. When aryl is phenyl, optionally substituted aryl is referred to herein as optionally substituted phenyl.

“Optionally substituted aralkyl” means -(alkylene)-R where R is optionally substituted aryl as defined above.

“Optionally substituted heteroaryl” means heteroaryl as defined above that is optionally substituted with one, two, or three substituents independently selected from alkyl, alkylsulfonyl, hydroxyl, cycloalkyl, carboxy, alkoxycarbonyl, hydroxy, alkoxy, halo, haloalkyl, haloalkoxy, amino, alkylamino, dialkylamino, and cyano.

“Optionally substituted heteroaralkyl” means -(alkylene)-R where R is optionally substituted heteroaryl as defined above.

“Optionally substituted heterocyclyl” means heterocyclyl as defined above that is optionally substituted with one, two, or three substituents independently selected from alkyl, alkylsulfonyl, alkylcarbonyl, hydroxyl, cycloalkyl, cycloalkylalkyl, carboxy, alkoxycarbonyl, hydroxy, hydroxyalkyl, alkoxy, alkoxyalkyl, aminoalkyl, cyanoalkyl, halo, haloalkyl, haloalkoxy, and cyano, unless stated otherwise.

“Optionally substituted heterocyclylalkyl” means -(alkylene)-R where R is optionally substituted heterocyclyl as defined above.

“Phosphinyl bicyclic heterocyclyl” means a saturated monovalent fused bicyclic ring of 8 to 12 ring atoms in which one ring atom is phosphorus atom which is substituted with ═(O)R (where R is alkyl as defined herein) and one additional ring atom can be a heteroatom selected from N, O, and S(O)ₙ, where n is an integer selected from 0 to 2, the remaining ring atoms being C, unless stated otherwise. Additionally, one or two ring carbon atoms in the bicyclic ring can optionally be replaced by a —CO— group. More specifically the term phosphinyl bicyclic heterocyclyl includes, but is not limited to,

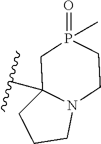 [SMILES: *C12CCCN1CCP(C)(=O)C2]

and the like.

“Phosphinyl bicyclic heterocyclylalkyl” means an -(alkylene)-R radical where R is phosphinyl bicyclic heterocyclyl as defined above. Examples include, but are not limited to,

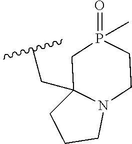 [SMILES: *CC12CCCN1CCP(C)(=O)C2]

and the like.

“Tricyclic heterocyclyl” means a saturated monovalent fused tricyclic ring of 9 to 14, preferably 12 to 14, ring atoms in which one or two ring atoms are heteroatom independently selected from N, O, and S(O)ₙ, where n is an integer from 0 to 2, the remaining ring atoms being C, unless stated otherwise. Additionally, one or two ring carbon atoms in the tricyclic ring can optionally be replaced by a —CO— group. In one embodiment, the tricyclic ring has 11 or 12 ring atoms. The term tricyclic heterocyclyl includes, but is not limited to,

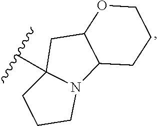 [SMILES: *C12CCCN1C1CCCOC1C2]

and the like.

“Tricyclic heterocyclylalkyl” means a -(alkylene)-R radical where R is tricyclic heterocyclyl as defined above. Examples include, but are not limited to,

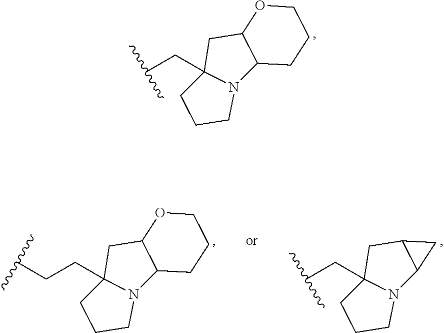 [SMILES: *CC12CCCN1C1CCCOC1C2 ; *CCC12CCCN1C1CCCOC1C2 ; *CC12CCCN1C1CC1C2]

and the like.

The present disclosure also includes protected derivatives of compounds of Formula(IIA1′), (IIA′), (II′), or (II) contain groups such as hydroxy, carboxy, or any group containing a nitrogen atom(s), these groups can be protected with suitable protecting groups. A comprehensive list of suitable protective groups can be found in T.W. Greene, Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes polymorphic forms and deuterated forms of the compound of Formula (IIA′), (IIA′), (II′), or (II) or a pharmaceutically acceptable salt thereof.

The term “prodrug” refers to a compound that is made more active in vivo. Certain compounds Formula (IIA1), (IIA′), (II′), or (II) may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the active compound. Prodrugs are often useful because, in some situations, they may be easier to administer than the compound, or parent drug. They may, for instance, be bioavailable by oral administration whereas the parent drug is not. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxvbenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, P A, 1985, which is incorporated herein by reference in its entirety.

The compounds of Formula (IIA1′), (IIA′), (II′), or (II) may have asymmetric centers. Compounds of Formula (IIA1′), (IIA′), (II′), or (II) containing an asymmetrically substituted atom may be isolated in optically active or racemic forms. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. All chiral, diastereomeric, all mixtures of chiral or diastereomeric forms, and racemic forms are within the scope of this disclosure, unless the specific stereochemistry or isomeric form is specifically indicated. It will also be understood by a person of ordinary skill in the art that when a compound is denoted as (R) stereoisomer, it may contain the corresponding (S) stereoisomer as an impurity and vice versa.

Certain compounds of Formula (IIA1′), (IIA′), (II′), or (II) can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl is substituted, it includes all the positional isomers albeit only a few examples are set forth. Furthermore, all hydrates of a compound of Formula (IIA′), (IIA′), (II′), or (II) are within the scope of this disclosure.

The compounds of Formula (IIAT), (IIA′), (II′), or (II) may also contain unnatural amounts of isotopes at one or more of the atoms that constitute such compounds. Unnatural amounts of an isotope may be defined as ranging from the amount found in nature to an amount 100% of the atom in question, that differ only in the presence of one or more isotopically enriched atoms. Exemplary isotopes that can be incorporated into compounds of the present invention, such as a compound of Formula (IIA1′), (IIA′), (II′), or (II) (and any embodiment thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶C, ¹²³I, and ¹²⁵I, respectively. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds of Formula (IIA1′), (IIA′), (II′), or (II), including in Compound Tables I below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use.

“A pharmaceutically acceptable carrier/excipient” as used in the specification and claims includes both one and more than one such excipient.

“Spiro cycloalkyl” means a saturated bicyclic monovalent ring having 5 to 10 ring atoms in in which the rings are connected through only one atom, the connecting atom is also called the spiroatom, most often a quaternary carbon (“spiro carbon”). Unless stated otherwise, spiro cycloalkyl is optionally substituted with one or two substituents independently selected from alkyl, halo, alkoxy, hydroxy, and cyano. Representative examples include, but are not limited to, spiro[3.3]heptane, spiro[3.4]octane, spiro[3.5]-nonane, and the like.

“Spiro heterocyclyl” means a saturated bicyclic monovalent ring having 6 to 10 ring atoms in which one, two, or three ring atoms are heteroatom selected from N, O, and S(O)ₙ, where n is an integer from 0 to 2, the remaining ring atoms being C and the rings are connected through only one atom, the connecting atom is also called the spiroatom, most often a quaternary carbon (“spiro carbon”). Unless stated otherwise, spiroheterocyclyl is optionally substituted with one or two substituents independently selected from alkyl, halo, alkoxy, hydroxy, and cyano. Examples include, but are not limited to, Representative examples include, but are not limited to, 2,6-diazaspiro[3.3]heptane, 2,6-diazaspiro[3.4]octane, 2-azaspiro[3.4]octane, 2-azaspiro[3.5]-nonane, 2,7-diazaspiro[4.4]nonane, and the like.

“Spiro heterocyclylalkyl” means a -(alkylene)-R radical where R is spiro heterocyclyl ring as defined above e.g., t2-azaspiro[3.4]octylmethyl, 2,6-diazaspiro[3.3]heptylmethyl, 2,6-diazaspiro[3.4]octylethyl, and the like.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

The phrase optionally substituted aryl in the definition of R^{dd} in Formula (IIA1′), (IIA′), (II′), or (II) (and similar phrases used to define other groups in Formula (IIA1′), (IIA′), (II′), or (II)) is intended to cover aryl that is unsubstituted and aryl that is substituted with substituents denoted in the definition thereof in this Application.

Certain structures provided herein are drawn with one or more floating substituents. Unless provided otherwise or otherwise clear from the context, the substituent(s) may be present on any atom of the ring through which they are drawn, where chemically feasible and valency rules permitting. For example, in the structure of Formula (IIA1′):

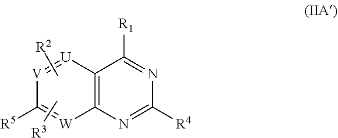

R² and R³ groups are floating substituents and can replace the hydrogen atom of any one of U, V. and W of the

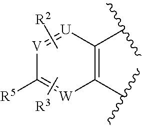

portion of the quinazoline ring when U, V, and W are CH.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a disease or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., delaying, arresting, or reducing the development or severity of the disease or its clinical symptoms; or
(3) relieving the disease, i.e., causing regression of the disease or its clinical symptoms.

In one embodiment, treating or treatment of a disease includes inhibiting the disease, i.e., delaying, arresting or reducing the development or severity of the disease or its clinical symptoms; or relieving the disease, i.e., causing regression of the disease or its clinical symptoms.

A “therapeutically effective amount” means the amount of a compound of the present disclosure or a pharmaceutically acceptable salt thereof that, when administered to a patient for treating a disease, is sufficient to affect such treatment for the disease. The “therapeutically effective amount” will vary depending on the compound, the disease and its severity and the age, weight, etc., of the mammal to be treated. The therapeutically effective amount of a K-Ras inhibitor disclosed herein can be administered to the patient in a single dosage form or multiples thereof. For example, 600 mg dose of a K-Ras inhibitor can be administered in a single 600 mg tablet or two 300 mg tablets.

The terms “inhibiting” and “reducing,” or any variation of these terms in relation of K-Ras G12D, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of K-Ras G12D GTPase activity; a decrease of G12D GTP binding affinity or an increase of G12D GDP binding affinity; an increase of GTP off rate or a decrease of GDP off rate; a decrease of signaling transduction molecules levels downstream in the K-Ras pathway, e.g., a decrease in pERK level; and/or a decrease of K-Ras complex binding to downstream signaling molecules compared to normal.

Representative compounds Formula (IIA1′) are provided in Compound Table 1 below:

Compound Table 1

| CPD | | |
|---|---|---|
| # | Structure | Name |
| 1 | 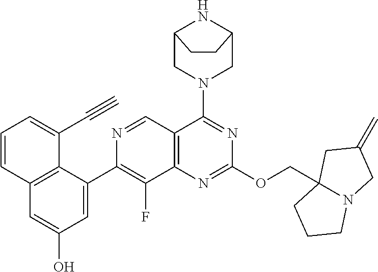 [SMILES: C#Cc1cccc2cc(O)cc(-c3ncc4c(N5CC6CCC(C5)N6)nc(OCC56CCCN5CC(=C)C6)nc4c3F)c12] | 4-(4-((1R,5S)-3,8-diazabicyclo[3.2.1]octan-3-yl)-8- fluoro-2-((2-methylenetetrahydro-1H-pyrrolizin- 7a(5H)-yl)methoxy)pyrido[4,3-d]pyrimidin-7-yl)-5- ethynylnaphthalen-2-ol |
| 2 | 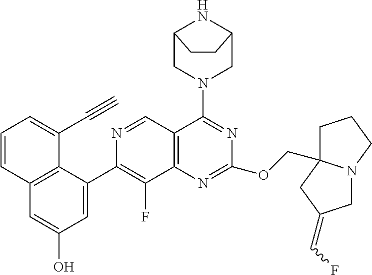 [SMILES: C#Cc1cccc2cc(O)cc(-c3ncc4c(N5CC6CCC(C5)N6)nc(OCC56CCCN5CC(=CF)C6)nc4c3F)c12] | 4-(4-((1R,5S)-3,8-diazabicyclo[3.2.1]octan-3-yl)-8- fluoro-2-((2-(fluoromethylene)tetrahydro-1H- pyrrolizin-7a(5H)-yl)methoxy)pyrido[4,3- d]pyrimidin-7-yl)-5-ethynylnaphthalen-2-ol |
| 3 | 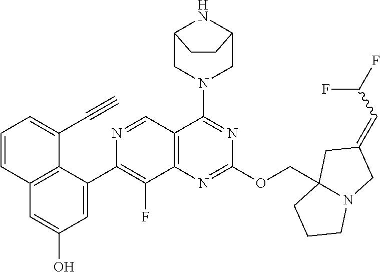 [SMILES: C#Cc1cccc2cc(O)cc(-c3ncc4c(N5CC6CCC(C5)N6)nc(OCC56CCCN5CC(=CC(F)F)C6)nc4c3F)c12] | 4-(4-((1R,5S)-3,8-diazabicyclo[3.2.1]octan-3-yl)-2- ((2-(2,2-difluoroethylidene)-tetrahydro-1H- pyrrolizin-7a(5H)-yl)methoxy)-8-fluoropyrido[4,3- d]pyrimidin-7-yl)-5-ethynylnaphthalen-2-ol |
| 4* | 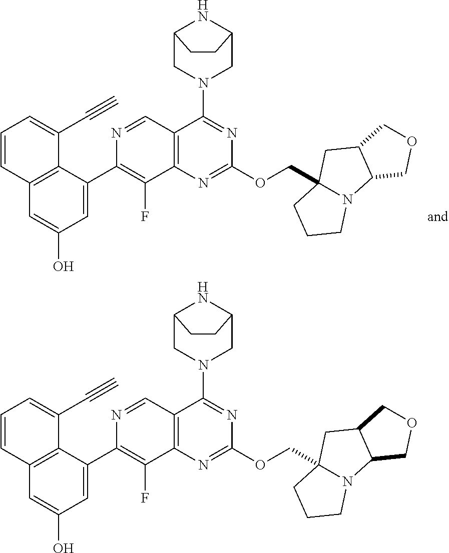 | a mixture of 4-(4-((1R,5S)-3,8-diazabicyclo- [3.2.1]octan-3-yl)-8-fluoro-2-(((3aR,7aR,8aS)- hexahydro-1H-furo[3,4-b]pyrrolizin-7a(5H)- yl)methoxy)-pyrido[4,3-d]pyrimidin-7-yl)-5- ethynylnaphthalen-2-ol and 4-(4-((1R,5S)-3,8-diazabicyclo[3.2.1]octan-3-yl)-8- fluoro-2-(((3aS,7aS,8aR)-hexahydro-1H-furo[3,4- b]pyrrolizin-7a(5H)-yl)methoxy)pyrido[4,3- d]pyrimidin-7-yl)-5-ethynylnaphthalen-2-ol |
| 5 | 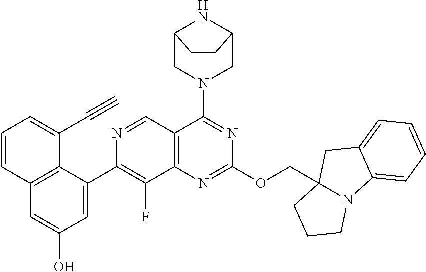 [SMILES: C#Cc1cccc2cc(O)cc(-c3ncc4c(N5CC6CCC(C5)N6)nc(OCC56CCCN5c5ccccc5C6)nc4c3F)c12] | 4-(4-((1R,5S)-3,8-diazabicyclo-[3.2.1]octan-3-yl)-2- ((2,3-dihydro-1H-pyrrolo[1,2-a]-indol-9a(9H)- yl)methoxy)-8-fluoropyrido[4,3-d]-pyrimidin-7-yl)- 5-ethynylnaphthalen-2-ol |
| 6 | 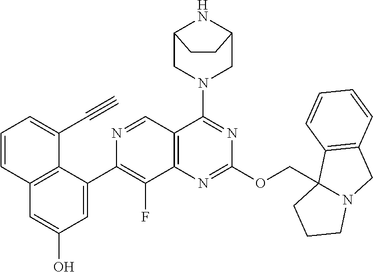 [SMILES: C#Cc1cccc2cc(O)cc(-c3ncc4c(N5CC6CCC(C5)N6)nc(OCC56CCCN5Cc5ccccc56)nc4c3F)c12] | 4-(4-((1R,5S)-3,8-diazabicyclo-[3.2.1]octan-3-yl)-2- ((2,3-dihydro-1H-pyrrolo[2,1-a]-isoindol-9b(5H)- yl)methoxy)-8-fluoropyrido[4,3-d]pyrimidin-7-yl)-5- ethynylnaphthalen-2-ol |
| 7 | 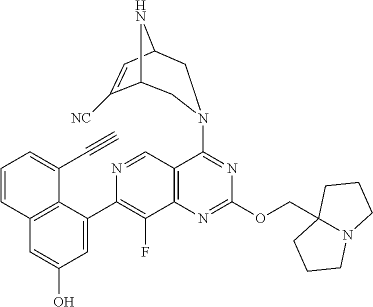 [SMILES: C#Cc1cccc2cc(O)cc(-c3ncc4c(N5CC6C=C(C#N)C(C5)N6)nc(OCC56CCCN5CCC6)nc4c3F)c12] | 3-(7-(8-ethynyl-3-hydroxynaphthalen-1-yl)-8-fluoro- 2-((tetrahydro-1H-pyrrolizin-7a(5H)-yl)methoxy)- pyrido[4,3-d]pyrimidin-4-yl)-3,8- diazabicyclo[3.2.1]oct-6-ene-6-carbonitrile |
| 8 | 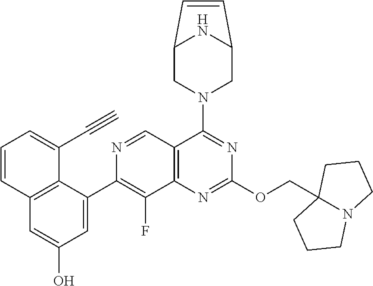 [SMILES: C#Cc1cccc2cc(O)cc(-c3ncc4c(N5CC6C=CC(C5)N6)nc(OCC56CCCN5CCC6)nc4c3F)c12] | 4-(4-(3,8-diazabicyclo-[3.2.1]oct-6-en-3-yl)-8-fluoro- 2-((tetrahydro-1H-pyrrolizin-7a(5H)-yl)methoxy)- pyrido[4,3-d]pyrimidin-7-yl)-5-ethynylnaphthalen-2- ol |
| 9 | 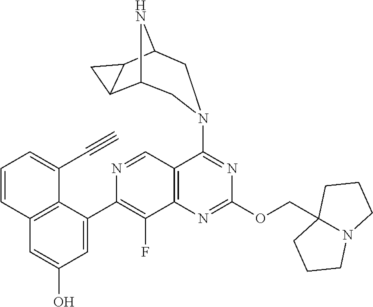 [SMILES: C#Cc1cccc2cc(O)cc(-c3ncc4c(N5CC6NC(C5)C5CC65)nc(OCC56CCCN5CCC6)nc4c3F)c12] | 5-ethynyl-4-(8-fluoro-2-((tetrahydro-1H-pyrrolizin- 7a(5H)-yl)methoxy)-4-(7,9-diazatricyclo[3.3.1.02,4]- nonan-7-yl)-pyrido[4,3-d]pyrimidin-7-yl)naphthalen- 2-o1 |
| 10 | 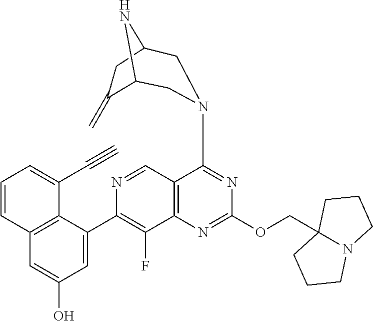 [SMILES: C#Cc1cccc2cc(O)cc(-c3ncc4c(N5CC6CC(=C)C(C5)N6)nc(OCC56CCCN5CCC6)nc4c3F)c12] | 5-ethynyl-4-(8-fluoro-4-(6-methylene-3,8- diazabicyclo[3.2.1]octan-3-yl)-2-((tetrahydro-1H- pyrrolizin-7a(5H)-yl)methoxy)pyrido[4,3- d]pyrimidin-7-yl)naphthalen-2-ol |
| 11 | 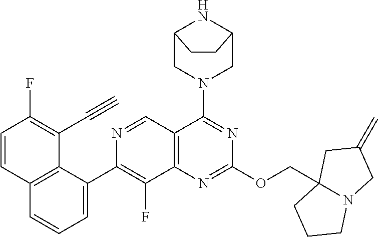 [SMILES: C#Cc1c(F)ccc2cccc(-c3ncc4c(N5CC6CCC(C5)N6)nc(OCC56CCCN5CC(=C)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-((2- methylenetetrahydro-1H-pyrrolizin-7a(5H)- yl)methoxy)pyrido[4,3-d]pyrimidine |
| 12 | 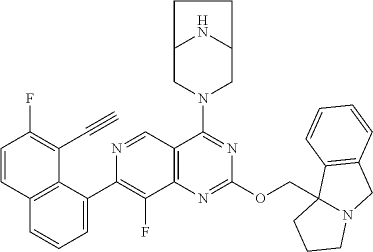 [SMILES: C#Cc1c(F)ccc2cccc(-c3ncc4c(N5CC6CCC(C5)N6)nc(OCC56CCCN5Cc5ccccc56)nc4c3F)c12] | 9b-(((4-(3,8-diazabicyclo[3.2.1]octan-3-yl)-7-(8- ethynyl-7-fluoronaphthalen-1-yl)-8-fluoropyrido[4,3- d]pyrimidin-2-yl)oxy)methyl)-2,3,5,9b-tetrahydro- 1H-pyrrolo[2,1-a]isoindole |
| 13 | 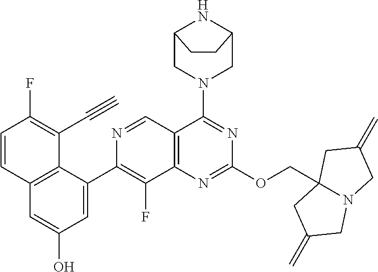 [SMILES: C#Cc1c(F)ccc2cc(O)cc(-c3ncc4c(N5CC6CCC(C5)N6)nc(OCC56CC(=C)CN5CC(=C)C6)nc4c3F)c12] | 4-(4-((1R,5S)-3,8-diazabicyclo[3.2.1]-octan-3-yl)-2- ((2,6-dimethylene-tetrahydro-1H-pyrrolizin-7a(5H)- yl)methoxy)-8-fluoropyrido[4,3-d]-pyrimidin-7-yl)- 5-ethynyl-6-fluoro-naphthalen-2-ol |
| 14 | 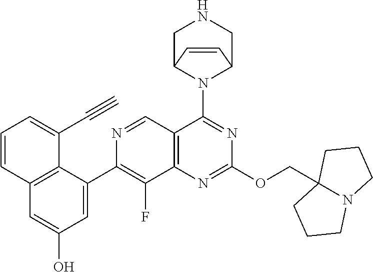 [SMILES: C#Cc1cccc2cc(O)cc(-c3ncc4c(N5C6C=CC5CNC6)nc(OCC56CCCN5CCC6)nc4c3F)c12] | 4-(4-((1R,5S)-3,8-diazabicyclo[3.2.1]oct-6-en-8-yl)- 8-fluoro-2-((tetrahydro-1H-pyrrolizin-7a(5H)- yl)methoxy)pyrido-[4,3-d]pyrimidin-7-yl)-5-ethynyl- naphthalen-2-ol |
| 15 | 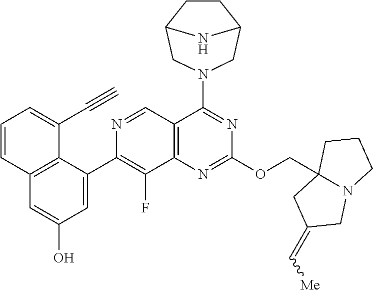 | 4-(4-(3,8-diazabicyclo[3.2.1]octan-3-yl)-2-((2- ethylidenetetrahydro-1H-pyrrolizin-7a(5H)- yl)methoxy)-8-fluoropyrido[4,3-d]pyrimidin-7-yl)-5- ethynylnaphthalen-2-ol |
| 16 | 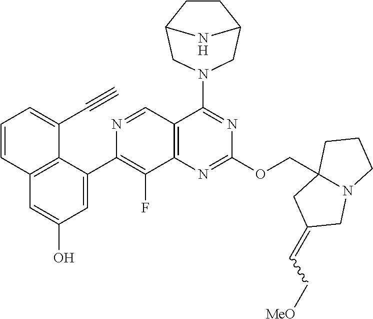 [SMILES: C#Cc1cccc2cc(O)cc(-c3ncc4c(N5CC6CCC(C5)N6)nc(OCC56CCCN5CC(=CCOC)C6)nc4c3F)c12] | 4-(4-(3,8-diazabicyclo[3.2.1]octan-3-yl)-8-fluoro-2- ((2-(2-methoxyethylidene)-tetrahydro-1H-pyrrolizin- 7a(5H)-yl)methoxy)pyrido[4,3-d]pyrimidin-7-yl)-5- ethynylnaphthalen-2-ol |
| 17 | 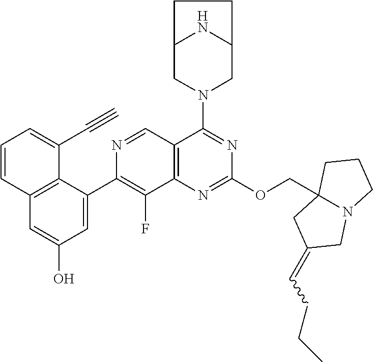 [SMILES: C#Cc1cccc2cc(O)cc(-c3ncc4c(N5CC6CCC(C5)N6)nc(OCC56CCCN5CC(=CCCC)C6)nc4c3F)c12] | 4-(4-(3,8-diazabicyclo[3.2.1]octan-3-yl)-2-((2- butylidenetetrahydro-1H-pyrrolizin-7a(5H)- yl)methoxy)-8-fluoropyrido[4,3-d]pyrimidin-7-yl)-5- ethynylnaphthalen-2-ol |
| 18 | 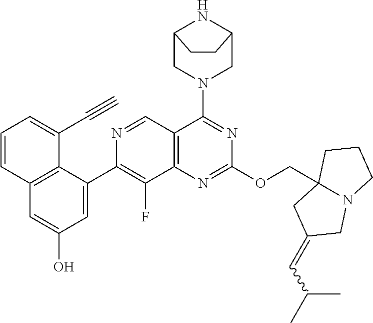 [SMILES: C#Cc1cccc2cc(O)cc(-c3ncc4c(N5CC6CCC(C5)N6)nc(OCC56CCCN5CC(=CC(C)C)C6)nc4c3F)c12] | 4-(4-((1R,5S)-3,8-diazabicyclo-[3.2.1]octan-3-yl)-8- fluoro-2-((2-(2-methylpropylidene)tetrahydro-1H- pyrrolizin-7a(5H)-yl)methoxy)-pyrido[4,3- d]pyrimidin-7-yl)-5-ethynyl-naphthalen-2-ol |
| 19 | 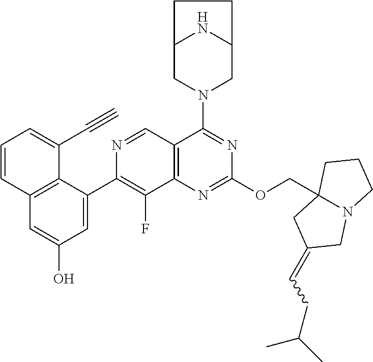 [SMILES: C#Cc1cccc2cc(O)cc(-c3ncc4c(N5CC6CCC(C5)N6)nc(OCC56CCCN5CC(=CCC(C)C)C6)nc4c3F)c12] | 4-(4-(3,8-diazabicyclo[3.2.1]octan-3-yl)-8-fluoro-2- ((2-(3-methylbutylidene)-tetrahydro-1H-pyrrolizin- 7a(5H)-yl)methoxy)pyrido[4,3-d]pyrimidin-7-yl)-5- ethynylnaphthalen-2-ol |
| 20 | 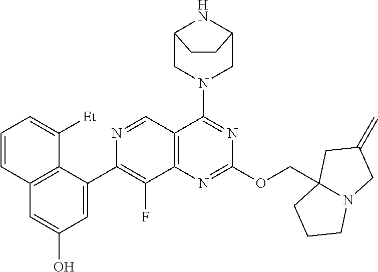 [SMILES: C=C1CN2CCCC2(COc2nc(N3CC4CCC(C3)N4)c3cnc(-c4cc(O)cc5cccc(CC)c45)c(F)c3n2)C1] | 4-(4-((1R,5S)-3,8-diazabicyclo-[3.2.1]octan-3-yl)-8- fluoro-2-((2-methylenetetrahydro-1H-pyrrolizin- 7a(5H)-yl)methoxy)pyrido[4,3-d]-pyrimidin-7-yl)-5- ethylnaphthalen-2-ol |
| 21 | 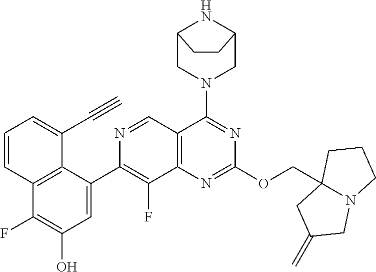 [SMILES: C#Cc1cccc2c(F)c(O)cc(-c3ncc4c(N5CC6CCC(C5)N6)nc(OCC56CCCN5CC(=C)C6)nc4c3F)c12] | 4-(4-((1R,5S)-3,8-diazabicyclo-[3.2.1]octan-3-yl)-8- fluoro-2-((2-methylenetetrahydro-1H-pyrrolizin- 7a(5H)-yl)methoxy)pyrido[4,3-d]-pyrimidin-7-yl)-5- ethynyl-1-fluoro-naphthalen-2-ol |
| 22 | 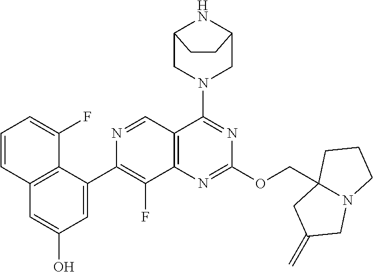 [SMILES: C=C1CN2CCCC2(COc2nc(N3CC4CCC(C3)N4)c3cnc(-c4cc(O)cc5cccc(F)c45)c(F)c3n2)C1] | 4-(4-((1R,5S)-3,8-diazabicyclo-[3.2.1]octan-3-yl)-8- fluoro-2-((2-methylenetetrahydro-1H-pyrrolizin- 7a(5H)-yl)methoxy)pyrido[4,3-d]-pyrimidin-7-yl)-5- fluoronaphthalen-2-ol |
| 23* | 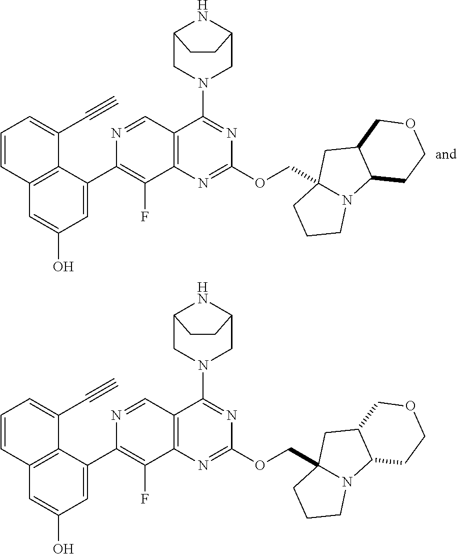 | a mixture of 4-(4-((1R,5S)-3,8-diazabicyclo- [3.2.1]octan-3-yl)-8-fluoro-2-(((4aR,8aS,9aR)- hexahydro-1H,3H-pyrano[3,4-b]pyrrolizin-8a(6H)- yl)methoxy)pyrido[4,3-d]pyrimidin-7-yl)-5- ethynylnaphthalen-2-ol and 4-(4-((1R,5S)-3,8-diazabicyclo-[3.2.1]octan-3-yl)-8- fluoro-2-(((4aS,8aR,9aS)-hexahydro-1H,3H- pyrano[3,4-b]pyrrolizin-8a(6H)- yl)methoxy)pyrido[4,3-d]pyrimidin-7-yl)-5- ethynylnaphthalen-2-ol |
| 24 | 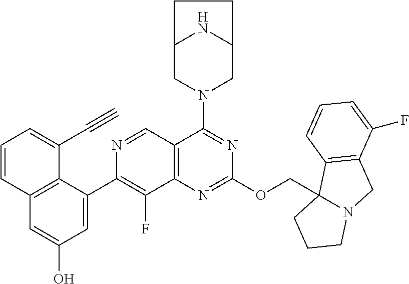 [SMILES: C#Cc1cccc2cc(O)cc(-c3ncc4c(N5CC6CCC(C5)N6)nc(OCC56CCCN5Cc5c(F)cccc56)nc4c3F)c12] | 4-(4-(3,8-diazabicyclo[3.2.1]octan-3-yl)-8-fluoro-2- ((6-fluoro-2,3-dihydro-1H-pyrrolo[2,1-a]isoindol- 9b(5H)-yl)-methoxy)pyrido[4,3-d]pyrimidin-7-yl)-5- ethynylnaphthalen-2-ol |
| 25 | 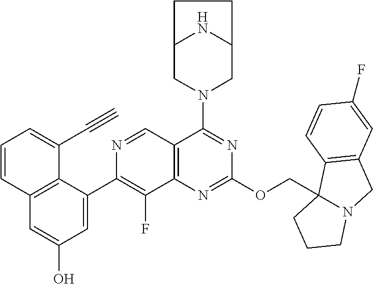 [SMILES: C#Cc1cccc2cc(O)cc(-c3ncc4c(N5CC6CCC(C5)N6)nc(OCC56CCCN5Cc5cc(F)ccc56)nc4c3F)c12] | 4-(4-(3,8-diazabicyclo[3.2.1]octan-3-yl)-8-fluoro-2- ((7-fluoro-2,3-dihydro-1H-pyrrolo[2,1-a]isoindol- 9b(5H)-yl)-methoxy)pyrido[4,3-d]pyrimidin-7-yl)-5- ethynylnaphthalen-2-ol |
| 26 | 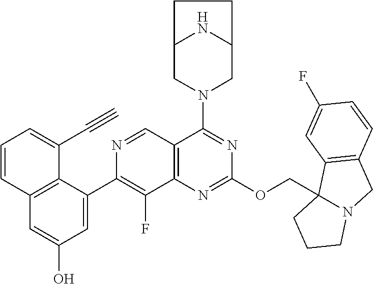 [SMILES: C#Cc1cccc2cc(O)cc(-c3ncc4c(N5CC6CCC(C5)N6)nc(OCC56CCCN5Cc5ccc(F)cc56)nc4c3F)c12] | 4-(4-(3,8-diazabicyclo[3.2.1]octan-3-yl)-8-fluoro-2- ((8-fluoro-2,3-dihydro-1H-pyrrolo[2,1-a]isoindol- 9b(5H)-yl)-methoxy)pyrido[4,3-d]pyrimidin-7-yl)-5- ethynylnaphthalen-2-ol |
| 27 | 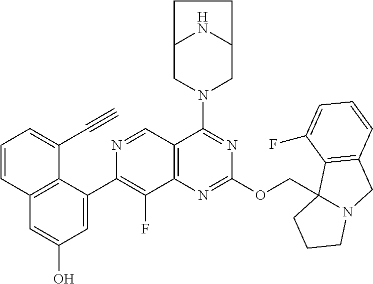 [SMILES: C#Cc1cccc2cc(O)cc(-c3ncc4c(N5CC6CCC(C5)N6)nc(OCC56CCCN5Cc5cccc(F)c56)nc4c3F)c12] | 4-(4-(3,8-diazabicyclo[3.2.1]octan-3-yl)-8-fluoro-2- ((9-fluoro-2,3-dihydro-1H-pyrrolo[2,1-a]isoindol- 9b(5H)-yl)-methoxy)pyrido[4,3-d]pyrimidin-7-yl)-5- ethynylnaphthalen-2-ol |
| 28 | 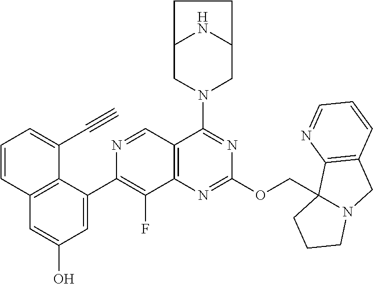 [SMILES: C#Cc1cccc2cc(O)cc(-c3ncc4c(N5CC6CCC(C5)N6)nc(OCC56CCCN5Cc5cccnc56)nc4c3F)c12] | 4-(4-(3,8-diazabicyclo[3.2.1]octan-3-yl)-2-((8,9- dihydro-5H-pyrido[2,3-a]-pyrrolizin-9a(7H)- yl)methoxy)-8-fluoropyrido[4,3-d]pyrimidin-7-yl)-5- ethynylnaphthalen-2-ol |
| 29 | 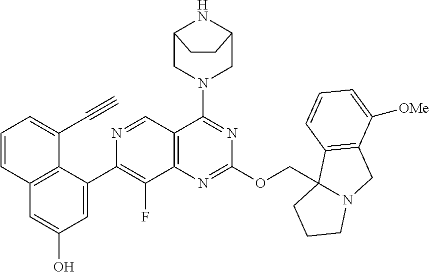 [SMILES: C#Cc1cccc2cc(O)cc(-c3ncc4c(N5CC6CCC(C5)N6)nc(OCC56CCCN5Cc5c(OC)cccc56)nc4c3F)c12] | 4-(4-((1R,5S)-3,8-diazabicyclo-[3.2.1]octan-3-yl)-8- fluoro-2-((6-methoxy-2,3-dihydro-1H-pyrrolo[2,1-a]- isoindol-9b(5H)-yl)methoxy)-pyrido[4,3-d]- pyrimidin-7-yl)-5-ethynylnaphthalen-2-ol |
| 30 | 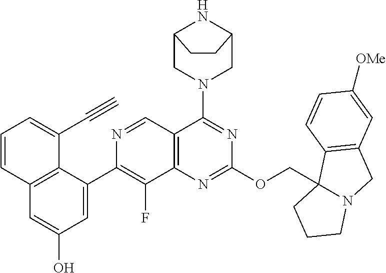 [SMILES: C#Cc1cccc2cc(O)cc(-c3ncc4c(N5CC6CCC(C5)N6)nc(OCC56CCCN5Cc5cc(OC)ccc56)nc4c3F)c12] | 4-(4-((1R,5S)-3,8-diazabicyclo-[3.2.1]octan-3-yl)-8- fluoro-2-((7-methoxy-2,3-dihydro-1H-pyrrolo[2,1- a]isoindol-9b(5H)-yl)methoxy)-pyrido[4,3-d]- pyrimidin-7-yl)-5-ethynylnaphthalen-2-ol |
| 31 | 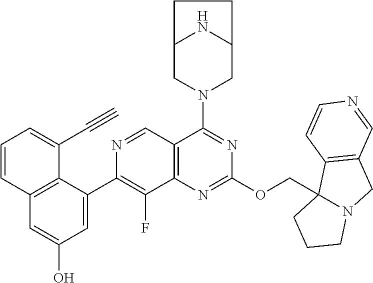 [SMILES: C#Cc1cccc2cc(O)cc(-c3ncc4c(N5CC6CCC(C5)N6)nc(OCC56CCCN5Cc5cnccc56)nc4c3F)c12] | 4-(4-(3,8-diazabicyclo[3.2.1]octan-3-yl)-2-((6,7- dihydro-5H-pyrido[4,3-a]pyrrolizin-4b(9H)- yl)methoxy)-8-fluoropyrido[4,3-d]pyrimidin-7-yl)-5- ethynylnaphthalen-2-ol |
| 32 | 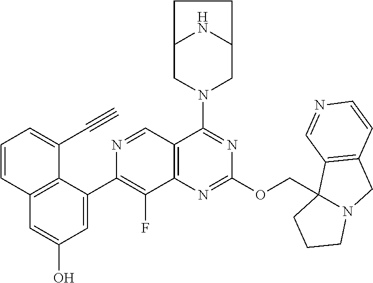 [SMILES: C#Cc1cccc2cc(O)cc(-c3ncc4c(N5CC6CCC(C5)N6)nc(OCC56CCCN5Cc5ccncc56)nc4c3F)c12] | 4-(4-(3,8-diazabicyclo[3.2.1]octan-3-yl)-2-((8,9- dihydro-5H-pyrido[3,4-a]pyrrolizin-9a(7H)- yl)methoxy)-8-fluoropyrido[4,3-d]pyrimidin-7-yl)-5- ethynylnaphthalen-2-ol |
| 33 | 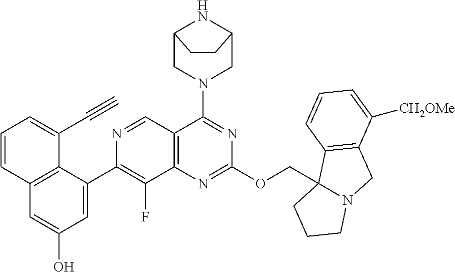 | 4-(4-((1R,5S)-3,8-diazabicyclo-[3.2.1]octan-3-yl)-8- fluoro-2-((6-(methoxymethyl)-2,3-dihydro-1H- pyrrolo[2,1-a]isoindol-9b(5H)-yl)methoxy)- pyrido[4,3-d]pyrimidin-7-yl)-5-ethynylnaphthalen-2- ol |
| 34 | 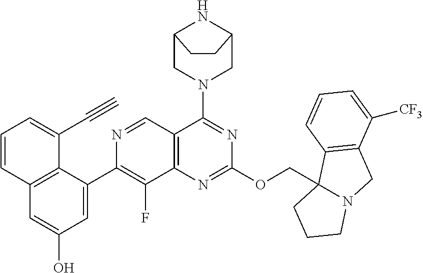 [SMILES: C#Cc1cccc2cc(O)cc(-c3ncc4c(N5CC6CCC(C5)N6)nc(OCC56CCCN5Cc5c(C(F)(F)F)cccc56)nc4c3F)c12] | 4-(4-((1R,5S)-3,8-diazabicyclo[3.2.1]octan-3-yl)-8- fluoro-2-((6-(trifluoromethyl)-2,3-dihydro-1H- pyrrolo[2,1-a]isoindol-9b(5H)-yl)methoxy)pyrido- [4,3-d]pyrimidin-7-yl)-5-ethynylnaphthalen-2-ol |
| 35a* | 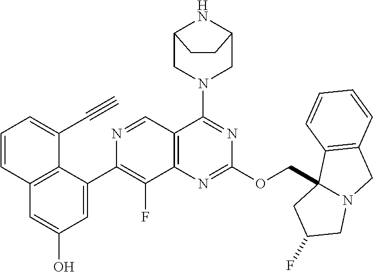 | 4-(4-((1R,5S)-3,8-diazabicyclo-[3.2.1]octan-3-yl)-8- fluoro-2-(((2R,9bR)-2-fluoro-2,3-dihydro-1H- pyrrolo[2,1-a]isoindol-9b(5H)-yl)methoxy)- pyrido[4,3-d]pyrimidin-7-yl)-5-ethynylnaphthalen-2- ol |
| 35b* | 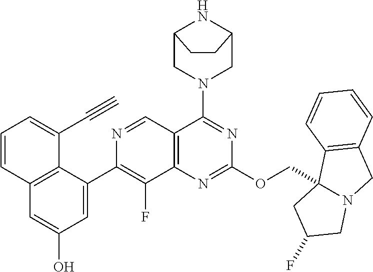 | 4-(4-((1R,5S)-3,8-diazabicyclo-[3.2.1]octan-3-yl)-8- fluoro-2-(((2R,9bS)-2-fluoro-2,3-dihydro-1H- pyrrolo[2,1-a]isoindol-9b(5H)-yl)methoxy)- pyrido[4,3-d]pyrimidin-7-yl)-5-ethynylnaphthalen-2- ol |
| 36 | 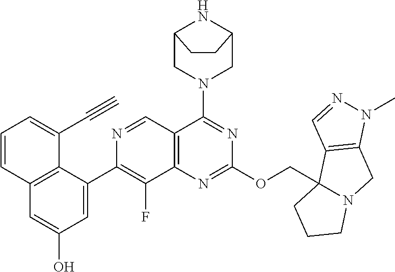 [SMILES: C#Cc1cccc2cc(O)cc(-c3ncc4c(N5CC6CCC(C5)N6)nc(OCC56CCCN5Cc5c6cnn5C)nc4c3F)c12] | 4-(4-((1R,5S)-3,8-diazabicyclo[3.2.1]octan-3-yl)-8- fluoro-2-((1-methyl-1,5,6,8-tetrahydropyrazolo[4,3- a]pyrrolizin-3b(4H)-yl)methoxy)pyrido[4,3- d]pyrimidin-7-yl)-5-ethynylnaphthalen-2-ol |
| 37* | 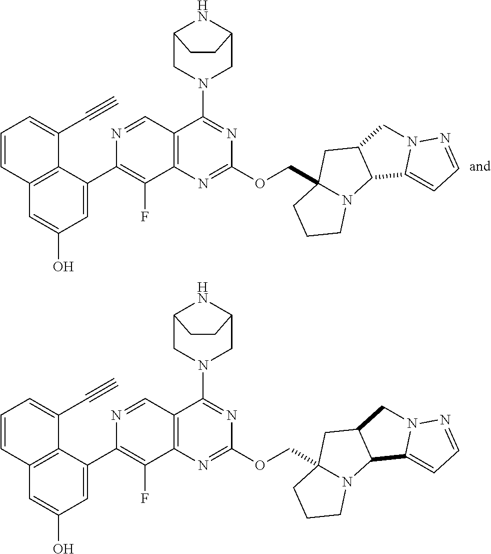 | a mixture of 4-(4-((1R,5S)-3,8-diazabicyclo- [3.2.1]octan-3-yl)-8-fluoro-2-(((3bR,7aR,8aR)- 6,7,8,8a-tetrahydro-5H,9H-pyrazolo- [1′,5′:1,5]pyrrolo[3,4-b]-pyrrolizin-7a(3bH)- yl)methoxy)-pyrido[4,3-d]pyrimidin-7-yl)-5- ethynylnaphthalen-2-ol and 4-(4-((1R,5S)-3,8-diazabicyclo-[3.2.1]octan-3-yl)-8- fluoro-2-(((3bS,7aS,8aS)-6,7,8,8a-tetrahydro-5H,9H- pyrazolo[1′,5′:1,5]pyrrolo[3,4-b]pyrrolizin-7a(3bH)- yl)methoxy)-pyrido[4,3-d]pyrimidin-7-yl)-5- ethynylnaphthalen-2-ol |
| 38* | 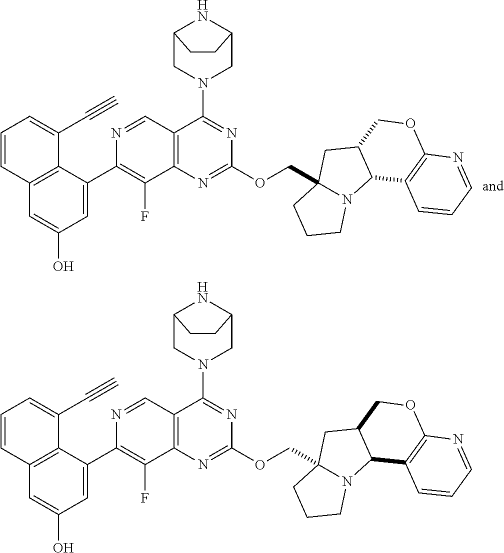 | a mixture of 4-(4-((1R,5S)-3,8-diazabicyclo- [3.2.1]octan-3-yl)-8-fluoro-2-(((6aS,7aR,11aR)- 6a,9,10,11a-tetrahydro-6H,7H-pyrido-[3′,2′:5,6]- pyrano[3,4-b]pyrrolizin-7a(8H)- yl)methoxy)pyrido[4,3-d]-pyrimidin-7-yl)-5- ethynylnaphthalen-2-ol and 4-(4-((1R,5S)-3,8-diazabicyclo-[3.2.1]-octan-3-yl)-8- fluoro-2-(((6aR,7aS,11aS)-6a,9,10,11a-tetrahydro- 6H,7H-pyrido[3′,2′:5,6]pyrano[3,4-b]pyrrolizin- 7a(8H)-yl)methoxy)-pyrido[4,3-d]pyrimidin-7-yl)-5- ethynylnaphthalen-2-ol |
| 39* | 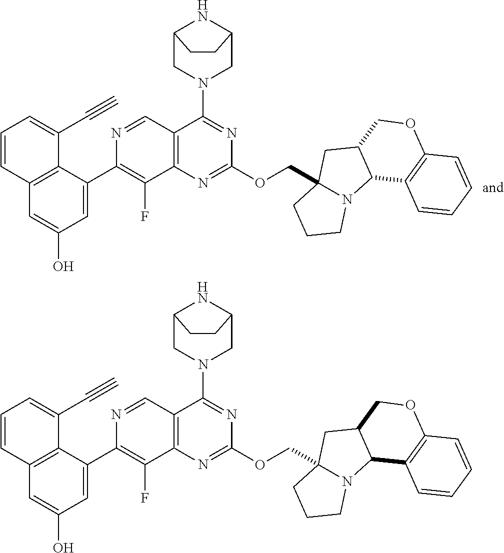 | a mixture of 4-(4-((1R,5S)-3,8-diazabicyclo- [3.2.1]octan-3-yl)-8-fluoro-2-(((6aS,7aR,11aR)- 6a,9,10,11a-tetrahydro-6H,7H-chromeno[3,4- b]pyrrolizin-7a(8H)-yl)methoxy)-pyrido[4,3- d]pyrimidin-7-yl)-5-ethynylnaphthalen-2-ol and 4-(4-((1R,5S)-3,8-diazabicyclo[3.2.1]-octan-3-yl)-8- fluoro-2-(((6aR,7aS,11aS)-6a,9,10,11a-tetrahydro- 6H,7H-chromeno[3,4-b]pyrrolizin-7a(8H)- yl)methoxy)pyrido[4,3-d]pyrimidin-7-yl)-5- ethynylnaphthalen-2-ol |
| 40* | 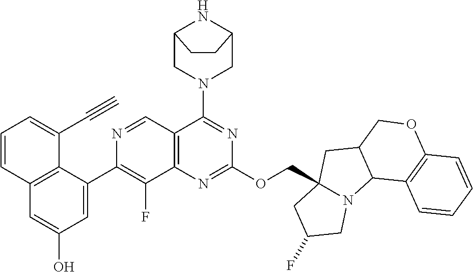 [SMILES: C#Cc1cccc2cc(O)cc(-c3ncc4c(N5CC6CCC(C5)N6)nc(OC[C@@]56CC7COc8ccccc8C7N5C[C@H](F)C6)nc4c3F)c12] | 4-(4-((1R,5S)-3,8-diazabicyclo[3.2.1]octan-3-yl)-8- fluoro-2-(((9R)-9-fluoro-6a,9,10,11a-tetrahydro- 6H,7H-chromeno[3,4-b]pyrrolizin-7a(8H)- yl)methoxy)pyrido[4,3-d]pyrimidin-7-yl)-5- ethynylnaphthalen-2-ol |
| 41* | 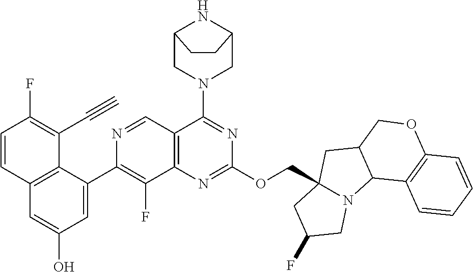 [SMILES: C#Cc1c(F)ccc2cc(O)cc(-c3ncc4c(N5CC6CCC(C5)N6)nc(OC[C@@]56CC7COc8ccccc8C7N5C[C@@H](F)C6)nc4c3F)c12] | 4-(4-((1R,5S)-3,8-diazabicyclo[3.2.1]octan-3-yl)-8- fluoro-2-(((9S)-9-fluoro-6a,9,10,11a-tetrahydro- 6H,7H-chromeno[3,4-b]pyrrolizin-7a(8H)- yl)methoxy)pyrido[4,3-d]pyrimidin-7-yl)-5-ethynyl- 6-fluoronaphthalen-2-ol |
| 42 | 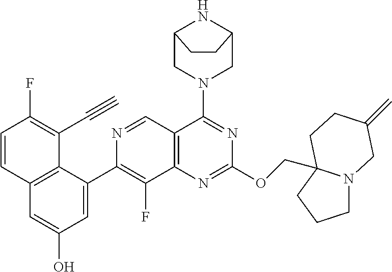 [SMILES: C#Cc1c(F)ccc2cc(O)cc(-c3ncc4c(N5CC6CCC(C5)N6)nc(OCC56CCCN5CC(=C)CC6)nc4c3F)c12] | 4-(4-((1R,5S)-3,8-diazabicyclo[3.2.1]octan-3-yl)-8- fluoro-2-((6-methylenehexahydroindolizin-8a(1H)- yl)methoxy)pyrido[4,3-d]pyrimidin-7-yl)-5-ethynyl- 6-fluoronaphthalen-2-ol |
| 43 | 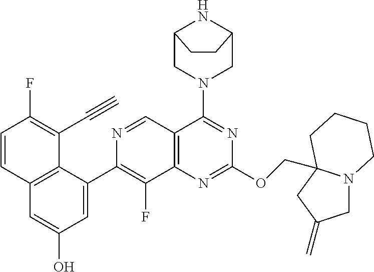 [SMILES: C#Cc1c(F)ccc2cc(O)cc(-c3ncc4c(N5CC6CCC(C5)N6)nc(OCC56CCCCN5CC(=C)C6)nc4c3F)c12] | 4-(4-((1R,5S)-3,8-diazabicyclo[3.2.1]octan-3-yl)-8- fluoro-2-((2-methylenehexahydroindolizin-8a(1H)- yl)methoxy)pyrido[4,3-d]pyrimidin-7-yl)-5-ethynyl- 6-fluoronaphthalen-2-ol |
| 44 | 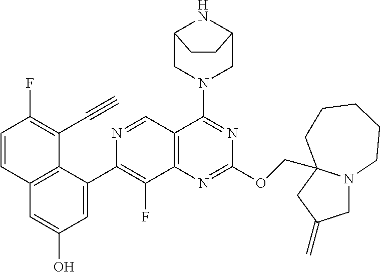 [SMILES: C#Cc1c(F)ccc2cc(O)cc(-c3ncc4c(N5CC6CCC(C5)N6)nc(OCC56CCCCCN5CC(=C)C6)nc4c3F)c12] | 4-(4-((1R,5S)-3,8-diazabicyclo[3.2.1]octan-3-yl)-8- fluoro-2-((2-methylenehexahydro-1H-pyrrolo[1,2- a]azepin-9a(5H)-yl)methoxy)pyrido[4,3-d]pyrimidin- 7-yl)-5-ethynyl-6-fluoronaphthalen-2-ol |
| 46 | 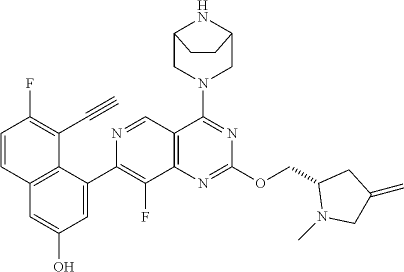 | 4-(4-((1R,5S)-3,8-diazabicyclo[3.2.1]octan-3-yl)-8- fluoro-2-(((S)-1-methyl-4-methylenepyrrolidin-2- yl)methoxy)pyrido[4,3-d]pyrimidin-7-yl)-5-ethynyl- 6-fluoronaphthalen-2-ol |
| 47 | 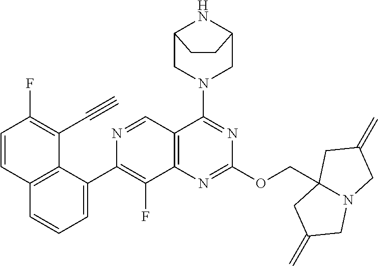 [SMILES: C#Cc1c(F)ccc2cccc(-c3ncc4c(N5CC6CCC(C5)N6)nc(OCC56CC(=C)CN5CC(=C)C6)nc4c3F)c12] | 4-((1R,5S)-3,8-diazabicyclo[3.2.1]octan-3-yl)-2- ((2,6-dimethylenetetrahydro-1H-pyrrolizin-7a(5H)- yl)methoxy)-7-(8-ethynyl-7-fluoronaphthalen-1-yl)- 8-fluoropyrido[4,3-d]pyrimidine |
| 48 | 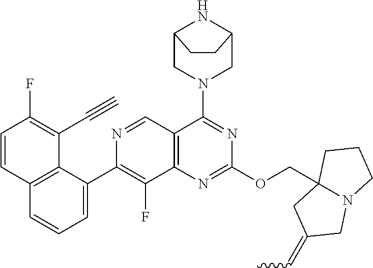 | 4-((1R,5S)-3,8-diazabicyclo[3.2.1]octan-3-yl)-2-((2- ethylidenetetrahydro-1H-pyrrolizin-7a(5H)- yl)methoxy)-7-(8-ethynyl-7-fluoronaphthalen-1-yl)- 8-fluoropyrido[4,3-d]pyrimidine |
| 49* | 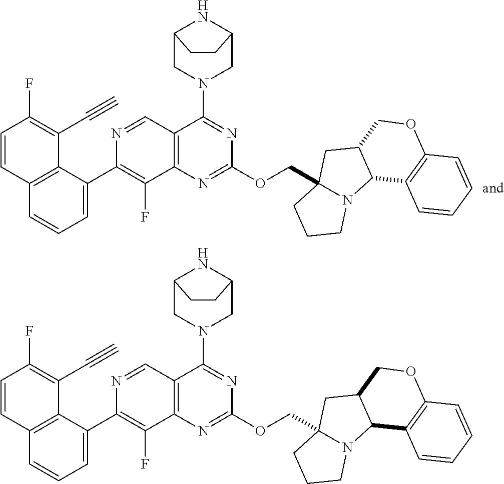 | a mixture of (6aS,7aR,11aR)-7a-(((4-((1R,5S)-3,8- diazabicyclo[3.2.1]octan-3-yl)-7-(8-ethynyl-7- fluoronaphthalen-1-yl)-8-fluoropyrido[4,3- d]pyrimidin-2-yl)oxy)methyl)-6a,7a,8,9,10,11a- hexahydro-6H,7H-chromeno[3,4-b]-pyrrolizine and (6aR,7aS,11aS)-7a-(((4-((1R,5S)-3,8- diazabicyclo[3.2.1]octan-3-yl)-7-(8-ethynyl-7- fluoronaphthalen-1-yl)-8-fluoropyrido[4,3- d]pyrimidin-2-yl)-oxy)methyl)-6a,7a,8,9,10,11a- hexahydro-6H,7H-chromeno[3,4-b]-pyrrolizine |
| 50 | 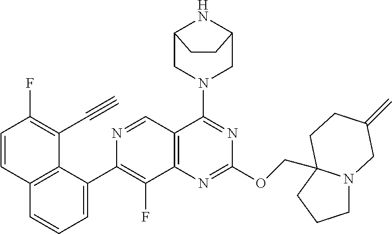 [SMILES: C#Cc1c(F)ccc2cccc(-c3ncc4c(N5CC6CCC(C5)N6)nc(OCC56CCCN5CC(=C)CC6)nc4c3F)c12] | 4-((1R,5S)-3,8-diazabicyclo[3.2.1]octan-3-yl)-7-(8- ethynyl-7-fluoronaphthalen-1-yl)-8-fluoro-2-((6- methylenehexahydroindolizin-8a(1H)-yl)methoxy)- pyrido[4,3-d]pyrimidine |
| 51 | 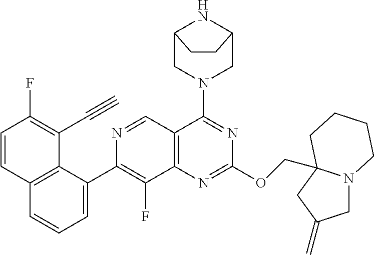 [SMILES: C#Cc1c(F)ccc2cccc(-c3ncc4c(N5CC6CCC(C5)N6)nc(OCC56CCCCN5CC(=C)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-((2- methylenehexahydroindolizin-8a(1H)- yl)methoxy)pyrido[4,3-d]pyrimidine |
| 52 | 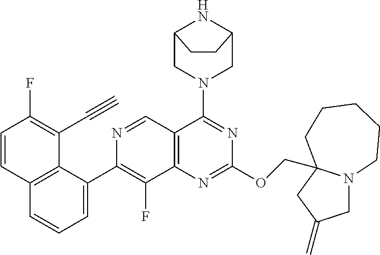 [SMILES: C#Cc1c(F)ccc2cccc(-c3ncc4c(N5CC6CCC(C5)N6)nc(OCC56CCCCCN5CC(=C)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-((2- methylenehexahydro-1H-pyrrolo[1,2-a]azepin- 9a(5H)-yl)methoxy)pyrido[4,3-d]pyrimidine |
| 53 | 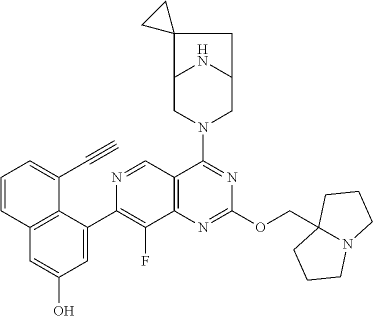 [SMILES: C#Cc1cccc2cc(O)cc(-c3ncc4c(N5CC6CC7(CC7)C(C5)N6)nc(OCC56CCCN5CCC6)nc4c3F)c12] | 5-ethynyl-4-(8-fluoro-4-(3,8- diazaspiro[bicyclo[3.2.1]octane-6,1′-cyclopropan]-3- yl)-2-((tetrahydro-1H-pyrrolizin-7a(5H)- yl)methoxy)pyrido[4,3-d]pyrimidin-7-yl)naphthalen- 2-ol |
| 54 | 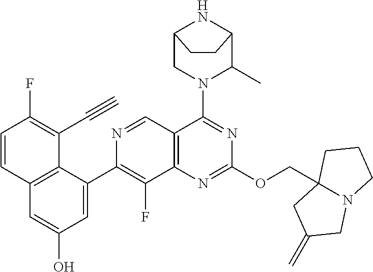 [SMILES: C#Cc1c(F)ccc2cc(O)cc(-c3ncc4c(N5CC6CCC(N6)C5C)nc(OCC56CCCN5CC(=C)C6)nc4c3F)c12] | 5-ethynyl-6-fluoro-4-(8-fluoro-4-((1S,5R)-2-methyl- 3,8-diazabicyclo[3.2.1]octan-3-yl)-2-((2-methylene- tetrahydro-1H-pyrrolizin-7a(5H)-yl)methoxy)- pyrido[4,3-d]pyrimidin-7-yl)naphthalen-2-ol |
| 55 | 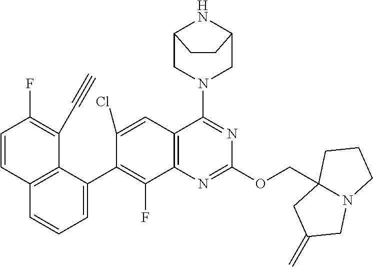 [SMILES: C#Cc1c(F)ccc2cccc(-c3c(Cl)cc4c(N5CC6CCC(C5)N6)nc(OCC56CCCN5CC(=C)C6)nc4c3F)c12] | 4-((1R,5S)-3,8-diazabicyclo[3.2.1]octan-3-yl)-6- chloro-7-(8-ethynyl-7-fluoronaphthalen-1-yl)-8- fluoro-2-((2-methylenetetrahydro-1H-pyrrolizin- 7a(5H)-yl)methoxy)quinazoline |
| 56 | 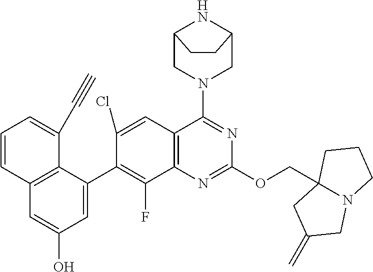 [SMILES: C#Cc1cccc2cc(O)cc(-c3c(Cl)cc4c(N5CC6CCC(C5)N6)nc(OCC56CCCN5CC(=C)C6)nc4c3F)c12] | 4-(4-((1R,5S)-3,8-diazabicyclo[3.2.1]octan-3-yl)-6- chloro-8-fluoro-2-((2-methylenetetrahydro-1H- pyrrolizin-7a(5H)-yl)methoxy)quinazolin-7-yl)-5- ethynylnaphthalen-2-ol |

*Stereochemistry not confirmed.

Contemplated compounds Formula (IIA1′) are provided in Compound Table 2 below:

Compound Table 2

| Cpd. # | Structure | Name |
|---|---|---|
| II-1 | 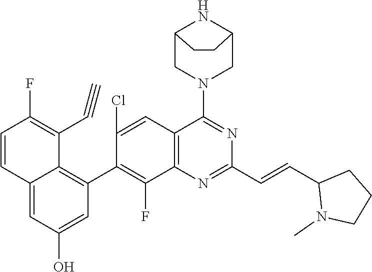 [SMILES: C#Cc1c(F)ccc2cc(O)cc(-c3c(Cl)cc4c(N5CC6CCC(C5)N6)nc(/C=C/C5CCCN5C)nc4c3F)c12] | 4-(4-((1R,5S)-3,8-diazabicyclo[3.2.1]octan-3-yl)-6-chloro- 8-fluoro-2-((E)-2-(1-methylpyrrolidin-2- yl)vinyl)quinazolin-7-yl)-5-ethynyl-6-fluoro-naphthalen-2- ol |
| II-2 | 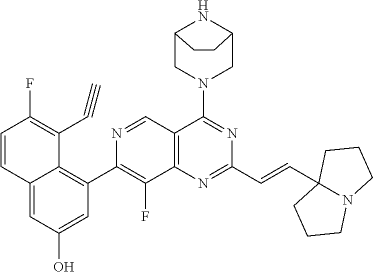 [SMILES: C#Cc1c(F)ccc2cc(O)cc(-c3ncc4c(N5CC6CCC(C5)N6)nc(/C=C/C56CCCN5CCC6)nc4c3F)c12] | 4-(4-((1R,5S)-3,8-diazabicyclo[3.2.1]octan-3-yl)-8-fluoro- 2-((E)-2-(tetrahydro-1H-pyrrolizin-7a(5H)- yl)vinyl)pyrido[4,3-d]pyrimidin-7-yl)-5-ethynyl-6- fluoronaphthalen-2-ol |
| II-3 | 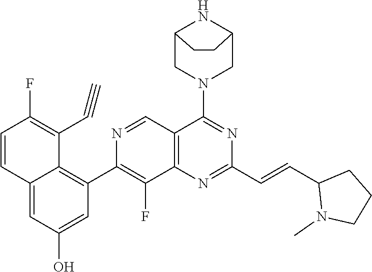 [SMILES: C#Cc1c(F)ccc2cc(O)cc(-c3ncc4c(N5CC6CCC(C5)N6)nc(/C=C/C5CCCN5C)nc4c3F)c12] | 4-(4-((1R,5S)-3,8-diazabicyclo[3.2.1]octan-3-yl)-8-fluoro- 2-((E)-2-(1-methylpyrrolidin-2-yl)vinyl)-pyrido[4,3- d]pyrimidin-7-yl)-5-ethynyl-6-fluoro-naphthalen-2-ol |
| II-4 | 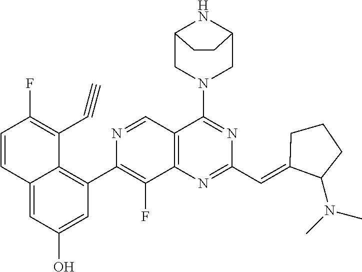 [SMILES: C#Cc1c(F)ccc2cc(O)cc(-c3ncc4c(N5CC6CCC(C5)N6)nc(/C=C5\CCCC5N(C)C)nc4c3F)c12] | 4-(4-((1R,5S)-3,8-diazabicyclo[3.2.1]octan-3-yl)-2-((E)-(2- (dimethylamino)cyclopentylidene)methyl)-8- fluoropyrido[4,3-d]pyrimidin-7-yl)-5-ethynyl-6- fluoronaphthalen-2-ol |
| II-5 | 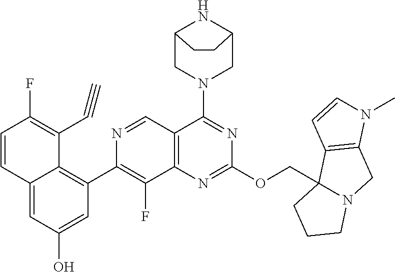 [SMILES: C#Cc1c(F)ccc2cc(O)cc(-c3ncc4c(N5CC6CCC(C5)N6)nc(OCC56CCCN5Cc5c6ccn5C)nc4c3F)c12] | 4-(4-((1R,5S)-3,8-diazabicyclo[3.2.1]octan-3-yl)-8-fluoro- 2-((1-methyl-1,5,6,8-tetrahydropyrrolo[3,2-a]pyrrolizin- 3b(4H)-yl)methoxy)pyrido[4,3-d]-pyrimidin-7-yl)-5- ethynyl-6-fluoronaphthalen-2-ol |
| II-6 | 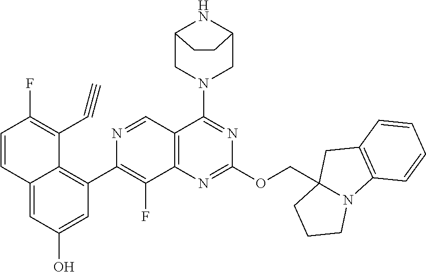 [SMILES: C#Cc1c(F)ccc2cc(O)cc(-c3ncc4c(N5CC6CCC(C5)N6)nc(OCC56CCCN5c5ccccc5C6)nc4c3F)c12] | 4-(4-((1R,5S)-3,8-diazabicyclo[3.2.1]octan-3-yl)-2-((2,3- dihydro-1H-pyrrolo[1,2-a]indol-9a(9H)-yl)-methoxy)-8- fluoropyrido[4,3-d]pyrimidin-7-yl)-5-ethynyl-6- fluoronaphthalen-2-ol |
| II-7 | 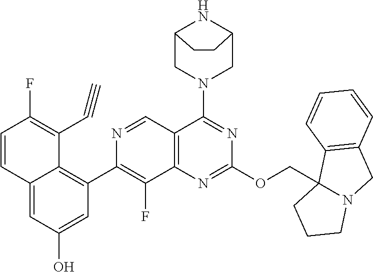 [SMILES: C#Cc1c(F)ccc2cc(O)cc(-c3ncc4c(N5CC6CCC(C5)N6)nc(OCC56CCCN5Cc5ccccc56)nc4c3F)c12] | 4-(4-((1R,5S)-3,8-diazabicyclo[3.2.1]octan-3-yl)-2-((2,3- dihydro-1H-pyrrolo[2,1-a]isoindol-9b(5H)-yl)methoxy)-8- fluoropyrido[4,3-d]pyrimidin-7-yl)-5-ethynyl-6- fluoronaphthalen-2-ol |
| II-8 | 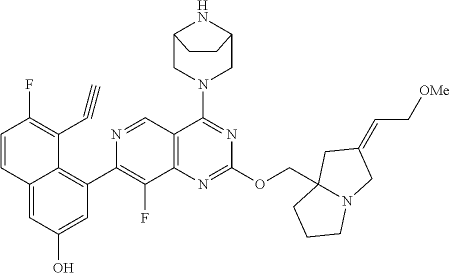 [SMILES: C#Cc1c(F)ccc2cc(O)cc(-c3ncc4c(N5CC6CCC(C5)N6)nc(OCC56CCCN5C/C(=C\COC)C6)nc4c3F)c12] | 4-(4-((1R,5S)-3,8-diazabicyclo[3.2.1]octan-3-yl)-8-fluoro- 2-(((Z)-2-(2-methoxyethylidene)tetrahydro-1H-pyrrolizin- 7a(5H)-yl)methoxy)pyrido[4,3-d]pyrimidin-7-yl)-5- ethynyl-6-fluoronaphthalen-2-ol |
| II-9 | 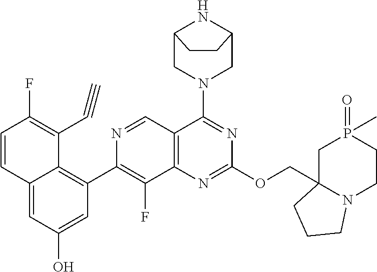 [SMILES: C#Cc1c(F)ccc2cc(O)cc(-c3ncc4c(N5CC6CCC(C5)N6)nc(OCC56CCCN5CCP(C)(=O)C6)nc4c3F)c12] | 8a-(((4-((1R,5S)-3,8-diazabicyclo[3.2.1]octan-3-yl)-7-(8- ethynyl-7-fluoro-3-hydroxynaphthalen-1-yl)-8- fluoropyrido[4,3-d]pyrimidin-2-yl)oxy)methyl)-2- methylheptahydropyrrolo[1,2-a][1,4]azaphosphinine 2- oxide |
| II-10 | 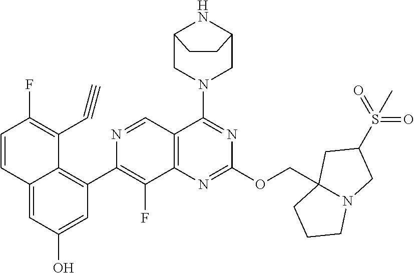 [SMILES: C#Cc1c(F)ccc2cc(O)cc(-c3ncc4c(N5CC6CCC(C5)N6)nc(OCC56CCCN5CC(S(C)(=O)=O)C6)nc4c3F)c12] | 4-(4-((1R,5S)-3,8-diazabicyclo[3.2.1]octan-3-yl)-8-fluoro- 2-((2-(methylsulfonyl)tetrahydro-1H-pyrrolizin-7a(5H)- yl)methoxy)pyrido[4,3-d]-pyrimidin-7-yl)-5-ethynyl-6- fluoronaphthalen-2-ol |
| II-11 | 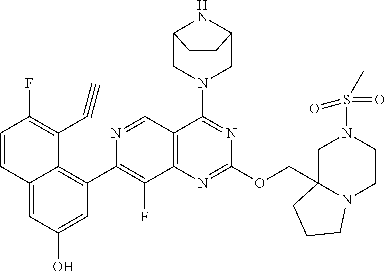 [SMILES: C#Cc1c(F)ccc2cc(O)cc(-c3ncc4c(N5CC6CCC(C5)N6)nc(OCC56CCCN5CCN(S(C)(=O)=O)C6)nc4c3F)c12] | 4-(4-((1R,5S)-3,8-diazabicyclo[3.2.1]octan-3-yl)-8-fluoro- 2-((2-(methylsulfonyl)hexahydropyrrolo[1,2-a]-pyrazin- 8a(6H)-yl)methoxy)pyrido[4,3-d]-pyrimidin-7-yl)-5- ethynyl-6-fluoronaphthalen-2-ol |
| II-12 | 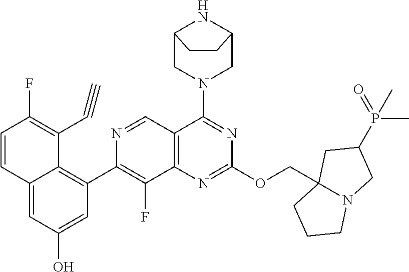 [SMILES: C#Cc1c(F)ccc2cc(O)cc(-c3ncc4c(N5CC6CCC(C5)N6)nc(OCC56CCCN5CC(P(C)(C)=O)C6)nc4c3F)c12] | (7a-(((4-((1R,5S)-3,8-diazabicyclo[3.2.1]octan-3-yl)-7-(8- ethynyl-7-fluoro-3-hydroxynaphthalen-1-yl)-8- fluoropyrido[4,3-d]pyrimidin-2-yl)oxy)methyl)- hexahydro-1H-pyrrolizin-2-yl)dimethylphosphine oxide |
| II-13 | 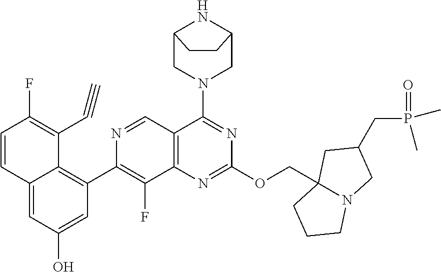 [SMILES: C#Cc1c(F)ccc2cc(O)cc(-c3ncc4c(N5CC6CCC(C5)N6)nc(OCC56CCCN5CC(CP(C)(C)=O)C6)nc4c3F)c12] | ((7a-(((4-((1R,5S)-3,8-diazabicyclo[3.2.1]octan-3-yl)-7-(8- ethynyl-7-fluoro-3-hydroxynaphthalen-1-yl)-8- fluoropyrido[4,3-d]pyrimidin-2-yl)oxy)methyl)- hexahydro-1H-pyrrolizin-2-yl)methyl)dimethylphosphine oxide |
| II-14 | 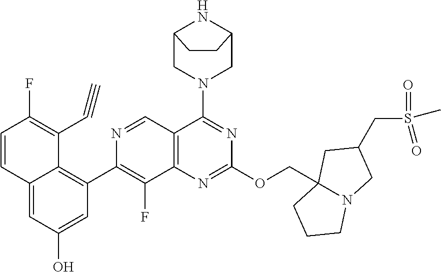 [SMILES: C#Cc1c(F)ccc2cc(O)cc(-c3ncc4c(N5CC6CCC(C5)N6)nc(OCC56CCCN5CC(CS(C)(=O)=O)C6)nc4c3F)c12] | 4-(4-((1R,5S)-3,8-diazabicyclo[3.2.1]octan-3-yl)-8-fluoro- 2-((2-((methylsulfonyl)methyl)tetrahydro-1H-pyrrolizin- 7a(5H)-yl)methoxy)pyrido[4,3-d]-pyrimidin-7-yl)-5- ethynyl-6-fluoronaphthalen-2-ol |
| II-15 | 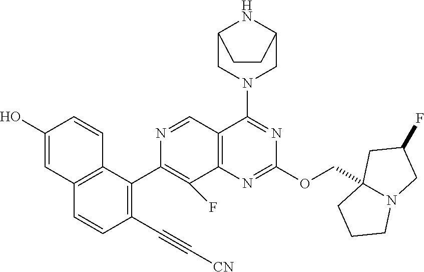 | 3-(1-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]-pyrimidin-7-yl)-6- hydroxynaphthalen-2-yl)-propiolonitrile |
| II-16 | 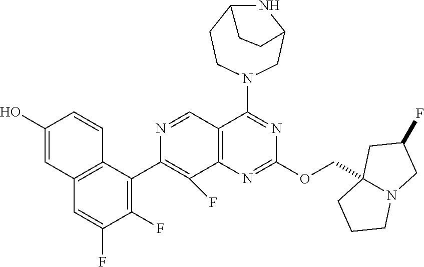 | 5-(4-(3,9-diazabicyclo[4.2.1]nonan-3-yl)-8-fluoro-2- (((2R,7aS)-2-fluorotetrahydro-1H-pyrrolizin-7a(5H)- yl)methoxy)pyrido[4,3-d]pyrimidin-7-yl)-6,7- difluoronaphthalen-2-ol |
| II-17 | 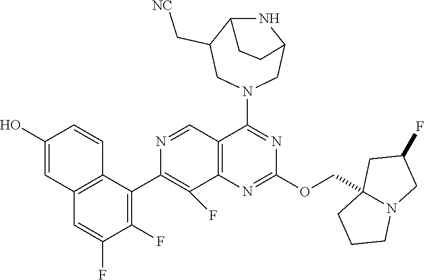 | 2-(3-(7-(2,3-difluoro-6-hydroxynaphthalen-1-yl)-8-fluoro- 2-(((2R,7aS)-2-fluorotetrahydro-1H-pyrrolizin-7a(5H)- yl)methoxy)pyrido[4,3-d]-pyrimidin-4-yl)-3,9- diazabicyclo[4.2.1]nonan-5-yl)acetonitrile |
| II-18 | 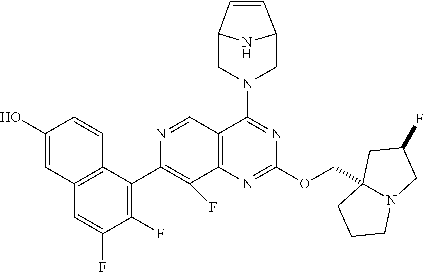 | 5-(4-((1R,5S)-3,8-diazabicyclo[3.2.1]oct-6-en-3-yl)-8- fluoro-2-(((2R,7aS)-2-fluorotetrahydro-1H-pyrrolizin- 7a(5H)-yl)methoxy)pyrido[4,3-d]pyrimidin-7-yl)-6,7- difluoronaphthalen-2-ol |
| II-19 | 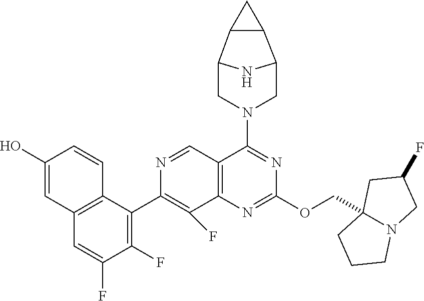 | 6,7-difluoro-5-(8-fluoro-2-(((2R,7aS)-2-fluoro-tetrahydro- 1H-pyrrolizin-7a(5H)-yl)methoxy)-4-((1R,2R,4S,5S)-7,9- diazatricyclo[3.3.1.02,4]nonan-7-yl)pyrido[4,3- d]pyrimidin-7-yl)naphthalen-2-ol |
| II-20 | 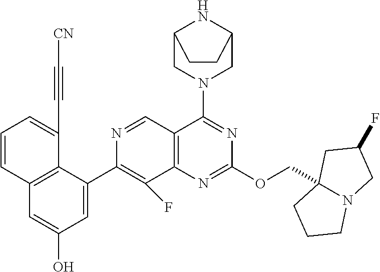 | 3-(8-(4-((1R,5S)-3,8-diazabicyclo[3.2.1]octan-3-yl)-8- fluoro-2-((2R,7aS)-2-fluorotetrahydro-1H-pyrrolizin- 7a(5H)-yl)methoxy)pyrido[4,3-d]-pyrimidin-7-yl)-6- hydroxynaphthalen-1-yl)propiolonitrile |
| 11-21 | 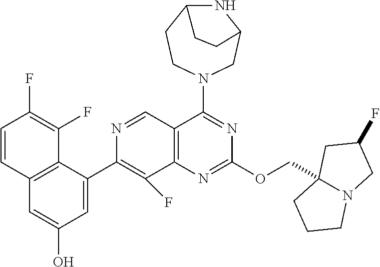 | 4-(4-(3,9-diazabicyclo[4.2.1]nonan-3-yl)-8-fluoro-2- (((2R,7aS)-2-fluorotetrahydro-1H-pyrrolizin-7a(5H)- yl)methoxy)pyrido[4,3-d]pyrimidin-7-yl)-5,6- difluoronaphthalen-2-ol |
| 11-22 | 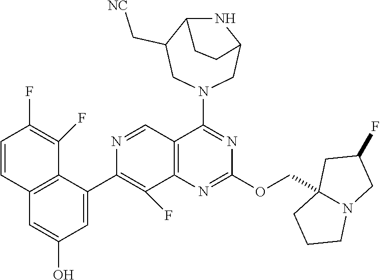 | 2-(3-(7-(7,8-difluoro-3-hydroxynaphthalen-1-yl)-8-fluoro- 2-(((2R,7aS)-2-fluorotetrahydro-1H-pyrrolizin-7a(5H)- yl)methoxy)pyrido[4,3-d]pyrimidin-4-yl)-3,9- diazabicyclo[4.2.1]nonan-5-yl)acetonitrile |
| 11-23 | 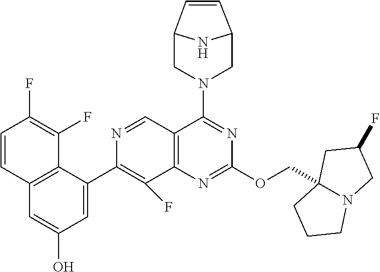 | 4-(4-((1R,5S)-3,8-diazabicyclo[3.2.1]oct-6-en-3-yl)-8- fluoro-2-(((2R,7aS)-2-fluorotetrahydro-1H-pyrrolizin- 7a(5H)-yl)methoxy)pyrido[4,3-d]pyrimidin-7-yl)-5,6- difluoronaphthalen-2-ol |
| 11-24 | 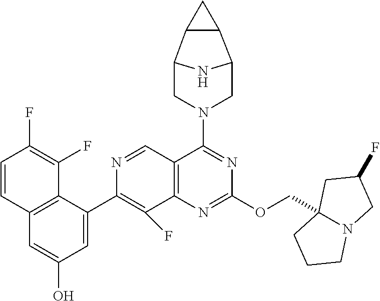 | 5,6-difluoro-4-(8-fluoro-2-(((2R,7aS)-2-fluoro-tetrahydro- 1H-pyrrolizin-7a(5H)-yl)methoxy)-4-((1R,2R,5S,5S)-7,9- diazatricyclo[3.3.1.02,4]nonan-7-yl)pyrido[4,3- d]pyrimidin-7-yl)naphthalen-2-ol |
| 11-25 | 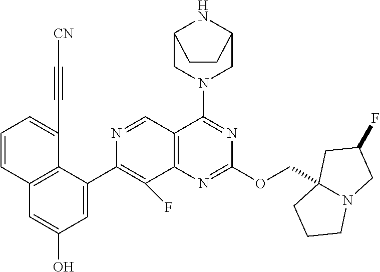 | 3-(8-(4-((1R,5S)-3,8-diazabicyclo[3.2.1]octan-3-yl)-8- fluoro-2-(((2R,7aS)-2-fluorotetrahydro-1H-pyrrolizin- 7a(5H)-yl)methoxy)pyrido[4,3-d]-pyrimidin-7-yl)-6- hydroxynaphthalen-1-yl)-propiolonitrile |
| 11-26 | 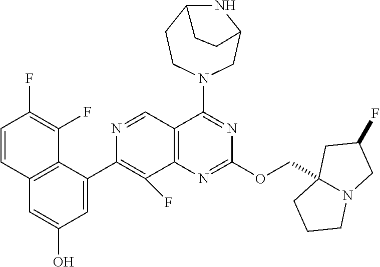 | 4-(4-(3,9-diazabicyclo[4.2.1]nonan-3-yl)-8-fluoro-2- (((2R,7aS)-2-fluorotetrahydro-1H-pyrrolizin-7a(5H)- yl)methoxy)pyrido[4,3-d]pyrimidin-7-yl)-5,6- difluoronaphthalen-2-ol |
| 11-27 | 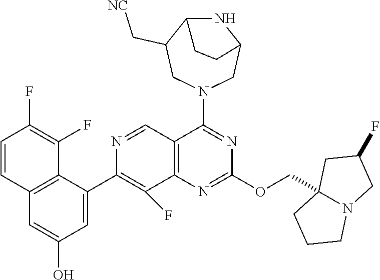 | 2-(3-(7-(7,8-difluoro-3-hydroxynaphthalen-1-yl)-8-fluoro- 2-(((2R,7aS)-2-fluorotetrahydro-1H-pyrrolizin-7a(5H)- yl)methoxy)pyrido[4,3-d]-pyrimidin-4-yl)-3,9- diazabicyclo[4.2.1]nonan-5-yl)acetonitrile |
| 11-28 | 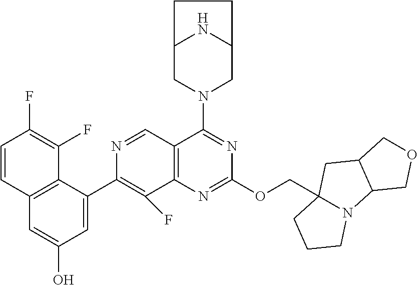 [SMILES: Oc1cc(-c2ncc3c(N4CC5CCC(C4)N5)nc(OCC45CCCN4C4COCC4C5)nc3c2F)c2c(F)c(F)ccc2c1] | 4-(4-(3,8-diazabicyclo[3.2.1]octan-3-yl)-8-fluoro-2- ((hexahydro-1H-furo[3,4-b]pyrrolizin-7a(5H)- yl)methoxy)pyrido[4,3-d]pyrimidin-7-yl)-5,6- difluoronaphthalen-2-ol |
| II-29 | 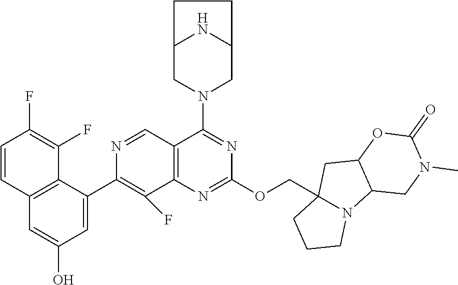 [SMILES: CN1CC2C(CC3(COc4nc(N5CC6CCC(C5)N6)c5cnc(-c6cc(O)cc7ccc(F)c(F)c67)c(F)c5n4)CCCN23)OC1=O] | 8a-(((4-(3,8-diazabicyclo[3.2.1]octan-3-yl)-7-(7,8-difluoro- 3-hydroxynaphthalen-1-yl)-8-fluoropyrido[4,3- d]pyrimidin-2-yl)oxy)methyl)-3-methyloctahydro- [1,3]oxazino[6,5-b]pyrrolizin-2(3H)-one |
| II-30 | 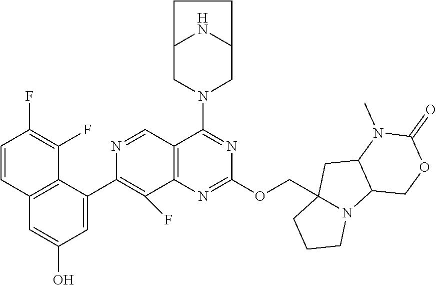 [SMILES: CN1C(=O)OCC2C1CC1(COc3nc(N4CC5CCC(C4)N5)c4cnc(-c5cc(O)cc6ccc(F)c(F)c56)c(F)c4n3)CCCN21] | 8a-(((4-(3,8-diazabicyclo[3.2.1]octan-3-yl)-7-(7,8-difluoro- 3-hydroxynaphthalen-1-yl)-8-fluoropyrido[4,3- d]pyrimidin-2-yl)oxy)methyl)-1-methyloctahydro- [1,3]oxazino[4,5-b]pyrrolizin-2(1H)-one |
| II-31 | 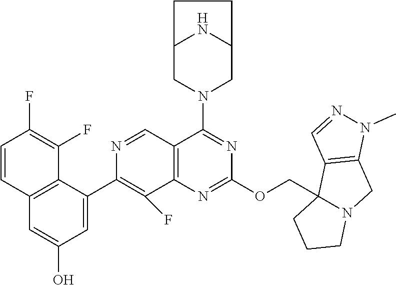 [SMILES: Cn1ncc2c1CN1CCCC21COc1nc(N2CC3CCC(C2)N3)c2cnc(-c3cc(O)cc4ccc(F)c(F)c34)c(F)c2n1] | 4-(4-(3,8-diazabicyclo[3.2.1]octan-3-yl)-8-fluoro-2-((1- methyl-1,5,6,8-tetrahydropyrazolo[4,3-a]-pyrrolizin- 3b(4H)-yl)methoxy)pyrido[4,3-d]-pyrimidin-7-yl)-5,6- difluoronapthalen-2-ol |
| II-32 | 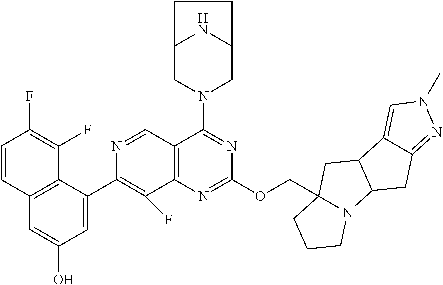 [SMILES: Cn1cc2c(n1)CC1C2CC2(COc3nc(N4CC5CCC(C4)N5)c4cnc(-c5cc(O)cc6ccc(F)c(F)c56)c(F)c4n3)CCCN12] | 4-(4-(3,8-diazabicyclo[3.2.1]octan-3-yl)-8-fluoro-2-((2- methyl-3b,4,6,7,8a,9-hexahydro-2H- pyrazolo[3′,4′:4,5]cyclopenta[1,2-b]pyrrolizin-4a(5H)- yl)methoxy)pyrido[4,3-d]pyrimidin-7-yl)-5,6- difluoronaphthalen-2-ol |
| II-33 | 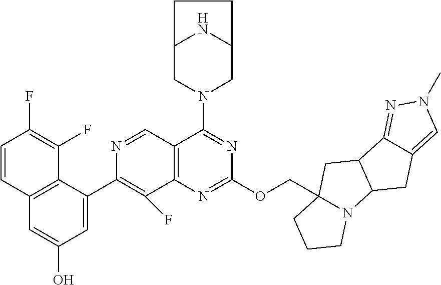 [SMILES: Cn1cc2c(n1)C1CC3(COc4nc(N5CC6CCC(C5)N6)c5cnc(-c6cc(O)cc7ccc(F)c(F)c67)c(F)c5n4)CCCN3C1C2] | 4-(4-(3,8-diazabicyclo[3.2.1]octan-3-yl)-8-fluoro-2-((2- methyl-4,4a,7,8,9,9a-hexahydro-2H- pyrazolo[4′,3′:4,5]cyclopenta[1,2-b]pyrrolizin-8a(6H)- yl)methoxy)pyrido[4,3-d]pyrimidin-7-yl)-5,6- difluoronaphthalen-2-ol |
| II-34 | 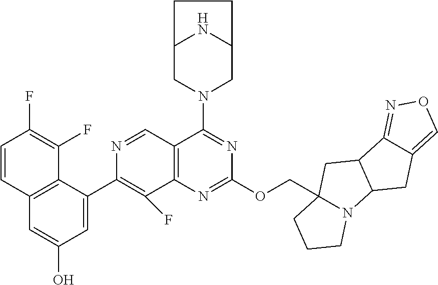 [SMILES: Oc1cc(-c2ncc3c(N4CC5CCC(C4)N5)nc(OCC45CCCN4C4Cc6conc6C4C5)nc3c2F)c2c(F)c(F)ccc2c1] | 4-(4-(3,8-diazabicyclo[3.2.1]octan-3-yl)-8-fluoro-2- ((4,4a,7,8,9,9a-hexahydroisoxazolo[4′,3′:4,5]- cyclopenta[1,2-b]pyrrolizin-8a(6H)-yl)methoxy)- pyrido[4,3-d]pyrimidin-7-yl)-5,6-difluoronaphthalen-2-ol |
| II-35 | 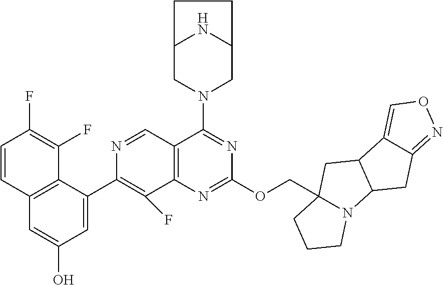 [SMILES: Oc1cc(-c2ncc3c(N4CC5CCC(C4)N5)nc(OCC45CCCN4C4Cc6nocc6C4C5)nc3c2F)c2c(F)c(F)ccc2c1] | 4-(4-(3,8-diazabicyclo[3.2.1]octan-3-yl)-8-fluoro-2- ((3b,4,6,7,8a,9-hexahydroisoxazolo[3′,4′:4,5]- cyclopenta[1,2-b]pyrrolizin-4a(5H)-yl)methoxy)- pyrido[4,3-d]pyrimidin-7-yl)-5,6-difluoronaphthalen-2-ol |
| II-36 | 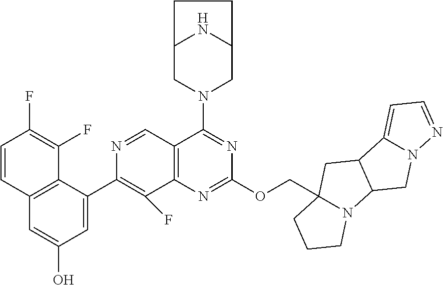 [SMILES: Oc1cc(-c2ncc3c(N4CC5CCC(C4)N5)nc(OCC45CCCN4C4Cn6nccc6C4C5)nc3c2F)c2c(F)c(F)ccc2c1] | 4-(4-(3,8-diazabicyclo[3.2.1]octan-3-yl)-8-fluoro-2- ((3b,4,6,7,8a,9-hexahydropyrazolo[1′,5′:1,2]-pyrrolo[3,4- b]pyrrolizin-4a(5H)-yl)methoxy)-pyrido[4,3-d]pyrimidin- 7-yl)-5,6-difluoronaphthalen-2-ol |
| II-37 | 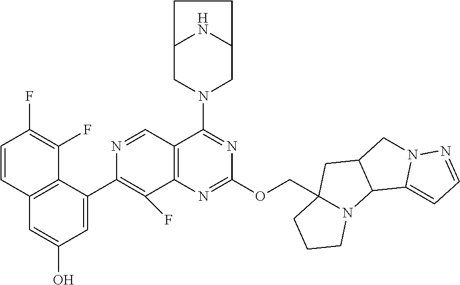 [SMILES: Oc1cc(-c2ncc3c(N4CC5CCC(C4)N5)nc(OCC45CCCN4C4c6ccnn6CC4C5)nc3c2F)c2c(F)c(F)ccc2c1] | 4-(4-(3,8-diazabicyclo[3.2.1]octan-3-yl)-8-fluoro-2- ((6,7,8,8a-tetrahydro-5H,9H-pyrazolo[1′,5′:1,5]- pyrrolo[3,4-b]pyrrolizin-7a(3bH)-yl)methoxy)-pyrido[4,3- d]pyrimidin-7-yl)-5,6-difluoronaphthalen-2-ol |
| II-38 | 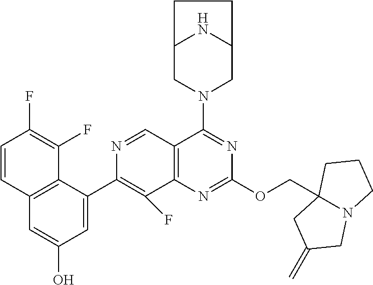 [SMILES: C=C1CN2CCCC2(COc2nc(N3CC4CCC(C3)N4)c3cnc(-c4cc(O)cc5ccc(F)c(F)c45)c(F)c3n2)C1] | 4-(4-(3,8-diazabicyclo[3.2.1]octan-3-yl)-8-fluoro-2-((2- methylenetetrahydro-1H-pyrrolizin-7a(5H)- yl)methoxy)pyrido[4,3-d]pyrimidin-7-yl)-5,6- difluoronaphthalen-2-ol |
| II-39 | 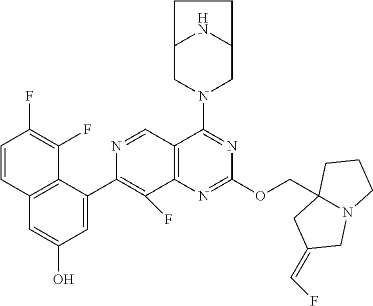 [SMILES: Oc1cc(-c2ncc3c(N4CC5CCC(C4)N5)nc(OCC45CCCN4C/C(=C\F)C5)nc3c2F)c2c(F)c(F)ccc2c1] | 4-(4-(3,8-diazabicyclo[3.2.1]octan-3-yl)-8-fluoro-2-((2- (fluoromethylene)tetrahydro-1H-pyrrolizin-7a(5H)- yl)methoxy)pyrido[4,3-d]pyrimidin-7-yl)-5,6- difluoronaphthalen-2-ol |
| II-40 | 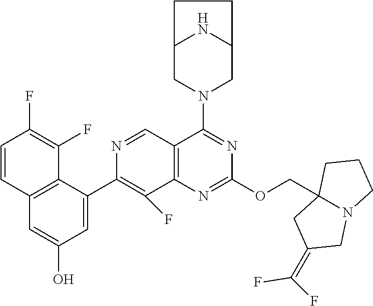 [SMILES: Oc1cc(-c2ncc3c(N4CC5CCC(C4)N5)nc(OCC45CCCN4CC(=C(F)F)C5)nc3c2F)c2c(F)c(F)ccc2c1] | 4-(4-(3,8-diazabicyclo[3.2.1]octan-3-yl)-2-((2- (difluoromethylene)tetrahydro-1H-pyrrolizin-7a(5H)- yl)methoxy)-8-fluoropyrido[4,3-d]pyrimidin-7-yl)-5,6- difluoronaphthalen-1-ol |
| II-41 | 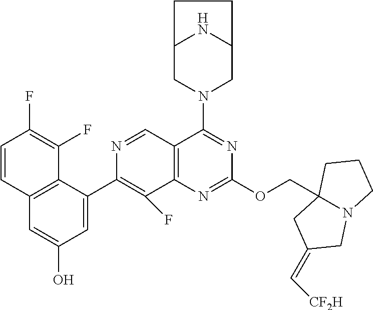 [SMILES: Oc1cc(-c2ncc3c(N4CC5CCC(C4)N5)nc(OCC45CCCN4C/C(=C\C(F)F)C5)nc3c2F)c2c(F)c(F)ccc2c1] | 4-(4-(3,8-diazabicyclo[3.2.1]octan-3-yl)-2-((2-(2,2- difluoroethylidene)tetrahydro-1H-pyrrolizin-7a(5H)- yl)methoxy)-8-fluoropyrido[4,3-d]pyrimidin-7-yl)-5,6- difluoronaphthalen-2-ol |
| II-42 | 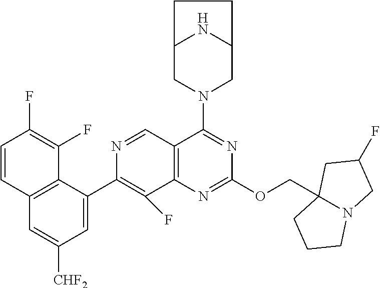 [SMILES: Fc1ccc2cc(C(F)F)cc(-c3ncc4c(N5CC6CCC(C5)N6)nc(OCC56CCCN5CC(F)C6)nc4c3F)c2c1F] | 4-(3,8-diazabicyclo[3.2.1]octan-3-yl)-7-(3- (difluoromethyl)-7,8-difluoronaphthalen-1-yl)-8-fluoro-2- ((2-fluorotetrahydro-1H-pyrrolizin-7a(5H)- yl)methoxy)pyrido[4,3-d]pyrimidine |
| II-43 | 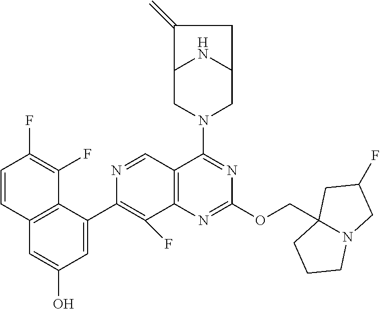 [SMILES: C=C1CC2CN(c3nc(OCC45CCCN4CC(F)C5)nc4c(F)c(-c5cc(O)cc6ccc(F)c(F)c56)ncc34)CC1N2] | 5,6-difluoro-4-(8-fluoro-2-((2-fluorotetrahydro-1H- pyrrolizin-7a(5H)-yl)methoxy)-4-(6-methylene-3,8- diazabicyclo[3.2.1]octan-3-yl)pyrido[4,3-d]-pyrimidin-7- yl)naphthalen-2-ol |
| II-44 | 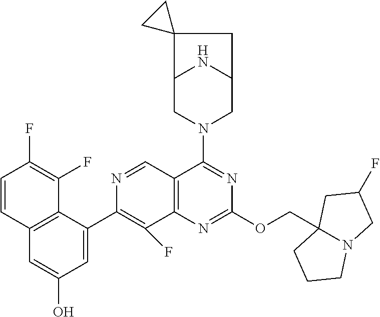 [SMILES: Oc1cc(-c2ncc3c(N4CC5CC6(CC6)C(C4)N5)nc(OCC45CCCN4CC(F)C5)nc3c2F)c2c(F)c(F)ccc2c1] | 5,6-difluoro-4-(8-fluoro-2-((2-fluorotetrahydro-1H- pyrrolizin-7a(5H)-yl)methoxy)-4-(3,8-diazaspiro- [bicyclo[3.2.1]octane-6,1′-cyclopropan]-3-yl)pyrido[4,3- d]pyrimidin-7-yl)naphthalen-2-ol |
| II-45 | 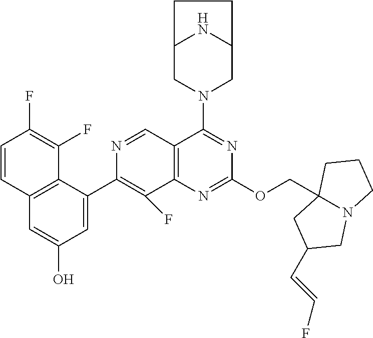 [SMILES: Oc1cc(-c2ncc3c(N4CC5CCC(C4)N5)nc(OCC45CCCN4CC(/C=C/F)C5)nc3c2F)c2c(F)c(F)ccc2c1] | (E)-4-(4-(3,8-diazabicyclo[3.2.1]octan-3-yl)-8-fluoro-2- ((2-(2-fluorovinyl)tetrahydro-1H-pyrrolizin-7a(5H)- yl)methoxy)pyrido[4,3-d]pyrimidin-7-yl)-5,6- difluoronaphthalen-2-ol |
| II-46 | 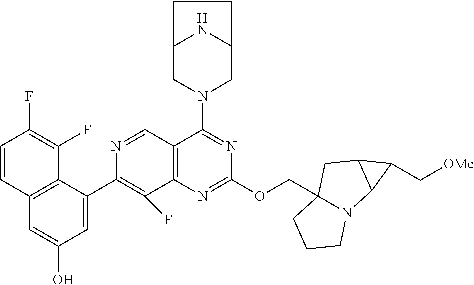 [SMILES: COCC1C2CC3(COc4nc(N5CC6CCC(C5)N6)c5cnc(-c6cc(O)cc7ccc(F)c(F)c67)c(F)c5n4)CCCN3C12] | 4-(4-(3,8-diazabicyclo[3.2.1]octan-3-yl)-8-fluoro-2-((1- (methoxymethyl)hexahydro-cyclopropa[b]-pyrrolizin- 5a(3H)-yl)methoxy)-pyrido[4,3-d]pyrimidin-7-yl)-5,6- difluoronaphthalen-2-ol |
| II-47 | 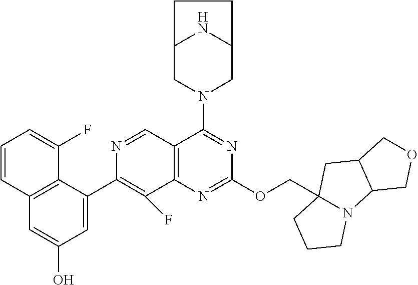 [SMILES: Oc1cc(-c2ncc3c(N4CC5CCC(C4)N5)nc(OCC45CCCN4C4COCC4C5)nc3c2F)c2c(F)cccc2c1] | 4-(4-(3,8-diazabicyclo[3.2.1]octan-3-yl)-8-fluoro-2- ((hexahydro-1H-furo[3,4-b]pyrrolizin-7a(5H)- yl)methoxy)pyrido[4,3-d]pyrimidin-7-yl)-5- fluoronaphthalen-1-ol |
| II-53 | 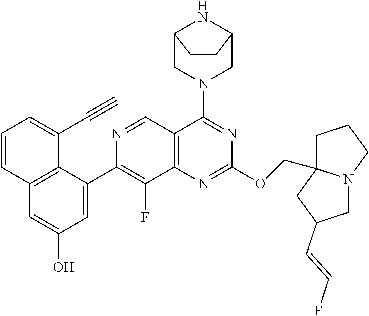 [SMILES: C#Cc1cccc2cc(O)cc(-c3ncc4c(N5CC6CCC(C5)N6)nc(OCC56CCCN5CC(/C=C/F)C6)nc4c3F)c12] | 4-(4-((1R,5S)-3,8-diazabicyclo[3.2.1]octan-3-yl)-8-fluoro- 2-((2-((E)-2-fluorovinyl)tetrahydro-1H-pyrrolizin-7a(5H)- yl)methoxy)pyrido[4,3-d]pyrimidin-7-yl)-5- ethynylnaphthalen-1-ol |
| II-54 | 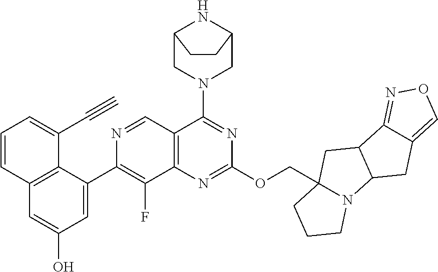 [SMILES: C#Cc1cccc2cc(O)cc(-c3ncc4c(N5CC6CCC(C5)N6)nc(OCC56CCCN5C5Cc7conc7C5C6)nc4c3F)c12] | 4-(4-((1R,5S)-3,8-diazabicyclo[3.2.1]octan-3-yl)-8-fluoro- 2-((4,4a,7,8,9,9a- hexahydroisoxazolo[4′,3′:4,5]cyclopenta[1,2-b]pyrrolizin- 8a(6H)-yl)methoxy)pyrido[4,3-d]pyrimidin-7-yl)-5- ethynylnaphthalen-2-ol |
| II-55 | 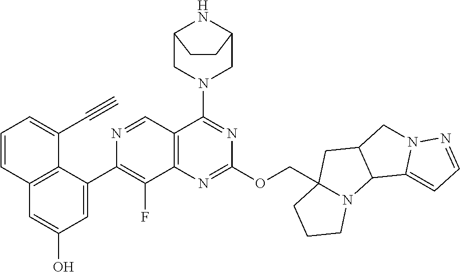 [SMILES: C#Cc1cccc2cc(O)cc(-c3ncc4c(N5CC6CCC(C5)N6)nc(OCC56CCCN5C5c7ccnn7CC5C6)nc4c3F)c12] | 4-(4-((1R,5S)-3,8-diazabicyclo[3.2.1]octan-3-yl)-8-fluoro- 2-((6,7,8,8a-tetrahydro-5H,9H- pyrazolo[1′,5′:1,5]pyrrolo[3,4-b]pyrrolizin-7a(3bH)- yl)methoxy)pyrido[4,3-d]pyrimidin-7-yl)-5- ethynylnaphthalen-2-ol |
| II-56 | 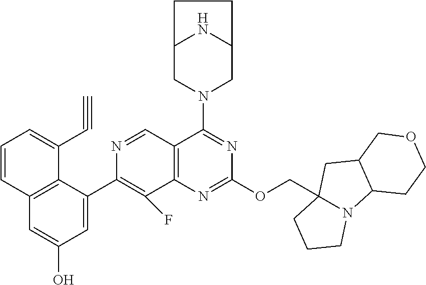 [SMILES: C#Cc1cccc2cc(O)cc(-c3ncc4c(N5CC6CCC(C5)N6)nc(OCC56CCCN5C5CCOCC5C6)nc4c3F)c12] | 4-(4-(3,8-diazabicyclo[3.2.1]octan-3-yl)-8-fluoro-2- ((hexahydro-1H,3H-pyrano[3,4-b]pyrrolizin-8a(6H)- yl)methoxy)pyrido[4,3-d]pyrimidin-7-yl)-5- ethynylnaphthalen-2-ol |
| II-57 | 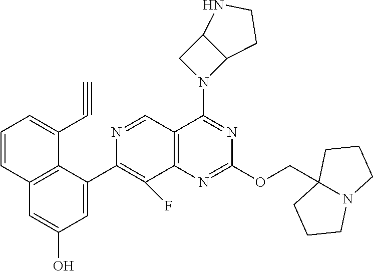 [SMILES: C#Cc1cccc2cc(O)cc(-c3ncc4c(N5CC6NCCC65)nc(OCC56CCCN5CCC6)nc4c3F)c12] | 4-(4-(2,6-diazabicyclo[3.2.0]heptan-6-yl)-8-fluoro-2- ((tetrahydro-1H-pyrrolizin-7a(5H)-yl)methoxy)pyrido[4,3- d]pyrimidin-7-yl)-5-ethynylnaphthalen-2-ol |
| II-58 | 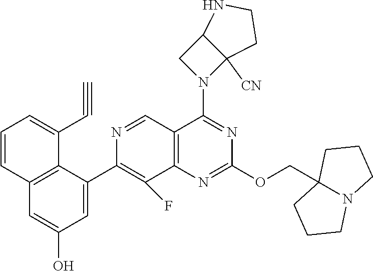 [SMILES: C#Cc1cccc2cc(O)cc(-c3ncc4c(N5CC6NCCC65C#N)nc(OCC56CCCN5CCC6)nc4c3F)c12] | 6-(7-(8-ethynyl-3-hydroxynaphthalen-1-yl)-8-fluoro-2- ((tetrahydro-1H-pyrrolizin-7a(5H)-yl)methoxy)pyrido[4,3- d]pyrimidin-4-yl)-2,6-diazabicyclo[3.2.0]heptane-5- carbonitrile |
| II-59 | 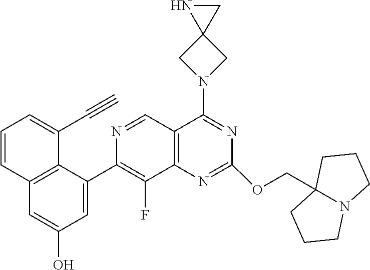 [SMILES: C#Cc1cccc2cc(O)cc(-c3ncc4c(N5CC6(CN6)C5)nc(OCC56CCCN5CCC6)nc4c3F)c12] | 5-ethynyl-4-(8-fluoro-4-(1,5-diazaspiro[2.3]hexan-5-yl)-2- ((tetrahydro-1H-pyrrolizin-7a(5H)-yl)methoxy)pyrido[4,3- d]pyrimidin-7-yl)naphthalen-2-ol |
| II-60 | 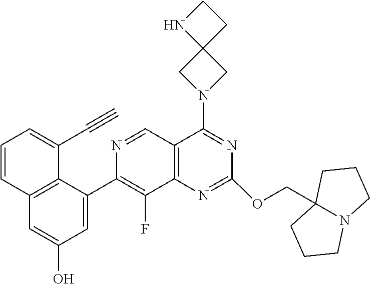 [SMILES: C#Cc1cccc2cc(O)cc(-c3ncc4c(N5CC6(CCN6)C5)nc(OCC56CCCN5CCC6)nc4c3F)c12] | 5-ethynyl-4-(8-fluoro-4-(1,6-diazaspiro[3.3]heptan-6-yl)-2- ((tetrahydro-1H-pyrrolizin-7a(5H)-yl)methoxy)pyrido[4,3- d]pyrimidin-7-yl)naphthalen-2-ol |
| II-61 | 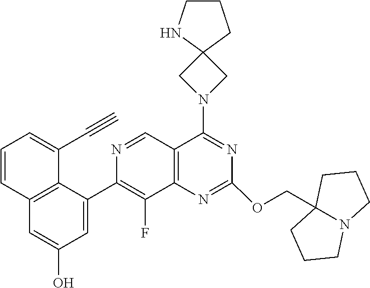 [SMILES: C#Cc1cccc2cc(O)cc(-c3ncc4c(N5CC6(CCCN6)C5)nc(OCC56CCCN5CCC6)nc4c3F)c12] | 5-ethynyl-4-(8-fluoro-4-(2,5-diazaspiro[3.4]octan-2-yl)-2- ((tetrahydro-1H-pyrrolizin-7a(5H)-yl)methoxy)pyrido[4,3- d]pyrimidin-7-yl)naphthalen-2-ol |
| II-62 | 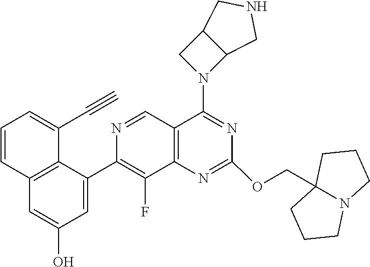 [SMILES: C#Cc1cccc2cc(O)cc(-c3ncc4c(N5CC6CNCC65)nc(OCC56CCCN5CCC6)nc4c3F)c12] | 4-(4-(3,6-diazabicyclo[3.2.0]heptan-6-yl)-8-fluoro-2- ((tetrahydro-1H-pyrrolizin-7a(5H)-yl)methoxy)pyrido[4,3- d]pyrimidin-7-yl)-5-ethynylnaphthalen-2-ol |
| II-63 | 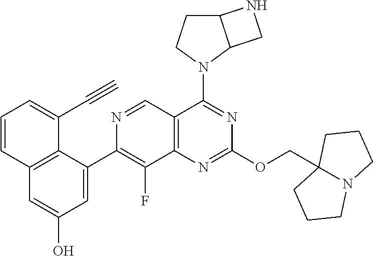 [SMILES: C#Cc1cccc2cc(O)cc(-c3ncc4c(N5CCC6NCC65)nc(OCC56CCCN5CCC6)nc4c3F)c12] | 4-(4-(2,6-diazabicyclo[3.2.0]heptan-2-yl)-8-fluoro-2- ((tetrahydro-1H-pyrrolizin-7a(5H)-yl)methoxy)pyrido[4,3- d]pyrimidin-7-yl)-5-ethynylnaphthalen-2-ol |
| II-64 | 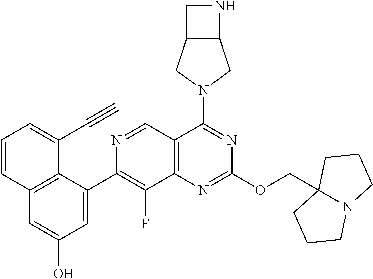 [SMILES: C#Cc1cccc2cc(O)cc(-c3ncc4c(N5CC6CNC6C5)nc(OCC56CCCN5CCC6)nc4c3F)c12] | 4-(4-(3,6-diazabicyclo[3.2.0]heptan-3-yl)-8-fluoro-2- ((tetrahydro-1H-pyrrolizin-7a(5H)-yl)methoxy)pyrido[4,3- d]pyrimidin-7-yl)-5-ethynylnaphthalen-2-ol |
| II-65 | 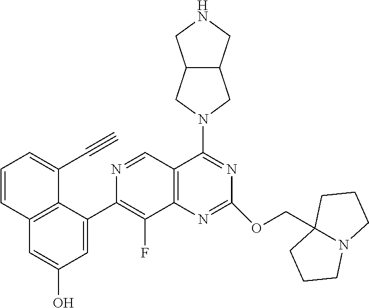 [SMILES: C#Cc1cccc2cc(O)cc(-c3ncc4c(N5CC6CNCC6C5)nc(OCC56CCCN5CCC6)nc4c3F)c12] | 5-ethynyl-4-(8-fluoro-4-(hexahydropyrrolo[3,4-c]pyrrol- 2(1H)-yl)-2-((tetrahydro-1H-pyrrolizin-7a(5H)- yl)methoxy)pyrido[4,3-d]pyrimidin-7-yl)naphthalen-2-ol |
| II-66 | 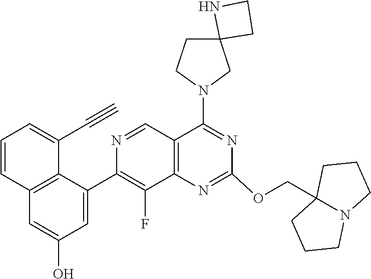 [SMILES: C#Cc1cccc2cc(O)cc(-c3ncc4c(N5CCC6(CCN6)C5)nc(OCC56CCCN5CCC6)nc4c3F)c12] | 5-ethynyl-4-(8-fluoro-4-(1,6-diazaspiro[3.4]octan-6-yl)-2- ((tetrahydro-1H-pyrrolizin-7a(5H)-yl)methoxy)pyrido[4,3- d]pyrimidin-7-yl)naphthalen-2-ol |
| II-67 | 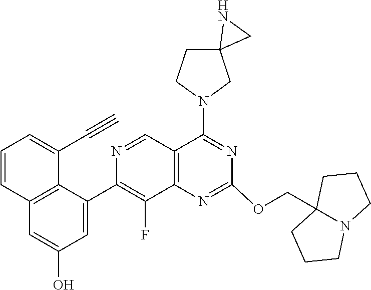 [SMILES: C#Cc1cccc2cc(O)cc(-c3ncc4c(N5CCC6(CN6)C5)nc(OCC56CCCN5CCC6)nc4c3F)c12] | 5-ethynyl-4-(8-fluoro-4-(1,5-diazaspiro[2.4]heptan-5-yl)-2- ((tetrahydro-1H-pyrrolizin-7a(5H)-yl)methoxy)pyrido[4,3- d]pyrimidin-7-yl)naphthalen-2-ol |
| II-68 | 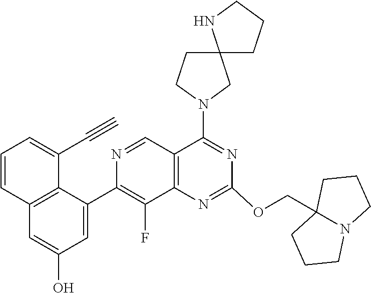 [SMILES: C#Cc1cccc2cc(O)cc(-c3ncc4c(N5CCC6(CCCN6)C5)nc(OCC56CCCN5CCC6)nc4c3F)c12] | 5-ethynyl-4-(8-fluoro-4-(1,7-diazaspiro[4.4]nonan-7-yl)-2- ((tetrahydro-1H-pyrrolizin-7a(5H)-yl)methoxy)pyrido[4,3- d]pyrimidin-7-yl)naphthalen-1-ol |
| II-69 | 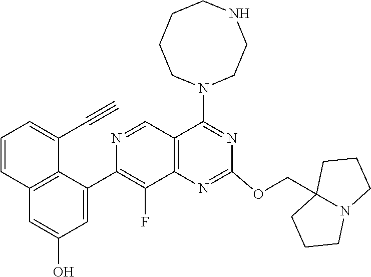 [SMILES: C#Cc1cccc2cc(O)cc(-c3ncc4c(N5CCCCNCC5)nc(OCC56CCCN5CCC6)nc4c3F)c12] | 4-(4-(1,4-diazocan-1-yl)-8-fluoro-2-((tetrahydro-1H- pyrrolizin-7a(5H)-yl)methoxy)pyrido[4,3-d]pyrimidin-7- yl)-5-ethynylnaphthalen-2-ol |
| II-70 | 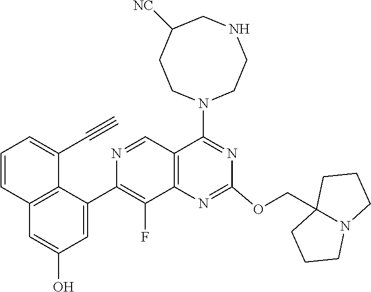 [SMILES: C#Cc1cccc2cc(O)cc(-c3ncc4c(N5CCNCC(C#N)CC5)nc(OCC56CCCN5CCC6)nc4c3F)c12] | 1-(7-(8-ethynyl-3-hydroxynaphthalen-1-yl)-8-fluoro-2- ((tetrahydro-1H-pyrrolizin-7a(5H)-yl)methoxy)pyrido[4,3- d]pyrimidin-4-yl)-1,4-diazocane-6-carbonitrile |
| II-71 | 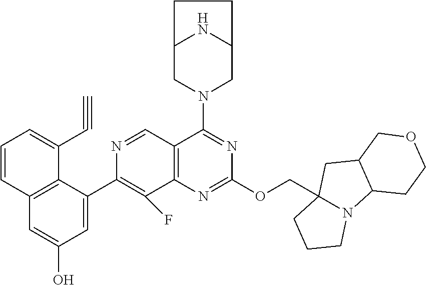 [SMILES: C#Cc1cccc2cc(O)cc(-c3ncc4c(N5CC6CCC(C5)N6)nc(OCC56CCCN5C5CCOCC5C6)nc4c3F)c12] | 4-(4-(3,8-diazabicyclo[3.2.1]octan-3-yl)-8-fluoro-2- ((hexahydro-1H,3H-pyrano[3,4-b]pyrrolizin-8a(6H)- yl)methoxy)pyrido[4,3-d]pyrimidin-7-yl)-5- ethynylnaphthalen-2-ol |
| II-72 | 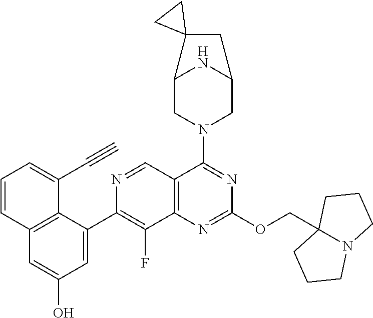 [SMILES: C#Cc1cccc2cc(O)cc(-c3ncc4c(N5CC6CC7(CC7)C(C5)N6)nc(OCC56CCCN5CCC6)nc4c3F)c12] | 5-ethynyl-4-(8-fluoro-4-(3,8-diazaspiro[bicyclo- [3.2.1]octane-6,1′-cyclopropan]-3-yl)-2-((tetrahydro-1H- pyrrolizin-7a(5H)-yl)methoxy)pyrido[4,3-d]pyrimidin-7- yl)naphthalen-2-ol |
| II-73 | 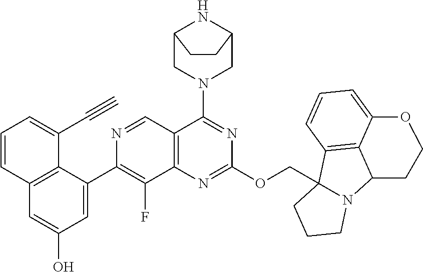 [SMILES: C#Cc1cccc2cc(O)cc(-c3ncc4c(N5CC6CCC(C5)N6)nc(OCC56CCCN5C5CCOc7cccc6c75)nc4c3F)c12] | 4-(4-((1R,5S)-3,8-diazabicyclo[3.2.1]octan-3-yl)-8-fluoro- 2-((1,8,9,10a-tetrahydro-2H-pyrano[4,3,2-cd]pyrrolo[2,1- a]isoindol-6b(7H)-yl)methoxy)pyrido[4,3-d]pyrimidin-7- yl)-5-ethynylnaphthalen-2-ol |
| II-74 | 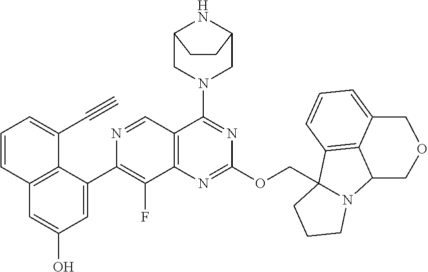 [SMILES: C#Cc1cccc2cc(O)cc(-c3ncc4c(N5CC6CCC(C5)N6)nc(OCC56CCCN5C5COCc7cccc6c75)nc4c3F)c12] | 4-(4-((1R,5S)-3,8-diazabicyclo[3.2.1]octan-3-yl)-8-fluoro- 2-((3,8,9,10a-tetrahydro-1H-pyrano[3,4,5-cd]pyrrolo[2,1- a]isoindol-6b(7H)-yl)methoxy)pyrido[4,3-d]pyrimidin-7- yl)-5-ethynylnaphthalen-2-ol |
| II-75 | 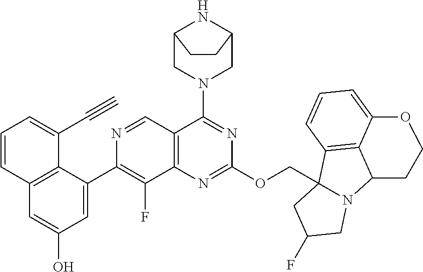 [SMILES: C#Cc1cccc2cc(O)cc(-c3ncc4c(N5CC6CCC(C5)N6)nc(OCC56CC(F)CN5C5CCOc7cccc6c75)nc4c3F)c12] | 4-(4-((1R,5S)-3,8-diazabicyclo[3.2.1]octan-3-yl)-8-fluoro- 2-((8-fluoro-1,8,9,10a-tetrahydro-2H-pyrano[4,3,2- cd]pyrrolo[2,1-a]isoindol-6b(7H)-yl)methoxy)pyrido[4,3- d]pyrimidin-7-yl)-5-ethynylnaphthalen-2-ol |
| II-76 | 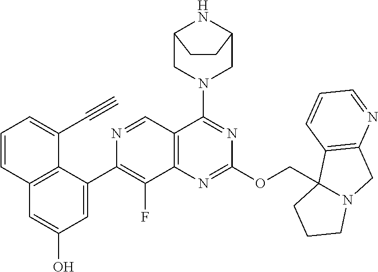 [SMILES: C#Cc1cccc2cc(O)cc(-c3ncc4c(N5CC6CCC(C5)N6)nc(OCC56CCCN5Cc5ncccc56)nc4c3F)c12] | 4-(4-((1R,5S)-3,8-diazabicyclo[3.2.1]octan-3-yl)-2-((6,7- dihydro-5H-pyrido[3,2-a]pyrrolizin-4b(9H)-yl)methoxy)- 8-fluoropyrido[4,3-d]pyrimidin-7-yl)-5-ethynylnaphthalen- 2-ol |
| II-77 | 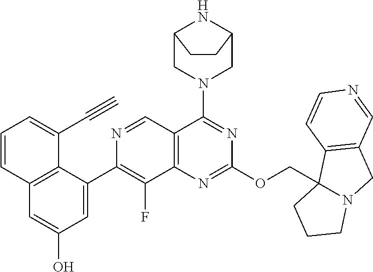 [SMILES: C#Cc1cccc2cc(O)cc(-c3ncc4c(N5CC6CCC(C5)N6)nc(OCC56CCCN5Cc5cnccc56)nc4c3F)c12] | 4-(4-((1R,5S)-3,8-diazabicyclo[3.2.1]octan-3-yl)-2-((6,7- dihydro-5H-pyrido[4,3-a]pyrrolizin-4b(9H)-yl)methoxy)- 8-fluoropyrido[4,3-d]pyrimidin-7-yl)-5-ethynylnaphthalen- 2-ol |
| II-78 | 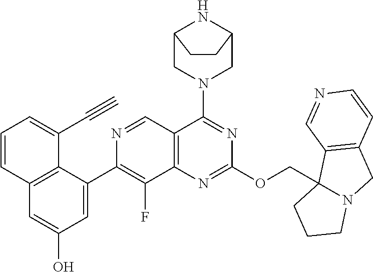 [SMILES: C#Cc1cccc2cc(O)cc(-c3ncc4c(N5CC6CCC(C5)N6)nc(OCC56CCCN5Cc5ccncc56)nc4c3F)c12] | 4-(4-((1R,5S)-3.8-diazabicyclo[3.2.1]octan-3-yl)-2-((8,9- dihydro-5H-pyrido[3,4-a]pyrrolizin-9a(7H)-yl)methoxy)- 8-fluoropyrido[4,3-d]pyrimidin-7-yl)-5-ethynylnaphthalen- 2-ol |
| II-79 | 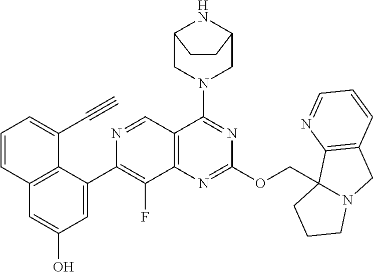 [SMILES: C#Cc1cccc2cc(O)cc(-c3ncc4c(N5CC6CCC(C5)N6)nc(OCC56CCCN5Cc5cccnc56)nc4c3F)c12] | 4-(4-((1R,5S)-3,8-diazabicyclo[3.2.1]octan-3-yl)-2-((8,9- dihydro-5H-pyrido[2,3-a]pyrrolizin-9a(7H)-yl)methoxy)- 8-fluoropyrido[4,3-d]pyrimidin-7-yl)-5-ethynylnaphthalen- 2-ol |
| II-80 | 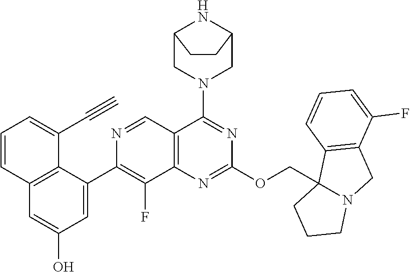 [SMILES: C#Cc1cccc2cc(O)cc(-c3ncc4c(N5CC6CCC(C5)N6)nc(OCC56CCCN5Cc5c(F)cccc56)nc4c3F)c12] | 4-(4-((1R,5S)-3,8-diazabicyclo[3.2.1]octan-3-yl)-8-fluoro- 2-((6-fluoro-2,3-dihydro-1H-pyrrolo[2,1-a]isoindol- 9b(5H)-yl)methoxy)pyrido[4,3-d]pyrimidin-7-yl)-5- ethynylnaphthalen-1-ol |
| II-81 | 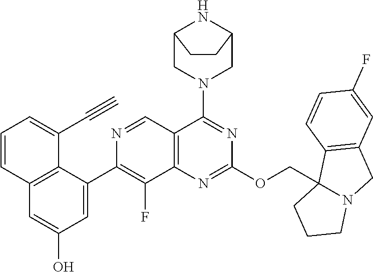 [SMILES: C#Cc1cccc2cc(O)cc(-c3ncc4c(N5CC6CCC(C5)N6)nc(OCC56CCCN5Cc5cc(F)ccc56)nc4c3F)c12] | 4-(4-((1R,5S)-3,8-diazabicyclo[3.2.1]octan-3-yl)-8-fluoro- 2-((7-fluoro-2,3-dihydro-1H-pyrrolo[2,1-a]isoindol- 9b(5H)-yl)methoxy)pyrido[4,3-d]pyrimidin-7-yl)-5- ethynylnaphthalen-2-ol |
| II-82 | 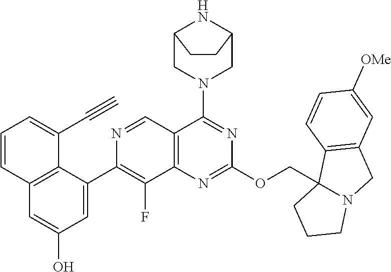 [SMILES: C#Cc1cccc2cc(O)cc(-c3ncc4c(N5CC6CCC(C5)N6)nc(OCC56CCCN5Cc5cc(OC)ccc56)nc4c3F)c12] | 4-(4-((1R,5S)-3,8-diazabicyclo[3.2.1]octan-3-yl)-8-fluoro- 2-((7-methoxy-2,3-dihydro-1H-pyrrolo[2,1-a]isoindol- 9b(5H)-yl)methoxy)pyrido[4,3-d]pyrimidin-7-yl)-5- ethynylnaphthalen-2-ol |
| II-83 | 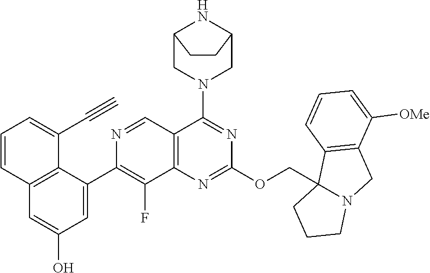 [SMILES: C#Cc1cccc2cc(O)cc(-c3ncc4c(N5CC6CCC(C5)N6)nc(OCC56CCCN5Cc5c(OC)cccc56)nc4c3F)c12] | 4-(4-((1R,5S)-3,8-diazabicyclo[3.2.1]octan-3-yl)-8-fluoro- 2-((6-methoxy-2,3-dihydro-1H-pyrrolo[2,1-a]isoindol- 9b(5H)-yl)methoxy)pyrido[4,3-d]pyrimidin-7-yl)-5- ethynylnaphthalen-2-ol |
| II-84 | 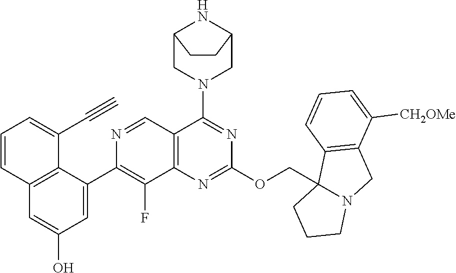 | 4-(4-((1R,5S)-3,8-diazabicyclo[3.2.1]octan-3-yl)-8-fluoro- 2-((6-(methoxymethyl)-2,3-dihydro-1H-pyrrolo[2,1- a]isoindol-9b(5H)-yl)methoxy)pyrido[4,3-d]pyrimidin-7- yl)-5-ethynylnaphthalen-2-ol |
| II-85 | 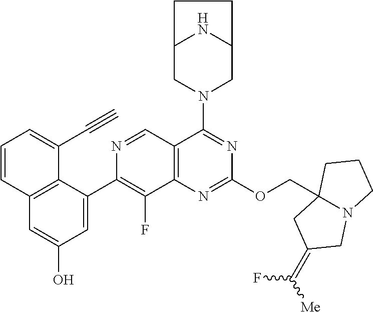 | 4-(4-(3,8-diazabicyclo[3.2.1]octan-3-yl)-8-fluoro-2-((2-(1- fluoroethylidene)tetrahydro-1H-pyrrolizin-7a(5H)- yl)methoxy)pyrido[4,3-d]pyrimidin-7-yl)-5- ethynylnaphthalen-2-ol |
| II-86 | 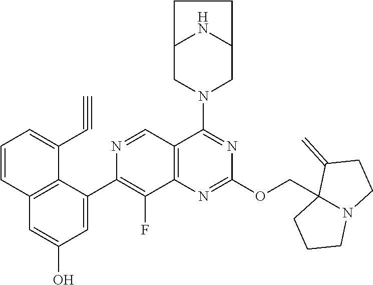 [SMILES: C#Cc1cccc2cc(O)cc(-c3ncc4c(N5CC6CCC(C5)N6)nc(OCC56CCCN5CCC6=C)nc4c3F)c12] | 4-(4-(3,8-diazabicyclo[3.2.1]octan-3-yl)-8-fluoro-2-((1- methylenetetrahydro-1H-pyrrolizin-7a(5H)- yl)methoxy)pyrido[4,3-d]pyrimidin-7-yl)-5- ethynylnaphthalen-2-ol |
| II-87 | 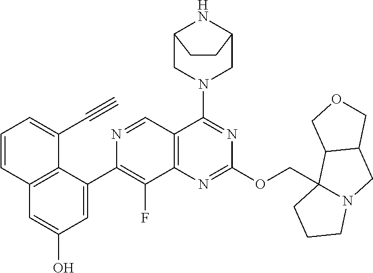 [SMILES: C#Cc1cccc2cc(O)cc(-c3ncc4c(N5CC6CCC(C5)N6)nc(OCC56CCCN5CC5COCC56)nc4c3F)c12] | 4-(4-((1R,5S)-3,8-diazabicyclo[3.2.1]octan-3-yl)-8-fluoro- 2-((hexahydro-3H-furo[3,4-a]pyrrolizin-8a(6H)- yl)methoxy)pyrido[4,3-d]pyrimidin-7-yl)-5- ethynylnaphthalen-2-ol |

### Embodiments A

In further embodiments 1A-48A below, the present disclosure includes:

1A. In embodiment 1A, provided is a compound of Formula (II) as defined in the third aspect of the Summary, or a pharmaceutically acceptable salt thereof.

1a1. In embodiment 1a1, the compound of embodiment 1A, or a pharmaceutically acceptable salt thereof, is wherein:

R⁴ is:

(i) —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is aryl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl; or

(ii) —CR³³═CR³⁴R³⁵ where R³³ and R³⁴ are independently selected from hydrogen, deuterium, alkyl, halo, and haloalkyl; and

R³⁵ is hydrogen, deuterium, alkyl, halo, haloalkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, or spiro heterocyclyl; or

R³⁴ and R³⁵ together with the carbon atom to which are attached form cycloalkyl, bridged cycloalkyl, fused cycloalkyl, spiro cycloalkyl, heterocyclyl, bridged heterocyclyl, fused heterocyclyl, or spiro heterocyclyl, wherein:

(a) the groups alkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, and spiro heterocyclyl of R³⁵; and (b) the groups cycloalkyl, bridged cycloalkyl, fused cycloalkyl, spiro cycloalkyl, heterocyclyl, bridged heterocyclyl, fused heterocyclyl, or spiro heterocyclyl formed by R³⁴ and R³⁵ together, are independently substituted with R^{h}, Rⁱ, and R^{j} independently selected from hydrogen, alkyl, halo, haloalkyl, hydroxyalkyl, alkoxyalkyl, cyano, cycloalkyl, bridged cycloalkyl, optionally substituted heterocyclyl, alkylsulfonylalkyl, dialkyl(oxo)phosphinyl, dialkyl(oxo)phosphinylalkyl, —O(alk)_{z1}R^{k}, —O(alk)OR^{l}, —S(O)R^{m}, —S(O)₂Rⁿ, —NR^{p}C(O)R^{o}, —NR^{r}SO₂R^{q}, —OC(O)NR^{s}R^{t}, —C(O)NR^{u}R^{v}, —S(O)₂NR^{w}R^{x}, and —NR^{y}R^{z}, where z1 is 0 or 1, alk is alkylene, and R^{k}, R^{l}, R^{m}, Rⁿ, R^{o}, R^{p}, R^{q}, R^{r}, R^{s}, R^{t}, R^{u}, R^{v}, R^{w}, R^{x}, R^{y}, and R^{z} are independently selected from hydrogen, alkyl, cycloalkyl, cycloalkylalkyl, halo, hydroxyalkyl, alkoxyalkyl, and aminoalkyl.

2A. In embodiment 2A, the compound of embodiment 1A or Tal, or a pharmaceutically acceptable salt thereof, is wherein R¹ is a ring of formula

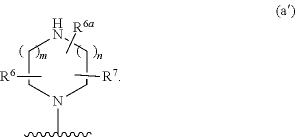

2a1. In embodiment 2a1, the compound of embodiment 1A, 1a1, or 2A, or a pharmaceutically acceptable salt thereof, is wherein m and n are each 1, or one of m and n is 1 and the other of m and n is 2.

2b1. In embodiment 2b1, the compound of embodiment 1A, 1a1, or 2A, or a pharmaceutically acceptable salt thereof, is wherein m and n are each 1, or one of m and n is 1.

2c1. In embodiment 2c1, the compound of embodiment 1A, 1a1, or 2A, or a pharmaceutically acceptable salt thereof, is wherein one of m and n is 1 and the other of m and n is 2.

2d1. In embodiment 2d1, the compound of embodiment 1A, 1a1, or 2A, or a pharmaceutically acceptable salt thereof, is wherein m and n are each 1.

2e1. In embodiment 2e1, the compound of embodiment 1A, 1a1, 2A, 2a1, 2b1, 2c1, or

2d1, or a pharmaceutically acceptable salt thereof, is wherein R⁶ and R⁷ are independently selected from hydrogen, methyl, and ethyl.

2f1. In embodiment 2f1, the compound of embodiment 1A, 1a1, 2A, 2a1, 2b1, 2c1, or 2d1, or a pharmaceutically acceptable salt thereof, is wherein R⁶ is cyanomethyl and R⁷ is hydrogen, methyl, or ethyl, preferably R⁷ is hydrogen.

2g1. In embodiment 2g1, the compound of embodiment 1A, 1a1, 2A, 2a1, 2b1, 2c1, or 2d1, or a pharmaceutically acceptable salt thereof, is wherein R⁶ and R⁷ are attached to the carbon atoms of the ring that are opposite or diagonal to each other and combine to form —(CH₂)_{z}— where z is 1, 2, or 3.

2h1. In embodiment 2h1, the compound of embodiment 1A, 1a1, or 2A, or 2g1, or a pharmaceutically acceptable salt thereof, is wherein R¹ is:

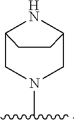 [SMILES: *N1CC2CCC(C1)N2]

2i1. In embodiment 2i1, the compound of embodiment 1A, 1a1, or 2A, or 2g1, or a pharmaceutically acceptable salt thereof, is wherein R¹ is:

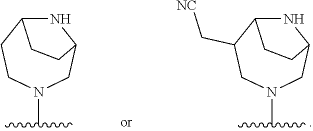 [SMILES: *N1CCC2CCC(C1)N2 ; *N1CC2CCC(N2)C(CC#N)C1]

3A. In embodiment 3A, the compound of embodiment 1A, or a pharmaceutically acceptable salt thereof, is wherein R¹ is a ring of formula

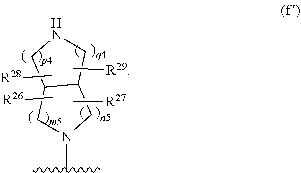

3a2. In embodiment 3a2, the compound of embodiment 1A or 3A, or a pharmaceutically acceptable salt thereof, is wherein each m5, n5, p4 and q4 is 1.

3b2. In embodiment 3b2, the compound of embodiments 1A or 6C, or a pharmaceutically acceptable salt thereof, is wherein m5 is 1, n5 is 0, p⁴ is 0, and q4 is 2.

3b2a. In embodiment 3b2a, the compound of embodiments 1A or 3A, or a pharmaceutically acceptable salt thereof, is wherein one of m5 and n5 is 1 or 2 and the other of m5 and n5 is 0, 1, or 2; and one of p4 and q4 is 1 or 2 and the other of p4 and q4 is 0, 1, or 2.

3c2. In embodiment 3c2, the compound of any one of the embodiments 1A, 3A, 3a2, 3b2 and 3b2a, or a pharmaceutically acceptable salt thereof, is wherein R²⁶ to R²⁹ are each hydrogen.

3d2. In embodiment 3d2, the compound of any one of the embodiments 1A, 3A, 3a2, 3b2 and 3b2a, or a pharmaceutically acceptable salt thereof, is wherein one or two of of R²⁶ to R²⁹ are methyl and the other of R²⁶ to R²⁹ are hydrogen.

3e2. In embodiment 3e2, the compound of any one of the embodiments 1A, 3A, 3b2, and 3b2a, or a pharmaceutically acceptable salt thereof, is wherein R²⁸ is cyano and is attached to the bridgehead carbon that is adjacent to ring N attaching ring (f′) to the remainder of the compound of Formula (II), and R²⁶, R²⁷, and R²⁹ are hydrogen.

4A. In embodiment 4A, of embodiment 1A or 3A, or a pharmaceutically acceptable salt thereof, is wherein R¹ is a ring of formula:

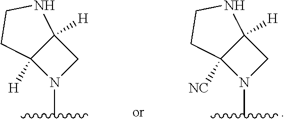 [SMILES: *N1C[C@@H]2NCC[C@@H]21 ; *N1C[C@@H]2NCC[C@@]21C#N]

5A. In embodiment 5A, the compound of embodiment 1A, or a pharmaceutically acceptable salt thereof, is wherein R¹ is a ring of formula

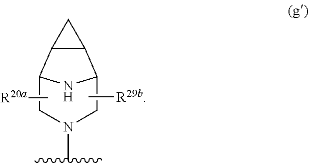

6A. In embodiment 6A, the compound of Formula (II) of any one of embodiments 1A to 5A, or a pharmaceutically acceptable salt thereof, has a structure of formula (IIa), as follows:

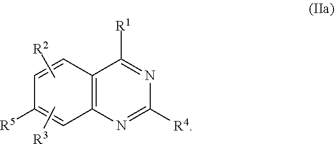

7A. In embodiment 7A, the compound of Formula (II) of any one of embodiments 1A to 5A, or a pharmaceutically acceptable salt thereof, has a structure of formula (IIb) respectively, as follows:

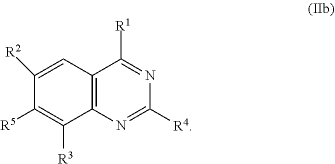

8A. In embodiment 8A, the compound of Formula (II) of any one of embodiments 1A to 5A, or a pharmaceutically acceptable salt thereof, has a structure of formula (IIc) respectively, as follows:

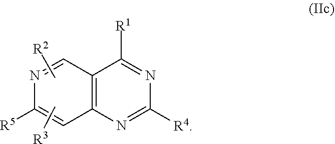

9A. In embodiment 9A, the compound of Formula (II) of any one of embodiments 1A to 5A, or a pharmaceutically acceptable salt thereof, has a structure of formula (IId) respectively, as follows:

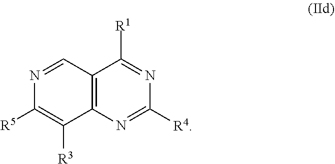

10A. In embodiment 10A, the compound of Formula (II) of any one of embodiments 1A to 5A, or a pharmaceutically acceptable salt thereof, has a structure of formula (IIe) respectively, as follows:

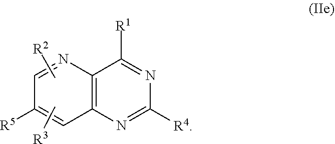

11A. In embodiment 11A, the compound of Formula (II) of any one of embodiments 1A to 5A, or a pharmaceutically acceptable salt thereof, has a structure of formula (IIf respectively, as follows:

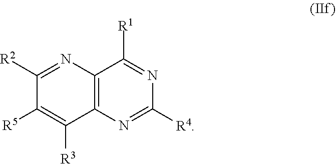

12A. In embodiment 12A, the compound of any one of embodiments 1A to 11A, or a pharmaceutically acceptable salt thereof, is wherein R⁵ is -Q-R³⁶ where Q is bond and R³⁶ is cycloalkyl, fused cycloalkyl, aryl, aralkyl, heteroaryl, or heteroaralkyl wherein aryl, aryl in aralkyl, heteroaryl, and heteroaryl in heteroaralkyl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

13A. In embodiment 13A, the compound of any one of embodiments 1A to 11A, or a pharmaceutically acceptable salt thereof, is wherein R⁵ is -Q-R³⁶ where Q is alkylene and R³⁶ is cycloalkyl, aryl, aralkyl, heteroaryl, or heteroaralkyl wherein aryl, aryl in aralkyl, heteroaryl, and heteroaryl in heteroaralkyl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

14A. In embodiment 14A, the compound of any one of embodiments 1A to 11A, or a pharmaceutically acceptable salt thereof, is wherein R⁵ is -Q-R³⁶ where Q is —C(O)— and R³⁶ is cycloalkyl, aryl, aralkyl, heteroaryl, or heteroaralkyl wherein aryl, aryl in aralkyl, heteroaryl, and heteroaryl in heteroaralkyl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

15A. In embodiment 15A, the compound of any one of embodiments 1A to 11A, or a pharmaceutically acceptable salt thereof, is wherein R³⁶ is cycloalkyl, fused cycloalkyl, aryl, aralkyl, heteroaryl, or heteroaralkyl wherein aryl, aryl in aralkyl, heteroaryl, and heteroaryl in heteroaralkyl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

16A. In embodiment 16A, the compound of any one of embodiments 1A to 11A, or a pharmaceutically acceptable salt thereof, is wherein R⁵ is -Q-R³⁶ where Q is bond and R³⁶ is phenyl or naphthyl substituted with R^{aa}, R^{bb}, R^{cc} and R^{dd}.

17A. In embodiment 17A, the compound of any one of embodiments 1A to 11A, or a pharmaceutically acceptable salt thereof, is wherein R⁵ is -Q-R³⁶ where Q is bond and R³⁶ is phenyl or naphthyl substituted with R^{aa}, R^{bb}, and R^{dd} where R^{aa} and R^{bb} are independently selected from hydrogen, alkyl, halo, haloalkyl, hydroxy, alkoxy, haloalkoxy, cycloalkyl, amino, cyano, and hydroxyalkyl and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

18A. In embodiment 18A, the compound of any one of embodiments 1A to 16A, or a pharmaceutically acceptable salt thereof, is wherein R^{aa} and R^{bb} independently selected from hydrogen, methyl, ethyl, fluoro, chloro, trifluoromethyl, difluoromethyl, trifluoromethoxy, hydroxy, methyl, ethoxy, cyclopropyl, amino, cyano, and hydroxymethyl, R^{cc} is hydrogen, ethynyl, 2-cyanoethyn-1-yl, or fluoro, and R^{dd} is hydrogen, methyl, fluoro, amino, or cyclopropyl.

19A. In embodiment 19A, the compound of any one of embodiments 1A to 11A, or a pharmaceutically acceptable salt thereof, is wherein R⁵ is -Q-R³⁶ where Q is bond and R³⁶ is heteroaryl substituted with R^{aa}, R^{bb}, R^{cc} and R^{dd}.

20A. In embodiment 20A, the compound of embodiment 1A to 11A and 19A, or a pharmaceutically acceptable salt thereof, is wherein R⁵ is -Q-R³⁶ where Q is bond and R³⁶ is a monocyclic heteroaryl (e.g., pyridyl, pyrimidinyl) substituted with R^{aa}, R^{bb}, R^{cc} and R^{dd}.

21A. In embodiment 21A, the compound of embodiment 1A to 11A and 19A, or a pharmaceutically acceptable salt thereof, is wherein R⁵ is -Q-R³⁶ where Q is bond and R³⁶ is bicyclic heteroaryl (e.g, quinolinyl, isoquinolinyl, or indazolyl), substituted with R^{aa}, R^{bb}, R^{cc} and R^{dd}.

22A. In embodiment 22A, the compound of any one of embodiments 1A to 11A and 19A to 21A, or a pharmaceutically acceptable salt thereof, is wherein the heteroaryl is substituted with R^{aa}, R^{bb}, and R^{dd} where R^{aa} and R^{bb} independently selected from hydrogen, alkyl, halo, haloalkyl, hydroxy, alkoxy, haloalkoxy, cycloalkyl, amino, cyano, and hydroxyalkyl and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

23A. In embodiment 23A, the compound of any one of embodiments 1A to 11A and 19A to 21A, or a pharmaceutically acceptable salt thereof, is wherein R^{aa} and R^{bb} are independently selected from hydrogen, methyl, ethyl, fluoro, chloro, trifluoromethyl, difluoromethyl, trifluoromethoxy, hydroxy, methyl, ethoxy, cyclopropyl, amino, cyano, and hydroxymethyl, R^{cc} is hydrogen or fluoro, and R^{dd} is hydrogen, methyl, fluoro, amino, or cyclopropyl.

24A. In embodiment 24A, the compound of any one of embodiments 1A to 11A, or a pharmaceutically acceptable salt thereof, is wherein R⁵ is -Q-R³⁶ where Q is bond and R³⁶ is:

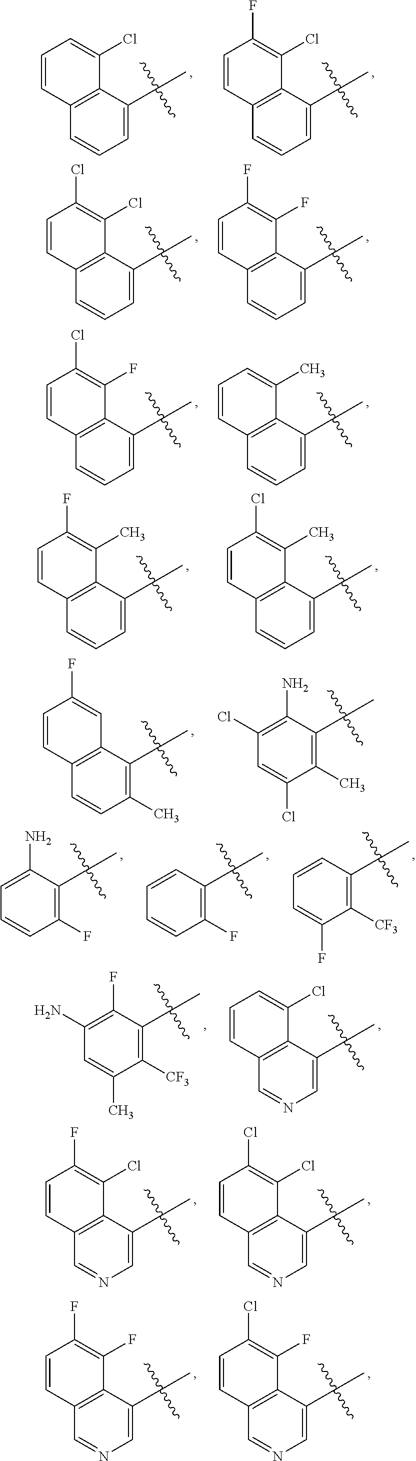

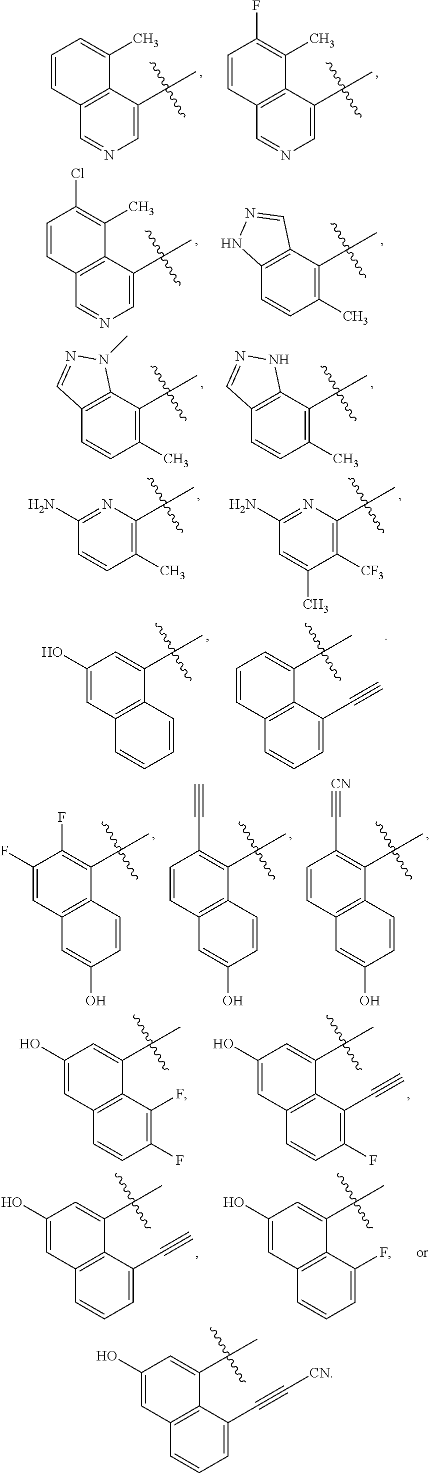

25A. In embodiment 25A, the compound of any one of embodiments 1A to 11A, or a pharmaceutically acceptable salt thereof, is wherein R⁵ is -Q-R³⁶ where Q is bond and R³⁶ is:

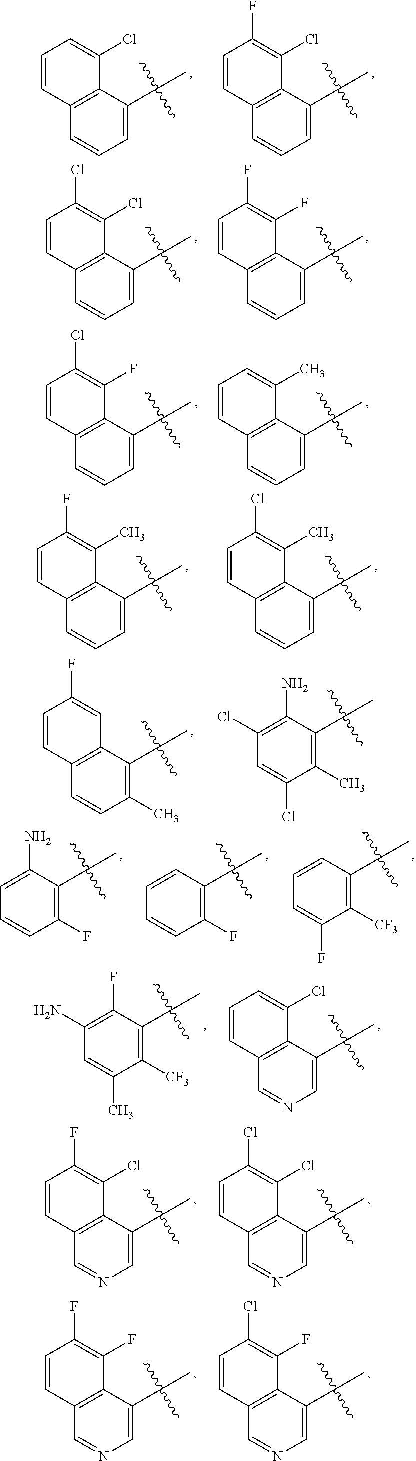

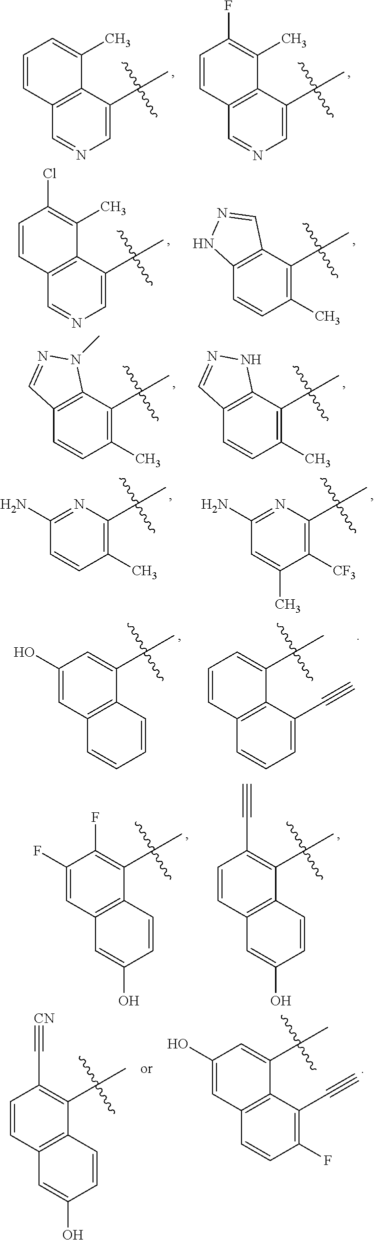

26A. In embodiment 26A, the compound of any one of embodiments 1A to 25A, or a pharmaceutically acceptable salt thereof, is wherein R² is hydrogen, halo, or alkyl, and R³ hydrogen, halo, cycloalkyloxy, or alkyl.

27A. In embodiment 27A, the compound of any one of embodiments 1A to 26A, or a pharmaceutically acceptable salt thereof, is wherein R² and R³ are each hydrogen.

28A. In embodiment 28A, the compound of any one of embodiments 1A to 26A, or a pharmaceutically acceptable salt thereof, is wherein R² is hydrogen or chloro and R³ is hydrogen, fluoro, or cyclopropyloxy.

29A. In embodiment 29A, the compound of any one of embodiments 6A to 28A, or a pharmaceutically acceptable salt thereof, is wherein R⁴ is —Z—R³⁰ where Z is a bond, O, NH, N(alkyl), or S; and R³⁰ is alkyl, hydroxyalkyl, -(alkylene)-NR³¹R³² (where alkylene is substituted with R^{a}, R^{b}, and R^{c} independently selected from hydrogen, alkyl, cycloalkyl, halo, haloalkyl, hydroxy, alkoxy, cyano, oxo, hydroxyalkyl, alkylamino, dialkylamino, dialkylaminocarbonylalkyl, aryl, heteroaryl, and heterocyclyl, R³¹ is hydrogen or alkyl, and R³² is hydrogen, alkyl, acyl, hydroxyalkyl, or heteroalkyl), aryl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkoxyalkyl, alkoxyalkyloxy, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl.

30A. In embodiment 30A, the compound of any one of the embodiments 1A, 1a1, 2A to 2i1, and 3A to 29A, or a pharmaceutically acceptable salt thereof, is wherein Z is O.

31A. In embodiment 31A, the compound of any one of the embodiments 1A, 1a1, 2A to 2i1, and 3A to 29A, or a pharmaceutically acceptable salt thereof, is wherein Z is NH.

32A. In embodiment 32A, the compound of any one of the embodiments 1A, 1a1, 2A to 2i1, and 3A to 29A, or a pharmaceutically acceptable salt thereof, is wherein Z is bond.

33A. In embodiment 33A, the compound of any one of embodiments 6A to 32A, or a pharmaceutically acceptable salt thereof, is wherein R³⁰ is hydroxyalkyl, -(alkylene)-NR³¹R³² (where alkylene is substituted with R^{a}, R^{b}, and R^{c} independently selected from hydrogen, alkyl, hydroxy, and hydroxyalkyl, R³¹ is hydrogen or alkyl, and R³² is hydrogen, alkyl, or hydroxyalkyl), heteroaralkyl, heterocyclyl, heterocyclylalkyl, bicyclic heterocyclyl, bicyclic heterocyclylalkyl, bridged heterocyclylalkyl, fused heterocyclylalkyl, and spiro heterocyclylalkyl, wherein heterocyclyl, by itself or as part of heterocyclylalkyl, bicyclic heterocyclyl, by itself or as part of bicyclic heterocyclylalkyl, bridged heterocyclyl as part of bridged heterocyclylalkyl, fused heterocyclyl, as part of fused heterocyclylalkyl, and spiro heterocyclyl as part of spiro heterocyclylalkyl, are substituted with R^{d}, R^{e}, and R^{f} independently selected from hydrogen, alkyl, cycloalkyl, cycloalkylalkyl, halo, hydroxy, alkoxy, alkoxyalkyl, acyl, hydroxyalkyl, alkylamino, dialkylamino, cyano, and optionally substituted aralkyl.

34A. In embodiment 34A, the compound of any one of embodiments 6A to 32A, or a pharmaceutically acceptable salt thereof, is wherein R³⁰ is 2-dimethylaminoethyl, diethylaminoethyl, 3-methylaminoprop-2-yl, 3-dimethylaminopropyl, 3-dimethylaminoprop-2-yl, 4-dimethylaminobut-2-yl, 4-dimethylaminobut-3-yl, 4-dimethylaminobutyl, 2-dimethylamino-3-hydroxypropyl, 2-dimethylaminoprop-1-yl, 4-methylpiperazin-1-yl, 4-(2-hydroxyethyl)piperazin-1-yl, 4-methylpiperazin-2-yl)methyl, 3-(4-methylpiperazin-1-yl)propyl, 4-dimethylaminopiperidin-1-yl, 1-methylpiperidin-4-yl, piperidin-2-ylmethyl, 2-piperidin-1-ylethyl, 3-piperidin-1-ylpropyl, 3-piperidin-1-ylprop-2-yl, 1-methylpiperidin-3-yl, 2-oxopiperidin-6-ylmethyl, 2-(4-cyanopiperidin-1-yl)ethyl, 2-(2-methylpiperidin-1-yl)ethyl, 2-(4-methylpiperidin-1-yl)ethyl, 3-methoxypiperidin-1-ylethyl, 4-methoxypiperidin-1-ylethyl, 1-cyclopropylpiperidin-4-yl, 2-(4,4-difluoropiperidin-1-yl)ethyl, 2-(3-fluoropiperidin-1-yl)ethyl, 1-methylpiperidin-3-ylmethyl, pyrrolidin-1-yl, pyrrolidin-2-ylmethyl, pyrrolidin-3-ylmethyl, pyrrolidin-1-ylethyl, 3-pyrrolidin-1-ylprop-2-yl, 1-methylpyrrolidin-3-yl, 3-pyrrolidin-1-ylpropyl, 3-fluoropyrrolidin-1-ylethyl, 3,3-difluoropyrrolidin-1-ylethyl, 3-dimethylaminopyrrolidin-1-yl, 2-oxopyrrolidin-5-ylmethyl, 2-(3-methoxypyrrolidin-1-yl)ethyl, 3-(3-methoxypyrrolidin-1-yl)propyl, 3-methoxypyrrolidin-1-yl)prop-2-yl, 3-(3-hydroxypyrrolidin-1-yl)prop-2-yl, 1-methylpyrrolidin-3-ylmethyl, 1-methylpyrrolidin-2-ylmethyl, 1-ethylpyrrolidin-2-ylmethyl, 1-methylpyrrolidin-3-ylmethyl, 2-(1-methylpyrrolidin-2-yl)ethyl, 1-(2-hydroxyethyl)pyrrolidin-3-ylmethyl, 1-(2-methoxyethyl)-pyrrolidin-3-ylmethyl, 1-isopropylpyrrolidin-3-ylmethyl, 5,5-dimethylpyrrolidin-2-yl, 1-benzylpyrrolidin-3-ylmethyl, 1-cyclopropylpyrrolidin-3-ylmethyl, 3-(3,4-difluoropyrrolidin-1-yl)propyl, 3-hydroxy-1-methylpyrrolidin-2-ylmethyl, 4-hydroxy-1-methylpyrrolidin-2-ylmethyl, 3-fluoro-1-methylpyrrolidin-2-ylmethyl, 4-fluoro-1-methylpyrrolidin-2-ylmethyl, 4,4-difluoro-1-methylpyrrolidin-2-ylmethyl, 4-methoxy-1-methylpyrrolidin-2-ylmethyl, 1,2-dimethylpyrrolidin-2-ylmethyl, 1-isopropylpyrrolidin-2-ylmethyl, 1-cyclopropylmethylpyrrolidin-2-ylmethyl, 1,5,5-trimethylpyrrolidin-2-yl, 4-methoxy-1-methylpyrrolidin-2-ylmethyl, 4-methoxy-1-ethylpyrrolidin-2-ylmethyl, morpholin-4-yl, 2-morpholin-4-ylethyl, 3-morpholin-4-ylpropyl, 3-morpholin-3-ylprop-2-yl, 4-morpholin-4-ylbutyl, 4-morpholin-4-ylbut-2-yl, 4-methylmorpholin-2-ylmethyl, 4-methylmorpholin-3-ylmethyl, 5-methylmorpholin-3-ylmethyl, 5,5-dimethylmorpholin-3-ylmethyl, 2-((1S,4R)-2-azabicyclo[2.2.1]heptan-2-yl)ethyl, 2-pyridin-2-ylethyl, or 3-(3-azabicyclo[3.1.0]-hexan-3-ylpropyl.

35A. In embodiment 35A, the compound of any one of embodiments 1A, 2A, 2a1 to 2i1, 3A, 3a2 to 3e2, 4A to 28A, or a pharmaceutically acceptable salt thereof, is wherein R⁴ is —Z—R³⁰ where Z is a bond, O, NH, N(alkyl), or S; and R³⁰ is phenyl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein phenyl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}. R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl.

36A. In embodiment 36A, the compound of any one of embodiments 1A, 2A, 2a1 to 2i1, 3A to 28A, and 30A to 32A, or a pharmaceutically acceptable salt thereof, is wherein R³⁰ is 4-methylpiperazin-1-yl, 4-(2-hydroxyethyl)piperazin-1-yl, 4-methylpiperazin-2-yl)methyl, 3-(4-methylpiperazin-1-yl)propyl, 4-dimethylaminopiperidin-1-yl, 1-methylpiperidin-4-yl, piperidin-2-ylmethyl, 2-piperidin-1-ylethyl, 3-piperidin-1-ylpropyl, 3-piperidin-1-ylprop-2-yl, 1-methylpiperidin-3-yl, 2-oxopiperidin-6-ylmethyl, 2-(4-cyanopiperidin-1-yl)ethyl, 2-(2-methylpiperidin-1-yl)ethyl, 2-(4-methylpiperidin-1-yl)ethyl, 3-methoxypiperidin-1-ylethyl, 4-methoxypiperidin-1-ylethyl, 1-cyclopropylpiperidin-4-yl, 2-(4,4-difluoropiperidin-1-yl)ethyl, 2-(3-fluoropiperidin-1-yl)ethyl, 1-methylpiperidin-3-ylmethyl, pyrrolidin-1-yl, pyrrolidin-2-ylmethyl, pyrrolidin-3-ylmethyl, pyrrolidin-1-ylethyl, 3-pyrrolidin-1-ylprop-2-yl, 1-methylpyrrolidin-3-yl, 3-pyrrolidin-1-ylpropyl, 3-fluoropyrrolidin-1-ylethyl, 3,3-difluoropyrrolidin-1-ylethyl, 3-dimethylaminopyrrolidin-1-yl, 2-oxopyrrolidin-5-ylmethyl, 2-(3-methoxypyrrolidin-1-yl)ethyl, 3-(3-methoxypyrrolidin-1-yl)propyl, 3-methoxypyrrolidin-1-yl)prop-2-yl, 3-(3-hydroxypyrrolidin-1-yl)prop-2-yl, 1-methylpyrrolidin-3-ylmethyl, 1-methylpyrrolidin-2-ylmethyl, 1-ethylpyrrolidin-2-ylmethyl, 1-methylpyrrolidin-3-ylmethyl, 2-(1-methylpyrrolidin-2-yl)ethyl, 1-(2-hydroxyethyl)pyrrolidin-3-ylmethyl, 1-(2-methoxyethyl)-pyrrolidin-3-ylmethyl, 1-isopropylpyrrolidin-3-ylmethyl, 5,5-dimethylpyrrolidin-2-yl, 1-benzylpyrrolidin-3-ylmethyl, 1-cyclopropylpyrrolidin-3-ylmethyl, 3-(3,4-difluoropyrrolidin-1-yl)propyl, 3-hydroxy-1-methylpyrrolidin-2-ylmethyl, 4-hydroxy-1-methylpyrrolidin-2-ylmethyl, 3-fluoro-1-methylpyrrolidin-2-ylmethyl, 4-fluoro-1-methylpyrrolidin-2-ylmethyl, 4,4-difluoro-1-methylpyrrolidin-2-ylmethyl, 4-methoxy-1-methylpyrrolidin-2-ylmethyl, 1,2-dimethylpyrrolidin-2-ylmethyl, 1-isopropylpyrrolidin-2-ylmethyl, 1-cyclopropylmethylpyrrolidin-2-ylmethyl, 1,5,5-trimethylpyrrolidin-2-yl, 4-methoxy-1-methylpyrrolidin-2-ylmethyl, 4-methoxy-1-ethylpyrrolidin-2-ylmethyl, morpholin-4-yl, 2-morpholin-4-ylethyl, 3-morpholin-4-ylpropyl, 3-morpholin-3-ylprop-2-yl, 4-morpholin-4-ylbutyl, 4-morpholin-4-ylbut-2-vl, 4-methylmorpholin-2-ylmethyl, 4-methylmorpholin-3-ylmethyl, 5-methylmorpholin-3-ylmethyl, 5,5-dimethylmorpholin-3-ylmethyl, 2-((1S,4R)-2-azabicyclo[2.2.1]heptan-2-yl)ethyl, 2-pyridin-2-ylethyl, or 3-(3-azabicyclo[3.1.0]-hexan-3-ylpropyl.

37A. In embodiment 37A, the compound of any one of embodiments 1A to 28A, or a pharmaceutically acceptable salt thereof, is wherein R⁴ is:

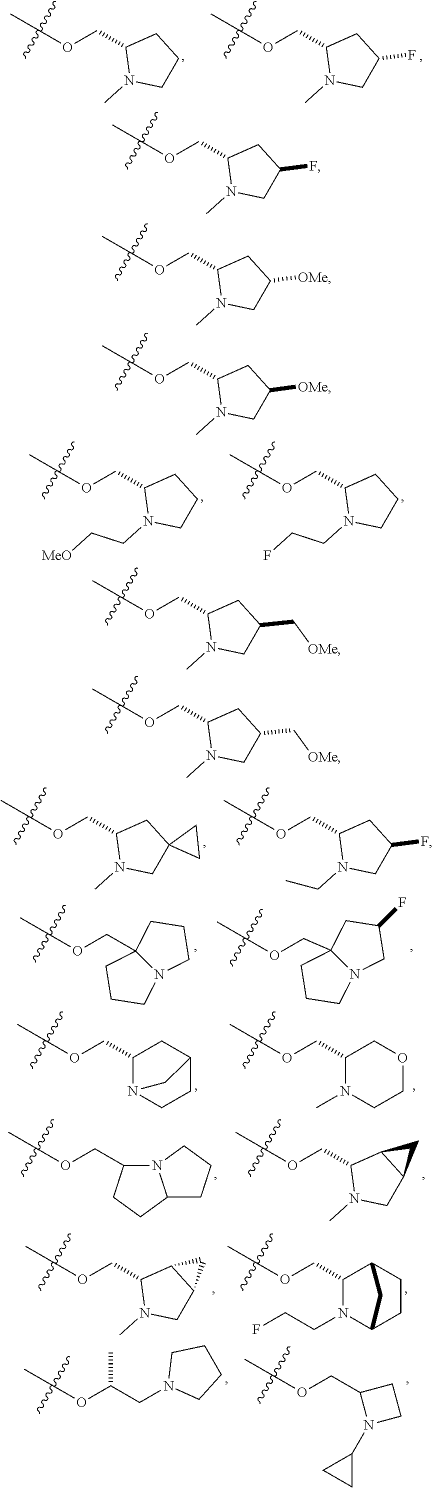

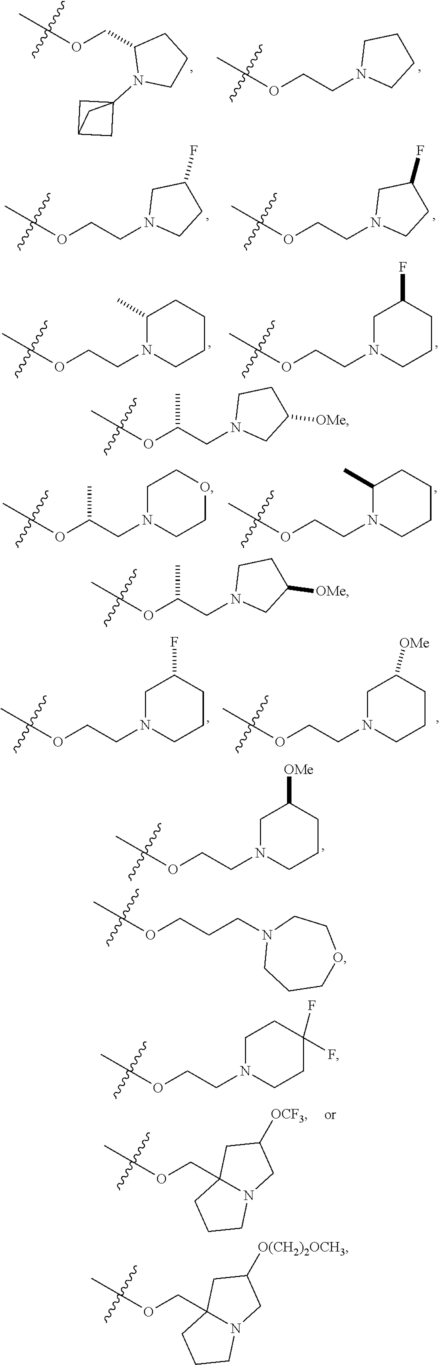

38A. In embodiment 38A, the compound of any one of embodiments 1A to 28A, or a pharmaceutically acceptable salt thereof, is wherein R⁴ is —CR³³═CR³⁴R³⁵ where R³³ and R³⁴ are independently selected from hydrogen, deuterium, alkyl, halo, and haloalkyl; and R³⁵ is hydrogen, deuterium, alkyl, halo, haloalkyl, cycloalkyl, bridged cycloalkyl, fused cycloalkyl, spiro cycloalkyl, heterocyclyl, bicyclic heterocyclyl, bridged heterocyclyl, fused heterocyclyl, or spiro heterocyclyl; or

R³⁴ and R³⁵ together with the carbon atom to which are attached form cycloalkyl, bridged cycloalkyl, fused cycloalkyl, spiro cycloalkyl, heterocyclyl, bridged heterocyclyl, fused heterocyclyl, or spiro heterocyclyl, wherein:

(a) the groups alkyl, cycloalkyl, bridged cycloalkyl, fused cycloalkyl, spiro cycloalkyl, heterocyclyl, bicyclic heterocyclyl, bridged heterocyclyl, fused heterocyclyl, and spiro heterocyclyl of R³⁵; and (b) the groups cycloalkyl, bridged cycloalkyl, fused cycloalkyl, spiro cycloalkyl, heterocyclyl, bridged heterocyclyl, fused heterocyclyl, or spiro heterocyclyl formed by R³⁴ and R³⁵ together, are independently substituted with R^{h}, Rⁱ, and R^{j} where R^{h}, Rⁱ and R^{j} are independently selected from hydrogen, alkyl, halo, haloalkyl, hydroxyalkyl, alkoxyalkyl, cyano, cycloalkyl, bridged cycloalkyl, heterocyclyl, —O(alk)_{z1}R^{k}, —O(alk)OR^{l}, —S(O)R^{m}, —S(O)₂Rⁿ, —NR^{p}C(O)R^{o}, —NR^{r}SO₂R^{q}, —OC(O)NR^{s}R^{t}, —C(O)NR^{u}R^{v}, —S(O)₂NR^{w}R^{x}, and —NR^{y}R^{z}, where z1 is 0 or 1, alk is alkylene, and R^{k}, R^{l}, R^{m}, Rⁿ, R^{o}, R^{P}, R^{q}, R^{r}, R^{s}, R^{t}, R^{u}, R^{v}, R^{w}, R^{x}, R^{y}, and R^{z} are independently selected from hydrogen, alkyl, cycloalkyl, cycloalkylalkyl, halo, hydroxyalkyl, alkoxyalkyl, and aminoalkyl.

39A. In embodiment 39A, the compound of any one of embodiments 1A to 28A, or a pharmaceutically acceptable salt thereof, is wherein R⁴ is —CR³³═CR³⁴R³⁵ where R³³ and R³⁴ are independently hydrogen, deuterium, or alkyl; and R³⁵ is heterocyclyl, bicyclic heterocyclyl, bridged heterocyclyl, fused heterocyclyl, or spiro heterocyclyl wherein:

heterocyclyl, bicyclic heterocyclyl, bridged heterocyclyl, fused heterocyclyl, and spiro heterocyclyl of R³⁵ are substituted with R^{h}, Rⁱ, and R^{j} independently selected from hydrogen, alkyl, halo, haloalkyl, hydroxyalkyl, alkoxyalkyl, cyano, cycloalkyl, bridged cycloalkyl, optionally substituted heterocyclyl, —O(alk)_{z1}R^{k}, —O(alk)OR^{l}, and —NR^{y}R^{z}, where z1 is 0 or 1, alk is alkylene, and R^{k}, R^{l}, R^{y}, and R^{z} are independently selected from hydrogen, alkyl, cycloalkyl, cycloalkylalkyl, halo, hydroxyalkyl, alkoxyalkyl, and aminoalkyl.

40A. In embodiment 40A, the compound of any one of embodiments 1A to 28A, or a pharmaceutically acceptable salt thereof, is wherein R⁴ is:

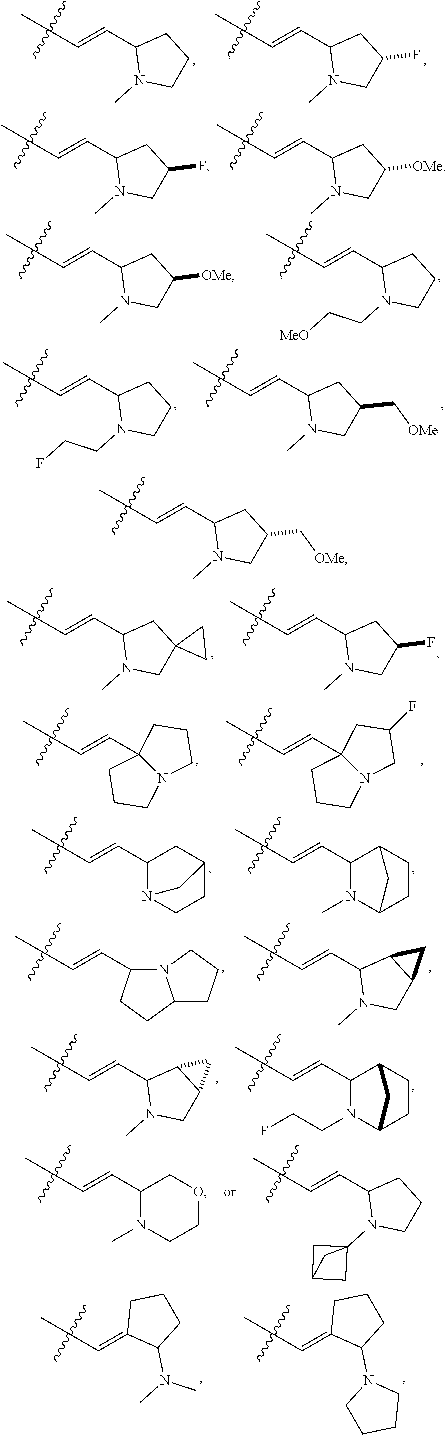

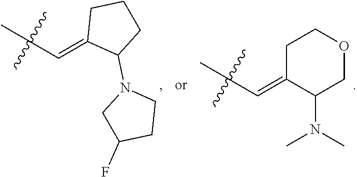 [SMILES: */C=C1\CCCC1N1CCC(F)C1 ; */C=C1\CCOCC1N(C)C]

41A. In embodiment 41A, the compound of any one of embodiments 1A to 28A, or a pharmaceutically acceptable salt thereof, is wherein R⁴ is —CR³³═CR³⁴R³⁵ where R³³ is hydrogen, deuterium, or alkyl and R³⁴ and R³⁵ together with the carbon atom to which are attached form cycloalkyl, bridged cycloalkyl, fused cycloalkyl, spiro cycloalkyl, heterocyclyl, bridged heterocyclyl, fused heterocyclyl, or spiro heterocyclyl, wherein:

cycloalkyl, bridged cycloalkyl, fused cycloalkyl, spiro cycloalkyl, heterocyclyl, bicyclic heterocyclyl, bridged heterocyclyl, fused heterocyclyl, and spiro heterocyclyl formed by R³⁴ and R³⁵ together, are independently substituted with R^{h}, Rⁱ, and R^{j} where R^{h}, Rⁱ and R^{j} are independently selected from hydrogen, alkyl, halo, haloalkyl, hydroxy alkyl, alkoxyalkyl, cyano, cycloalkyl, bridged cycloalkyl, optionally substituted heterocyclyl, —O(alk)_{z1}R^{k}, —O(alk)OR^{l}, and —NR^{y}R^{z}, where z1 is 0 or 1, Ak is alkylene, and R^{k}, R^{l}, R^{y}, and R^{z} are independently hydrogen or alkyl.

42A. In embodiment 42A, the compound of embodiment 40A, or a pharmaceutically acceptable salt thereof, is wherein R⁴ is:

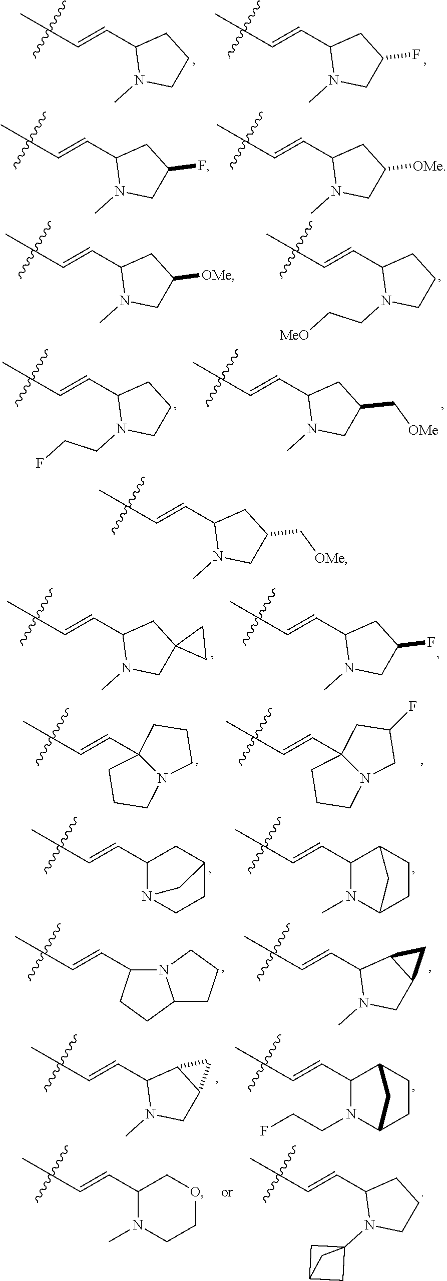

43A. In embodiment 43A, the compound of embodiment 41A, or a pharmaceutically acceptable salt thereof, is wherein R⁴ is:

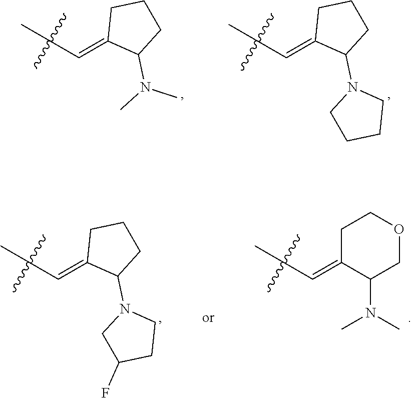

44A. In embodiment 44A, the compound of any one of embodiments 1A, 2A, 2a1 to 2i1, 3A to 28A, or a pharmaceutically acceptable salt thereof, is wherein R⁴ is —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that; when R³⁰ is aryl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

45A. In embodiment 45A, the compound of any one of embodiments 1A, 2A, 2a1 to 2i1, 3A to 28A, or a pharmaceutically acceptable salt thereof, is wherein R⁴ is —Z—R³⁰ where Z is a O; and R³⁰ is heterocyclylalkyl, bicyclic heterocyclylalkyl, phosphinyl bicyclic heterocyclyl, phosphinyl bicyclic heterocyclylalkyl, or fused bicyclic heterocyclylalkyl, wherein heterocyclyl as part of heterocyclylalkyl, bicyclic heterocyclyl as part of bicyclic heterocyclylalkyl, phosphinyl bicyclic heterocyclyl, by itself or as part of phosphinyl bicyclic heterocyclylalkyl, or fused bicyclic heterocyclyl as part of fused bicyclic heterocyclylalkyl are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that; when R³⁰ is heterocyclylalkyl or bicyclic heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

46A. In embodiment 46A, the compound of any one of embodiments 1A, 2A, 2a1 to 2i1, 3A to 28A, or a pharmaceutically acceptable salt thereof, is wherein R⁴ is —Z—R³⁰ where Z is a bond, O, NH, N(alkyl), or S; and R³⁰ is phosphinyl bicyclic heterocyclylalkyl or bicyclic heterocyclylalkyl, wherein bicyclic heterocyclyl as part of bicyclic heterocyclylalkyl and phosphinyl bicyclic heterocyclyl as part of phosphinyl bicyclic heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is bicyclic heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

47A. In embodiment 47A, the compound of any one of embodiments 1A, 2A, 2a1 to 2i1, 3A to 28A, or a pharmaceutically acceptable salt thereof, is wherein R⁴ is —Z—R³⁰ where Z is O; and R³⁰ is phosphinyl bicyclic heterocyclylalkyl or bicyclic heterocyclylalkyl, wherein bicyclic heterocyclyl as part of bicyclic heterocyclylalkyl and phosphinyl bicyclic heterocyclyl as part of phosphinyl bicyclic heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is bicyclic heterocyclylalkyl, then at least one of R^{d}. R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

48A. In embodiment 48A, the compound of any one of embodiments 46A and 47A, or a pharmaceutically acceptable salt thereof, is wherein R⁴ is

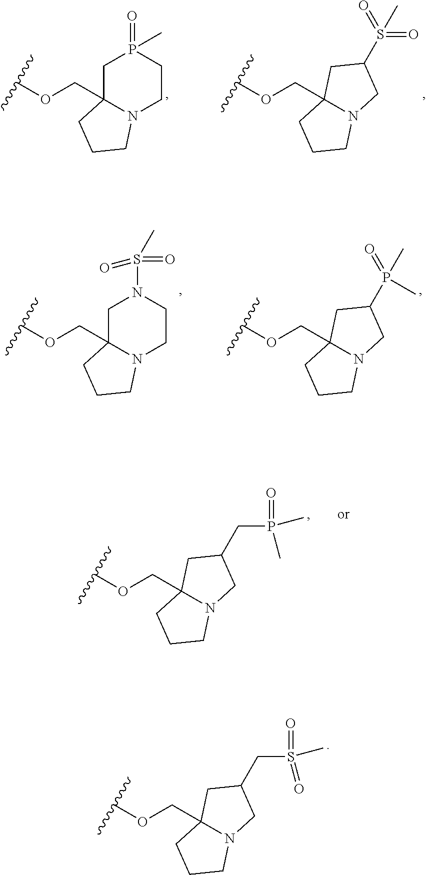 [SMILES: *OCC12CCCN1CCP(C)(=O)C2 ; *OCC12CCCN1CC(S(C)(=O)=O)C2 ; *OCC12CCCN1CCN(S(C)(=O)=O)C2 ; *OCC12CCCN1CC(P(C)(C)=O)C2 ; *OCC12CCCN1CC(CP(C)(C)=O)C2 ; *OCC12CCCN1CC(CS(C)(=O)=O)C2]

### Embodiments B

In further embodiments B1A′-B53A below, the present disclosure includes:

B1A1′. In embodiment B1A1′, provided is a compound of Formula (IIA1′) as defined in the first embodiment of first aspect of the Summary.

B1A′. In embodiment B1A′, provided is a compound of Formula (IIA′) as defined in the second embodiment of first aspect of the Summary.

B1A. In embodiment B1A, provided is a compound of Formula (II′) as defined in second aspect of the Summary.

B1a1. In embodiment B1a1, the compound of embodiment B1A1′, B1A′ or B1A is wherein R⁴ is:

(i) —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, tricyclic heterocyclyl, tricyclic heterocyclylalkyl, fused tricyclic heterocyclyl, fused tricyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, tricyclic heterocyclyl, by itself or as part of tricyclic heterocyclylalkyl, fused tricyclic heterocyclyl, by itself or as part of fused tricyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, alkenyl, haloalkenyl, cycloalkyl, cycloalkyloxy, cycloalkylalkyl, bridged cycloalkyl, halo, haloalkyl, haloalkoxy, hydroxy, alkoxy, alkylidinyl, haloalkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is aryl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, then at least one of R^{d}, R^{e}, and R^{f} is alkylidenyl, haloalkylidenyl, alkoxyalkylidenyl, alkylsulfonyl, alkylsulfonylalkyl, dialkyl(oxo)phosphinyl, or dialkyl(oxo)phosphinylalkyl; or

(ii) —CR³═CR³⁴R³⁵ where R³³ and R³⁴ are independently selected from hydrogen, deuterium, alkyl, halo, and haloalkyl; and

R³⁵ is hydrogen, deuterium, alkyl, halo, haloalkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, or spiro heterocyclyl; or

R³⁴ and R³⁵ together with the carbon atom to which are attached form cycloalkyl, bridged cycloalkyl, fused cycloalkyl, spiro cycloalkyl, heterocyclyl, bridged heterocyclyl, fused heterocyclyl, or spiro heterocyclyl, wherein:

(a) the groups alkyl, cycloalkyl, bridged cycloalkyl, fused cycloalkyl, spiro cycloalkyl, heterocyclyl, bicyclic heterocyclyl, phosphinyl bicyclic heterocyclyl, bridged heterocyclyl, fused heterocyclyl, and spiro heterocyclyl of R³⁵; and (b) the groups cycloalkyl, bridged cycloalkyl, fused cycloalkyl, spiro cycloalkyl, heterocyclyl, bridged heterocyclyl, fused heterocyclyl, or spiro heterocyclyl formed by R³⁴ and R³ together, are independently substituted with R^{h}, Rⁱ, and R^{j} independently selected from hydrogen, alkyl, halo, haloalkyl, hydroxyalkyl, alkoxyalkyl, cyano, cycloalkyl, bridged cycloalkyl, optionally substituted heterocyclyl, alkylsulfonylalkyl, dialkyl(oxo)phosphinyl, dialkyl(oxo)phosphinylalkyl, —O(alk)_{z1}R^{k}, —O(alk)OR^{l}, —S(O)R^{m}, —S(O)₂Rⁿ, —NR^{p}C(O)R^{o}, —NR^{r}SO₂R^{q}, —OC(O)NR^{s}R^{t}, —C(O)NR^{u}R^{v}, —S(O)₂NR^{w}R^{x}, and —NR^{y}R^{z}, where z1 is 0 or 1, alk is alkylene, and R^{k}, R^{l}, R^{m}, Rⁿ, R^{o}, R^{p}, R^{q}, R^{r}, R^{s}, R^{t}, R^{u}, R^{v}, R^{w}, R^{x}, R^{y}, and R^{z} are independently selected from hydrogen, alkyl, cycloalkyl, cycloalkylalkyl, halo, hydroxyalkyl, alkoxyalkyl, and aminoalkyl; and

R⁵ is -Q-R³⁶ where Q is bond, alkylene, or —C(═O)—; and R³⁶ is hydrogen, cycloalkyl, fused cycloalkyl, aryl, aralkyl, heteroaryl, or heteroaralkyl wherein aryl, aryl in aralkyl, heteroaryl, and heteroaryl in heteroaralkyl are independently substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

B2A. In embodiment B2A, the compound of embodiment B1A1′, B1A′, B1A, or B1a1, or a pharmaceutically acceptable salt thereof, is wherein R¹ is a ring of formula

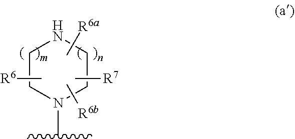

where m and n are independently 0, 1, or 2.

B2a1. In embodiment B2a1, the compound of embodiment B1A1′, B1A′, B1A, B1a1, or B2A, or a pharmaceutically acceptable salt thereof, is wherein m and n are each 1, or one of m and n is 1 and the other of m and n is 2.

B2b1. In embodiment B2b1, the compound of embodiment B1A1′, B1A′, B1A, B1a1, or B2A, or a pharmaceutically acceptable salt thereof, is wherein m and n are each 1, or one of m and n is 1.

B2c1. In embodiment B2c1, the compound of embodiment B1A1′, B1A′, B1A, B1a1, or B2A, or a pharmaceutically acceptable salt thereof, is wherein one of m and n is 1 and the other of m and n is 2.

B2d1. In embodiment B2d1, the compound of embodiment B1A1′, B1A′, B1A, B1a1, or B2A, or a pharmaceutically acceptable salt thereof, is wherein m and n are each 1.

B2e1. In embodiment B2e1, the compound of embodiment B1A1′, B1A′, B1A, B1a1, B2A, B2a1, B2b1, B2c1, or B2d1, or a pharmaceutically acceptable salt thereof, is wherein R⁶ and Rare independently selected from hydrogen, methyl, and ethyl, and R^{6a} and R^{6b} are hydrogen.

B2f1. In embodiment B2f1, the compound of embodiment B1A1′, B1A′, B1A, B1a1, B2A, B2a1, B2b1, B2c1, or B2d1, or a pharmaceutically acceptable salt thereof, is wherein R⁶ is cyanomethyl and R⁷ is hydrogen, methyl, or ethyl, preferably R′ is hydrogen, and R^{6a} and R^{6b} are hydrogen.

B2g1. In embodiment B2g1, the compound of embodiment B1A1′, B1A′, B1A, B1a1, B2A, B2a1, B2b1, B2c1, or B2d1, or a pharmaceutically acceptable salt thereof, is wherein R⁶ and R? are attached to the carbon atoms of the ring that are opposite or diagonal to each other and combine to form —(CH₂)_{z}— where z is 1, 2, or 3, preferably z is 2, and R^{6a} and R^{6b} are hydrogen.

B2h1. In embodiment B2h1, the compound of embodiment B1A1′, B1A′, B1A, B1a1, B2A, B2a1, B2b1, B2c1, or B2d1, or a pharmaceutically acceptable salt thereof, is wherein R⁶ and Rare attached to the carbon atoms of the ring that are opposite or diagonal to each other and combine to form —CH═CH— and R^{6a} and R^{6b} are hydrogen.

B2i1. In embodiment B2i1, the compound of embodiment B1A1′, B1A′, B1A, B1a1, B2A, B2a1, B2b1, B2c1, or B2d1, or a pharmaceutically acceptable salt thereof, is wherein R⁶ and R⁷ are attached to the carbon atoms of the ring that are opposite or diagonal to each other and combine to form —(CH₂)_{z}— where z is 1, 2, or 3, preferably 2, and R^{6a} is alkylidienyl, preferably ═CH₂, and R^{6b} is hydrogen.

B2j1. In embodiment B2j1, the compound of embodiment B1A1′, B1A′, B1A, B1a1, B2A, B2a1, B2b1, B2c1, or B2d1, or a pharmaceutically acceptable salt thereof, is wherein R⁶ and R⁷ are attached to the carbon atoms of the ring that are opposite or diagonal to each other and combine to form —(CH₂)_{z}— where z is 1, 2, or 3, preferably 2, and R^{6a} and R^{6b} are attached to the same carbon of the —(CH₂)_{z}— group and are combined to form cycloalkylene, preferably 1,1-cyclopropylene.

B2k1. In embodiment B2k1, the compound of embodiment B1A1l, B1A′, B1A, B1a1, or B2A, or a pharmaceutically acceptable salt thereof, is wherein R¹ is:

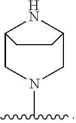 [SMILES: *N1CC2CCC(C1)N2]

B2l1. In embodiment B2l1, the compound of embodiment B1A1, B1A′, B1A, B1a1, or B2A, or a pharmaceutically acceptable salt thereof, is wherein R¹ is:

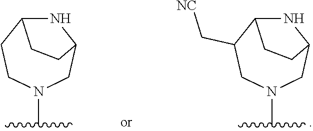 [SMILES: *N1CCC2CCC(C1)N2 ; *N1CC2CCC(N2)C(CC#N)C1]

B2 m1. In embodiment B2 m1, the compound of embodiment B1A1, B1A′, B1A, B1a1, or B2A, or a pharmaceutically acceptable salt thereof, is wherein R¹ is:

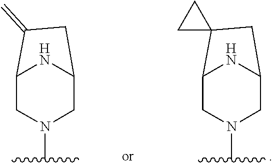

B2n1. In embodiment B2n1, the compound of embodiment B1A1′, B1A′, B1A, B1a1, or B2A, or a pharmaceutically acceptable salt thereof, is wherein R¹ is:

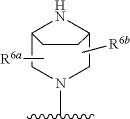

where R^{6a} and R^{6b} are other than hydrogen.

B2o1. In embodiment B2o1, the compound of embodiment B1A1′, B1A′, B1A, B1a1, or B2A, or a pharmaceutically acceptable salt thereof, is wherein R¹ is:

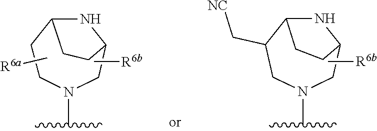

where R^{6b} is other than hydrogen.

B2A1. In embodiment B2A1, the compound of embodiment B1A1′, B1A′ or B1a1, or a pharmaceutically acceptable salt thereof, is wherein R¹ is a ring of formula

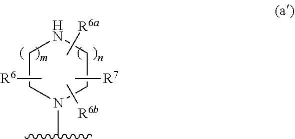

where m is 0, 1, or 2 and n is 3.

B2A1a. In embodiment B2A1a, the compound of embodiment B1A1′, B1A′, B2A1, or B1a1, or a pharmaceutically acceptable salt thereof, is wherein m is 0.

B2A1b. In embodiment B2A1b, the compound of embodiment B1A1′, B1A′, B2A1, or B1a1, or a pharmaceutically acceptable salt thereof, is wherein m is 1.

B2A1c. In embodiment B2A1c, the compound of embodiment B1A1′, B1A′, B2A1, B1a1, B2A1a, or B2A1b, or a pharmaceutically acceptable salt thereof, is wherein R⁶ and R⁷ are independently selected from hydrogen, methyl, and ethyl, and R^{6a} and R^{6b} are hydrogen.

B2A1d. In embodiment B2A1d, the compound of embodiment B1A1′, B1A′, B2A1, B1a1, B2A1a, or B2A1b, or a pharmaceutically acceptable salt thereof, is wherein R⁶ is cyanomethyl and R⁷ is hydrogen, methyl, or ethyl, preferably R⁷ is hydrogen, and R^{6a} and R^{6b} are hydrogen.

B2A1e. In embodiment B2A1e, the compound of embodiment B1A1′, B1A′, B2A1, B1a1, B2A1a, or B2A1b, or a pharmaceutically acceptable salt thereof, is wherein R⁶ and R⁷ are attached to the carbon atoms of the ring that are opposite or diagonal to each other and combine to form —(CH₂)_{z}— where z is 1, 2, or 3, preferably z is 2, and R^{6a} and R^{6b} are hydrogen.

B2A1f. In embodiment B2A1f, the compound of embodiment B1A1′, B1A′, B2A1, B1a1, B2A1a, or B2A1b, or a pharmaceutically acceptable salt thereof, is wherein R⁶ and R⁷ are attached to the carbon atoms of the ring that are opposite or diagonal to each other and combine to form —CH═CH— and R^{6a} is hydrogen.

B2A1g. In embodiment B2A1g, the compound of embodiment B1A1′, B1A′, B2A1, B1a1, B2A1a, or B2A1b, or a pharmaceutically acceptable salt thereof, is wherein R⁶ and R⁷ are attached to the carbon atoms of the ring that are opposite or diagonal to each other and combine to form —(CH₂)_{z}— where z is 1, 2, or 3, preferably 2, and R^{6a} is alkylidenyl, preferably ═CH₂, and R^{6b} is hydrogen.

B2A1h. In embodiment B2A1h, the compound of embodiment B1A1′, B1A′, B2A1, B1a1, B2A1a, or B2A1b, or a pharmaceutically acceptable salt thereof, is wherein R⁶ and R⁷ are attached to the carbon atoms of the ring that are opposite or diagonal to each other and combine to form —(CH₂)_{z}— where z is 1, 2, or 3, preferably 2, and R^{6a} and R^{6b} are attached to the same carbon of the —(CH₂)_{z}— group and are combined to form cycloalkylene, preferably 1,1-cyclopropylene.

B2A1i. In embodiment B2A1i, the compound of embodiment B1A1′, B1A′, B2A1, B1a1, B2A1a, or B2A1b, or a pharmaceutically acceptable salt thereof, is wherein R⁶ is cyano and R⁷ is hydrogen, methyl, or ethyl, preferably R⁷ is hydrogen, and R^{6a} and R^{6b} are hydrogen.

B2A1j. In embodiment B2A1j, the compound of embodiment B1A1′, B1A′, B1a1, or B2A1, or a pharmaceutically acceptable salt thereof, is wherein R¹ is:

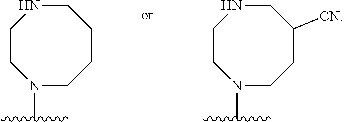 [SMILES: *N1CCCCNCC1 ; "or" ; *N1CCNCC(C#N)CC1]

B2A1k. In embodiment B2A1k, the compound of embodiment B1A1′, B1A′ or B2A1, or a pharmaceutically acceptable salt thereof, is wherein R¹ is:

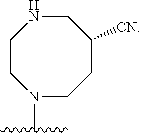 [SMILES: *N1CCNC[C@H](C#N)CC1]

B3A. In embodiment B3A, the compound of embodiment B1A1′, B1A′ or B1a1, or a pharmaceutically acceptable salt thereof, is wherein R¹ is a ring of formula

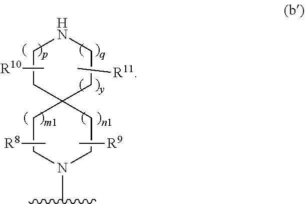

B3a1. In embodiment B3a1, the compound of embodiment B1A1′, B1A′, B1a1, or B3A, or a pharmaceutically acceptable salt thereof, is wherein each of m1, n1, p, q, and y is 1.

B3b1. In embodiment B3b1, the compound of embodiment B1A1′, B1A′, B1a1, or B3A, or a pharmaceutically acceptable salt thereof, is wherein each of m1, n1, p, and q is 0 and y is 1.

B3c1. In embodiment B3c1, the compound of embodiment B1A1′, B1A′, B1a1, or B3A, or a pharmaceutically acceptable salt thereof, is wherein each of m1 and n1 are 1, p is 0, 1, or 2, q is 0, and y is 0 or 1; provided that when p is 0 then y is 1.

B3d1. In embodiment B3d1, the compound of embodiment B1A1′, B1A′, B1a1, or B3A, or a pharmaceutically acceptable salt thereof, is wherein m1 is 0 or 1, n1 is 0, p is 0, 1, or 2, q is 0 and y is 1.

B3e1. In embodiment B3e1, the compound of any one of the embodiments B1A1′, B1A′, B1a1, B3A, and B3a1 to B3d1, or a pharmaceutically acceptable salt thereof, is wherein R⁸, R⁹, R¹⁰ and R¹¹ are hydrogen.

B3f1. In embodiment B3f1, the compound of any one of the embodiments B1A1′, B1A′, B1a1, B3A, and B3a1 to B3d1, or a pharmaceutically acceptable salt thereof, is wherein one or two of R⁸, R⁹, R¹⁰ and R¹¹ are independently methyl, or ethyl and the other of R⁸, R⁹, R¹⁰ and R¹¹ are hydrogen.

B3g1. In embodiment B3g1, the compound of any one of the embodiments B1A1′, B1A′, B1a1, B3A, and B3a1 to B3d1, or a pharmaceutically acceptable salt thereof, is wherein one of R⁸ and R¹⁰ is cyanomethyl and the other is hydrogen and R⁹ and R¹¹ are hydrogen.

B4A. In embodiment B4A, the compound of embodiment B1A1′, B1A′, B1a1, or B1A, or a pharmaceutically acceptable salt thereof, is wherein R¹ is a ring of formula

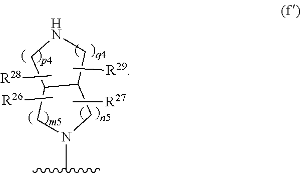

B4a2. In embodiment B4a2, the compound of embodiment B1A1′, B1A′, B1A, B1a1, or B4C, or a pharmaceutically acceptable salt thereof, is wherein each m5, n5, p⁴ and q4 is 1.

B4b2. In embodiment B4b2, the compound of embodiments B1A1′, B1A′, B1A, B1a1, or B4A, or a pharmaceutically acceptable salt thereof, is wherein m5 is 1, n5 is 0, p⁴ is 0, and q4 is 2.

B4b2a. In embodiment B4b2a, the compound of embodiments B1A1′, B1A′, B1A, B1a1, or B4A, or a pharmaceutically acceptable salt thereof, is wherein one of m5 and n5 is 1 or 2 and the other of m5 and n5 is 0, 1, or 2; and one of p4 and q4 is 1 or 2 and the other of p4 and q4 is 0, 1, or 2.

B4c2. In embodiment B4c2, the compound of any one of the embodiments B1A1′, B1A′, B1A, B1a1, B4A, B4a2, B4b2 and B4b2a, or a pharmaceutically acceptable salt thereof, is wherein R²⁶ to R²⁹ are each hydrogen.

B4d2. In embodiment B4d2, the compound of any one of the embodiments B1A1′, B1A′, B1A, B1a1, B4A, and B4a2, B4b2, and b4b2a, or a pharmaceutically acceptable salt thereof, is wherein one or two of of R²⁶ to R²⁹ are methyl and the other of R²⁶ to R²⁹ are hydrogen.

B4e2. In embodiment B4e2, the compound of any one of the embodiments B1A1′, B1A′, B1A, B1a1, B4A, and B4a2, B4b2, and B4b2a, or a pharmaceutically acceptable salt thereof, is wherein R²⁸ is cyano and is attached to the bridgehead carbon that is adjacent to ring N attaching ring (f′) to the remainder of the compound of Formula (IIA′) or (II′), and R²⁶, R²⁷, and R²⁹ are hydrogen.

B4f2. In embodiment B4f2, the compound of embodiment B1A1′, B1A′, B1A, B1a1, or B4A, or a pharmaceutically acceptable salt thereof, is wherein R¹ is:

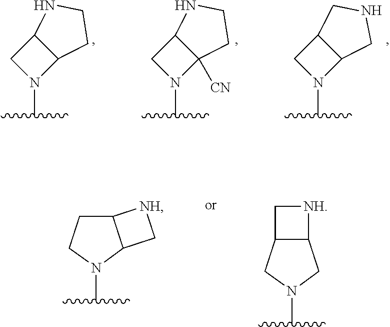 [SMILES: *N1CC2NCCC21 ; *N1CC2NCCC21C#N ; *N1CC2CNCC21 ; *N1CCC2NCC21 ; "or" ; *N1CC2CNC2C1]

B5A. In embodiment B5A, the compound of embodiment B1A1′, B1′, B1a1, or B3A, or a pharmaceutically acceptable salt thereof, is wherein R¹ is a ring of formula:

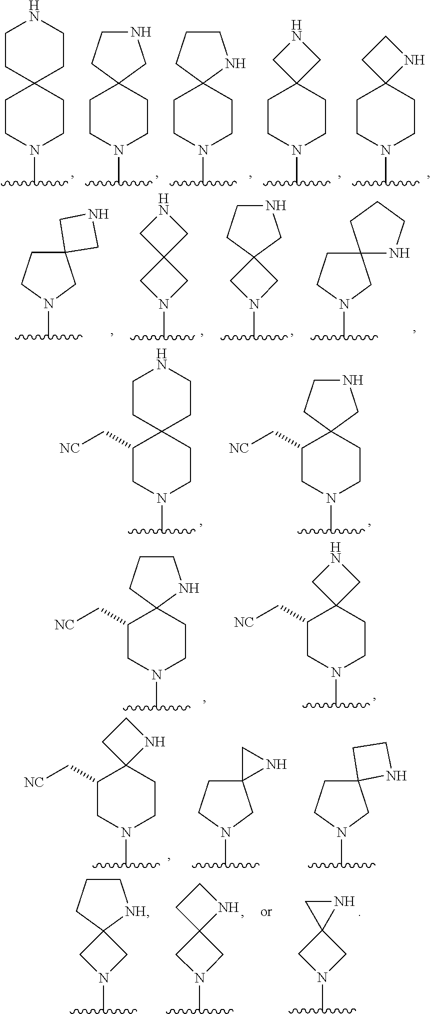

B6A. In embodiment B6A, the compound of embodiment B1A1′, B1′A, B1A, B1a1, or B4A, or a pharmaceutically acceptable salt thereof, is wherein R¹ is a ring of formula:

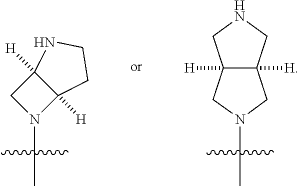

B7A. In embodiment B7A, of embodiment B1A1′, B1A′, B1A, B1a1,

or B4A, or a pharmaceutically acceptable salt thereof, is wherein R¹ is a ring of formula:

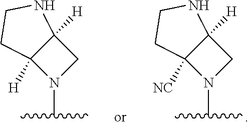 [SMILES: *N1C[C@@H]2NCC[C@@H]21 ; *N1C[C@@H]2NCC[C@@]21C#N]

B8A. In embodiment B8A, the compound of embodiment B1A1′, B1A′ or B1A, or a pharmaceutically acceptable salt thereof, is wherein R¹ is a ring of formula

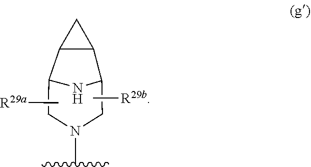

In one subembodiment, R^{29a} and R^{29b} are hydrogen.

B9A. In embodiment B9A, the compound of Formula (IIA1′), (IIA′) and (II′) of any one of embodiments B1A1′, and B1A′ to B8A. or a pharmaceutically acceptable salt thereof, has a structure of formula (II′a) as follows:

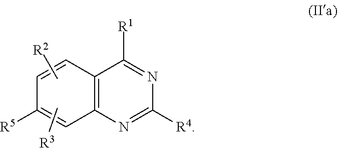

B10A. In embodiment B10A, the compound of Formula (IIA1′), (IIA′) and (II′) of any one of embodiments B1A1′ and B1A′ to B8A, or a pharmaceutically acceptable salt thereof, has a structure of formula (IIb′) as follows:

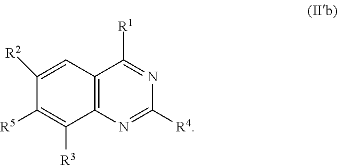

B11A. In embodiment B11A, the compound of Formula (IIA1′), (IIA′) and (II′) of any one of embodiments B1A1′ and B1A′ to B8A, or a pharmaceutically acceptable salt thereof, has a structure of formula (II′c) as follows:

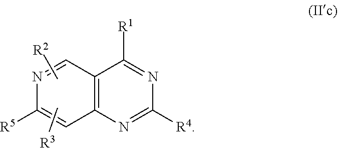

B12A. In embodiment B12A, the compound of Formula (IIA1′), (IIA′) and (II′) of any one of embodiments B1A1′ and B1A′ to B8A, or a pharmaceutically acceptable salt thereof, has a structure of formula (II′d) as follows:

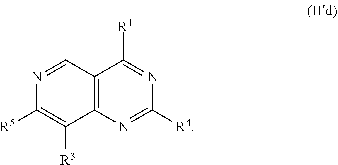

B13A. In embodiment B13A, the compound of Formula (IIA1′), (IIA′) and (II′) of any one of embodiments B1A1′ and B1A′ to B8A, or a pharmaceutically acceptable salt thereof, has a structure of formula (II′e) as follows:

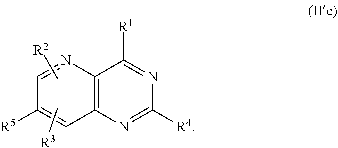

B14A. In embodiment B14A, the compound of Formula (IIA1′), (IIA′) and (II′) of any one of embodiments B1A1′ and B1A′ to B8A, or a pharmaceutically acceptable salt thereof, has a structure of formula (II′f) as follows:

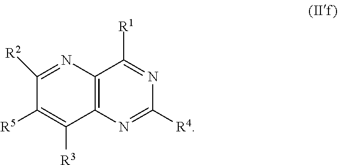

B15A. In embodiment B15A, the compound of any one of embodiments B1A1′ and B1A′ to B14A, or a pharmaceutically acceptable salt thereof, is wherein R⁵ is -Q-R³⁶ where Q is bond and R³⁶ is cycloalkyl, fused cycloalkyl, aryl, aralkyl, heteroaryl, or heteroaralkyl wherein aryl, aryl in aralkyl, heteroaryl, and heteroaryl in heteroaralkyl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and Rid is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

B16A. In embodiment B16A, the compound of any one of embodiments B1A1′ and B1A′ to B14A, or a pharmaceutically acceptable salt thereof, is wherein R⁵ is -Q-R³⁶ where Q is alkylene and R³⁶ is cycloalkyl, aryl, aralkyl, heteroaryl, or heteroaralkyl wherein aryl, aryl in aralkyl, heteroaryl, and heteroaryl in heteroaralkyl are substituted with R^{aa}, R^{bb}, R^{cc} and R^{dd} wherein R^{aa} and R^{b}t are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

B17A. In embodiment B17A, the compound of any one of embodiments B1A1′ and B1A′ to B14A, or a pharmaceutically acceptable salt thereof, is wherein R⁵ is -Q-R³⁶ where Q is —C(O)— and R³⁶ is cycloalkyl, aryl, aralkyl, heteroaryl, or heteroaralkyl wherein aryl, aryl in aralkyl, heteroaryl, and heteroaryl in heteroaralkyl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{o} is hydrogen or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

B18A. In embodiment B18A, the compound of any one of embodiments B1A1′ and BA′ to B14A, or a pharmaceutically acceptable salt thereof, is wherein R³⁶ is cycloalkyl, fused cycloalkyl, aryl, aralkyl, heteroaryl, or heteroaralkyl wherein aryl, aryl in aralkyl, heteroaryl, and heteroaryl in heteroaralkyl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

B19A. In embodiment B19A, the compound of any one of embodiments B1A1′ and B1A′ to B14A, or a pharmaceutically acceptable salt thereof, is wherein R⁵ is -Q-R³⁶ where Q is bond and R³⁶ is phenyl or naphthyl substituted with R^{aa}, R^{bb}, R^{cc} and R^{d}d.

B20A. In embodiment B20A, the compound of any one of embodiments B1A1′ and B1A′ to B14A, or a pharmaceutically acceptable salt thereof, is wherein R⁵ is -Q-R³⁶ where Q is bond and R³⁶ is phenyl or naphthyl substituted with R^{aa}, R^{bb}, and R^{dd} where R^{aa} and R^{bb} are independently selected from hydrogen, alkyl, halo, haloalkyl, hydroxy, alkoxy, haloalkoxy, cycloalkyl, amino, cyano, and hydroxyalkyl and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

B21A. In embodiment B21A, the compound of any one of embodiments B1A1′ and B1A′ to B19A, or a pharmaceutically acceptable salt thereof, is wherein R^{aa} and R^{bb} independently selected from hydrogen, methyl, ethyl, fluoro, chloro, trifluoromethyl, difluoromethyl, trifluoromethoxy, hydroxy, methyl, ethoxy, cyclopropyl, amino, cyano, and hydroxymethyl, R^{cc} is hydrogen, ethynyl, 2-cyanoethyn-1-yl, or fluoro, and R^{dd} is hydrogen, methyl, fluoro, amino, or cyclopropyl.

B22A. In embodiment B22A, the compound of any one of embodiments B1A1′ and B1A′ to B14A, or a pharmaceutically acceptable salt thereof, is wherein R⁵ is -Q-R³⁶ where Q is bond and R³⁶ is heteroaryl substituted with R^{aa}, R^{bb}, R^{cc} and R^{dd}.

B23A. In embodiment B23A, the compound of embodiment B1A1′, B1A′ to B14A and B22A, or a pharmaceutically acceptable salt thereof, is wherein R⁵ is -Q-R³⁶ where Q is bond and R³⁶ is a monocyclic heteroaryl (e.g., pyridyl, pyrimidinyl) substituted with R^{aa}, R^{bb}, R^{cc} and R^{dd}.

B24A. In embodiment B24A, the compound of embodiment B1A′ to B14A and B22A, or a pharmaceutically acceptable salt thereof, is wherein R⁵ is -Q-R³⁶ where Q is bond and R³⁶ is bicyclic heteroaryl (e.g., quinolinyl, isoquinolinyl, or indazolyl), substituted with R^{aa}, R^{bb}, R^{cc} and R^{dd}.

B25A. In embodiment B25A, the compound of any one of embodiments B1A1′, B1A′ to B14A and B22A to B24A, or a pharmaceutically acceptable salt thereof, is wherein the heteroaryl is substituted with R^{aa}, R^{bb}, and R^{dd} where R^{aa} and R^{bb} independently selected from hydrogen, alkyl, halo, haloalkyl, hydroxy, alkoxy, haloalkoxy, cycloalkyl, amino, cyano, and hydroxyalkyl and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

B26A. In embodiment B26A, the compound of any one of embodiments B1A1′, B1A′ to B14A and B22A to B24A, or a pharmaceutically acceptable salt thereof, is wherein R^{aa} and R^{bb} are independently selected from hydrogen, methyl, ethyl, fluoro, chloro, trifluoromethyl, difluoromethyl, trifluoromethoxy, hydroxy, methyl, ethoxy, cyclopropyl, amino, cyano, and hydroxymethyl, R^{cc} is hydrogen or fluoro, and R^{dd} is hydrogen, methyl, fluoro, amino, or cyclopropyl.

B27A1. In embodiment B27A1, the compound of any one of embodiments B1A1′, B1A′ to B14A, or a pharmaceutically acceptable salt thereof, is wherein R⁵ is -Q-R³⁶ where Q is bond and R³⁶ is:

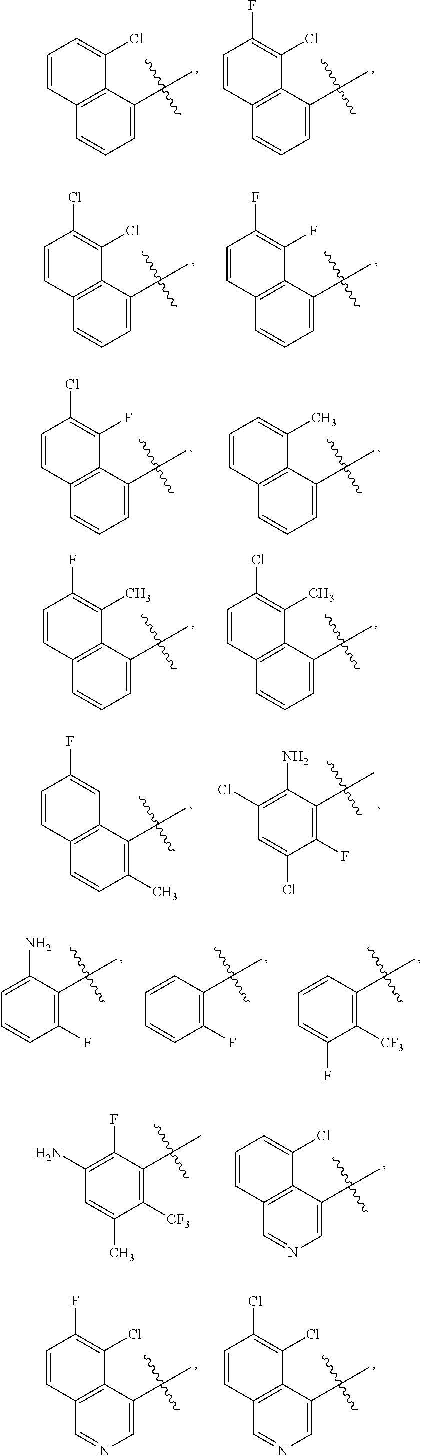

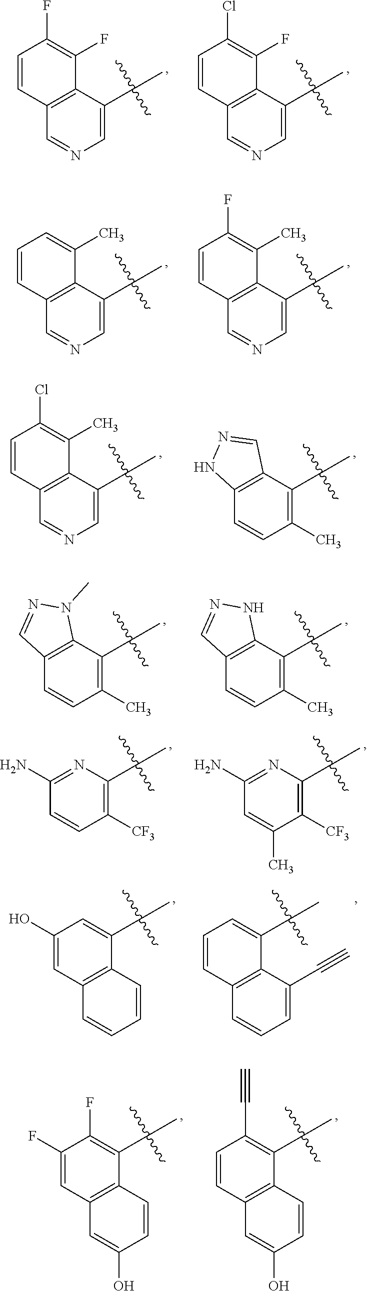

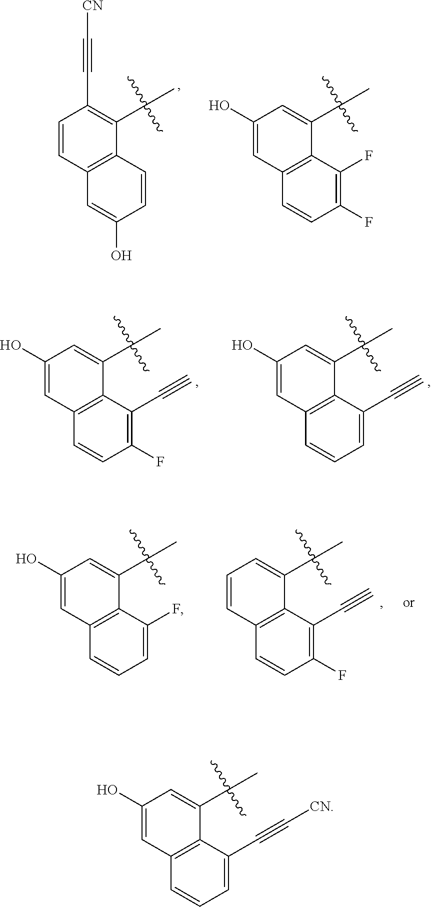

B27A. In embodiment B27A, the compound of any one of embodiments B1A1′, B1A′ to B14A, or a pharmaceutically acceptable salt thereof, is wherein R⁵ is -Q-R³⁶ where Q is bond and R³⁶ is.

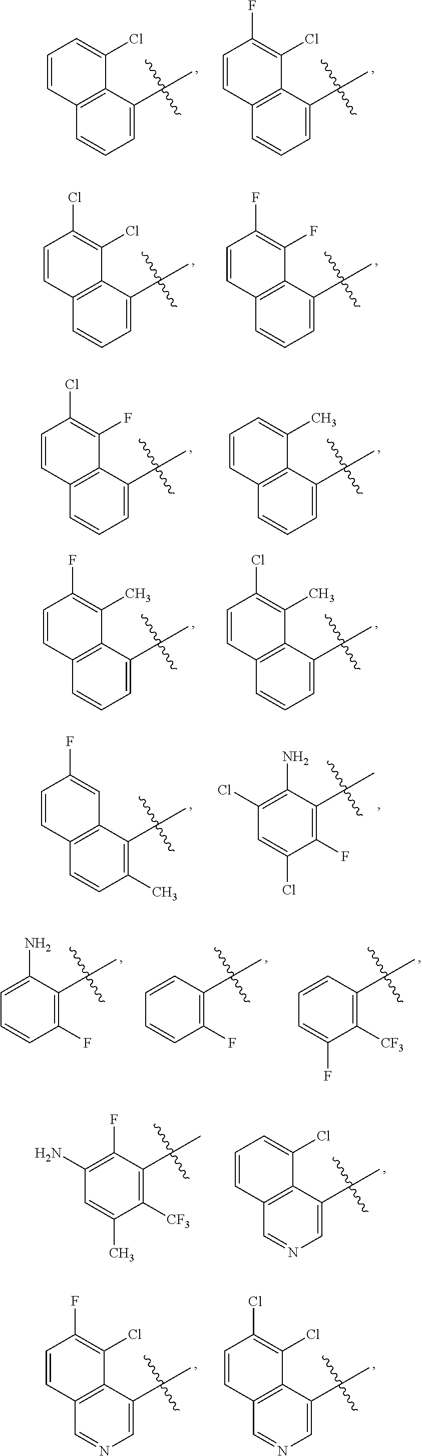

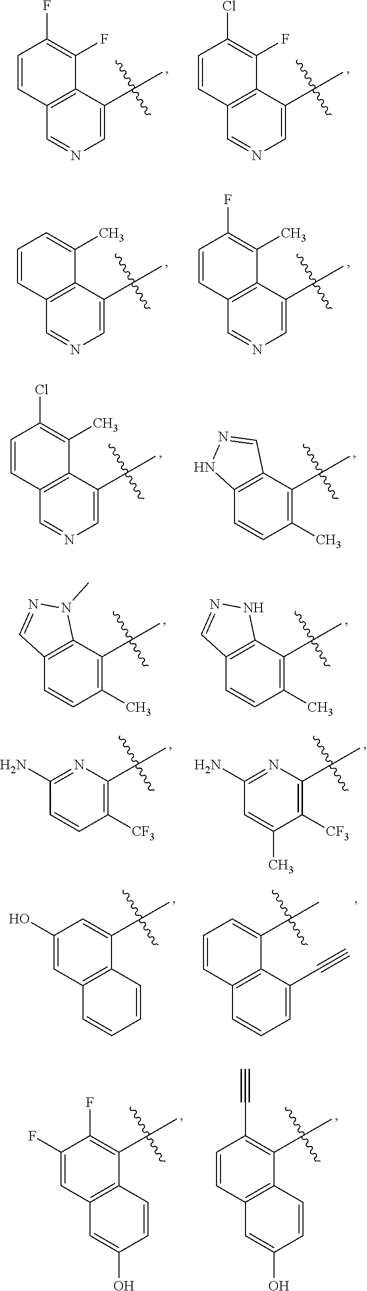

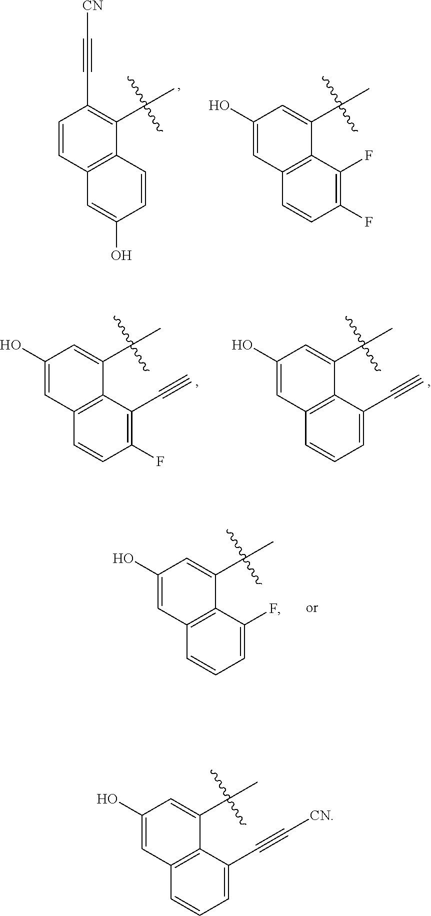 [SMILES: *c1c(C#CC#N)ccc2cc(O)ccc12 ; *c1cc(O)cc2ccc(F)c(F)c12 ; *c1cc(O)cc2ccc(F)c(C#C)c12 ; *c1cc(O)cc2cccc(C#C)c12 ; *c1cc(O)cc2cccc(F)c12 ; *c1cc(O)cc2cccc(C#CC#N)c12]

B28A. In embodiment B28A, the compound of any one of embodiments B1A1′, B1A′ to B27A, or a pharmaceutically acceptable salt thereof, is wherein R² is hydrogen, halo, or alkyl, and R³ hydrogen, halo, cycloalkyloxy, or alkyl.

B29A. In embodiment B29A, the compound of any one of embodiments B1A1′ and B1A′ to B28A, or a pharmaceutically acceptable salt thereof, is wherein R² and R³ are each hydrogen.

B30A1. In embodiment B30A1, the compound of any one of embodiments B1A1′ and B1A′ to B28A, or a pharmaceutically acceptable salt thereof, is wherein R² is hydrogen or chloro and R³ is hydrogen, fluoro, or cyclopropyloxy.

B30A. In embodiment B30A, the compound of any one of embodiments B1A1′ and B1A′ to B28A, or a pharmaceutically acceptable salt thereof, is wherein R² is hydrogen and R³ is fluoro.

B31A. In embodiment B31A, the compound of any one of embodiments B1A1′ and B9A to B30A, or a pharmaceutically acceptable salt thereof, is wherein R⁴ is —Z—R³⁰ where Z is a bond, O, NH, N(alkyl), or S; and R³⁰ is alkyl, hydroxyalkyl, -(alkylene)-NR³¹R³² (where alkylene is substituted with R^{a}, R^{b}, and R^{c} independently selected from hydrogen, alkyl, cycloalkyl, halo, haloalkyl, hydroxy, alkoxy, cyano, oxo, hydroxyalkyl, alkylamino, dialkylamino, dialkylaminocarbonylalkyl, aryl, heteroaryl, and heterocyclyl, R³¹ is hydrogen or alkyl, and R³² is hydrogen, alkyl, acyl, hydroxyalkyl, or heteroalkyl), aryl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkoxyalkyl, alkoxyalkyloxy, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl.

B32A. In embodiment B32A, the compound of any one of the embodiments B1A1′ and B1A′ to B31A, or a pharmaceutically acceptable salt thereof, is wherein Z is O.

B33A. In embodiment B33A, the compound of any one of the embodiments B1A1′ and B1A′ to B31A, or a pharmaceutically acceptable salt thereof, is wherein Z is NH.

B34A. In embodiment B34A, the compound of any one of the embodiments B1A1′ and B1A′ to B31A, or a pharmaceutically acceptable salt thereof, is wherein Z is bond.

B35A. In embodiment B35A, the compound of any one of embodiments B1A1′ and B9A to B30A and B32A to B34A, or a pharmaceutically acceptable salt thereof, is wherein R³⁰ is hydroxyalkyl, -(alkylene)-NR³¹R³² (where alkylene is substituted with R^{a}, R^{b}, and R^{c} independently selected from hydrogen, alkyl, hydroxy, and hydroxyalkyl, R³¹ is hydrogen or alkyl, and R³² is hydrogen, alkyl, or hydroxyalkyl), heteroaralkyl, heterocyclyl, heterocyclylalkyl, bicyclic heterocyclyl, bicyclic heterocyclylalkyl, bridged heterocyclylalkyl, fused heterocyclylalkyl, and spiro heterocyclylalkyl, wherein heterocyclyl, by itself or as part of heterocyclylalkyl, bicyclic heterocyclyl, by itself or as part of bicyclic heterocyclylalkyl, bridged heterocyclyl as part of bridged heterocyclylalkyl, fused heterocyclyl, as part of fused heterocyclylalkyl, and spiro heterocyclyl as part of spiro heterocyclylalkyl, are substituted with R^{d}, R^{e}, and R^{f} independently selected from hydrogen, alkyl, cycloalkyl, cycloalkylalkyl, halo, hydroxy, alkoxy, alkoxyalkyl, acyl, hydroxyalkyl, alkylamino, dialkylamino, cyano, and optionally substituted aralkyl.

B36A. In embodiment B36A, the compound of any one of B9A to B30A and B32A to B34A, or a pharmaceutically acceptable salt thereof, is wherein R³⁰ is 2-dimethylaminoethyl, diethylaminoethyl, 3-methylaminoprop-2-yl, 3-dimethylaminopropyl, 3-dimethylaminoprop-2-yl, 4-dimethylaminobut-2-yl, 4-dimethylaminobut-3-yl, 4-dimethylaminobutyl, 2-dimethylamino-3-hydroxypropyl, 2-dimethylaminoprop-1-yl, 4-methylpiperazin-1-yl, 4-(2-hydroxyethyl)piperazin-1-yl, 4-methylpiperazin-2-yl)methyl, 3-(4-methylpiperazin-1-yl)propyl, 4-dimethylamino-piperidin-1-yl, 1-methylpiperidin-4-yl, piperidin-2-ylmethyl, 2-piperidin-1-ylethyl, 3-piperidin-1-ylpropyl, 3-piperidin-1-ylprop-2-yl, 1-methylpiperidin-3-yl, 2-oxopiperidin-6-ylmethyl, 2-(4-cyanopiperidin-1-yl)ethyl, 2-(2-methylpiperidin-1-yl)ethyl, 2-(4-methylpiperidin-1-yl)ethyl, 3-methoxypiperidin-1-ylethyl, 4-methoxypiperidin-1-ylethyl, 1-cyclopropylpiperidin-4-yl, 2-(4,4-difluoropiperidin-1-yl)ethyl, 2-(3-fluoropiperidin-1-yl)ethyl, 1-methylpiperidin-3-ylmethyl, pyrrolidin-1-yl, pyrrolidin-2-ylmethyl, pyrrolidin-3-ylmethyl, pyrrolidin-1-ylethyl, 3-pyrrolidin-1-ylprop-2-yl, 1-methylpyrrolidin-3-yl, 3-pyrrolidin-1-ylpropyl, 3-fluoropyrrolidin-1-ylethyl, 3,3-difluoropyrrolidin-1-ylethyl, 3-dimethylaminopyrrolidin-1-yl, 2-oxopyrrolidin-5-ylmethyl, 2-(3-methoxypyrrolidin-1-yl)ethyl, 3-(3-methoxypyrrolidin-1-yl)propyl, 3-methoxypyrrolidin-1-yl)prop-2-yl, 3-(3-hydroxypyrrolidin-1-yl)prop-2-yl, 1-methylpyrrolidin-3-ylmethyl, 1-methylpyrrolidin-2-ylmethyl, 1-ethylpyrrolidin-2-ylmethyl, 1-methylpyrrolidin-3-ylmethyl, 2-(1-methylpyrrolidin-2-yl)ethyl, 1-(2-hydroxyethyl)pyrrolidin-3-ylmethyl, 1-(2-methoxyethyl)-pyrrolidin-3-ylmethyl, 1-isopropylpyrrolidin-3-ylmethyl, 5,5-dimethylpyrrolidin-2-yl, 1-benzylpyrrolidin-3-ylmethyl, 1-cyclopropylpyrrolidin-3-ylmethyl, 3-(3,4-difluoropyrrolidin-1-yl)propyl, 3-hydroxy-1-methylpyrrolidin-2-ylmethyl, 4-hydroxy-1-methylpyrrolidin-2-ylmethyl, 3-fluoro-1-methylpyrrolidin-2-ylmethyl, 4-fluoro-1-methylpyrrolidin-2-ylmethyl, 4,4-difluoro-1-methylpyrrolidin-2-ylmethyl, 4-methoxy-1-methylpyrrolidin-2-ylmethyl, 1,2-dimethylpyrrolidin-2-ylmethyl, 1-isopropylpyrrolidin-2-ylmethyl, 1-cyclopropylmethylpyrrolidin-2-ylmethyl, 1,5,5-trimethylpyrrolidin-2-yl, 4-methoxy-1-methylpyrrolidin-2-ylmethyl, 4-methoxy-1-ethylpyrrolidin-2-ylmethyl, morpholin-4-yl, 2-morpholin-4-ylethyl, 3-morpholin-4-ylpropyl, 3-morpholin-3-ylprop-2-yl, 4-morpholin-4-ylbutyl, 4-morpholin-4-ylbut-2-yl, 4-methylmorpholin-2-ylmethyl, 4-methylmorpholin-3-ylmethyl, 5-methylmorpholin-3-ylmethyl, 5,5-dimethylmorpholin-3-ylmethyl, 2-((1S,4R)-2-azabicyclo[2.2.1]heptan-2-yl)ethyl, 2-pyridin-2-ylethyl, or 3-(3-azabicyclo[3.1.0]-hexan-3-ylpropyl.

B37A. In embodiment B37A, the compound of any one of embodiments B1A1′, B1A′, B1A, B2A, 132a1 to B2o1, B2A1, B2A1a to B2A1k, B3A, B3a1 to B3g1, B4A to B30A, and B32A to B34A, or a pharmaceutically acceptable salt thereof, is wherein R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, tricyclic heterocyclyl, tricyclic heterocyclylalkyl, fused tricyclic heterocyclyl, fused tricyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, tricyclic heterocyclyl, by itself or as part of tricyclic heterocyclylalkyl, fused tricyclic heterocyclyl, by itself or as part of fused tricyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, alkenyl, haloalkenyl, cycloalkyl, cycloalkyloxy, cycloalkylalkyl, bridged cycloalkyl, halo, haloalkyl, haloalkoxy, hydroxy, alkoxy, alkylidenyl, haloalkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl.

B38A. In embodiment B38A, the compound of any one of embodiments B1A1′, B1A′, B2A to B30A and B32A to B34A, or a pharmaceutically acceptable salt thereof, is wherein R³⁰ is heterocyclyl fused bicyclic heterocyclyl or heterocyclyl fused bicyclic heterocyclylalkyl wherein heterocyclyl fused bicyclic heterocyclyl, by itself of as part of heterocyclyl fused bicyclic heterocyclylalkyl are substituted with R^{d}, R^{e}, and R^{f} independently selected from hydrogen, alkyl, alkenyl, haloalkenyl, cycloalkyl, cycloalkyloxy, cycloalkylalkyl, bridged cycloalkyl, halo, haloalkyl, haloalkoxy, hydroxy, alkoxy, alkylidenyl, haloalkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl.

B39A. In embodiment B39A, the compound of any one of embodiments B1A1′, B1A′, and B2A to B30A, or a pharmaceutically acceptable salt thereof, is wherein R⁴ is:

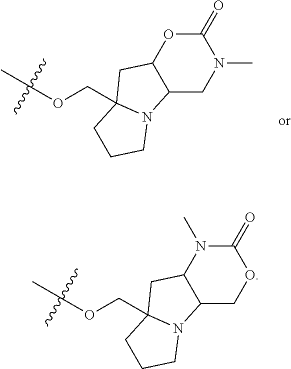 [SMILES: *OCC12CCCN1C1CN(C)C(=O)OC1C2 ; *OCC12CCCN1C1COC(=O)N(C)C1C2]

B40A. In embodiment B40A, the compound of any one of embodiments B1A1′, B1A′. B1A, B2A to B30A, and B32A to B34A, or a pharmaceutically acceptable salt thereof, is wherein R³⁰ is 4-methylpiperazin-1-yl, 4-(2-hydroxyethyl)piperazin-1-yl, 4-methylpiperazin-2-yl)methyl, 3-(4-methylpiperazin-1-yl)propyl, 4-dimethylaminopiperidin-1-yl, 1-methylpiperidin-4-yl, piperidin-2-ylmethyl, 2-piperidin-1-ylethyl, 3-piperidin-1-ylpropyl, 3-piperidin-1-ylprop-2-yl, 1-methylpiperidin-3-yl, 2-oxopiperidin-6-ylmethyl, 2-(4-cyanopiperidin-1-yl)ethyl, 2-(2-methylpiperidin-1-yl)ethyl, 2-(4-methylpiperidin-1-yl)ethyl, 3-methoxypiperidin-1-ylethyl, 4-methoxypiperidin-1-ylethyl, 1-cyclopropylpiperidin-4-yl, 2-(4,4-difluoropiperidin-1-yl)ethyl, 2-(3-fluoropiperidin-1-yl)ethyl, 1-methylpiperidin-3-ylmethyl, pyrrolidin-1-yl, pyrrolidin-2-ylmethyl, pyrrolidin-3-ylmethyl, pyrrolidin-1-ylethyl, 3-pyrrolidin-1-ylprop-2-yl, 1-methylpyrrolidin-3-yl, 3-pyrrolidin-1-ylpropyl, 3-fluoropyrrolidin-1-ylethyl, 3,3-difluoropyrrolidin-1-ylethyl, 3-dimethylaminopyrrolidin-1-yl, 2-oxopyrrolidin-5-ylmethyl, 2-(3-methoxypyrrolidin-1-yl)ethyl, 3-(3-methoxypyrrolidin-1-yl)propyl, 3-methoxypyrrolidin-1-yl)prop-2-yl. 3-(3-hydroxypyrrolidin-1-yl)prop-2-yl, 1-methylpyrrolidin-3-ylmethyl, 1-methylpyrrolidin-2-ylmethyl, 1-ethylpyrrolidin-2-ylmethyl, 1-methylpyrrolidin-3-ylmethyl, 2-(1-methylpyrrolidin-2-yl)ethyl, 1-(2-hydroxyethyl)pyrrolidin-3-ylmethyl, 1-(2-methoxyethyl)-pyrrolidin-3-ylmethyl, 1-isopropylpyrrolidin-3-ylmethyl, 5,5-dimethylpyrrolidin-2-yl, 1-benzylpyrrolidin-3-ylmethyl, 1-cyclopropylpyrrolidin-3-ylmethyl, 3-(3,4-difluoropyrrolidin-1-yl)propyl, 3-hydroxy-1-methylpyrrolidin-2-ylmethyl, 4-hydroxy-1-methylpyrrolidin-2-ylmethyl, 3-fluoro-1-methylpyrrolidin-2-ylmethyl, 4-fluoro-1-methylpyrrolidin-2-ylmethyl, 4,4-difluoro-1-methylpyrrolidin-2-ylmethyl, 4-methoxy-1-methylpyrrolidin-2-ylmethyl, 1,2-dimethylpyrrolidin-2-ylmethyl, 1-isopropylpyrrolidin-2-ylmethyl, 1-cyclopropylmethylpyrrolidin-2-ylmethyl, 1,5,5-trimethylpyrrolidin-2-yl, 4-methoxy-1-methylpyrrolidin-2-ylmethyl, 4-methoxy-1-ethylpyrrolidin-2-ylmethyl, morpholin-4-yl, 2-morpholin-4-ylethyl, 3-morpholin-4-ylpropyl, 3-morpholin-3-ylprop-2-yl, 4-morpholin-4-ylbutyl, 4-morpholin-4-ylbut-2-yl, 4-methylmorpholin-2-ylmethyl, 4-methylmorpholin-3-ylmethyl, 5-methylmorpholin-3-ylmethyl, 5,5-dimethylmorpholin-3-ylmethyl, 2-((1S,4R)-2-azabicyclo[2.2.1]heptan-2-yl)ethyl, 2-pyridin-2-ylethyl, or 3-(3-azabicyclo[3.1.0]-hexan-3-ylpropyl.

B41A. In embodiment B41A, the compound of any one of embodiments B1A1′ and B1A′ to B30A, or a pharmaceutically acceptable salt thereof, is wherein R⁴ is:

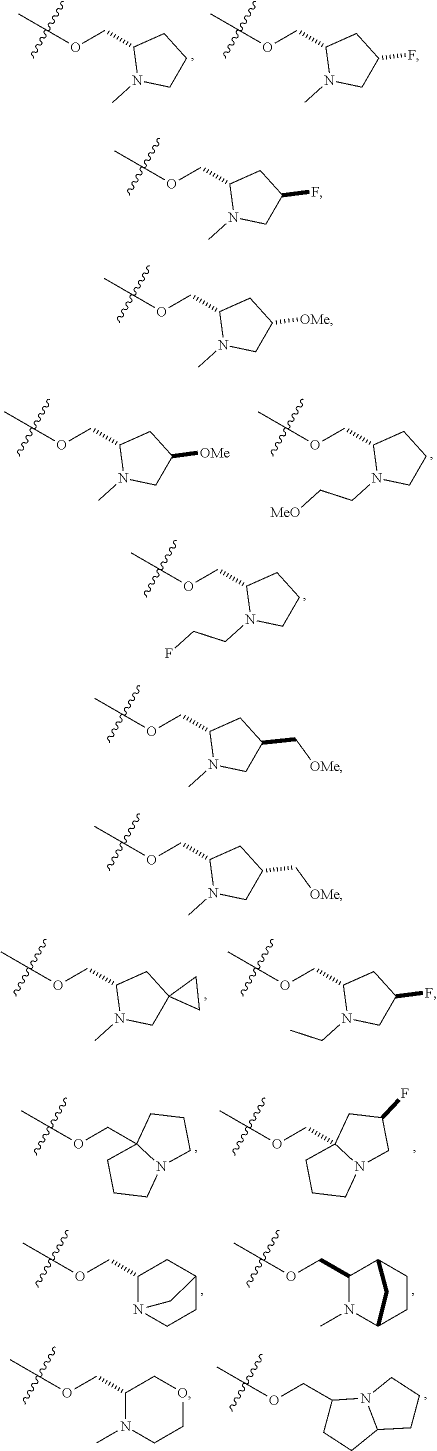

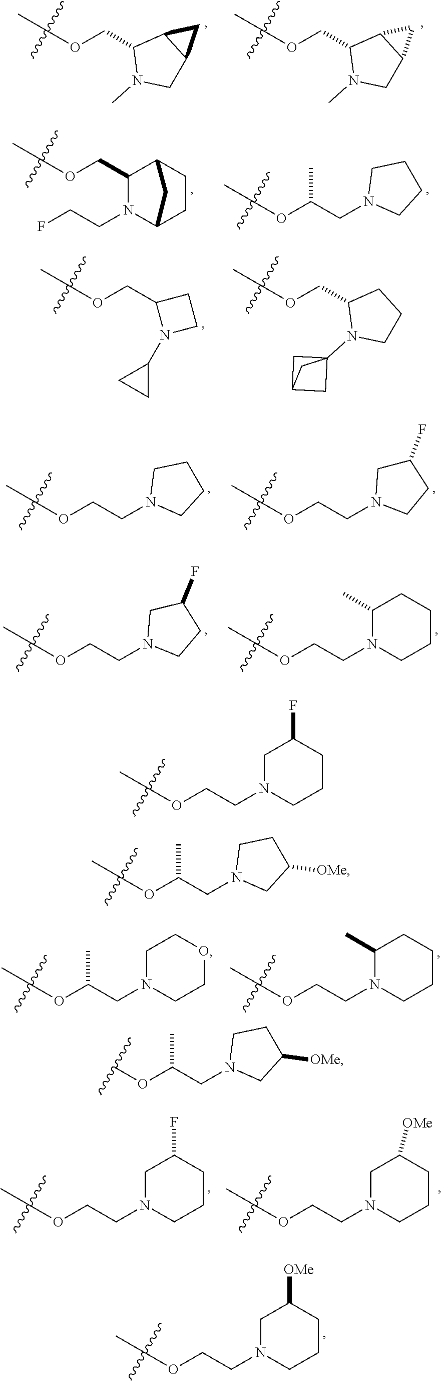

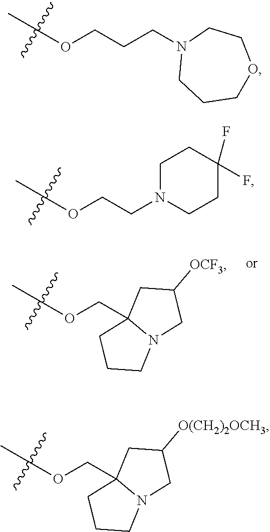

B42A. In embodiment B42A, the compound of any one of embodiments B1A1′ and B1A′ to B30A, or a pharmaceutically acceptable salt thereof, is wherein R⁴ is —CR³³═CR³⁴R³⁵ where R³³ and R³⁴ are independently selected from hydrogen, deuterium, alkyl, halo, and haloalkyl; and R³⁵ is hydrogen, deuterium, alkyl, halo, haloalkyl, cycloalkyl, bridged cycloalkyl, fused cycloalkyl, spiro cycloalkyl, heterocyclyl, bicyclic heterocyclyl, bridged heterocyclyl, fused heterocyclyl, or spiro heterocyclyl; or

R³⁴ and R³⁵ together with the carbon atom to which are attached form cycloalkyl, bridged cycloalkyl, fused cycloalkyl, spiro cycloalkyl, heterocyclyl, bridged heterocyclyl, fused heterocyclyl, or spiro heterocyclyl, wherein:

(a) the groups alkyl, cycloalkyl, bridged cycloalkyl, fused cycloalkyl, spiro cycloalkyl, heterocyclyl, bicyclic heterocyclyl, bridged heterocyclyl, fused heterocyclyl, and spiro heterocyclyl of R³⁵; and (b) the groups cycloalkyl, bridged cycloalkyl, fused cycloalkyl, spiro cycloalkyl, heterocyclyl, bridged heterocyclyl, fused heterocyclyl, or spiro heterocyclyl formed by R³⁴ and R³⁵ together, are independently substituted with R^{h}, Rⁱ, and R^{j} where R^{h}, Rⁱ and R^{j} are independently selected from hydrogen, alkyl, halo, haloalkyl, hydroxyalkyl, alkoxyalkyl, cyano, cycloalkyl, bridged cycloalkyl, heterocyclyl, —O(alk)_{z1}R^{k}, —O(alk)OR^{l}, —S(O)R^{m}, —S(O)₂Rⁿ, —NR^{p}C(O)R^{o}, —NR^{r}SO₂R^{q}, —OC(O)NR^{s}R^{t}, —C(O)NR^{u}R^{v}, —S(O)₂NR^{w}R^{x}, and —NR^{y}R^{z}, where z1 is 0 or 1, alk is alkylene, and R^{k}, R^{l}, R^{m}, Rⁿ, R^{o}, R^{p}, R^{q}, R^{r}, R^{s}, R^{t}, R^{u}, R^{v}, R^{w}, R^{x}, R^{y}, and R^{z} are independently selected from hydrogen, alkyl, cycloalkyl, cycloalkylalkyl, halo, hydroxyalkyl, alkoxyalkyl, and aminoalkyl.

B43A. In embodiment B43A, the compound of any one of embodiments B1A1′ and B1A′ to B30A, or a pharmaceutically acceptable salt thereof, is wherein R⁴ is —CR³³═CR³⁴R³⁵ where R³³ and R³⁴ are independently hydrogen, deuterium, or alkyl; and R³⁵ is heterocyclyl, bicyclic heterocyclyl, bridged heterocyclyl, fused heterocyclyl, or spiro heterocyclyl wherein:

heterocyclyl, bicyclic heterocyclyl, bridged heterocyclyl, fused heterocyclyl, and spiro heterocyclyl of R³⁵ are substituted with R^{h}, Rⁱ, and R^{j} independently selected from hydrogen, alkyl, halo, haloalkyl, hydroxyalkyl, alkoxyalkyl, cyano, cycloalkyl, bridged cycloalkyl, optionally substituted heterocyclyl, —O(alk)_{z1}R^{k}, —O(alk)OR^{l}, and —NR^{y}R^{z}, where z1 is 0 or 1, alk is alkylene, and R^{k}, R^{l}, R^{y}, and R^{z} are independently selected from hydrogen, alkyl, cycloalkyl, cycloalkylalkyl, halo, hydroxyalkyl, alkoxyalkyl, and aminoalkyl.

B44A. In embodiment B44A, the compound of any one of embodiments B1A1′ and B1A′ to B30A, or a pharmaceutically acceptable salt thereof, is wherein R⁴ is:

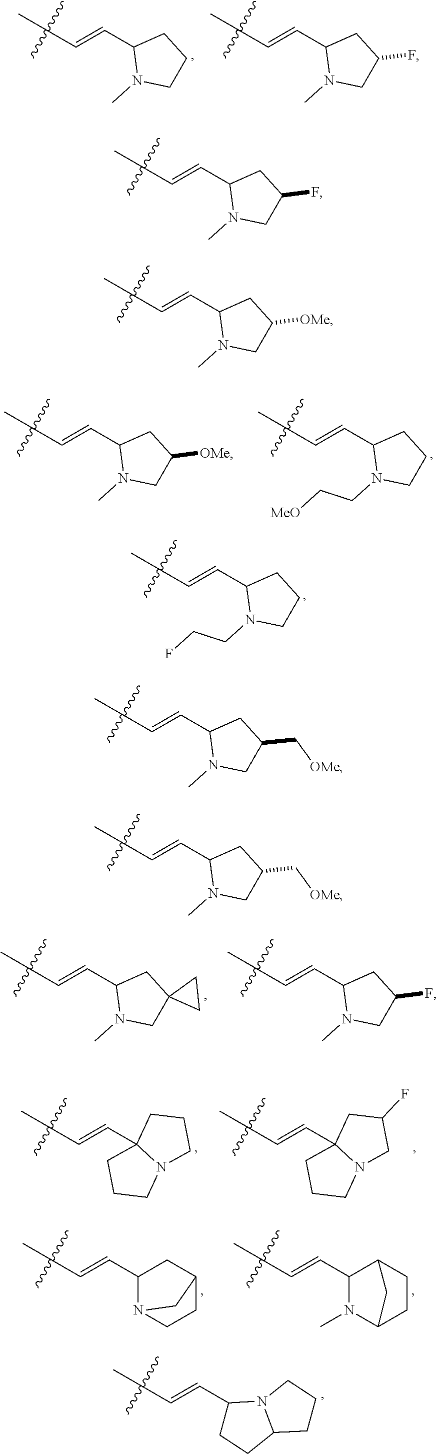

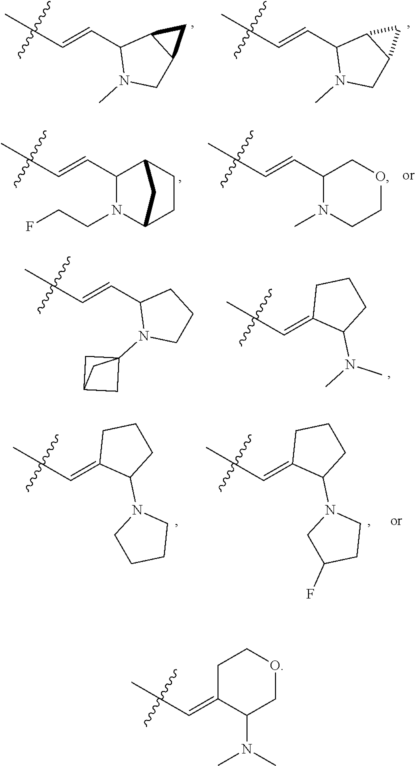

B45A. In embodiment B45A, the compound of any one of embodiments B1A1′ and B1A′ to B30A, or a pharmaceutically acceptable salt thereof, is wherein R⁴ is —CR³³═CR³⁴R³⁵ where R³³ is hydrogen, deuterium, or alkyl and R³⁴ and R³⁵ together with the carbon atom to which are attached form cycloalkyl, bridged cycloalkyl, fused cycloalkyl, spiro cycloalkyl, heterocyclyl, bridged heterocyclyl, fused heterocyclyl, or spiro heterocyclyl, wherein:

cycloalkyl, bridged cycloalkyl, fused cycloalkyl, spiro cycloalkyl, heterocyclyl, bicyclic heterocyclyl, bridged heterocyclyl, fused heterocyclyl, and spiro heterocyclyl formed by R³⁴ and R³⁵ together, are independently substituted with R^{h}, Rⁱ, and R^{j} where R^{h}, Rⁱ and R^{j} are independently selected from hydrogen, alkyl, halo, haloalkyl, hydroxyalkyl, alkoxyalkyl, cyano, cycloalkyl, bridged cycloalkyl, optionally substituted heterocyclyl, —O(alk)_{z1}R^{k}, —O(alk)OR^{l}, and —NR^{y}R^{z}, where z1 is 0 or 1, alk is alkylene, and R^{k}, R^{l}, R^{y}, and R^{z} are independently hydrogen or alkyl.

B46A. In embodiment B46A, the compound of embodiment B44A, or a pharmaceutically acceptable salt thereof, is wherein R⁴ is:

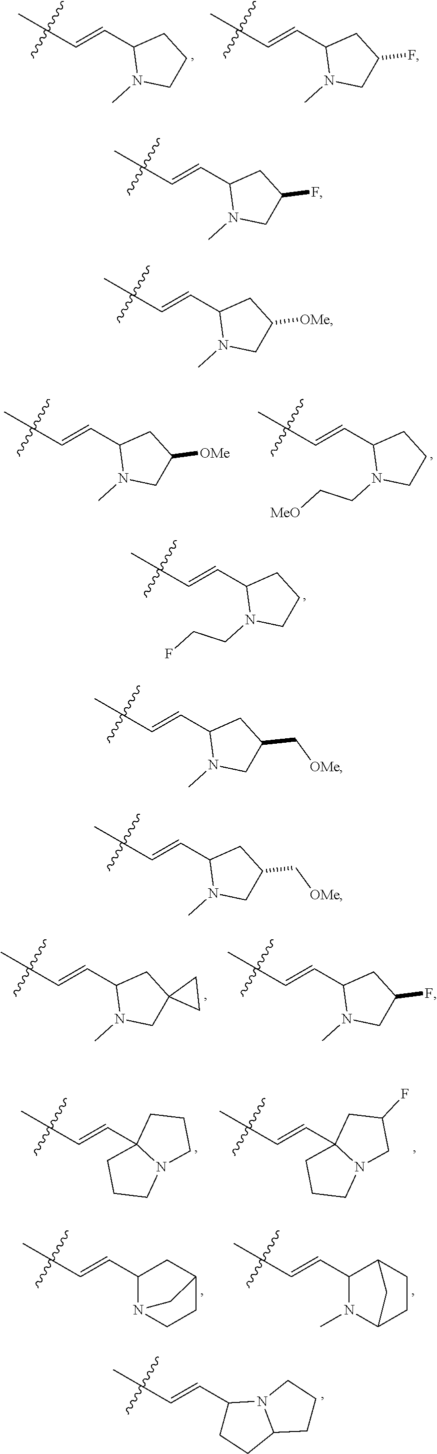

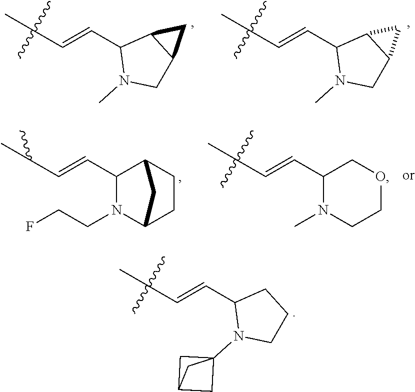

B47A. In embodiment B47A, the compound of embodiment B45A, or a pharmaceutically acceptable salt thereof, is wherein R⁴ is:

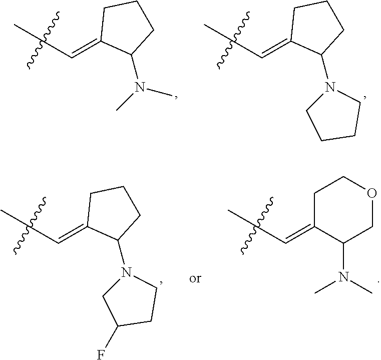

B48A. In embodiment B48A, the compound of any one of embodiments B1A1′, B1A′, B1A, B2A to B30A, or a pharmaceutically acceptable salt thereof, is wherein R⁴ is —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}. R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that; when R³⁰ is aryl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, then at least one of R^{d}. R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

B49A. In embodiment B49A, the compound of any one of embodiments B1A1′, B1A′, B1A, B2A to B30A, or a pharmaceutically acceptable salt thereof, is wherein R⁴ is —Z—R³⁰ where Z is a O, and R³⁰ is heterocyclylalkyl, bicyclic heterocyclylalkyl, phosphinyl bicyclic heterocyclyl, phosphinyl bicyclic heterocyclylalkyl, or fused bicyclic heterocyclylalkyl, wherein heterocyclyl as part of heterocyclylalkyl, bicyclic heterocyclyl as part of bicyclic heterocyclylalkyl, phosphinyl bicyclic heterocyclyl, by itself or as part of phosphinyl bicyclic heterocyclylalkyl, or fused bicyclic heterocyclyl as part of fused bicyclic heterocyclylalkyl are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that; when R³⁰ is heterocyclylalkyl or bicyclic heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl. In a subembodiment of embodiment B49A, R⁴ is

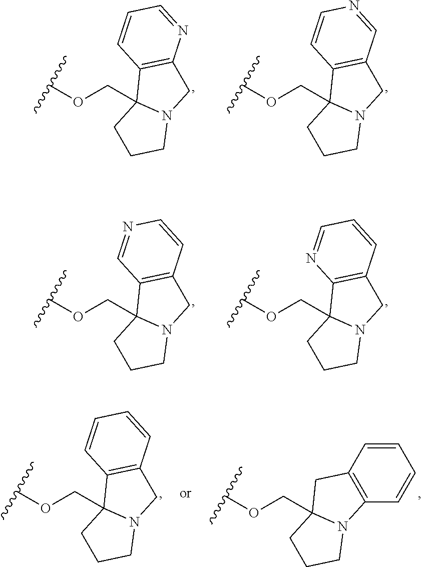

each ring is substituted with R^{d}, R^{e}, and R^{f} independently selected from hydrogen, alkyl, cycloalkyl, cycloalkyloxy halo, haloalkyl, haloalkoxy, hydroxy, alkoxy, alkoxyalkyl, alkoxyalkyloxy, alkylsulfonyl, acyl, cyano, hydroxyalkyl, alkylamino, dialkylamino, or dialkylaminocarbonylalkyl.

B50A. In embodiment B50A, the compound of any one of embodiments B1A1′, B1A′, B1A, B2A to B30A, or a pharmaceutically acceptable salt thereof, is wherein R⁴ is —Z—R³⁰ where Z is a bond, O, NH, N(alkyl), or S; and R³⁰ is phosphinyl bicyclic heterocyclylalkyl or bicyclic heterocyclylalkyl, wherein bicyclic heterocyclyl as part of bicyclic heterocyclylalkyl and phosphinyl bicyclic heterocyclyl as part of phosphinyl bicyclic heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is bicyclic heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

B51A. In embodiment B51A, the compound of any one of embodiments B1A1′, B1A′, B1A, B2A to B30A, or a pharmaceutically acceptable salt thereof is wherein R⁴ is —Z—R³⁰ where Z is O; and R³⁰ is phosphinyl bicyclic heterocyclylalkyl or bicyclic heterocyclylalkyl, wherein bicyclic heterocyclyl as part of bicyclic heterocyclylalkyl and phosphinyl bicyclic heterocyclyl as part of phosphinyl bicyclic heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is bicyclic heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

B52A. In embodiment B52A, the compound of any one of embodiments B50A and B51A, or a pharmaceutically acceptable salt thereof, is wherein R⁴ is:

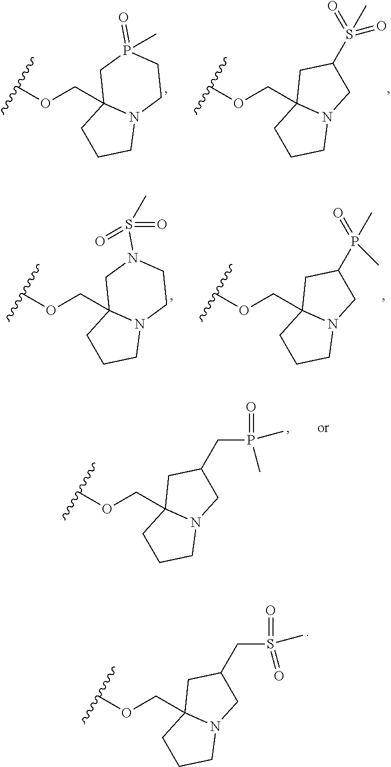 [SMILES: *OCC12CCCN1CCP(C)(=O)C2 ; *OCC12CCCN1CC(S(C)(=O)=O)C2 ; *OCC12CCCN1CCN(S(C)(=O)=O)C2 ; *OCC12CCCN1CC(P(C)(C)=O)C2 ; *OCC12CCCN1CC(CP(C)(C)=O)C2 ; *OCC12CCCN1CC(CS(C)(=O)=O)C2]

B53A. In embodiment B53A, the compound of any one of embodiments B1A1′, B1A′, B1A, and B2A to B30A, or a pharmaceutically acceptable salt thereof, is wherein R⁴ is —Z—R³⁰ where Z is O; and R³⁰ is tricyclic heterocyclyl, tricyclic heterocyclylalkyl, fused tricyclic heterocyclyl, fused tricyclic heterocyclylalkyl, bicyclic heterocyclyl, or bicyclic heterocyclylalkyl wherein tricyclic heterocyclyl, by itself or as part of tricyclic heterocyclylalkyl and fused tricyclic heterocyclyl, by itself or as part of fused tricyclic heterocyclylalkyl are substituted with R^{d}, R^{e}, and R^{f} independently selected from hydrogen, alkyl, alkenyl, haloalkenyl, cycloalkyl, cycloalkyloxy, cycloalkylalkyl, bridged cycloalkyl, halo, haloalkyl, haloalkoxy, hydroxy, alkoxy, alkylidenyl, haloalkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; and bicyclic heterocyclyl and bicyclic heterocyclyl as part of bicyclic heterocyclylalkyl are substituted with alkylidene or haloalkylidene. In one subembodiment of embodiment B53A, R⁴ is:

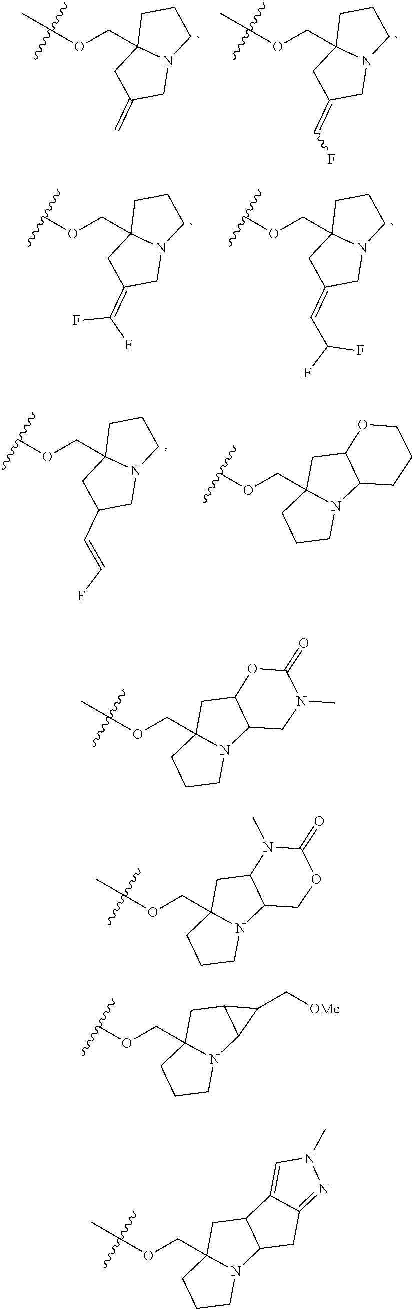

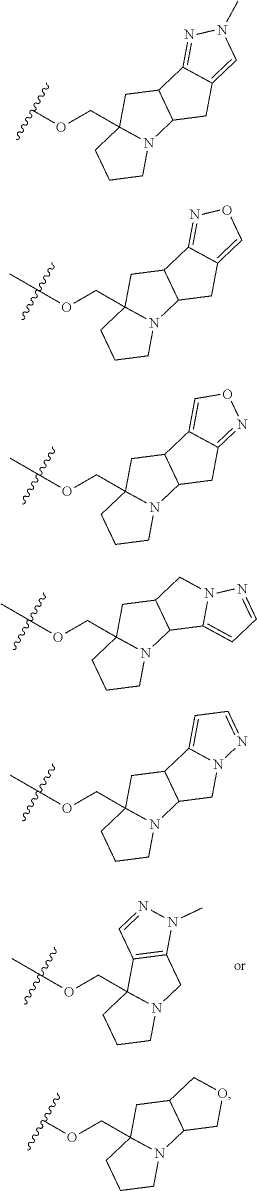

preferably,

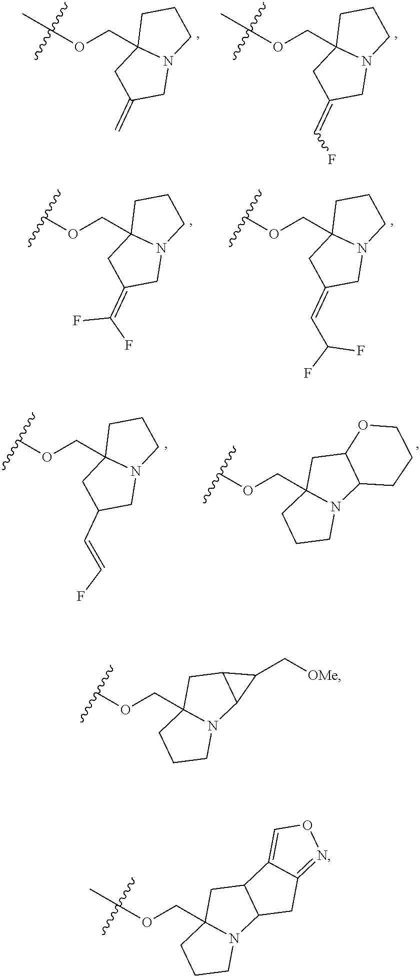

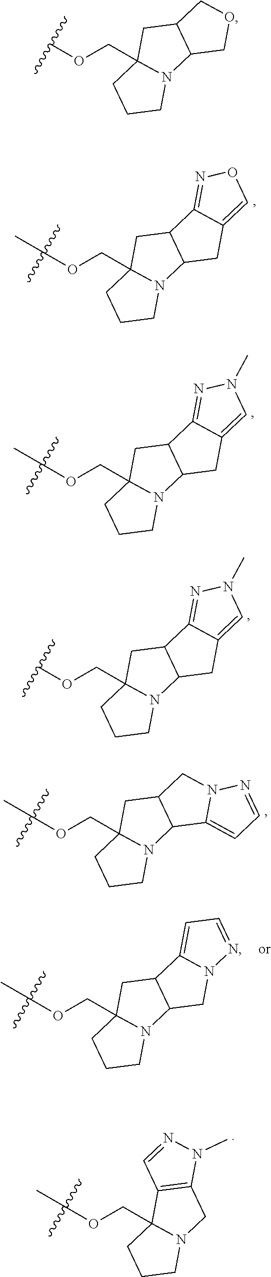

In a second subembodiment of embodiment B53A, R⁴ is

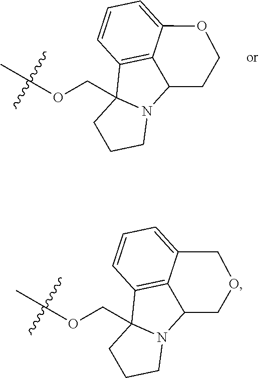 [SMILES: *OCC12CCCN1C1CCOc3cccc2c31 ; *OCC12CCCN1C1COCc3cccc2c31]

where each ring is substituted with R^{d}, R^{e}, and R^{f} independently selected from hydrogen, alkyl, alkenyl, haloalkenyl, cycloalkyl, cycloalkyloxy, halo, haloalkyl, haloalkoxy, hydroxy, alkoxy, alkoxyalkyl, alkoxyalkyloxy, alkylsulfonyl, acyl, cyano, hydroxyalkyl, alkylamino, dialkylamino, or dialkylaminocarbonylalkyl, preferably R^{d}, R^{e}, and R^{f} independently selected from hydrogen, alkyl, haloalkenyl, cycloalkyl, cycloalkyloxy, halo, haloalkyl, haloalkoxy, hydroxy, alkoxy, alkoxyalkyl, or alkoxyalkyloxy.

B54A. In embodiment B54A, the compound of any one of embodiments B1A1′, B1A′, B1A, and B2A to B30A, or a pharmaceutically acceptable salt thereof, is wherein R⁴ is —Z—R³⁰ where Z is O; and R³⁰ is tricyclic heterocyclylalkyl, fused tricyclic heterocyclylalkyl, or bicyclic heterocyclylalkyl wherein the tricyclic heterocyclyl as part of tricyclic heterocyclylalkyl, and fused tricyclic heterocyclyl as part of fused tricyclic heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkoxyalkyl, alkoxyalkyloxy, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; and the bicyclic heterocyclyl as part of bicyclic heterocyclylalkyl is substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, haloalkylidenyl, alkoxyalkylidenyl, alkoxyalkyl, alkoxyalkyloxy, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is bicyclic heterocyclyalkyl, then at least one of R^{d}, R^{e}, and R^{f} alkylidene, alkoxyalkylidene, or haloalkylidene.

B55A. In embodiment B55A, the compound any one of embodiments B1A1′, B1A′, B1A, and B2A to B30A, andB54A, or a pharmaceutically acceptable salt thereof, is wherein R³⁰ is tricyclic heterocyclylmethylene, fused tricyclic heterocyclylmethylene, or bicyclic heterocyclylmethylene independently substituted with R^{d}, R^{e}, and R^{f} as defined therein.

B56A. In embodiment B56A, the compound of any one of embodiments B1A1′, B1A′, B1A, and B2A to B30A, and B54A, is wherein R³⁰ is tricyclic heterocyclylmethylene wherein tricyclic heterocyclyl as part of tricyclic heterocyclylmethylene is substituted with R^{d}, R^{e}, and R^{f} as defined therein.

B57A. In embodiment B57A, the compound of any one of embodiments B1A1′, B1A′, B1A, and B2A to B30A, and B54A, or a pharmaceutically acceptable salt thereof, is wherein R³⁰ is fused tricyclic heterocyclylmethylene wherein fused tricyclic heterocyclyl as part of fused tricyclic heterocyclylmethylene is substituted with R^{d}, R^{e}, and R^{f} as defined therein.

B58A. In embodiment B58A, the compound any one of embodiments B1A1′, B1A′, B1A, and B2A to B30A, andB54A, or a pharmaceutically acceptable salt thereof, is wherein R³⁰ is bicyclic heterocyclylmethylene wherein bicyclic heterocyclyl as part of bicyclic heterocyclylmethylene is substituted with R^{d}, R^{e}, and R^{f} as defined therein

B59A. In embodiment B59A, the compound of any one of embodiments B54A to B58A, or a pharmaceutically acceptable salt thereof, is wherein R^{d} is hydrogen.

B60A. In embodiment B60A, the compound of any one of embodiments B1A1′, B1A′, B1A and B2A to B30A, B54A, B55A and B58A, or a pharmaceutically acceptable salt thereof, is wherein R⁴ is —Z—R³⁰ selected from:

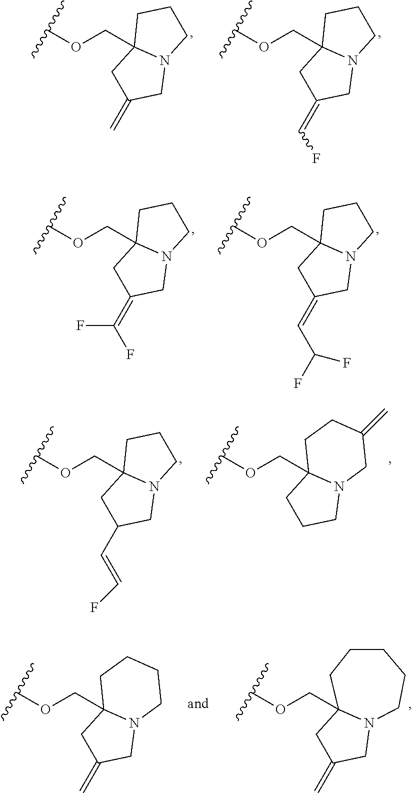

each ring optionally substituted with R^{e} selected from hydrogen, methyl, ethyl, isopropyl, cyclopropyl, cyclopropyloxy, fluoro, chloro, difluoromethyl, trifluoromethyl, difluoromethyloxy, trifluoromethoxy, methoxy, ethoxy, methoxymethyl, methoxymethyloxy, cyano, methylamino, dimethylamino, diethylamino, hydroxymethyl, phenyl, and benzyl.

B61A. In embodiment B61A, the compound of any one of embodiments B1A1′, B1A′, B1A and B2A to B30A, B54A, B55A and B58A, or a pharmaceutically acceptable salt thereof, is wherein R⁴ is —Z—R³⁰ selected from s:

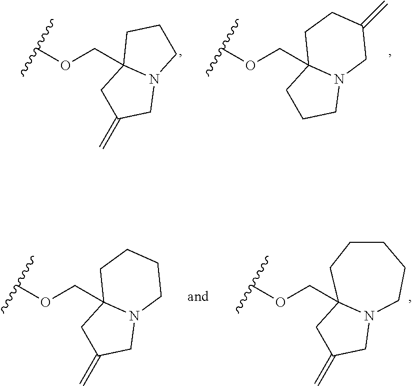

each ring optionally substituted with R^{e} selected from hydrogen, methyl, ethyl, isopropyl, cyclopropyl, cyclopropyloxy, fluoro, chloro, difluoromethyl, trifluoromethyl, difluoromethyloxy, trifluoromethoxy, methoxy, ethoxy, methoxymethyl, methoxymethyloxy, cyano, methylamino, dimethylamino, diethylamino, hydroxymethyl, phenyl, and benzyl.

B62A. In embodiment B62A, the compound of any one of embodiments B1A1′, B1A′, B1A and B2A to B30A, B54A, B55A and B58A, or a pharmaceutically acceptable salt thereof, is wherein R⁴ is —Z—R³⁰ selected from:

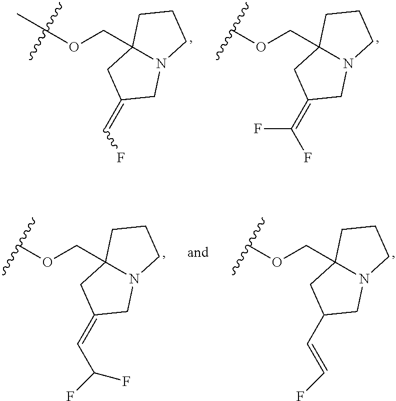 [SMILES: *OCC12CCCN1CC(=CF)C2 ; *OCC12CCCN1CC(=C(F)F)C2 ; *OCC12CCCN1C/C(=C\C(F)F)C2 ; *OCC12CCCN1CC(/C=C/F)C2]

each ring optionally substituted with R^{e} selected from hydrogen, methyl, ethyl, isopropyl, cyclopropyl, cyclopropyloxy, fluoro, chloro, difluoromethyl, trifluoromethyl, difluoromethyloxy, trifluoromethoxy, methoxy, ethoxy, methoxymethyl, methoxymethyloxy, cyano, methylamino, dimethylamino, diethylamino, hydroxymethyl, phenyl, and benzyl.

B63A. In embodiment B63A, the compound of any one of B1A1′, B1A′, B1A and B2A to B30A, B54A, B55A and B56A, or a pharmaceutically acceptable salt thereof, is wherein R⁴ is —Z—R³⁰ selected from:

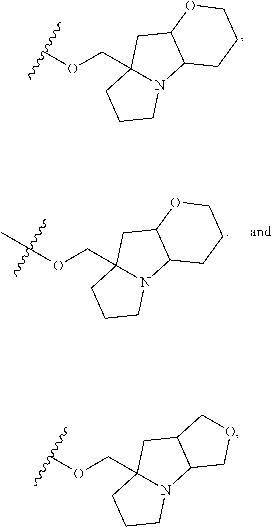 [SMILES: *OCC12CCCN1C1CCCOC1C2 ; *OCC12CCCN1C1CCCOC1C2 ; *OCC12CCCN1C1COCC1C2]

each ring optionally substituted with R^{e} selected from hydrogen, hydrogen, methyl, ethyl, isopropyl, cyclopropyl, cyclopropyloxy, fluoro, chloro, difluoromethyl, trifluoromethyl, difluoromethyloxy, trifluoromethoxy, methoxy, ethoxy, methoxymethyl, methoxymethyloxy, cyano, methylamino, dimethylamino, diethylamino, hydroxymethyl, phenyl, and benzyl.

B64A. In embodiment B64A, the compound of any one of embodiments B1A1′, B1A′, B1A and B2A to B30A, B54A, B55A and B57A, or a pharmaceutically acceptable salt thereof, is wherein R⁴ is —Z—R³⁰ selected from:

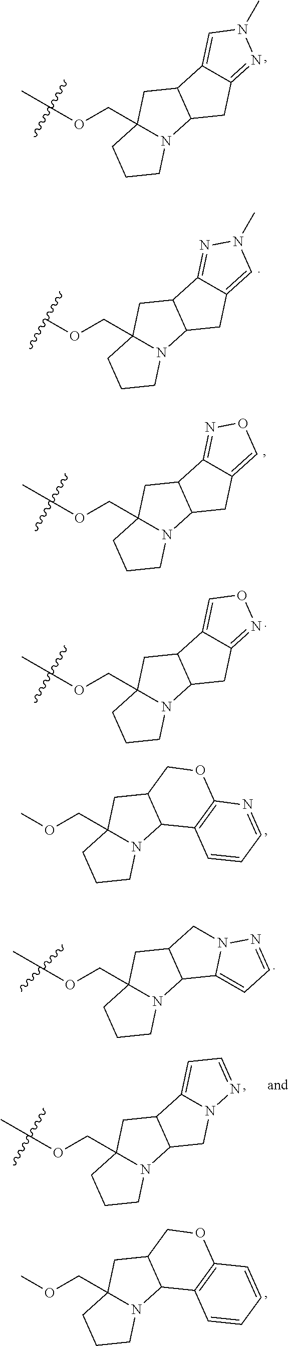

each ring optionally substituted with R^{e} selected from hydrogen, methyl, ethyl, isopropyl, cyclopropyl, cyclopropyloxy, fluoro, chloro, difluoromethyl, trifluoromethyl, difluoromethyloxy, trifluoromethoxy, methoxy, ethoxy, methoxymethyl, methoxymethyloxy, cyano, methylamino, dimethylamino, diethylamino, hydroxymethyl, phenyl, and benzyl.

B65A. In embodiment B65A, the compound of any one of embodiments B1A1′, B1A′, B1A, B2A to B30A, and B54A to B64A, or a pharmaceutically acceptable salt thereof, is wherein R^{e} is hydrogen.

B66A. In embodiment B66A, the compound of any one of embodiments embodiments B1A1′, B1A′, B1A, B2A to B30A, and B54A to B65A, or a pharmaceutically acceptable salt thereof, is wherein R^{f} is hydrogen.

B67A. In embodiment B67A, the compound of any one of embodiments B1A1′ to B66A, or a pharmaceutically acceptable salt thereof, is wherein R⁵ is

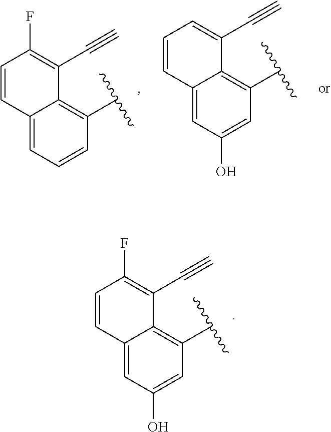 [SMILES: *c1cccc2ccc(F)c(C#C)c12 ; *c1cc(O)cc2cccc(C#C)c12 ; *c1cc(O)cc2ccc(F)c(C#C)c12]

### Embodiments C

In further embodiments 1 to 35 below, the present disclosure includes:

1. In embodiment 1, provided is a compound of Formula (IIA′):

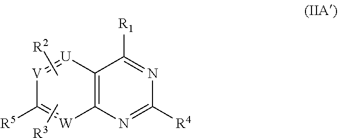

is wherein:

U, V, and W are CH; or one or two of U, V, and W are N and the other of U, V, and W are CH;

R¹ is a ring of formula:

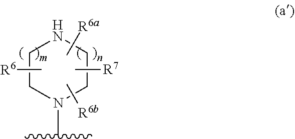

where:

m and n are independently 0, 1, or 2;

R⁶ is hydrogen, deuterium, alkyl, alkoxy, halo, haloalkyl, hydroxy, hydroxylalkyl, alkoxyalkyl, cyano, or cyanomethyl, provided R⁶ is not attached to the ring —NH—;

R⁷ is hydrogen, deuterium, alkyl, alkoxy, halo, haloalkyl, hydroxy, hydroxylalkyl, or alkoxyalkyl, provided R⁷ is not attached to the ring —NH—; or

when R⁶ and R⁷ are attached to the carbon atoms of the ring that are opposite or diagonal to each other, then R⁶ and R⁷ can combine to form —(CH₂)_{z}— where (z is 1, 2, or 3), or —CH═CH—;

R^{6a} is hydrogen, deuterium, alkyl, alkylidienyl, alkoxy, halo, haloalkyl, hydroxy, hydroxyalkyl, alkoxyalkyl, cyano, or cyanomethyl, provided R^{6a} is not attached to the ring —NH—;

R^{6b} is hydrogen or alkyl, provided R^{6b} is not attached to the ring —NH—; or

when R^{6a} and R^{6b} are attached to the same carbon of ring (a′), they can combine to form alkylidienyl or cycloalkylene;

R² is hydrogen, deuterium, alkyl, halo, haloalkyl, alkoxy, hydroxy, or cyano, provided that R² is absent when two of U, V, and W are N;

R³ is hydrogen, deuterium, alkyl, halo, haloalkyl, alkoxy, cycloalkyloxy, hydroxy, or cyano;

R⁴ is:

(i) —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, heterocyclyl fused bicyclic heterocyclyl, heterocyclyl fused bicyclic heterocyclylalkyl, tricyclic heterocyclyl, tricyclic heterocyclylalkyl, fused tricyclic heterocyclyl, fused tricyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, heterocyclyl fused bicyclic heterocyclyl, by itself of as part of heterocyclyl fused bicyclic heterocyclylalkyl, tricyclic heterocyclyl, by itself or as part of tricyclic heterocyclylalkyl, fused tricyclic heterocyclyl, by itself or as part of fused tricyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, alkenyl, haloalkenyl, cycloalkyl, cycloalkyloxy, cycloalkylalkyl, bridged cycloalkyl, halo, haloalkyl, haloalkoxy, hydroxy, alkoxy, alkylidienyl, haloalkylidienyl, alkoxyalkylidienyl, alkoxyalkyl, alkoxyalkyloxy, alkylsulfonyl, alkylsulfonylalkyl, dialkyl(oxo)phosphinyl, dialkyl(oxo)phosphinvlalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is aryl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, then at least one of R^{d}, R^{e}, and R^{f} is alkylidenyl, haloalkylidenyl, alkoxyalkylidenyl, alkylsulfonyl, alkylsulfonylalkyl, dialkyl(oxo)phosphinyl, or dialkyl(oxo)phosphinylalkyl; and

R⁵ is -Q-R³⁶ where Q is bond, alkylene, or —C(═O)—; and R³⁶ is hydrogen, cycloalkyl, fused cycloalkyl, aryl, aralkyl, heteroaryl, or heteroaralkyl wherein aryl, aryl in aralkyl, heteroaryl, and heteroaryl in heteroaralkyl are independently substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl; or

a pharmaceutically acceptable salt thereof.

2. In embodiment 2, the compound of embodiment 1, or a pharmaceutically acceptable salt thereof is wherein:

R¹ is a ring of formula:

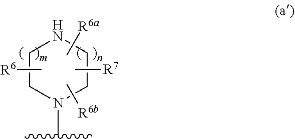

where:

R^{6a} is hydrogen, deuterium, alkyl, alkoxy, halo, haloalkyl, hydroxy, hydroxylalkyl, alkoxyalkyl, cyano, or cyanomethyl, provided R^{6a} is not attached to the ring —NH—;

R⁴ is:

(i) —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that; when R³⁰ is aryl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

3. In embodiment 3, the compound of embodiment 1 or 2, or a pharmaceutically acceptable salt thereof, is wherein R⁴ is —Z—R³⁰ where Z is O; and R³⁰ is phosphinyl bicyclic heterocyclylalkyl or bicyclic heterocyclylalkyl, wherein bicyclic heterocyclyl as part of bicyclic heterocyclylalkyl and phosphinyl bicyclic heterocyclyl as part of phosphinyl bicyclic heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenvl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is bicyclic heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

4. In embodiment 4, the compound of embodiment 2, or a pharmaceutically acceptable salt thereof, is wherein R⁴ is —Z—R³⁰ where Z is O; and R³⁰ is heterocyclylalkyl, bicyclic heterocyclylalkyl, phosphinyl bicyclic heterocyclyl, phosphinyl bicyclic heterocyclylalkyl, or fused bicyclic heterocyclylalkyl, wherein heterocyclyl as part of heterocyclylalkyl, bicyclic heterocyclyl as part of bicyclic heterocyclylalkyl, phosphinyl bicyclic heterocyclyl, by itself or as part of phosphinyl bicyclic heterocyclylalkyl, or fused bicyclic heterocyclyl as part of fused bicyclic heterocyclylalkyl are substituted with R^{d}, R^{e}, and R^{f}; provided that; when R³⁰ is heterocyclylalkyl or bicyclic heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

5. In embodiment 5, the compound of embodiment 1, or a pharmaceutically acceptable salt thereof, is wherein R⁴ is —Z—R³⁰ where Z is O; and R³⁰ is tricyclic heterocyclyl, tricyclic heterocyclylalkyl, fused tricyclic heterocyclyl, fused tricyclic heterocyclylalkyl, bicyclic heterocyclyl, or bicyclic heterocyclylalkyl wherein tricyclic heterocyclyl, by itself or as part of tricyclic heterocyclylalkyl and fused tricyclic heterocyclyl, by itself or as part of fused tricyclic heterocyclylalkyl, are substituted with R^{d}, R^{e}, and R^{f} independently selected from hydrogen, alkyl, alkenyl, haloalkenyl, cycloalkyl, cycloalkyloxy, cycloalkylalkyl, bridged cycloalkyl, halo, haloalkyl, haloalkoxy, hydroxy, alkoxy, alkylidenyl, haloalkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; and bicyclic heterocyclyl and bicyclic heterocyclyl as part of bicyclic heterocyclylalkyl are substituted with alkylidene or haloalkylidene.

6. In embodiment 6, the compound of embodiment 1, or a pharmaceutically acceptable salt thereof, is wherein R⁴ is —Z—R³⁰ where Z is O; and R³⁰ is tricyclic heterocyclylalkyl, fused tricyclic heterocyclylalkyl, or bicyclic heterocyclylalkyl wherein R³⁰ is tricyclic heterocyclylalkyl, fused tricyclic heterocyclylalkyl, or bicyclic heterocyclylalkyl wherein the tricyclic heterocyclyl as part of tricyclic heterocyclylalkyl, and fused tricyclic heterocyclyl as part of fused tricyclic heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkoxyalkyl, alkoxyalkyloxy, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; and the bicyclic heterocyclyl as part of bicyclic heterocyclylalkyl is substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, haloalkylidenyl, alkoxyalkylidenyl, alkoxyalkyl, alkoxyalkyloxy, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is bicyclic heterocyclyalkyl, then at least one of R^{d}, R^{e}, and R^{f} alkylidene, alkoxyalkylidene, or haloalkylidene.

7. In embodiment 7, the compound of embodiment 6, or a pharmaceutically acceptable salt thereof, is wherein R³⁰ is tricyclic heterocyclylmethylene, fused tricyclic heterocyclylmethylene, or bicyclic heterocyclylmethylene independently substituted with R^{d}, R^{e}, and R^{f}.

8. In embodiment 8, the compound of embodiment 1, 6, or 7, or a pharmaceutically acceptable salt thereof, is wherein R³⁰ is tricyclic heterocyclylmethylene wherein tricyclic heterocyclyl as part of tricyclic heterocyclylmethylene is substituted with R^{d}, R^{e}, and R^{f}.

9. In embodiment 9, the compound of embodiment 1, 6, or 7, or a pharmaceutically acceptable salt thereof, is wherein R³⁰ is fused tricyclic heterocyclylmethylene wherein fused tricyclic heterocyclyl as part of fused tricyclic heterocyclylmethylene is substituted with R^{d}, R^{e}, and R^{f}.

10. In embodiment 10, the compound of embodiment 1, 6, or 7, or a pharmaceutically acceptable salt thereof, is wherein R³⁰ is bicyclic heterocyclylmethylene wherein bicyclic heterocyclyl as part of bicyclic heterocyclylmethylene is substituted with R^{d}, R^{e}, and R^{f}.

11. In embodiment 11, the compound of any one of embodiments 1 to 10, or a pharmaceutically acceptable salt thereof, is wherein R^{d} is hydrogen.

11a. In embodiment 11a, the compound of any one of embodiments 1 to 7 and 10, or a pharmaceutically acceptable salt thereof, is wherein R⁴ is:

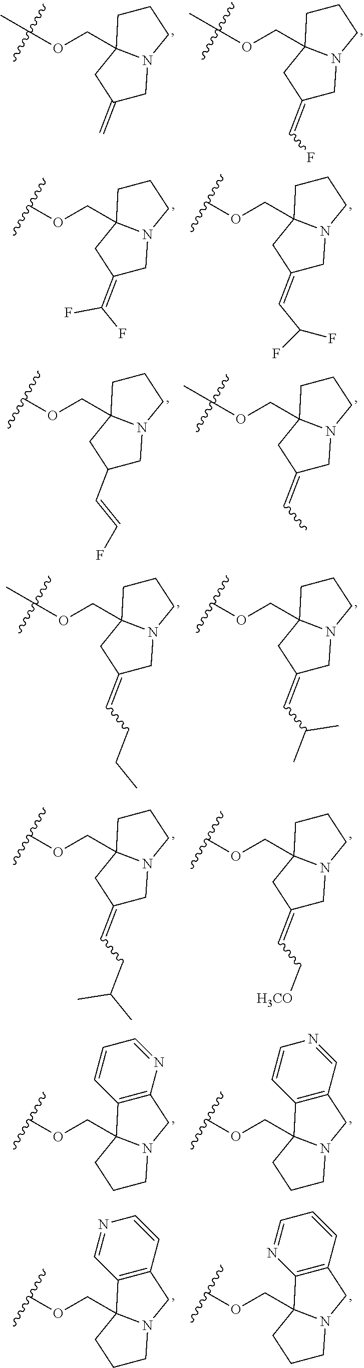

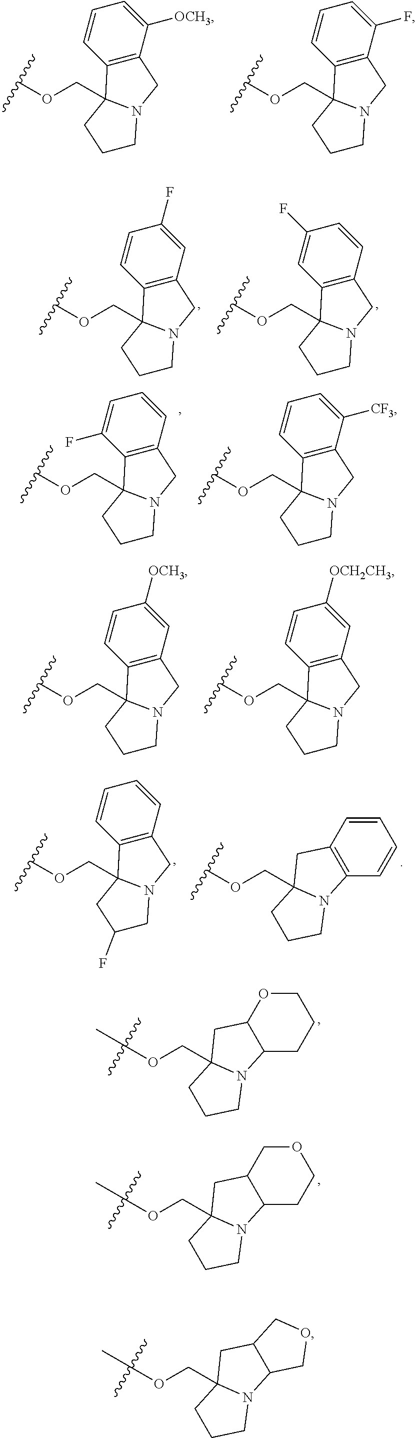

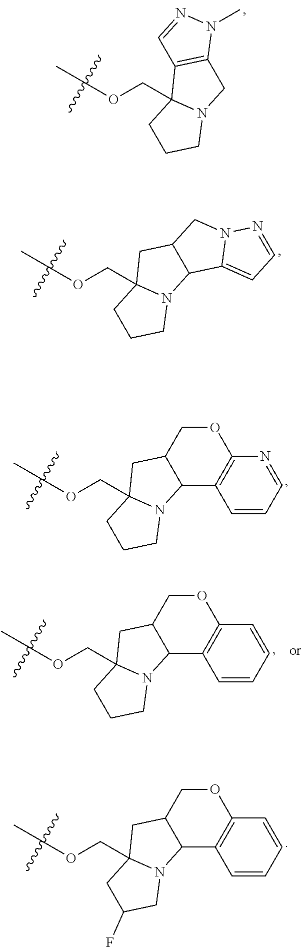 [SMILES: *OCC12CCCN1Cc1c2cnn1C ; *OCC12CCCN1C1c3ccnn3CC1C2 ; *OCC12CCCN1C1c3cccnc3OCC1C2 ; *OCC12CCCN1C1c3ccccc3OCC1C2 ; *OCC12CC(F)CN1C1c3ccccc3OCC1C2]

12. In embodiment 12, the compound of any one of embodiments 1 to 7 and 10, or a pharmaceutically acceptable salt thereof, is wherein R⁴ is:

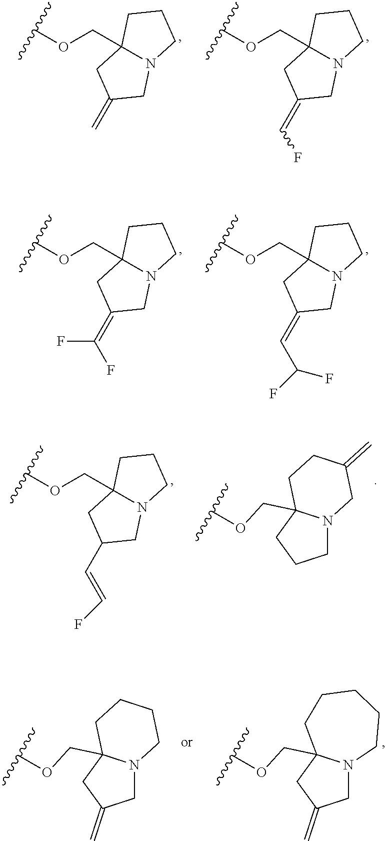

each ring optionally susbstituted with R^{e} selected from hydrogen, methyl, ethyl, isopropyl, cyclopropyl, cyclopropyloxy, fluoro, chloro, difluoromethyl, trifluoromethyl, difluoromethyloxy, trifluoromethoxy, methoxy, ethoxy, methoxymethyl, methoxymethyloxy, cyano, methylamino, dimethylamino, diethylamino, hydroxymethyl, phenyl, and benzyl.

13. In embodiment 13, the compound of any one of embodiments 1 to 7 and 8, or a pharmaceutically acceptable salt thereof, is wherein R⁴ is:

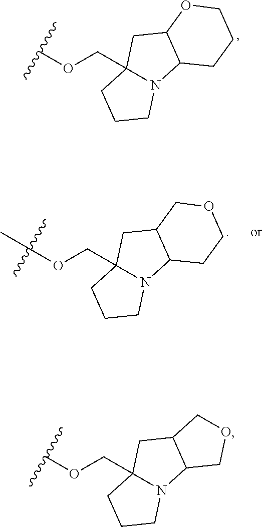 [SMILES: *OCC12CCCN1C1CCCOC1C2 ; *OCC12CCCN1C1CCOCC1C2 ; *OCC12CCCN1C1COCC1C2]

each ring optionally substituted with R^{e} selected from hydrogen, hydrogen, methyl, ethyl, isopropyl, cyclopropyl, cyclopropyloxy, fluoro, chloro, difluoromethyl, trifluoromethyl, difluoromethyloxy, trifluoromethoxy, methoxy, ethoxy, methoxymethyl, methoxymethyloxy, cyano, methylamino, dimethylamino, diethylamino, hydroxymethyl, phenyl, and benzyl.

14. In embodiment 14, the compound of any one of embodiments 1 to 13, or a pharmaceutically acceptable salt thereof, wherein R is hydrogen.

15. In embodiment 15, the compound of any of one of embodiments 1 to 14, or a pharmaceutically acceptable salt thereof, is wherein:

R¹ is:

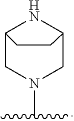 [SMILES: *N1CC2CCC(C1)N2]

16. In embodiment 16, the compound of any of one of embodiments 1 to 14, or a pharmaceutically acceptable salt thereof, is wherein:

R¹ is:

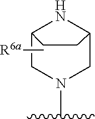

where R^{6a} is other than hydrogen.

17. In embodiment 17, the compound of any of one of embodiments 1 to 14, or a pharmaceutically acceptable salt thereof, is wherein:

R¹ is:

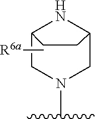

where R^{6a} is alkyl.

18. In embodiment 18, the compound of any one of embodiments 1 to 17, or a pharmaceutically acceptable salt thereof, has a structure of formula (II′c) as follows:

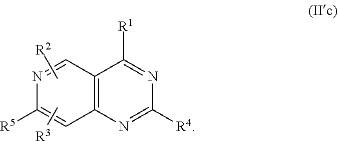

19. In embodiment 19, the compound of any one of embodiments 1 to 18, or a pharmaceutically acceptable salt thereof, has a structure of formula (II′d) as follows:

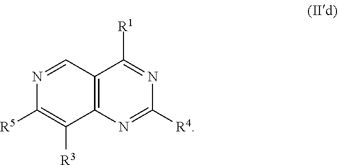

20. In embodiment 20, the compound of any one of embodiments 1 to 19, or a pharmaceutically acceptable salt thereof, is wherein R⁵ is -Q-R³⁶ where Q is bond and R³⁶ is cycloalkyl, fused cycloalkyl, aryl, aralkyl, heteroaryl, or heteroaralkyl wherein aryl, aryl in aralkyl, heteroaryl, and heteroaryl in heteroaralkyl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

21. In embodiment 21, the compound of any one of embodiments 1 to 20, or a pharmaceutically acceptable salt thereof, is wherein R⁵ is -Q-R³⁶ where Q is bond and R³⁶ is phenyl or naphthyl substituted with R^{aa}, R^{bb}, R^{cc} and R^{dd}.

22. In embodiment 22, the compound of any one of embodiments 1 to 20, or a pharmaceutically acceptable salt thereof, is wherein R⁵ is -Q-R³⁶ where Q is bond and R³⁶ is phenyl or naphthyl substituted with R^{aa}, R^{bb}, and R^{dd} where R^{aa} and R^{bb} are independently selected from hydrogen, alkyl, halo, haloalkyl, hydroxy, alkoxy, haloalkoxy, cycloalkyl, amino, cyano, and hydroxyalkyl and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

23. In embodiment 23, the compound of any one of embodiments 1 to 22, or a pharmaceutically acceptable salt thereof, is wherein R^{aa} and R^{bb} independently selected from hydrogen, methyl, ethyl, fluoro, chloro, trifluoromethyl, difluoromethyl, trifluoromethoxy, hydroxy, methyl, ethoxy, cyclopropyl, amino, cyano, and hydroxymethyl, R^{cc} is hydrogen, ethynyl, 2-cyanoethyn-1-yl, or fluoro, and R^{dd} is hydrogen, methyl, fluoro, amino, or cyclopropyl.

24. In embodiment 24, the compound of any one of embodiments 1 to 20, or a pharmaceutically acceptable salt thereof, is wherein R⁵ is -Q-R³⁶ where Q is bond and R³⁶ is heteroaryl substituted with R^{aa}, R^{bb}, R^{cc} and R^{d}d.

25. In embodiment 25, the compound of any one of embodiments 1 to 20 and 24, or a pharmaceutically acceptable salt thereof, is wherein R⁵ is -Q-R³⁶ where Q is bond and R³⁶ is a monocyclic heteroaryl substituted with R^{aa}, R^{bb}, R^{cc} and R^{dd}.

26. In embodiment 26, the compound of any one of embodiments 1 to 20 and 24, or a pharmaceutically acceptable salt thereof, is wherein R⁵ is -Q-R³⁶ where Q is bond and R³⁶ is bicyclic heteroaryl substituted with R^{aa}, R^{bb}, R^{cc} and R^{dd}.

27. In embodiment 27, the compound of any one of embodiments 1 to 20 and 24 to 26, or a pharmaceutically acceptable salt thereof, is wherein R^{aa} and R^{bb} are independently selected from hydrogen, methyl, ethyl, fluoro, chloro, trifluoromethyl, difluoromethyl, trifluoromethoxy, hydroxy, methyl, ethoxy, cyclopropyl, amino, cyano, and hydroxymethyl, R^{cc} is hydrogen or fluoro, and Rad is hydrogen, methyl, fluoro, amino, or cyclopropyl.

28. In embodiment 28, the compound of any one of embodiments 1 to 28, or a pharmaceutically acceptable salt thereof, is wherein R⁵ is -Q-R³⁶ where Q is bond and R³⁶ is:

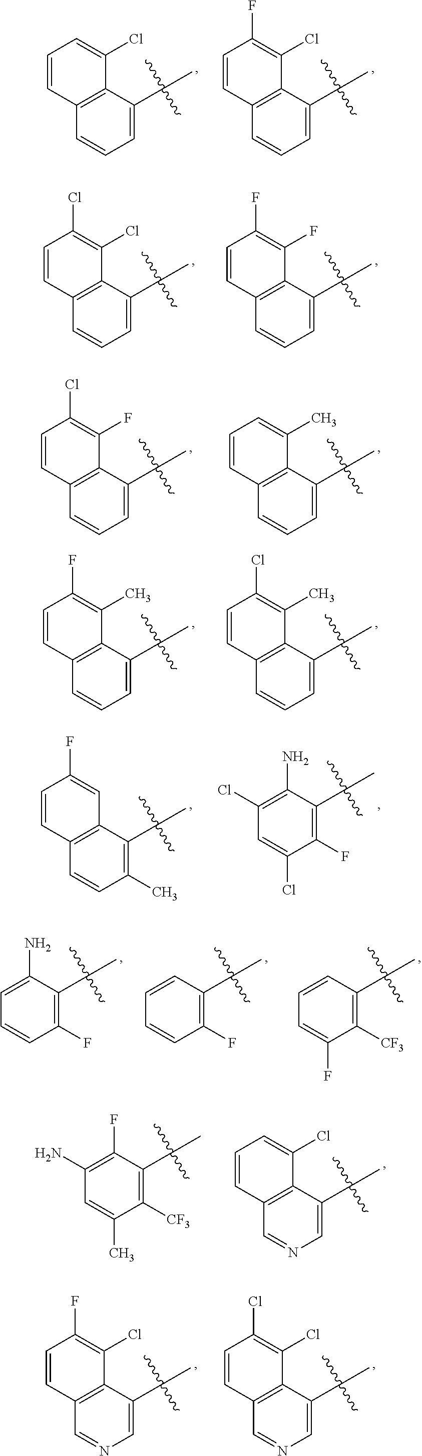

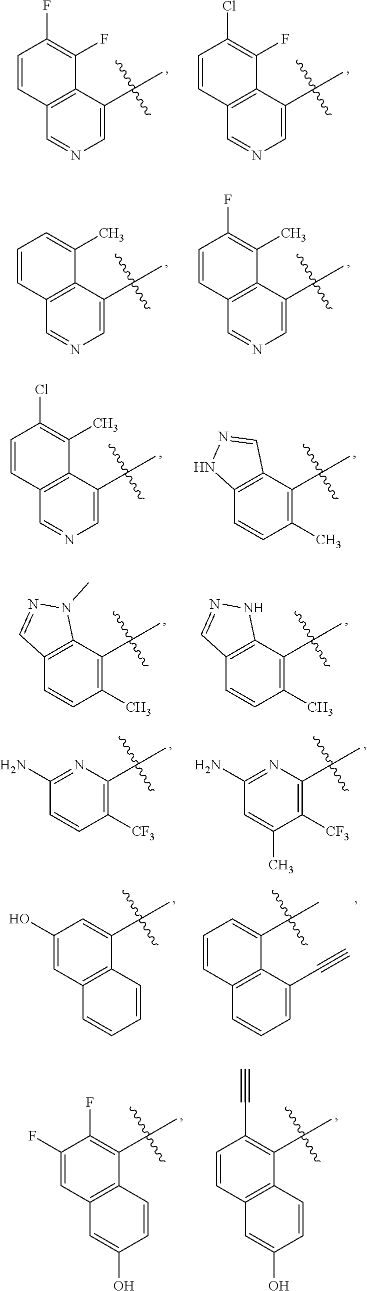

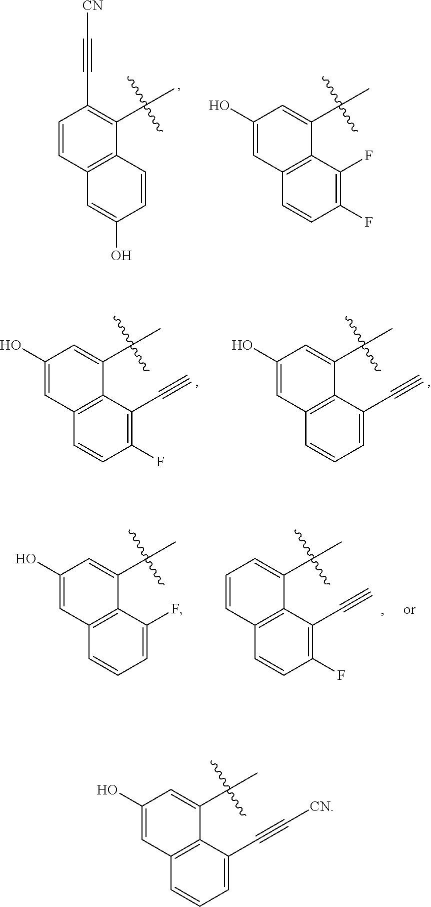

28a. In embodiment 28a, the compound of any one of embodiments 1 to 27, or a pharmaceutically acceptable salt thereof, is wherein R⁵ is -Q-R³⁶ where Q is bond and R³⁶ is:

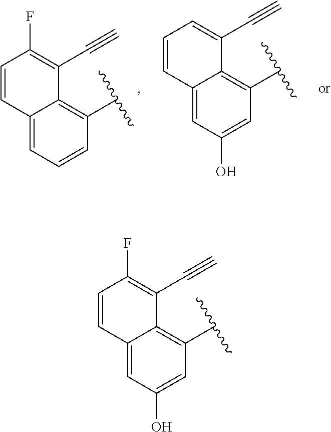 [SMILES: *c1cccc2ccc(F)c(C#C)c12 ; *c1cc(O)cc2cccc(C#C)c12 ; *c1cc(O)cc2ccc(F)c(C#C)c12]

29. In embodiment 29, the compound of any one of embodiments 1 to 27, or a pharmaceutically acceptable salt thereof, is wherein R⁵ is -Q-R³⁶ where Q is bond and R³⁶ is:

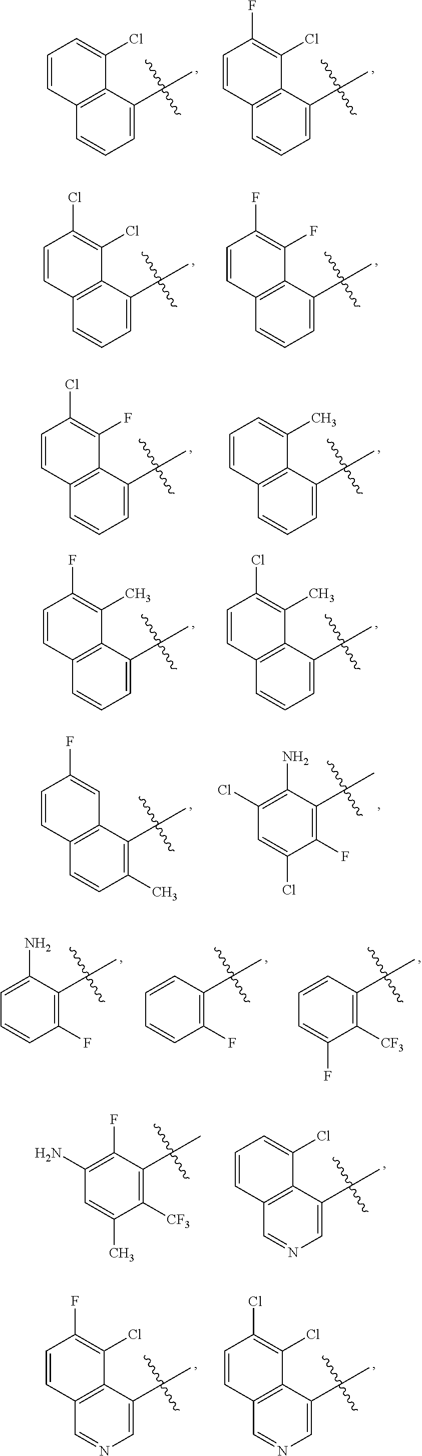

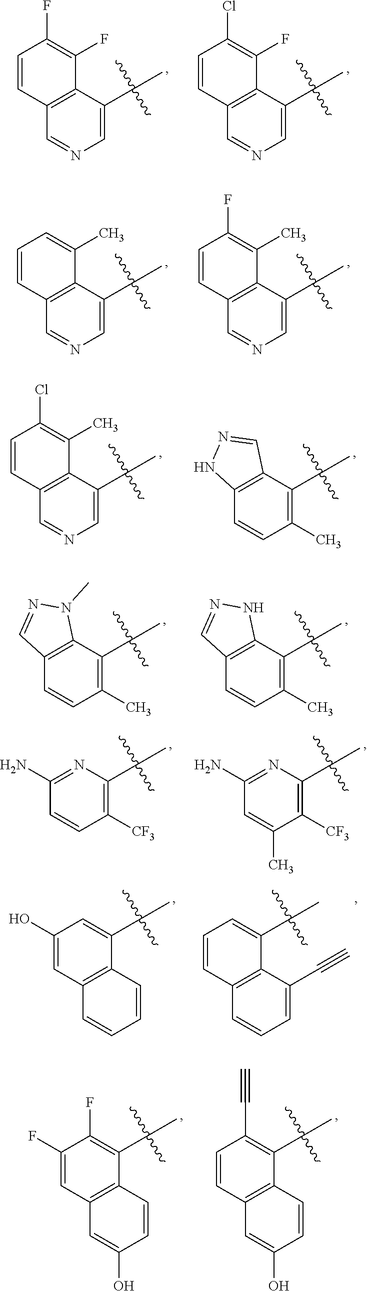

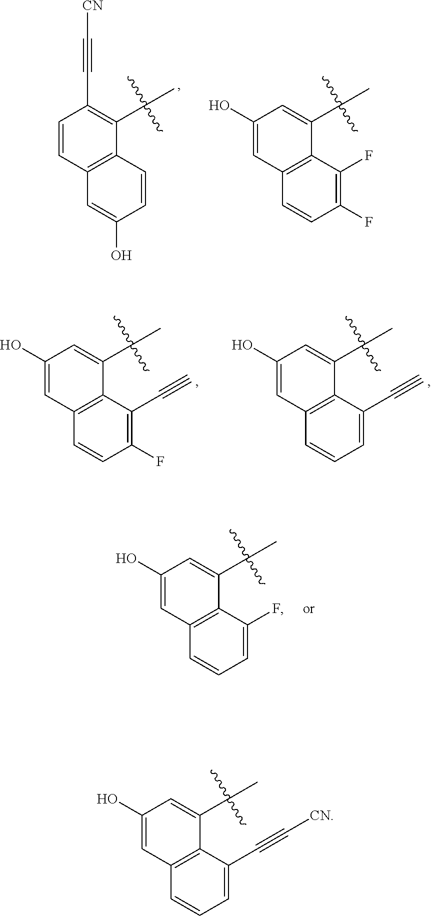 [SMILES: *c1c(C#CC#N)ccc2cc(O)ccc12 ; *c1cc(O)cc2ccc(F)c(F)c12 ; *c1cc(O)cc2ccc(F)c(C#C)c12 ; *c1cc(O)cc2cccc(C#C)c12 ; *c1cc(O)cc2cccc(F)c12 ; *c1cc(O)cc2cccc(C#CC#N)c12]

30. In embodiment 30, the compound of any one of embodiments 1 to 29, or a pharmaceutically acceptable salt thereof, is wherein, or a pharmaceutically acceptable salt thereof, is wherein R² is hydrogen, halo, or alkyl, and R³ hydrogen, halo, cycloalkyloxy, or alkyl.

31. In embodiment 31, the compound of any one of embodiments 1 to 30, or a pharmaceutically acceptable salt thereof, is wherein, or a pharmaceutically acceptable salt thereof, is wherein R² is hydrogen or chloro and R³ is hydrogen, fluoro, or cyclopropyloxy.

32. In embodiment 32, the compound of any one of embodiments 1 to 31, or a pharmaceutically acceptable salt thereof, is wherein, or a pharmaceutically acceptable salt thereof, is wherein R² is hydrogen and R³ is fluoro.

33. In embodiment 33, provided is a pharmaceutical composition comprising a compound of any one of claims 1 to 32 or a pharmaceutically acceptable salt thereof and a pharmaceutically acceptable excipient.

34. In embodiment 34, provided is a method of treating cancer in a patient, which method comprises administering to the patient, a therapeutically effective amount of a compound of any one of claims 1 to 32 or a pharmaceutically acceptable salt thereof in a pharmaceutical composition comprising a compound of any one of claims 1 to 32, or a pharmaceutically acceptable salt thereof, and a pharmaceutically acceptable excipient.

35. In embodiment 35, the method of embodiment 34, is wherein the cancer is non-small cell lung cancer, colorectal cancer, or pancreatic cancer.

36. In embodiment 36, the method of embodiment 34 or 35, is wherein the compound of any one of claims 1 to 32 or a pharmaceutically acceptable salt thereof is administered with one or more additional anticancer agents.

### General Synthetic Scheme

Compounds Formula (IIA1′) can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds Formula (IIA1′), (IIA′), (II′), or (II) can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (IIA1′) where R¹ is, for example, a ring of formula (a′) where R⁴ is —O—R³⁰, and other groups are as defined in the Summary can be prepared as illustrated and described in Scheme 1 below.

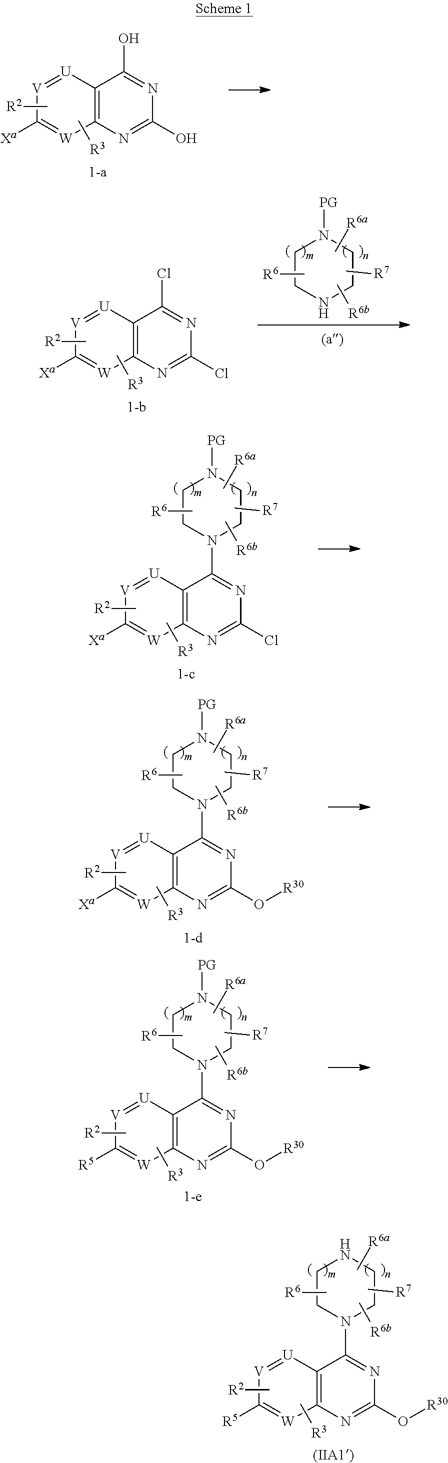

Chlorination of a compound of formula 1-a where X^{a} is a halogen, and other groups as defined in the Summary with a suitable chlorination reagent such as POCl₃ optionally in presence of a base such as DIPEA provides a 2,4-dichloro compound of formula 1-b. Compounds of Formula 1-a is either commercially available or they can be prepared by method well known in the art. Once such method is illustrated and described in Method A (i) and (ii) below.

Treatment of compound 1-b with an amine of formula (a″) where m, n, R^{6a}, R^{6b}, R⁶ and R⁷ are as defined in the Summary and PG is a suitable amino protecting group such as Boc, CBz, and the like, in the presence of a base such as DEA or DBU and the like, provides a 2-chloro compound of formula 1-c. Displacement of chloro group at C-2 position in compound 1-c with a hydroxy compound of formula R³⁰—OH where R³⁰ is as defined in the Summary provides a compound of formula 1-d. Hydroxy compounds of formula R³⁰—OH are either commercially available or can be made by methods known in the art. For example, 2-(pyrrolidin-1-yl)ethan-1-ol, (S)-(1-methylpyrrolidin-2-yl)methanol and (hexahydropentalen-3a(1H)-yl)methanol are commercially available or can be prepared by methods disclosed in PCT application publication Nos. WO2019099524 and WO2020146613 or as illustrated and described in methods (b) to (d) below. Compounds of formula 1-d where R⁴ is other than —O—R³⁰ can be prepared by methods well known in the art such as PCT application publication No. WO2019099524.

Amines of formula (a″) are either commercially available or can be made by methods known in the art. For example, benzyl 2-(cyanomethyl)piperazine-1-carboxylate, tert-butyl 2-(cyanomethyl)piperazine-1-carboxylate, benzyl 2,5-dimethylpiperazine-1-carboxylate, tert-butyl 2-methylpiperazine-1-carboxylate, tert-butyl piperazine-1-carboxylate, benzyl piperazine-1-carboxylate are commercially available. Others can be prepared by methods well known in the art.

Various R⁵ group, other than hydrogen, can be installed in compound 1-d by reacting compound 1-d and a suitable organometallic reagent of formula R⁵-M where R⁵ is alkyl, cycloalkyl, heterocyclyl, aryl or heteroaryl and M is boronic acid, boronic ester, or stannane, under Suzuki, Negeshi, and Stille reaction conditions to provide a compound of formula 1-e.

Removal of amino protecting group PG in 1-e under standard reaction condition provides a compound of Formula (IIA1′). It will be apparent to a person of ordinary skilled in the art, that compounds of Formula (IIA1′) where R¹ is a group of formula (f′) or (g′) can be similarly prepared by using appropriate mono-protected amines. Others can be prepared by methods disclosed in PCT application publication No. WO2019099524 or by methods well known in the art. Some such methods are described in Methods (e) and (f) below.

Compounds of formula 1-a can be prepared by methods well known in the art. For example,

1. Compounds of Formula 1-a where X^{a} is halogen, U is CH, V is N, W is CH, R² and R³ are as defined in the Summary (or any embodiments thereof) can be prepared as illustrated and described below.

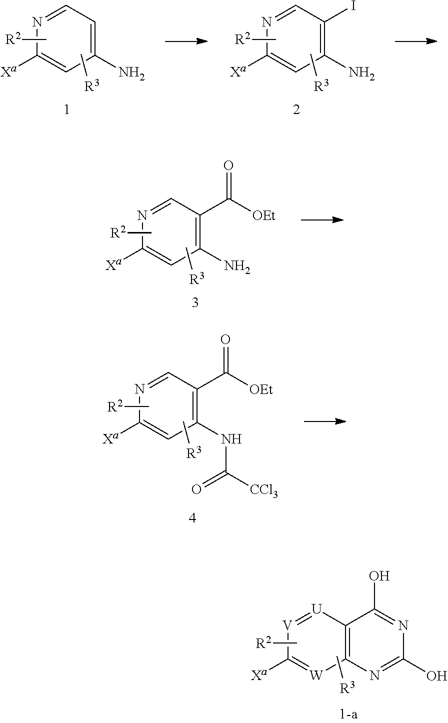

Iodination of a compound of formula 1 where X^{a} is a halo and R² and R³ are as defined in the Summary, with NIS and a suitable acid such as TsOH provides a compound of formula 2. The iodine in 2 can be converted to ethyl carboxylate under carbonylation condition including Pd catalyst such as Pd(PPh₃)₄ in carbon monoxide atmosphere and ethanol solvent to provide a compound of formula 3. Compound 3 can react with triphosgene to provide trichloroacetamido compound of formula 4, which upon treatment with ammonia in an organic solvent such as methanol, undergoes cyclization to provide compound of formula 1-a. Compounds of formula 1 are either commercially available or can be made by methods known in the art. For example, 2-chloro-3-fluoropyridin-4-amine and 2-chloropyridin-4-amine are commercially available.

2. Compounds of Formula 1-a where X^{a} is halogen, U, V and W are CH, R² and R³ are as defined in the Summary (or any embodiments thereof) can be by reacting a compound of formula

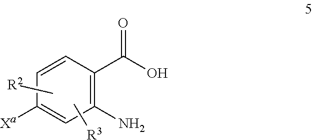

with urea at elevated temperature. Compounds of formula 5 are either commercially available or can be made by methods known in the art. For example, 2-amino-4-bromo-5-chloro-3-fluorobenzoic acid, 2-amino-4-bromo-3-fluorobenzoic acid and 2-amino-4-bromobenzoic acid are commercially available.

Certain compound of formula R³⁰—OH can be prepared as described below in Method (b) to (d) below.

### Method (b):

R³⁰—OH where R³⁰ is 4,4a,7,8,9,9a-hexahydroisoxazolo[4′, 3′: 4,5]cyclopenta-[1,2-b]-pyrrolizin-8a(6H)-yl can be synthesized by the method (b) below.

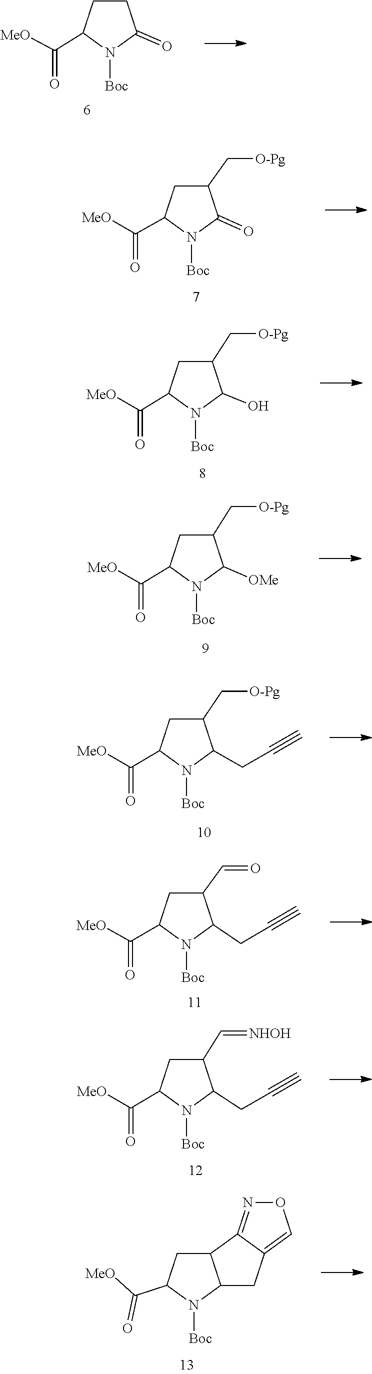

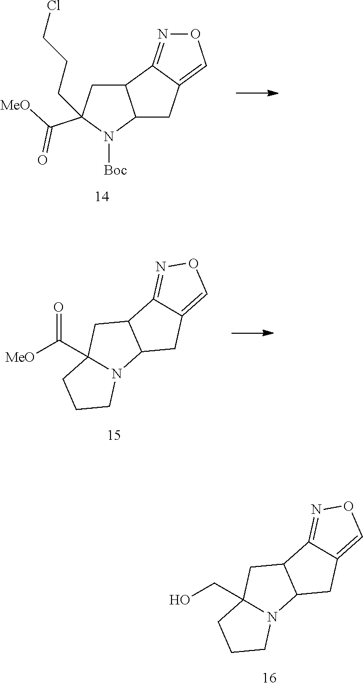 [SMILES: COC(=O)C1(CCCCl)CC2c3nocc3CC2N1C(=O)OC(C)(C)C ; COC(=O)C12CCCN1C1Cc3conc3C1C2 ; OCC12CCCN1C1Cc3conc3C1C2]

Aldol reaction between compound 6 and formaldehyde, followed by protecting the resulted alcohol with a suitable protecting group Pg such as PMB or TBS, provides compound of formula 7. Reduction of the keto group in 7 with a reducing reagent such as DIBAL can provide a hemiaminal compound 8. Subsequent treatment of 8 with acidic methanol can afford the methoxy analogue of formula 9. Treatment of 9 with a Lewis acid such as BF₃·OEt and allenyltributylstannane can provide compound 10. The alcohol protecting group P-g in 10 can be removed and resulting alcohol can be oxidized under standard oxidation condition such Dess-Martin or Swem condition to provide compound 11. Compound 11 can be converted to oxime 12 by reacting 11 with hydroxyamine under conditions well known in the art. Chlorination of 12 with a chlorinating agent such as NCS, followed by treating with a suitable base such as DIPEA or TEA can cause cyclization of 12 to provide compound 13. Compound 13 can be deprotonated with a suitable base such as LDA and then alkylated with 1-bromo-3-chloropropane to provide compound 14. The Boc group can be removed under acid condition such as TFA in DCM and the resulting amine compound can be cyclized in the presence of a base such as K₂CO₃ to provide 15, which can be reduced with a suitable base such as LiBH₄ to provide compound 16.

### Method (c):

R³⁰—OH where R³⁰ is 6,7,8,8a-tetrahydro-5H,9H-pyrazolo[1′,5′: 1,5]pyrrolo[3,4-b]-pyrrolizin-7a(3bH)-yl can be synthesized by the method (c) below:

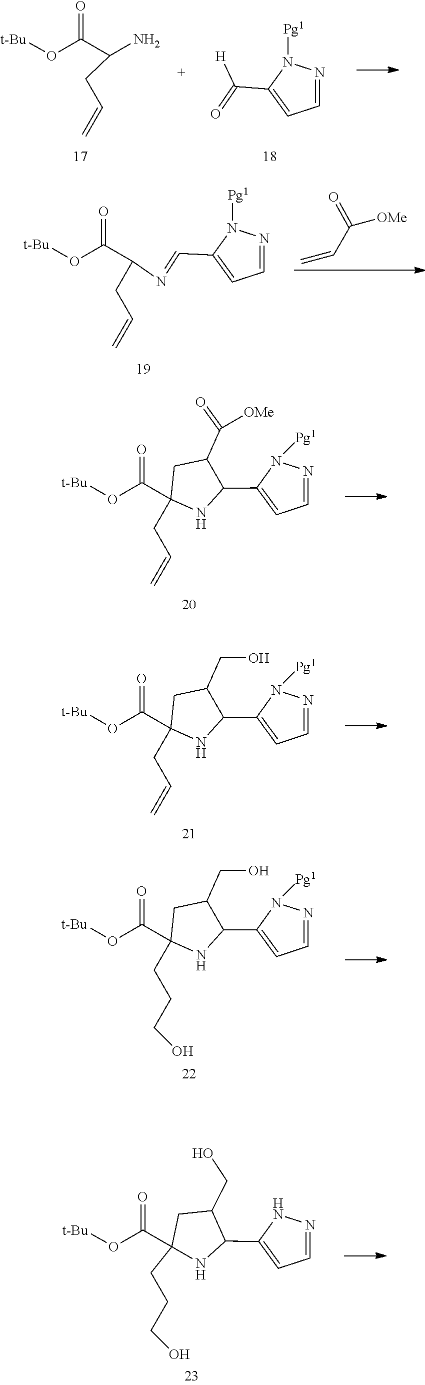

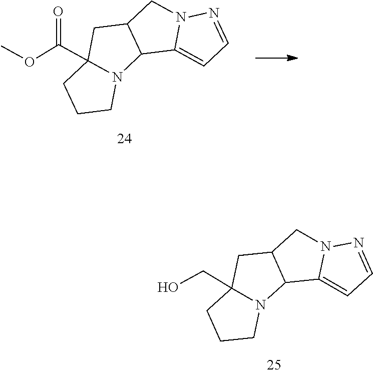 [SMILES: COC(=O)C12CCCN1C1c3ccnn3CC1C2 ; OCC12CCCN1C1c3ccnn3CC1C2]

Reaction between amine 17 and pyrazole aldehyde 18 where Pg¹ is a suitable amino protecting group can provide imine 19 which can undergo [3+2] cycloaddition with methyl acrylate to provide compound 20. The methyl ester of compound 20 can be selectively reduced with a reducing reagent such as LiBH₄, followed by hydroboration of the resulting alcohol 21 can provide compound 22. Removal of the amino protecting group in 22, followed cyclization of the resulting diol 23 by converting the hydroxyl groups to leaving groups such as halide or mesylate provides compound 24. Reduction of the ester group in 24 with a suitable reducing reagent such as LiBH₄ provides compound 25.

### Method (d):

R³⁰—OH where R³ is 1H-pyrrolizin-7a(5H)-yl substituted with alkylidenyl, haloalkylidenyl, or alkoxyalkylidenyl can be synthesized by the method (d) below:

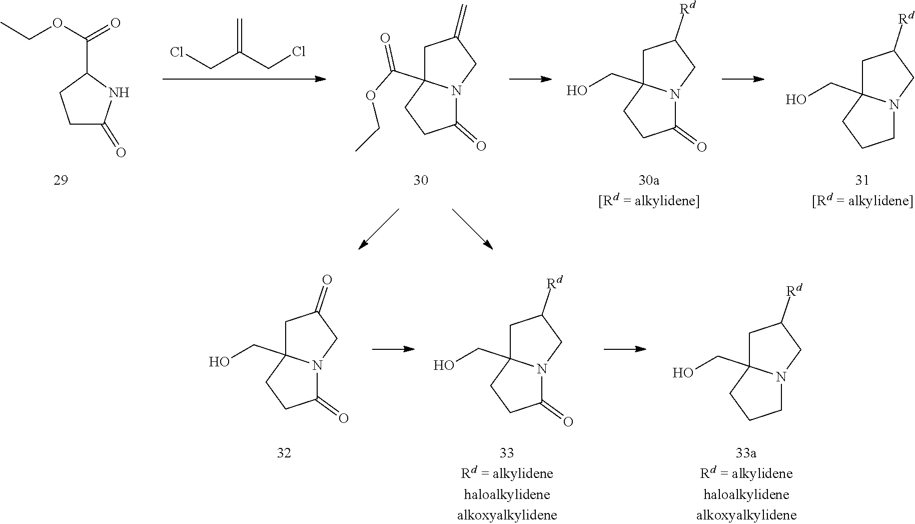

Compound 29 can undergo cyclization with 3-chloro-2-(chloromethyl)prop-1-ene in the presence of a base such as LHMDS to provide compound ethyl 2-methylidene-5-oxotetrahydro-1H-pyrrolizine-7a(5H)-carboxylate 30. Methylidene of Compound 30 can be replaced by other alkylidenes by treating compound 30 with suitable alkene in the presence of an olefin metathesis catalyst such as Hoveyda-Grubbs Catalyst® [CAS No. 301224-40-8] or Zhan catalyst [CAS No. 918870-76-5] to provide 30a where R^{d} is alkylidene other than methylidene. Reduction of the keto group in compound 30 and 30a with a suitable reducing reagent such as LiAlH₄ provides compound 31 where R^{d} is alkylidene.

Compound of formula 33a where R^{d} is alkylidene, haloalkylidene, or alkoxyalkylidene can be prepared by cleavage of the olefin in 30 under oxidative cleavage condition such as NaIO₄ and catalytic amount of RuCl3 to provide diketo compound 32. Compound 32 is converted to 33 where R^{d} is alkylidene, haloalkylidene, or alkoxyalkylidene under standard Wittig olefination condition. Compound 33 can also be prepared directly by treatment of compound 30 by alkylidene replacement described above. Compound 33 is converted to compound 33a as described above.

Certain amines that can be used to synthesize compounds of Formula (IIA1′) where R¹ is a group of formula (f) and (f′) and (a) and (a′) respectively, can be prepared as described below in Methods (e) and (f) below:

### Method e:

(i)

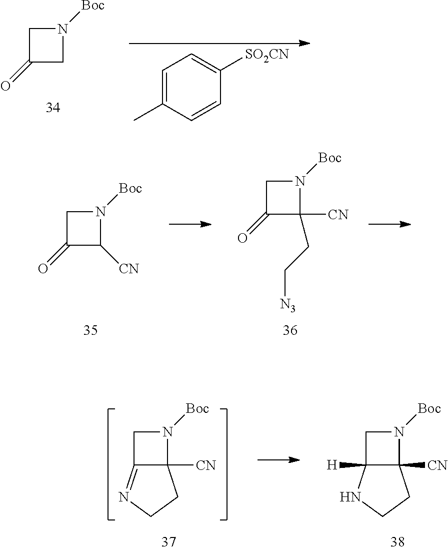

Deprotonation of Boc protected oxoazetidine 34 with a strong base such as LDA followed by treatment with 4-methylbenzenesulfonyl cyanide affords CN substituted oxoazetidine 35, which then is deprotonated with a base such as NaH, followed by alkylation with 1-azido-2-bromoethane provides bi-substituted oxoazetidine 36. The azide group of 36 can be reduced by catalytic hydrogenation to form NH₂ group which reacts with ketone group intramolecularly to form the imine intermediate 37. Further reduction of imine group on 37 by catalytical hydrogenation or sodium triacetoxyborohydride provides bicyclic amine 38.

(ii)

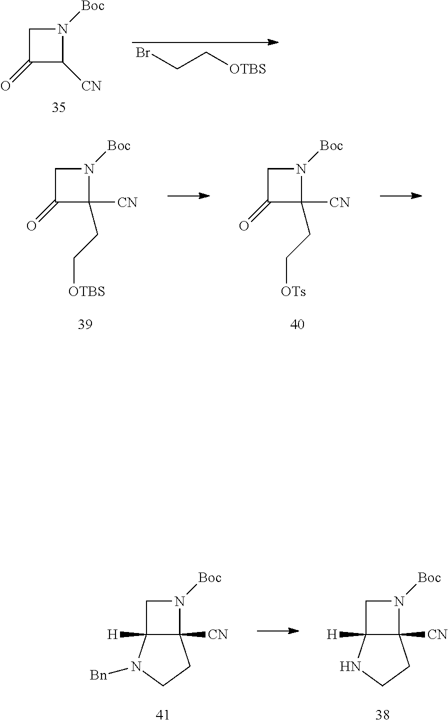 [SMILES: CC(C)(C)OC(=O)N1CC(=O)C1C#N ; CC(C)(C)[Si](C)(C)OCCBr ; CC(C)(C)OC(=O)N1CC(=O)C1(C#N)CCO[Si](C)(C)C(C)(C)C ; Cc1ccc(S(=O)(=O)OCCC2(C#N)C(=O)CN2C(=O)OC(C)(C)C)cc1 ; CC(C)(C)OC(=O)N1C[C@H]2N(Cc3ccccc3)CC[C@]21C#N ; CC(C)(C)OC(=O)N1C[C@H]2NCC[C@]21C#N]

Alternatively, compound 35 can be deprotonated an then alkylated with (2-bromo-ethoxy)(tert-butyl)dimethylsilane to afford the bi-substituted oxoazetidine 39. Removal of the TBS group of 39 under conditions well known the art, followed by conversion of the hydroxy into a suitable leaving group such as tosylate provides compound 40. Reaction of 40 with benzylamine under reductive amination conditions, followed by intramolecular cyclization of the resulting amine compound provides bicyclic amine 41, which upon removal of the benzyl protecting group provides compound 38.

### Method (f):

(i)

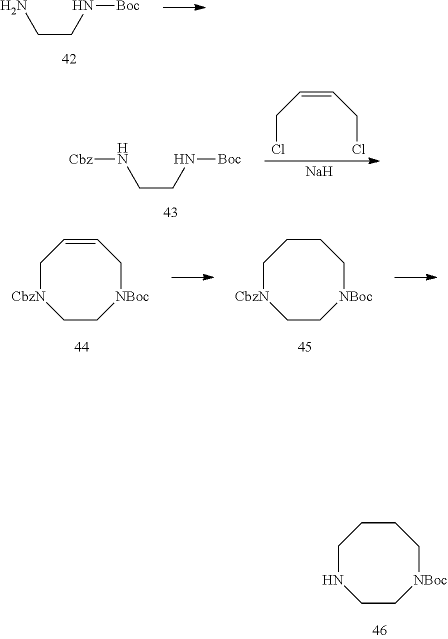

Protection of the free amine group of compound 42 with Cbz group provides 43. Deprotonation of 43 with a strong base such as NaH, followed by cyclization with (Z)-1,4-dichlorobut-2-ene provides alkene 44. The double bond of 44 is then hydrogenated to produce 1,4-diazocane 45, which upon removal of the Cbz group under conditions well known in the art, provides an amine of formula 46.

(ii)

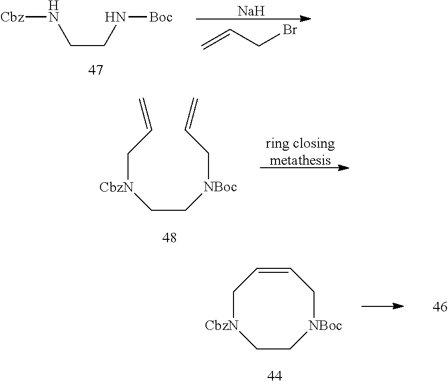

Alternatively, alkene 44 can be made from diene 48 by intramolecular ring closing metathesis. Compound 48 can be prepared by treating compound 47 with a deprotonating agent such as sodium hydride and treating the resulting deprotonated intermediate with allyl bromide. Compound 44 can then be converted to compound 46 as described above.

(iii)

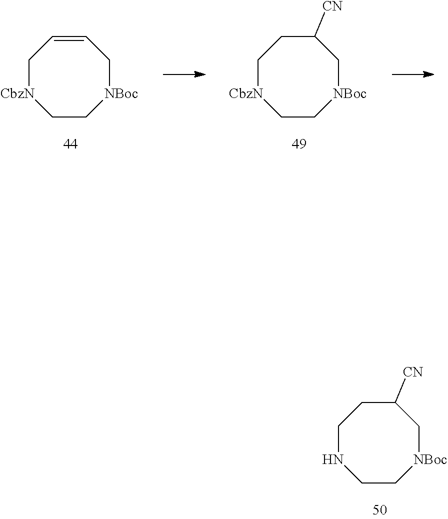 [SMILES: CC(C)(C)OC(=O)N1C/C=C\CN(C(=O)OCc2ccccc2)CC1 ; CC(C)(C)OC(=O)N1CCN(C(=O)OCc2ccccc2)CCC(C#N)C1 ; CC(C)(C)OC(=O)N1CCNCCC(C#N)C1]

Ni-catalyzed hydrocyanation of compound 44 in the presence of zinc powder and formamide provides nitrile 49, which upon removal of the Cbz group provides compound 50.

Compounds of Formula (IIA1′) where R¹ is, for example, a ring of formula (a′) where R^{6a} and R^{6b} are hydrogen, R⁴ is —CR³³═CR³⁴R³⁵ and other groups are as defined in the Summary can be prepared as described in Scheme 2 below.

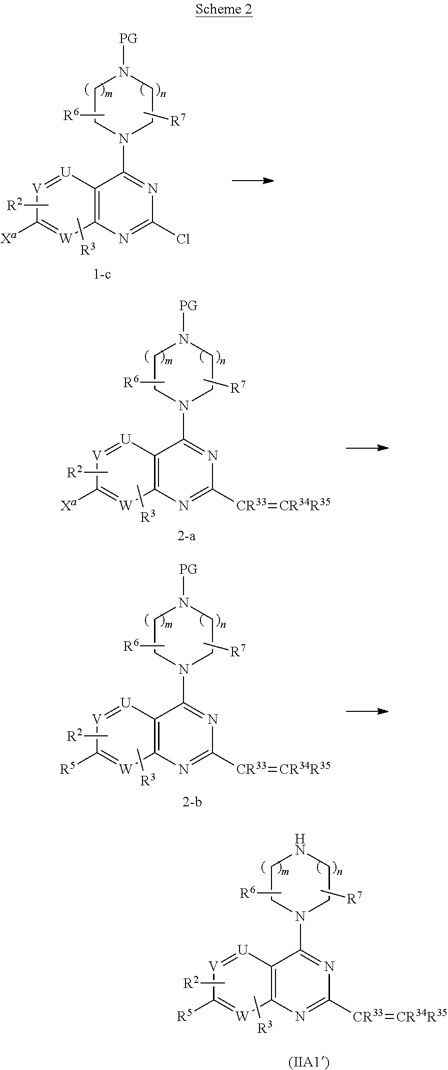

Treatment of a compound of formula 1-c with a boronic ester of formula CR³⁴R³¹═CR³³B(OR)₂ where R is alkyl or CR³⁴R³⁵═CR³³B(—OC(CH₃)₂CH₃)₂)₂—O—) where R³³, R³⁴ and R³⁵ as defined in Summary, under Suzuki coupling conditions provides a compound of formula 2-a, which can be converted to a compound of Formula (IIA1′) as described in Scheme 1 above.

Boronic esters of formula CR³⁴R³⁵═CR³³B(OR)₂ are either commercially available, or they can be prepared by methods well known in the art. For example, 2-methyl-1-(4,4,5,5-tetramethyl-1,3,2-dioxaborolan-2-yl)-1-propene and 2-(cyclopentylidenemethyl)-4,4,5,5-tetramethyl-1,3,2-dioxaborolane are commercially available.

It will be readily apparent to a person skilled in the art that based on the nature of the R² to R⁵, R³³R³⁴ and R³⁵ groups in a compound of Formula (IIA1′), the process illustrated above may include additional optional steps such as additional addition and removal of protecting groups and/or modification of R² to R⁵ to other R² to R⁵ groups as defined in the Summary. For example, compound of Formula (IIA1′) where R⁴ is —CR³³═CR³⁴R³⁵ where —CR³³═CR³⁴R³⁵ is

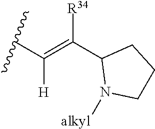

where R³⁴ is hydrogen or alkyl and R³⁵ is 1-alkylpyrrolidin-2-yl, can be prepared as shown below:

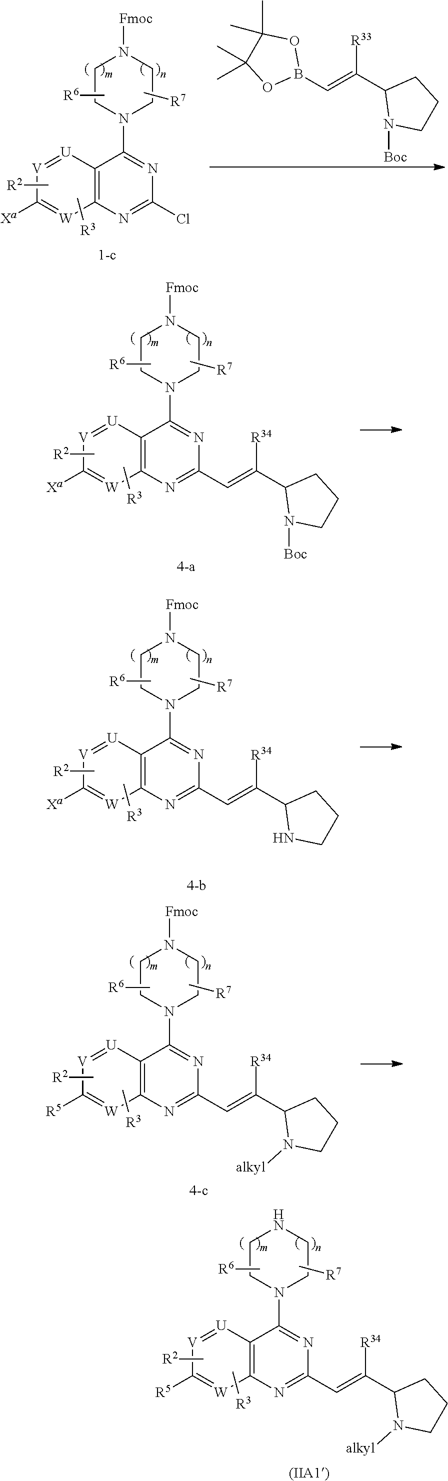

Boronic ester of formula

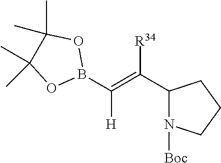

where R³⁴ is hydrogen or alkyl can be prepared by deprotonation of bis(4,4,5,5-tetramethyl-1,3,2-dioxaborolan-2-yl)methane with LiTMP, followed by reacting the resulting anion with a compound of formula

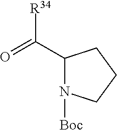

where R³⁴ is hydrogen or alkyl.

Alternatively, compounds of Formula (IIA1′) where R¹ is, for example, a ring of formula (a′) where R⁴ is —O—R³⁰, and other groups are as defined in the Summary can be prepared as illustrated and described in Scheme 3 below.

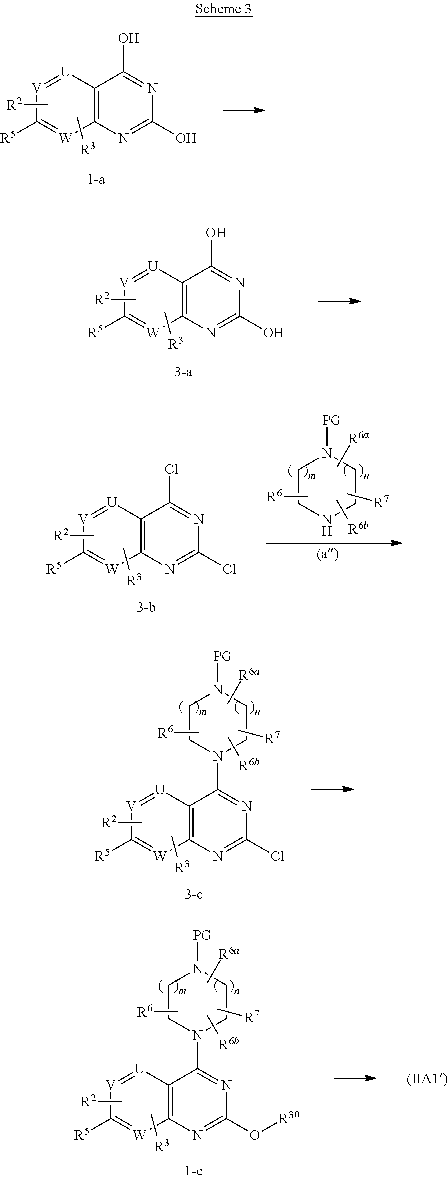

Coupling reaction between Compound 1-a and a suitable organometallic reagent of formula R⁵-M where R⁵ is alkyl, cycloalkyl, heterocyclyl, aryl or heteroaryl and M is boronic acid, boronic ester, or stannane, under Suzuki, Negeshi, and Stille reaction conditions respectively, to provide a compound of formula 3-a. Chlorination of a compound of formula 3-a with a suitable chlorination reagent such as POCl₃ optionally in presence of a base such as DIPEA provides compound of formula 3-b. Treatment of compound 3-b with an amine of formula (a″) in the presence of a base such as DEA or DBU and the like, provides a 2-chloro compound of formula 3-c. Displacement of chloro group at C-2 position in compound 3-c with a hydroxy compound of formula R³⁰—OH provides a compound of formula 1-e, which is converted to a compound of Formula (IIA1′) as described above.

Compounds of Formula (IIA1′) where R¹ is, for example, a ring of formula (a′) where R⁴ is —O—R³⁰, and other groups are as defined in the Summary can be prepared as illustrated and described in Scheme 4 below.

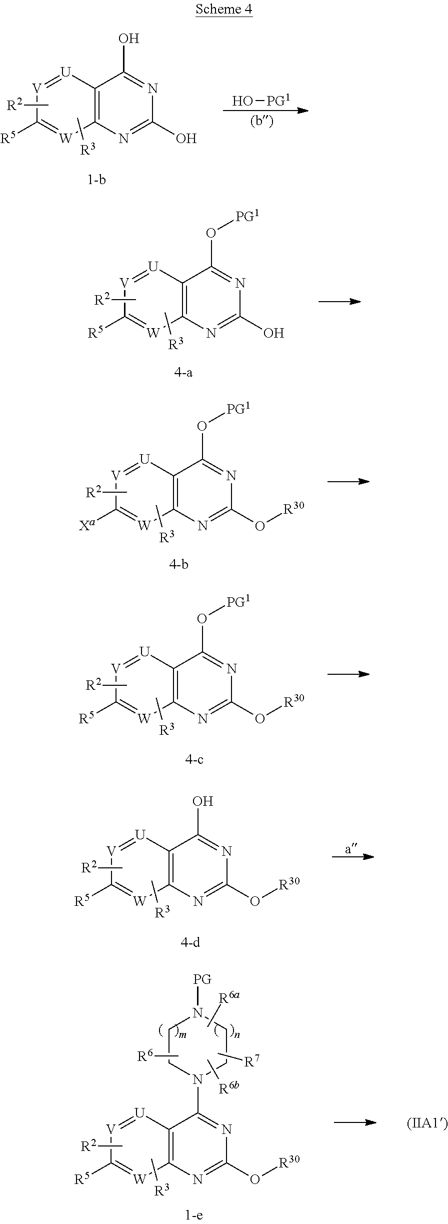

Reaction between compound of formula 1-b and with a compound of formula PG¹-OH where PG¹ is a protecting group such as (2-(trimethylsilyl)ethoxymethyl) for hydroxyl group in the presence of a base such as DEA or DBU and the like, provides a compound of formula 4-a which can be converted to a compound of formula 4-c as described above. Removal of the hydroxyl protecting group under suitable condition provides a compound of formula 4-d. For example, when PG is SEM, it can be cleaved by treating with CsF in organic solvent such as DMF. Compound of formula 4-d is then converted to a compound of Formula (IIA1′) as described above.

### Utility

The present disclosure provides treatment of cancer mediated by K-ras, in particular with G12D mutants. In some embodiments, the cancer is pancreatic cancer, colorectal cancer, lung cancer, gall bladder cancer, thyroid cancer, and bile duct cancer. In certain embodiments the lung cancer is a non-small cell lung carcinoma (NSCLC), for example adenocarcinoma, squamous-cell lung carcinoma or large-cell lung carcinoma. In some embodiments, the lung cancer is a small cell lung carcinoma. Other lung cancers treatable with the disclosed compounds include, but are not limited to, glandular tumors, carcinoid tumors and undifferentiated carcinomas.

K-ras G12D mutations are observed in hematological malignancies that affect blood, bone marrow, and/or lymph nodes. As such the compounds of Formula (IIA1′), (IIA′), (II′), or (II) or a pharmaceutically acceptable salt thereof can be used for the treatment of acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias, lymphomas such as all subtypes of Hodgkins lymphoma or non-Hodgkins lymphoma, plasma cell malignancies such as multiple myeloma, mantle cell lymphoma, and Waldenstrom's macroglubunemia.

The compounds of Formula (IIA1′), (IIA′), (II′), or (II), or a pharmaceutically acceptable salt thereof can be used for the treatment of a hyperproliferative disorder or metastasis in human who suffers from a cancer such as acute myeloid leukemia, cancer in adolescents, adrenocortical carcinoma childhood, AIDS related cancers (e.g. Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. The compounds of Formula (IIA1′), (IIA′), (II′), or (II), or a pharmaceutically acceptable salt thereof can also be used for the treatment of a non-cancerous hyperproliferative disorder such as benign hyperplasia of the skin (e. g., psoriasis), restenosis, or prostate (e. g., benign prostatic hypertrophy (BPH)).

### Testing

The K-Ras G12C and/or G12D activity of the compounds of Formula (IIA1′), (IIA′), (II′), or (II), or a pharmaceutically acceptable salt thereof can be tested using the in vitro assay described in Biological Examples 1 below.

### Pharmaceutical Compositions

In general, the compounds Formula (IIA1′), (IIA′), (II′), or (II) (unless stated otherwise, reference to compound/compounds of Formula (IIA1′), (IIA′), (II′), or (II) herein includes any embodiments thereof described herein or a pharmaceutically acceptable salt thereof) will be administered in a therapeutically effective amount by any of the accepted modes of administration for agents that serve similar utilities. Therapeutically effective amounts of compounds Formula (IIA1′), (IIA′), (II′), or (II) may range from about 0.01 to about 500 mg per kg patient body weight per day, which can be administered in single or multiple doses. A suitable dosage level may be from about 0.1 to about 250 mg/kg per day or about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound Formula (IIA1′), (IIA′), (II′), or (II), i.e., the active ingredient, will depend upon numerous factors such as the severity of the disease to be treated, the age and relative health of the patient, the potency of the compound being utilized, the route and form of administration, and other factors.

In general, compounds Formula (IIA1′), (IIA′), (II′), or (II) will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a compound of Formula (IIA1′), (IIA′), (II′), or (II) in combination with at least one pharmaceutically acceptable excipient. Acceptable excipients are generally non-toxic, aid administration, and do not adversely affect the therapeutic benefit of the compound of Formula (IIA1′), (IIA′), (II′), or (II). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

The compounds of Formula (IIA1′), (IIA′), (II′), or (II) may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds of Formula (IIA1′), (IIA′), (II′), or (II) may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds of Formula (IIA1′), (IIA′), (IF), or (II) may also be formulated in rectal compositions such as suppositories or retention enemas, e.g., containing conventional suppository bases such as cocoa butter, polyethylene glycol, or other glycerides.

Certain compounds of Formula (IIA1′), (IIA′), (II′), or (II) may be administered topically, that is by non-systemic administration. This includes the application of a compound of Formula (IIA1′), (IIA′), (II′), or (II) externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds of Formula (IIA1′), (IIA′), (II′), or (II) may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds of Formula (IIA1′), (IIA′), (II′), or (II) may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator. Other suitable pharmaceutical excipients and their formulations are described in Remington's Pharmaceutical Sciences, edited by E. W. Martin (Mack Publishing Company, 20th ed., 2000).

The level of the compound of Formula (IIA1′), (IIA′), (II′), or (II) in a formulation can vary within the full range employed by those skilled in the art. Typically, the formulation will contain, on a weight percent (wt. %) basis, from about 0.01-99.99 wt. % of a compound of Formula _{(IIA}1′), (IIA′), (II′), or (II) based on the total formulation, with the balance being one or more suitable pharmaceutical excipients. For example, the compound is present at a level of about 1-80 wt. %.

### Combinations and Combination Therapies

The compounds of Formula (IIA1′), (IIA′), (II′), or (II) or a pharmaceutically acceptable salt thereof may be used in combination with one or more other drugs in the treatment of diseases or conditions for which compounds of Formula (IIA1′), (IIA′), (II′), or (II) or the other drugs may have utility. Such other drug(s) may be administered, by a route and in an amount commonly used therefore, contemporaneously or sequentially with a compound of Formula (IIA1′), (IIA′), (II′), or (II) or a pharmaceutically acceptable salt thereof. When a compound of Formula (IIA1′), (IIA′), (II′), or (II) or a pharmaceutically acceptable salt thereof is used contemporaneously with one or more other drugs, a pharmaceutical composition in unit dosage form containing such other drugs and the compound of Formula (IIA1′), (IIA′), (II′), or (II) or a pharmaceutically acceptable salt thereof can be used. Accordingly, the pharmaceutical compositions of the present disclosure also include those that contain one or more other drugs, in addition to a compound of Formula (IIA1′), (IIA′), (II′), or (II) or a pharmaceutically acceptable salt thereof The combination therapy may also include therapies in which the compound of Formula (IIA1′), (IIA′), (II′), or (II) or a pharmaceutically acceptable salt thereof and one or more other drugs are administered on different overlapping schedules. It is also contemplated that when used in combination with one or more other active ingredients, the compounds of Formula (IIA1′), (IIA′), (II′), or (II) and the other active ingredients may be used in lower doses than when each is used singly. The weight ratio of the compound of this disclosure to the second active ingredient may be varied and will depend upon the effective dose of each ingredient. Generally, an effective dose of each will be used.

Where the subject in need is suffering from or at risk of suffering from cancer, the patient can be treated with a compound of Formula (IIA1′), (IIA′), (II′), or (II) or a pharmaceutically acceptable salt thereof in any combination with one or more other anti-cancer agents including but not limited to:

MAP kinase pathway (RAS/RAF/MEK/ERK) inhibitors including but not limited to: Vemurafanib (PLX4032, CAS No. 918504-65-1), Dabrafenib (CAS No. 1195765-45-7), Encorafenib (LGX818 CAS No. 1269440-17-6), TQ-B3233, XL-518 (Cas No. 1029872-29-4, available from ACC Corp); trametinib (CAS No. 871700-17-3), selumetinib (AZD6244 CAS No. 606143-52-6), TQ-B3234, PD184352 (CAS No. 212631-79-3), PD325901 (CAS No. 391210-10-9), TAK-733 (CAS No. 1035555-63-5), pimasertinib (CAS No. 1236699-92-5), binimetinib (CAS No. 606143-89-9), refametinib (CAS No. 923032-37-5), cobimetinib (GDC-0973 CAS No. 934660-93-2), AZD8330 (CAS No. 869357-68-6), BVD-523 (CAS No. 869886-67-9), LTT462 (CAS No. 869886-67-9), AMG510 (CAS No. 2296729-00-3), ARS853 (CAS No. 1629268-00-3), and any RAS inhibitors disclosed in patents WO2016049565, WO2016164675, WO2016168540, WO2017015562, WO2017058728, WO2017058768, WO2017058792, WO2017058805, WO2017058807, WO2017058902, WO2017058915, WO2017070256, WO2017087528, WO2017100546, WO2017172979, WO2017201161, WO2018064510, WO2018068017, and WO2018119183;

SHP2 inhibitors including but not limited to: SHP099 (CAS No. 2200214-93-1), TNO155 (CAS No. 1801765-04-7), RMC4630, JAB-3312, JAB-3068 and ERAS-601;

SOS1 inhibitors including but not limited to BI1701963 and BAY-293;

CSF1R inhibitors (PLX3397, LY3022855) and CSF1R antibodies (IMC-054, RG7155);

TGF beta receptor kinase inhibitor such as LY2157299;

BTK inhibitor such as ibrutinib; BCR-ABL inhibitors: Imatinib (CAS No. 152459-95-5); Inilotinib hydrochloride; Nilotinib (CAS No. 923288-95-3); Dasatinib (BMS-345825 CAS No. 302962-49-8); Bosutinib (SKI-606 CAS No. 380843-75-4); Ponatinib (AP24534 CAS No. 943319-70-8); Bafetinib (INNO406 CAS No. 859212-16-1); Danusertib (PHA-739358 CAS No. 827318-97-8), AT9283 (CAS No. 896466-04-9); Saracatinib (AZD0530 CAS No. 379231-04-6); and PF-03814735 (CAS 942487-16-3);

ALK inhibitors: PF-2341066 (XALKOPJ®; crizotinib); 5-chloro-N4-(2-(isopropyl-sulfonyl)phenyl)-N2-(2-methoxy-4-(4-(4-methylpiperazin-1-yl)piperidin-1-yl)phenyl)pyrimidine-2,4-diamine; GSK1838705A (CAS No. 1116235-97-2); CH5424802 (CAS No. 1256580-46-7); Ceritinib (ZYKADIA CAS No. 1032900-25-6); TQ-B3139, and TQ-B3101;

PI3K inhibitors: 4-[2-(1H-indazol-4-yl)-6-[[4-(methylsulfonyl)-piperazin-1-yl]methyl]thieno[3,2-d]pyrimidin-4-yl]morpholine (also known as GDC 0941 and described in PCT Publication Nos. WO 09/036082 and WO 09/055730), BEZ235 or NVP-BEZ235 (CAS No. 915019-65-7), disclosed in PCT Publication No. WO 06/122806);

Vascular Endothelial Growth Factor (VEGF) receptor inhibitors: Bevacizumab (sold under the trademark Avastin® by Genentech/Roche), axitinib, (N-methyl-2-[[3-[(E)-2-pyridin-2-ylethenyl]-1H-indazol-6-yl]sulfanyl]benzamide, also known as AG013736, and described in PCT Publication No. WO 01/002369), Brivanib Alaninate ((S)—((R)-1-(4-(4-fluoro-2-methyl-4H-indol-5-yloxy)-5-methylpyrrolo[2,1-f][1,2,4]triazin-6-yloxy)propan-2-yl)2-aminopropanoate, also known as BMS-582664), motesanib (N-(2,3-dihydro-3,3-dimethyl-1H-indol-6-yl)-2-[(4-pyridinylmethyl)amino]-3-pyridinecarboxamide, and described in PCT Publication No. WO 02/066470), pasireotide (also known as SOM230, and described in PCT Publication No. WO 02/010192), sorafenib (sold under the tradename Nexavar®, CAS No. 284461-73-0); or AL-2846;

MET inhibitor such as foretinib (CAS No. 849217-64-7), cabozantinib (CAS No. 1140909-48-3), capmatinib (CAS No. 1029712-80-8), tepotinib (CAS No. 1100598-32-0), savolitinib (CAS No. 1313725-88-0, or crizotinib (CAS No. 877399-52-5);

FLT3 inhibitors—sunitinib malate (CAS No. 341031-54-7, sold under the tradename Sutent® by Pfizer); PKC412 (CAS No. 120685-11-2, midostaurin); tandutinib (CAS No. 387867-13-2), sorafenib (CAS No. 284461-73-0), lestaurtinib (CAS No.:1 11358-88-4), KW-2449 (CAS No. 1000669-72-6), quizartinib (AC220, CAS No. 950769-58-1), or crenolanib (CAS No. 670220-88-9);

Epidermal growth factor receptor (EGFR) inhibitors: Gefitnib (sold under the tradename Iressa®), N-[4-[(3-chloro-4-fluorophenyl)amino]-7-[[(3S)-tetrahydro-3-furanyl]oxy]-6-quinazolinyl]-4(dimethylamino)-2-butenamide, sold under the tradename Tovok® by Boehringer Ingelheim), cetuximab (sold under the tradename Erbitux® by Bristol-Myers Squibb), or panitumumab (sold under the tradename Vectibix® by Amgen);

HER2 receptor inhibitors: Trastuzumab (sold under the trademark Herceptin® by Genentech/Roche), neratinib (also known as HKI-272, (2E)-N-[4-[[3-chloro-4-[(pyridin-2-yl)methoxy]phenyl]amino]-3-cyano-7-ethoxyquinolin-6-yl]-4-(d imethylamino)but-2-enamide, and described PCT Publication No. WO 05/028443), lapatinib (CAS No. 231277-92-2) or lapatinib ditosylate (CAS No: 388082-77-7) (sold under the trademark Tykerb® by GlaxoSmithKline); or Trastuzumab emtansine (in the United States, ado-trastuzumab emtansine, trade name Kadcyla)—an antibody-drug conjugate consisting of the monoclonal antibody trastuzumab (Herceptin) linked to the cytotoxic agent mertansine (DM1);

HER dimerization inhibitors: Pertuzumab (sold under the trademark Omnitarg®, by Genentech);

FGFR inhibitors: Erdafitinib (CAS No. 1346242-81-6), Pemigatinib (CAS No. 1513857-77-6) or Infigratinib (CAS No. 872511-34-7)

Aurora kinase inhibitors: TAS-119 (CAS No. 1453099-83-6), LY3295668 (CAS No. 1919888-06-4), or alisertib (CAS No. 1028486-01-2);

CD20 antibodies: Rituximab (sold under the trademarks Riuxan® and MabThera® by Genentech/Roche), tositumomab (sold under the trademarks Bexxar® by GlaxoSmithKline), or ofatumumab (sold under the trademark Arzerra® by GlaxoSmithKline);

Tyrosine kinase inhibitors: Erlotinib hydrochloride (CAS No. 183319-69-9, sold under the trademark Tarceva® by Genentech/Roche), Linifanib (N-[4-(3-amino-1H-indazol-4-yl)phenyl]-N′-(2-fluoro-5-methylphenyl)urea, also known as ABT 869, available from Genentech), sunitinib malate (CAS No. 341031-54-7, sold under the tradename Sutent® by Pfizer), bosutinib (4-[(2,4-dichloro-5-methoxyphenyl)amino]-6-methoxy-7-[3-(4-methylpiperazin 4-yl)propoxy]quinoline-3-carbonitrile, also known as SKI-606, and described in U.S. Pat. No. 6,780,996), dasatinib (CAS No. 302962-49-8, sold under the tradename Sprycel® by Bristol-Myers Squibb), armala (CAS No. 444731-52-6, also known as pazopanib, sold under the tradename Votrient® by GlaxoSmithKline), imatinib (CAS No. 152459-95-5) and imatinib mesylate (CAS No. 220127-57-1) (sold under the tradenames Gilvec®) and Gleevec® by Novartis);

DNA Synthesis inhibitors: Capecitabine (CAS No. 154361-50-9) (sold under the trademark Xeloda® by Roche), gemcitabine hydrochloride (CAS No. 122111-03-9) (sold under the trademark Gemzar® by Eli Lilly and Company), or nelarabine ((2R3S,4R,5R)-2-(2-amino-6-methoxy-purin-9-yl)-5-(hydroxymet hyl)oxolane-3,4-diol, sold under the tradenames Arranon® and Atriance® by GlaxoSmithKline);

Antineoplastic agents: oxaliplatin (CAS No. 61825-94-3) (sold under the tradename Eloxatin® ay Sanofi-Aventis and described in U.S. Pat. No. 4,169,846);

Human Granulocyte colony-stimulating factor (G-CSF) modulators: Filgrastim (sold under the tradename Neupogen® by Amgen);

Immunomodulators: Afutuzumab (available from RocheR), pegfilgrastim (sold under the tradename Neulasta® by Amgen), lenalidomide (CAS No. 191732-72-6, also known as CC-5013, sold under the tradename Revlimid®), or thalidomide (CAS No. 50-35-1, sold under the tradename Thalomid®);

CD40 inhibitors: Dacetuzumab (also known as SGN-40 or huS2C6, available from Seattle Genetics, Inc);

Pro-apoptotic receptor agonists (PARAs): Dulanermin (also known as AMG-951, available from Amgen/Genentech);

Hedgehog antagonists: 2-chloro-N-[4-chloro-3-(2-pyridinyl)phenyl]-4-(methylsulfonyl)-benzamide (also known as GDC-0449, and described in PCT Publication No. WO 06/028958);

Phospholipase A2 inhibitors: Anagrelide (CAS No. 58579-51-4, sold under the tradename Agrylin®);

BCL-2 inhibitors: 4-[4-[[2-(4-chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386);

MCL-1 inhibitors: MIK665 (CAS No. 1799631-75-6, S64315), AMG 397, and AZD5991 (CAS No. 2143010-83-5); Aromatase inhibitors: Exemestane (CAS No. 107868-30-4, sold under the trademark Aromasin® by Pfizer), letrozole (CAS No. 112809-51-5, sold under the tradename Femara® by Novartis), or anastrozole (CAS No. 120511-73-1, sold under the tradename Arimidex®);

Topoisomerase I inhibitors: Irinotecan (CAS No. 97682-44-5, sold under the trademark Camptosar® by Pfizer), topotecan hydrochloride (CAS No. 119413-54-6, sold under the tradename Hycamtin® by GlaxoSmithKline);

Topoisomerase II inhibitors: etoposide (CAS No. 33419-42-0, also known as VP-16 and Etoposide phosphate, sold under the tradenames Toposar®, VePesid® and Etopophos®), or teniposide (CAS No. 29767-20-2, also known as VM-26, sold under the tradename Vumon®);

mTOR inhibitors: Temsirolimus (CAS No. 162635-04-3, sold under the tradename Torisel® by Pfizer), ridaforolimus (CAS No. 572924-54-0, formally known as deferolimus, AP23573 and MK8669, and described in PCT Publication No. WO 03/064383), or everolimus (CAS No. 159351-69-6, sold under the tradename Afmitor® by Novartis);

Proteasome inhibitor such as carfilzomib (CAS No. 868540-17-4), MLN9708 (CAS No. 1201902-80-8), delanzomib (CAS No. 847499-27-8), or bortezomib (CAS No. 179324-69-7);

BET inhibitors such as INCB054329 (CAS No. 1628607-64-6), OTX015 (CAS No. 202590-98-5), or CPI-0610 (CAS No. 1380087-89-7);

LSD1 inhibitors such as GSK2979552, or INCB059872;

HIF-2a inhibitors such as PT2977 (1672668-24-4), NKT2152 (CAS No. 2511247-29-1), or PT2385 (CAS No. 1672665-49-4);

Osteoclastic bone resorption inhibitors: 1-hydroxy-2-imidazol-1-yl-phosphonoethyl) phosphonic acid monohydrate (sold under the tradename Zometa® by Novartis);

CD33 Antibody Drug Conjugates: Gemtuzumab ozogamicin (sold under the tradename Mylotarg® by Pfizer/Wyeth);

CD22 Antibody Drug Conjugates: Inotuzumab ozogamicin (also referred to as CMC-544 and WAY-207294, available from Hangzhou Sage Chemical Co., Ltd.);

CD20 Antibody Drug Conjugates: Ibritumomab tiuxetan (sold under the tradename Zevalin®);

Somatostain analogs: octreotide (also known as octreotide acetate, sold under the tradenames Sandostatin® and Sandostatin LAR®);

Synthetic Interleukin-11 (IL-11): oprelvekin (sold under the tradename Neumega® by Pfizer/Wyeth);

Synthetic erythropoietin: Darbepoetin alfa (sold under the tradename Aranesp® by Amgen);

Receptor Activator for Nuclear Factor k B (RANK) inhibitors: Denosumab (sold under the tradename Prolia® by Amgen);

Thrombopoietin mimetic peptibodies: Romiplostim (sold under the tradename

Nplatet by Amgen;

Cell growth stimulators: Palifermin (sold under the tradename Kepivance® by Amgen);

Anti-insulin-like Growth Factor-1 receptor (IGF-1R) antibodies: Figitumumab (also known as CP-751,871, available from ACC Corp), robatumumab (CAS No. 934235-44-6);

Anti-CSl antibodies: Elotuzumab (HuLuc63, CAS No. 915296-00-3);

CD52 antibodies: Alemtuzumab (sold under the tradename Campath®);

Histone deacetylase inhibitors: Voninostat (sold under the tradename Zolinza® by Merck);

Alkylating agents: Temozolomide (sold under the tradenames Temodar® and Temodal® by Schering-Plough/Merck), dactinomycin (also known as actinomycin-D and sold under the tradename Cosmegen®), melphalan (also known as L-PAM, L-sarcolysin, and phenylalanine mustard, sold under the tradename Alkeran®), altretamine (also known as hexamethylmelamine (HMM), sold under the tradename Hexalen®), carmustine (sold under the tradename BiCNU®), bendamustine (sold under the tradename Treanda®), busulfan (sold under the tradenames Busulfex® and Myleran®), carboplatin (sold under the tradename Paraplatin®), lomustine (also known as CCNU, sold under the tradename CeeNU®), cisplatin (also known as CDDP, sold under the tradenames Platinol® and Platinol®-AQ), chlorambucil (sold under the tradename Leukeran®), cyclophosphamide (sold under the tradenames Cytoxan® and Neosar®), dacarbazine (also known as DTIC, DIC and imidazole carboxamide, sold under the tradename DTIC-Dome®), altretamine (also known as hexamethylmelamine (HMM) sold under the tradename Hexalen®), ifosfamide (sold under the tradename Ifex®), procarbazine (sold under the tradename Matulane®), mechlorethamine (also known as nitrogen mustard, mustine and mechloroethamine hydrochloride, sold under the tradename Mustargen®), streptozocin (sold under the tradename Zanosar®), thiotepa (also known as thiophosphoamide, TESPA and TSPA, sold under the tradename Thioplex®;

Biologic response modifiers: bacillus calmette-guerin (sold under the tradenames theraCys® and TICE® BCG), or Denileukin diftitox (sold under the tradename Ontak®);

Anti-tumor antibiotics: doxorubicin (sold under the tradenames Adriamycin® and Rubex®), bleomycin (sold under the tradename Lenoxane®), daunorubicin (also known as dauorubicin hydrochloride, daunomycin, and rubidomycin hydrochloride, sold under the tradename Cerubidine®), daunorubicin liposomal (daunorubicin citrate liposome, sold under the tradename DaunoXome®), mitoxantrone (also known as DHAD, sold under the tradename Novantrone®), epirubicin (sold under the tradename Ellence™), idarubicin (sold under the tradenames Idamycin®, Idamycin PFS®), or mitomycin C (sold under the tradename Mutamycin®);

Anti-microtubule agents: Estramustine (CAS No. 52205-73-9, sold under the tradename Emcyl®);

Cathepsin K inhibitors: Odanacatib (CAS No. 603139-19-1, also known as MK-0822 available from Lanzhou Chon Chemicals, ACC Corp., and ChemieTek, and described in PCT Publication no. WO 03/075836);

Epothilone B analogs: Ixabepilone (CAS No. 219989-84-1, sold under the tradename Lxempra® by Bristol-Myers Squibb);

Heat Shock Protein (HSP) inhibitors: Tanespimycin (17-allylamino-17-demethoxygeldanamycin, also known as KOS-953 and 17-AAG, available from SIGMA, and described in U.S. Pat. No. 4,261,989), NVP-HSP990 (CAS No. 934343-74-5), AUY922 (CAS No. 747412-49-3), AT13387 (CAS No. 912999-49-6), STA-9090 (CAS No. 888216-25-9), Debio 0932, KW-2478 (CAS No. 819812-04-9), XL888 (CAS No. 1149705-71-4), CNF2024 (CAS No. 848695-25-0), and TAS-116 (CAS No. 1260533-36-5);

TpoR agonists: Eltrombopag (sold under the tradenames Promacta® and Revolade® by GlaxoSmithKline);

Anti-mitotic agents: Docetaxel (CAS No. 114977-28-5, sold under the tradename Taxotere® by Sanofi-Aventis); Adrenal steroid inhibitors: aminoglutethimide (CAS No. 125-84-8, sold under the tradename Cytadren®);

Anti-androgens: Nilutamide (CAS No. 63612-50-0, sold under the tradenames Nilandron® and Anandron®), bicalutamide (CAS No. 90357-06-5, sold under tradename Casodex®), or flutamide (CAS No. 13311-84-7, sold under the tradename Fulexin™);

Androgens: Fluoxymesterone (CAS No. 76-43-7, sold under the tradename Halotestin®);

CDK (CDK1, CDK2, CDK3, CDK5, CDK7, CDK8, or CDK9) inhibitors including but not limited to: Alvocidib (CAS No. 146426-40-6, pan-CDK inhibitor, also known as flovopirdol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002);

CDK2 inhibitor PF-07104091;

CDK4/6 inhibitors: pabociclib (CAS No. 827022-33-3), ribociclib (CAS No. 1211441-98-3), abemaciclib (CAS No. 1231929-97-7), PF-06873600 (CAS No. 2185857-97-8), NUV-422 and Trilaciclib (CAS No. 1374743-00-6);

CDK7 inhibitors CT7001 (CAS No. 1805789-54-1) and SY-1365 (CAS No. 1816989-16-8);

CDK9 inhibtiors AZD 4573 (CAS No. 2057509-72-3), P276-00 (CAS No. 920113-03-7), AT7519 (CAS No. 844442-38-2), CYC065 (CAS No. 1070790-89-4) or TP-1287;

Gonadotropin-releasing hormone (GnRH) receptor agonists: Leuprolide or leuprolide acetate (sold under the tradenames Viadure® by Bayer AG, Eligard® by Sanofi-Aventis and Lupron® by Abbott Lab);

Taxane anti-neoplastic agents: Cabazitaxel (1-hydroxy-7,10-dimethoxy-9-oxo-5,20-epoxytax-11-ene-2a,4,13a-triyl-4-acetate-2-benzoate-13-[(2R,3S)-3-{[(tert-butoxy)carbonyl]-amino}-2-hydroxy-3-phenylpropanoate), or larotaxel ((2a,3x,4a.5b,7a,10b,13a)-4,10-bis(acetyloxy)-13-({(2R,3S)-3-[(tert-butoxycarbonyl) amino]-2-hydroxy-3-phenylpropanoyl}oxy)-1-hydroxy-9-oxo-5,20-epoxy-7,19-cyclotax-11-en-2-yl benzoate);

5HTla receptor agonists: Xaliproden (also known as SR57746, 1-[2-(2-naphthyl)ethyl]-4-[3-(trifluoromethyl)phenyl]-1,2,3,6-tetrahydropyridine, and described in U.S. Pat. No. 5,266,573);

HPC vaccines: Cervarix® sold by GlaxoSmithKline, Gardasil® sold by Merck;

Iron Chelating agents: Deferasinox (CAS No. 201530-41-8, sold under the tradename Exjade® by Novartis);

Anti-metabolites: Claribine (2-chlorodeoxyadenosine, sold under the tradename Leustatin®), 5-fluorouracil (sold under the tradename Adrucil®), 6-thioguanine (sold under the tradename Purinethol®), pemetrexed (sold under the tradename Alimta®), cytarabine (also known as arabinosylcytosine (Ara-C), sold under the tradename Cytosar-U®), cytarabine liposomal (also known as Liposomal Ara-C, sold under the tradename DepoCyt™), decitabine (sold under the tradename Dacogen®), hydroxyurea (sold under the tradenames Hydrea®, Droxia™ and Mylocel™), fludarabine (sold under the tradename Fludara®), floxuridine (sold under the tradename FUDR®), cladribine (also known as 2-chlorodeoxyadenosine (2-CdA) sold under the tradename Leustatin™), methotrexate (also known as amethopterin, methotrexate sodim (MTX), sold under the tradenames RheumatrexR and Trexall™), or pentostatin (sold under the tradename Nipent®);

Bisphosphonates: Pamidronate (CAS No. 57248-88-1, sold under the tradename Aredia®), zoledronic acid CAS No. 118072-93-8 (sold under the tradename Zometa®);

Demethylating agents: 5-azacitidine (CAS No. 320-67-2, sold under the tradename Vidaza®), decitabine (CAS No. 2353-33-5, sold under the tradename Dacogen®);

Plant Alkaloids: Paclitaxel protein-bound (sold under the tradename Abraxane®), vinblastine (also known as vinblastine sulfate, vincaleukoblastine and VLB, sold under the tradenames Alkaban-AQ® and Velban®), vincristine (also known as vincristine sulfate, LCR, and VCR, sold under the tradenames Oncovin® and Vincasar Pfs®), vinorelbine (sold under the tradename Navelbine®), or paclitaxel (sold under the tradenames Taxol and Onxal™);

Retinoids: Alitretinoin (sold under the tradename Panretin®), tretinoin (all-trans retinoic acid, also known as ATRA, sold under the tradename Vesanoid®), Isotretinoin (13-cis-retinoic acid, sold under the tradenames Accutane®, Amnesteem®, Claravis®, Clarus®, Decutan®, Isotane®, Izotech®, Oratane®, Isotret®, and Sotret®), or bexarotene (sold under the tradename Targretin®);

Glucocorticosteroids: Hydrocortisone (also known as cortisone, hydrocortisone sodium succinate, hydrocortisone sodium phosphate, and sold under the tradenames A1a-Cort®, Hydrocortisone Phosphate, Solu-Cortef®, Hydrocort Acetate® and Lanacort®), dexamethazone ((8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one), prednisolone (sold under the tradenames Delta-Cortel®, Orapred®, Pediapred® and Prelone®), prednisone (sold under the tradenames Deltasone®, Liquid Red®, Meticorten® and Orasone®), or methylprednisolone (also known as 6-Methylprednisolone, Methylprednisolone Acetate, Methylprednisolone Sodium Succinate, sold under the tradenames Duralone®, Medralone®, Medrol®, M-Prednisol® and Solu-Medrol®);

Cytokines: interleukin-2 (also known as aldesleukin and IL-2, sold under the tradename Proleukin®), interleukin-11 (also known as oprevelkin, sold under the tradename Neumega®), alpha interferon alfa (also known as IFN-alpha, sold under the tradenames Intron® A, and Roferon-A®);

Estrogen receptor downregulators: Fulvestrant (CAS No. 129453-61-8, sold under the tradename Faslodex®);

Anti-estrogens: tamoxifen (CAS No. 10540-29-1, sold under the tradename Novaldex®); or Toremifene (CAS No. 89778-27-8, sold under the tradename Fareston®);

Selective estrogen receptor modulators (SERMs): Raloxifene (CAS No. 84449-90-1, sold under the tradename Evista®);

Leutinizing hormone releasing hormone (LFfRH) agonists: Goserelin (CAS No. 145781-92-6, sold under the tradename Zoladex®); Progesterones: megestrol (also known as megestrol acetate, CAS No. 595-33-5, sold under the tradename Megace®);

Miscellaneous cytotoxic agents: Arsenic trioxide (sold under the tradename Trisenox®), or asparaginase (also known as L-asparaginase, Erwinia L-asparaginase, sold under the tradenames Elspar® and Kidrolase®);

Exemplary immune checkpoint inhibitors include inhibitors (smack molecules or biologies) against immune checkpoint molecules such as CD27, CD28, CD40, CD 122, CD96, CD73, CD39, CD47, 0X40, GITR, CSFIR, JAK, PI3K delta, PI3K gamma, TAM kinase, arginase, CD137 (also known as 4-1BB), ICOS, A2AR, A2BR, HIF-2a, B7-H3, B7-H4, BTLA, CTLA-4, LAG3, TIM3, VISTA, CD96, TIGIT, PD-1, PD-L1 and PD-L2. In some embodiments, the immune checkpoint molecule is a stimulatory checkpoint molecule selected from CD27, CD28, CD40, ICOS, 0X40, GITR, CD137 and STING. In some embodiments, the immune checkpoint molecule is an inhibitory checkpoint molecule selected from B7-H3, B7-114, BTLA, CTLA-4, IDO, TDO, Arginase, KIR, LAG3, PD-1, TIM3, CD96, TIGIT and VISTA. In some embodiments, the compounds provided herein can be used in combination with one or more agents selected from KIR inhibitors, TIGIT inhibitors, LAIRI inhibitors, CD 160 inhibitors, 2B4 inhibitors and TGFR beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab, or pembrolizumab or PDR001. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab). In some embodiments, the anti-PD-L1 small molecule inhibitor is INCB86550.

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab. In some embodiments, the inhibitor of an immune checkpoint molecule is an inhibitor of LAG3, e.g., an anti-LAG3 antibody. In some embodiments, the anti-LAG3 antibody is BMS-986016 or LAG525. In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248. In some embodiments, the inhibitor of an immune checkpoint molecule is an inhibitor of 0X40, e.g., an anti-OX40 antibody or OX40L fusion protein. In some embodiments, the anti-OX40 antibody is MED 10562 or, INCAGN01949, GSK2831781, GSK-3174998, MOXR-0916, PF-04518600 or LAG525. In some embodiments, the OX40L fusion protein is MEDI6383.

Compounds of this disclosure can also be used to increase or enhance an immune response, including increasing the immune response to an antigen; to improve immunization, including increasing vaccine efficacy; and to increase inflammation. In some embodiments, the compounds of the invention can be sued to enhance the immune response to vaccines including, but not limited, Listeria vaccines, oncolytic viral vaccines, and cancer vaccines such as GVAX® (granulocyte-macrophage colony-stimulating factor (GM-CF) gene-transfected tumor cell vaccine). Anti-cancer vaccines include dendritic cells, synthetic peptides, DNA vaccines and recombinant viruses. Other immune-modulatory agents also include those that block immune cell migration such as antagonists to chemokine receptors, including CCR2 and CCR4; Sting agonists and Toll receptor agonists. Other anti-cancer agents also include those that augment the immune system such as adjuvants or adoptive T cell transfer. Compounds of this application may be effective in combination with CAR (Chimeric antigen receptor) T cell treatment as a booster for T cell activation.

A compound of this disclosure can also be used in combination with the following adjunct therapies: Anti-nausea drugs: NK-1 receptor antagonists: Casopitant (sold under the tradenames Rezonic® and Zunrisa® by GlaxoSmithKline); and Cytoprotective agents: Amifostine (sold under the tradename Ethyol®), leucovorin (also known as calcium leucovorin, citrovorum factor and folinic acid). The disclosure of the PCT applications referred to herein above are incorporated herein by reference in their entirety.

### EXAMPLES

The following preparations of intermediates and compounds of the disclosure are given to enable those skilled in the art to more clearly understand and to practice the present disclosure. They should not be considered as limiting the scope of the disclosure, but merely as being illustrative and representative thereof.

### SYNTHETIC EXAMPLES

### Intermediate 1

### Synthesis of (2,6-dimethylenetetrahydro-1H-pyrrolizin-7a(5H)-yl) methanol [Int-1]

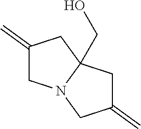

### Step 1: 1-(tert-butyl) 2-methyl 4-methylenepyrrolidine-1,2-dicarboxylate

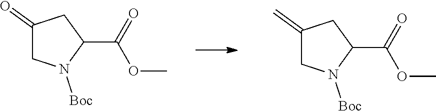

To a stirred solution of t-BuOK (5.2 g, 46.3 mmol, 1.4 eq.) in Et₂O (100 mL) was added methyltriphenylphosphonium bromide (13.7 g, 38.4 mmol, 1.2 eq.) in portions at 0° C. under nitrogen atmosphere. After stirring at 0° C. for 30 minutes, a solution of 1-tert-butyl 2-methyl 4-oxopyrrolidine-1,2-dicarboxylate (8.0 g, 32.9 mmol, 1.0 eq.) in Et₂O (100 mL) was added slowly. The resulting mixture was allowed to warm and stirred for additional 3 h at 35° C., then quenched with saturated NH₄Cl and then extracted with EtOAc. The combined organic layers were washed with water, brine, dried over anhydrous Na₂SO₄, filtered and then concentrated. The residue was purified by silica gel column chromatography, eluted with EA/PE (0-15%), to afford the title compound (3.0 g, 37.8%) as colorless oil.

### Step 2: 1-(tert-butyl) 2-methyl 2-(2-(chloromethyl)allyl)-4-methylenepyrrolidine-1,2-dicarboxylate

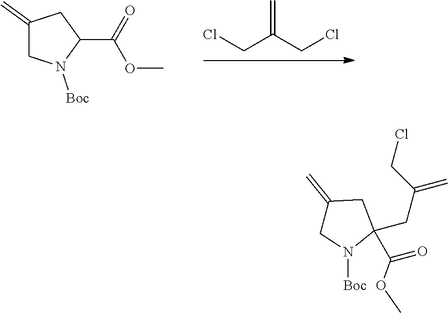

A solution of 1-(tert-butyl) 2-methyl 4-methylenepyrrolidine-1,2-dicarboxylate (3.0 g, 12.4 mmol, 1.0 eq.) in THF (30 mL) was added slowly to LiHMDS (25.0 mL, 25.0 mmol, 2.0 eq., 1.0 M in THF) at −78° C. under nitrogen atmosphere. After stirring for 1 h at −78° C., 3-chloro-2-(chloromethyl)prop-1-ene (3.9 g, 31.2 mmol, 2.5 eq.) was added dropwise at −78° C. under nitrogen atmosphere. The resulting mixture was warmed up and then stirred for 16 h at room temperature. The reaction mixture was diluted with water and then extracted with EtOAc. The combined organic layers were washed with water, brine, dried over anhydrous Na₂SO₄, filtered and then concentrated. The residue was purified by silica gel column chromatography, eluted with EA/PE (0-20%), to afford the title compound (2.0 g, 49.2%) as a yellow oil.

### Step 3: methyl 2,6-dimethylenetetrahydro-1H-pyrrolizine-7a(5H)-carboxylate

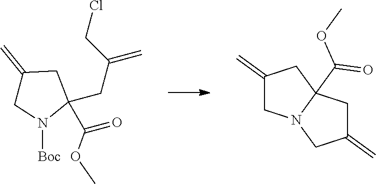

A solution of 1-(tert-butyl) 2-methyl 2-(2-(chloromethyl)allyl)-4-methylenepyrrolidine-1,2-dicarboxylate (2.1 g, 6.4 mmol, 1.0 eq.) and TFA (7.4 mL, 96.7 mmol, 15.1 eq.) in DCM (20 mL) was stirred for 16 h at room temperature. The reaction mixture was concentrated and then basified to pH=8 with NH₃/MeOH. The resulting mixture was concentrated, and the residue was purified by silica gel column chromatography, eluted with MeOH/DCM (0-10%), to afford the title compound (1.0 g, 81.3%) as a white solid.

### Step 4: (2,6-dimethylenetetrahydro-1H-pyrrolizin-7a(5H)-yl) methanol

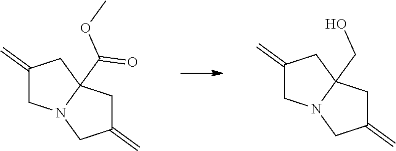

methyl 2,6-Dimethylenetetrahydro-1H-pyrrolizine-7a(5H)-carboxylate was converted to the title compound by proceeding analogously as described in Example 1, Step 2, below MS (ES, m/z): [M+H]⁺=166.2.

### Intermediate 3

### Synthesis of (2-ethylidenetetrahydro-1H-pyrrolizin-7a(5H)-yl)methanol [Int-3]

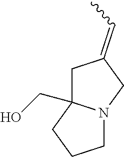

### Step 1: ethyl 2-ethylidene-5-oxotetrahydro-1H-pyrrolizine-7a(5H)-carboxylate

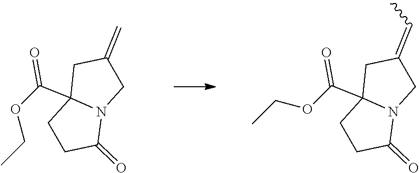

A mixture of ethyl 2-methylene-5-oxotetrahydro-TH-pyrrolizine-7a(5H)-carboxylate (1.5 g, 7.2 mmol, 1.0 eq.) and Grubbs 2nd generation catalyst (0.61 g, 0.74 mmol, 0.1 eq.) in DCM (10 mL) was stirred for 12 h at room temperature under propylene atmosphere. The resulting mixture was diluted with DCM, washed with brine, dried over anhydrous Na₂SO₄, filtered and then concentrated. The residue was purified by silica gel column chromatography, eluted with EA/PE (35%), to afford the title compound (500 mg, 30.6%) as a light-yellow oil.

### Step 2: (2-ethylidenetetrahydro-1H-pyrrolizin-7a(5H)-yl)methanol

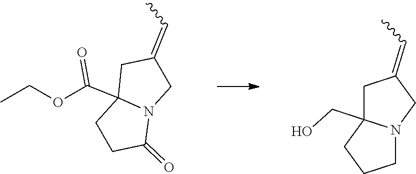

The title compound was prepared by proceeding analogously as described in Int-1 synthesis, Step 2. MS (ES, m/z): [M+H]⁺=168.2.

### Intermediate 4

### Synthesis of (2-(2-methoxyethylidene)tetrahydro-1H-pyrrolizin-7a(5H)-yl)methanol [Int-4]

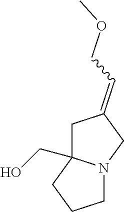

The title compound was prepared by proceeding analogously as described in Intermediate 3, using 3-methoxyprop-1-ene (5.0 eq.) instead of propylene in Step 1.

### Intermediate 5

### Synthesis of (2-butylidenetetrahydro-1H-pyrrolizin-7a(5H)-yl)methanol [Int-5]

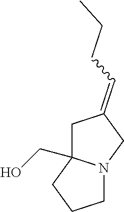

The title compound was prepared by proceeding analogously as described in Intermediate 3, using, using pent-1-ene (5.0 eq.) instead of propylene in Step 1.

### Intermediate 6

### Synthesis of (2-(2-methylpropylidene)tetrahydro-1H-pyrrolizin-7a(5H)-yl)methanol [Int-6]

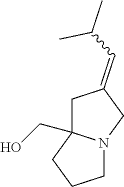

The title compound was prepared by proceeding analogously as described in Intermediate 3, using 3-methyl-1-butene (5.0 eq.) instead of propylene in Step 1. MS (ES, m/z): [M+1]⁺=196.1.

### Intermediate 7

### Synthesis of (2-(3-methylbutylidene)tetrahydro-1H-pyrrolizin-7a(5H)-yl)methanol [Int-7]

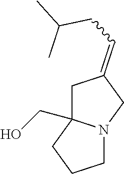

The title compound was prepared by proceeding analogously as described in Intermediate 3, using 3-methyl-1-pentene (5.0 eq.) instead of propylene in Step 1. MS (ES, m/z): [M+1]⁺=196.1.

### Intermediate 8

### Synthesis of ((4aS,8aR,9aS)-hexahydro-1H,3H-pyrano[3,4-b]pyrrolizin-8a(6H)-yl)methanol [Int-8a] and ((4aR,8aS,9aR)-hexahydro-1H,3H-pyrano[3,4-b]pyrrolizin-8a(6H)-yl)methanol [Int-8a]

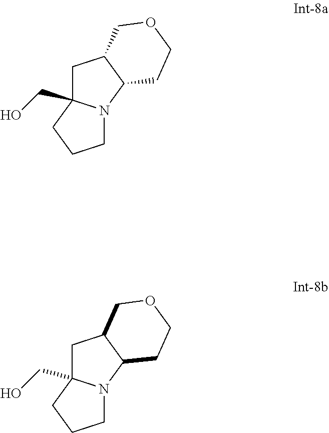

### Step 1: 7a-(tert-butyl) 2-methyl (2S,3S,7aR)-3-(2-(benzyloxy)ethyl)tetrahydro-1H-pyrrolizine-2,7a(5H)-dicarboxylate and 7a-(tert-butyl) 2-methyl (2R,3R,7aS)-3-(2-(benzyloxy)ethyl)-tetrahydro-1H-pyrrolizine-2,7a(5H)-dicarboxylate

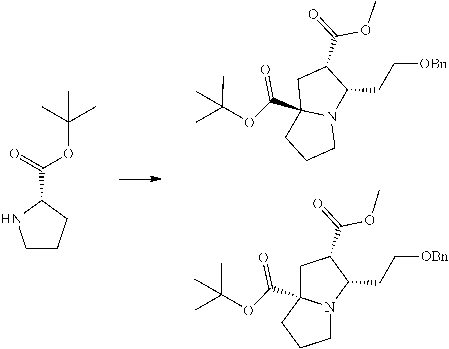 [SMILES: CC(C)(C)OC(=O)[C@@H]1CCCN1 ; COC(=O)[C@H]1C[C@]2(C(=O)OC(C)(C)C)CCCN2[C@H]1CCOCc1ccccc1 ; COC(=O)[C@H]1C[C@@]2(C(=O)OC(C)(C)C)CCCN2[C@H]1CCOCc1ccccc1]

To a stirred solution of tert-butyl L-prolinate (15.0 g, 87.6 mmol, 1.0 eq.) in toluene (600 mL) was added silver acetate (0.73 g, 4.4 mmol, 0.050 eq.) in one portion, 3-(benzyloxy)propanal (14.4 g, 87.7 mmol, 1.0 eq.), methyl acrylate (7.5 g, 87.1 mmol, 1.0 eq.) and triethylamine (9.8 g, 96.8 mmol, 1.1 eq.) at room temperature. After stirring at room temperature for 16 h under dark, the reaction mixture was concentrated. The residue was purified twice by silica gel column chromatography, eluted with EA/PE (0-30%), to afford the title compounds (2.3 g, 6.5%) as a light-yellow oil.

### Step 2: tert-butyl (2S,3S,7aR)-3-(2-(benzyloxy)ethyl)-2-(hydroxymethyl)tetrahydro-1H-pyrrolizine-7a(5H)-carboxylate and tert-butyl (2R,3R,7aS)-3-(2-(benzyloxy)ethyl)-2-(hydroxymethyl)tetrahydro-1H-pyrrolizine-7a(5H)-carboxylate

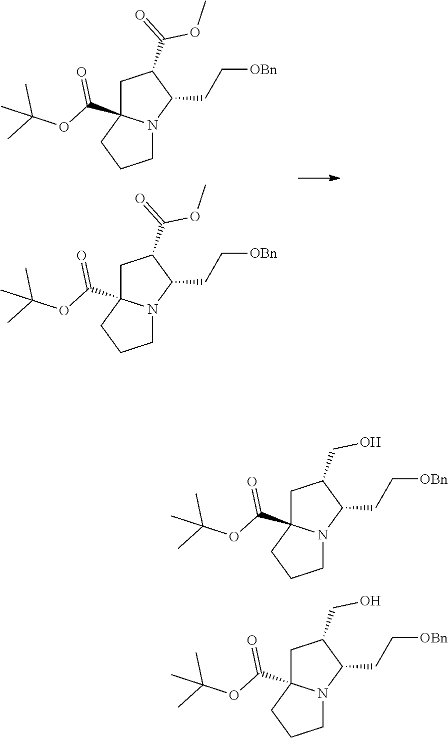

To a stirred solution of 7a-(tert-butyl) 2-methyl (2S,3S,7aR)-3-(2-(benzyloxy)ethyl) tetrahydro-1H-pyrrolizine-2,7a(5H)-dicarboxylate and 7a-(tert-butyl) 2-methyl (2R,3R,7aS)-3-(2-(benzyloxy)ethyl) tetrahydro-1H-pyrrolizine-2,7a(5H)-dicarboxylate (1.5 g, 3.7 mmol, 1.0 eq.) in THF (15 mL) was added DIBAL-H (7.5 mL, 7.5 mmol, 1.0 M, 2.0 eq.) dropwise at 5° C. under nitrogen atmosphere. After stirring for 30 min, additional DIBAL-H (3.7 mL, 3.7 mmol, 1.0 M, 1.0 eq.) was added dropwise. The reaction mixture was quenched with water, diluted with THF, and then the addition of Na₂SO₄·10H₂O. The resulting mixture was filtered, and the filter cake was washed with THF. The filtrate was concentrated to remove most organic solvent, and the resulting aqueous residue was extracted with DCM. The combined organic layers were washed with brine, dried over anhydrous Na₂SO₄, filtered and then concentrated. The residue was purified by silica gel column chromatography, eluted with MeOH/DCM (0-10%), to afford the title compounds (600 mg, 43.2%) as a light-yellow oil.

### Step 3: tert-butyl (2S,3S,7aR)-3-(2-hydroxyethyl)-2-(hydroxymethyl)tetrahydro-1H-pyrrolizine-7a(5H)-carboxylate and tert-butyl (2R,3R,7aS)-3-(2-hydroxyethyl)-2-(hydroxymethyl)tetrahydro-1H-pyrrolizine-7a(5H)-carboxylate

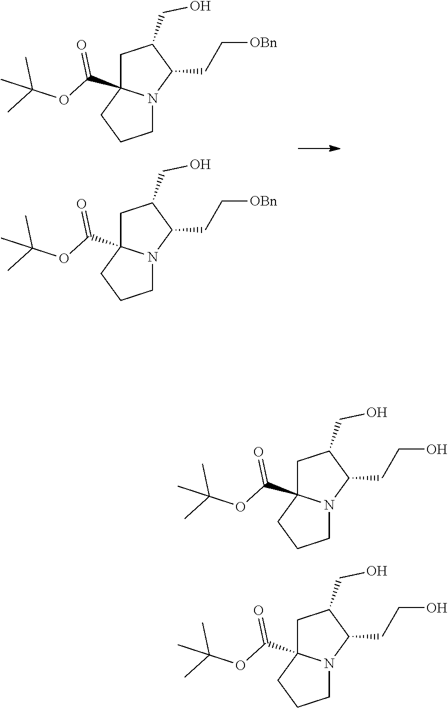

A mixture of tert-butyl (2S,3S,7aR)-3-(2-(benzyloxy)ethyl)-2-(hydroxymethyl) tetrahydro-1H-pyrrolizine-7a(5H)-carboxylate and tert-butyl (2R,3R,7aS)-3-(2-(benzyloxy)ethyl)-2-(hydroxymethyl) tetrahydro-1H-pyrrolizine-7a(5H)-carboxylate (350 mg, 0.93 mmol, 1.0 eq.), HCl(g) in MeOH (50 mg) and 10% Pd/C (525 mg) in MeOH (17.5 mL) was stirred for 16 h at 50° C. under 20 atm hydrogen atmosphere. The reaction mixture was filtered and the filter cake was washed with MeOH. The filtrate was concentrated, and the residue was purified by silica gel column chromatography, eluted with MeOH/DCM (0-20%), to afford the titles compound (150 mg, 57.0%) as a light yellow oil.

### Step 4: tert-butyl (4aS,8aR,9aS)-hexahydro-1H,3H-pyrano[3,4-b]pyrrolizine-8a(6H)-carboxylate and tert-butyl (4aR,8aS,9aR)-hexahydro-1H,3H-pyrano[3,4-b]pyrrolizine-8a(6H)-carboxylate

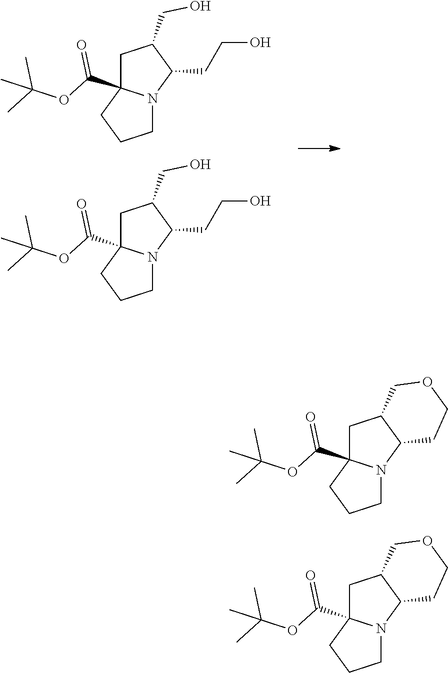

To a stirred solution of tert-butyl (2S,3S,7aR)-3-(2-hydroxyethyl)-2-(hydroxymethyl)-tetrahydro-1H-pyrrolizine-7a(5H)-carboxylate tert-butyl (2R,3R,7aS)-3-(2-hydroxyethyl)-2-(hydroxymethyl)tetrahydro-1H-pyrrolizine-7a(5H)-carboxylate (150 mg, 0.53 mmol, 1.0 eq.) in THF (4.5 mL) was added 60% sodium hydride (74 mg, 1.85 mmol, 3.5 eq.) in portions at 5° C. under nitrogen atmosphere, followed by a solution of methanesulfonyl chloride (60 mg, 0.52 mmol, 1.0 eq.) in THF (0.5 mL) dropwise at 5° C. The resulting mixture was stirred for additional 16 h at room temperature, quenched with water at 5° C., and then extracted with EtOAc. The combined organic layers were washed with brine, dried over anhydrous Na₂SO₄, filtered and then concentrated. The residue was purified by silica gel column chromatography, eluted with EA/PE (0-100%), to afford the title compounds (60 mg, 42.3%) as a light-yellow oil.

### Step 5: ((4aS,8aR,9aS)-hexahydro-1H,3H-pyrano[3,4-b]pyrrolizin-8a(6H)-yl)methanol and ((4aR,8aS,9aR)-hexahydro-1H,3H-pyrano[3,4-b]pyrrolizin-8a(6H)-yl)methanol

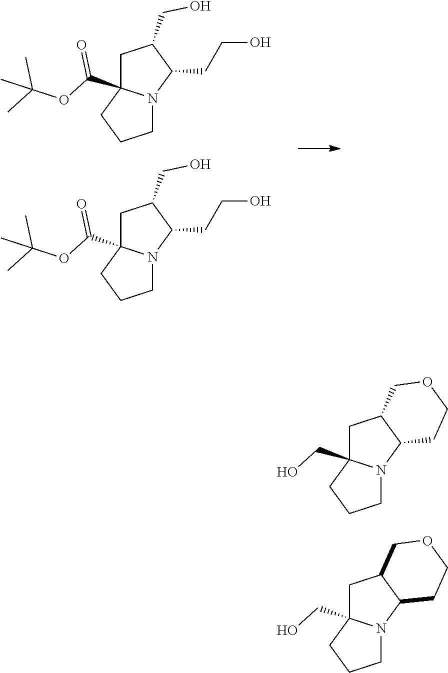

The title compounds was prepared by proceeding analogously as described in Example 1, Step 2. MS (ES, m/z): [M+H]⁺=198.2.

### Intermediate 9

### Synthesis of (6-fluoro-2,3-dihydro-1H-pyrrolo[2,1-a]isoindol-9b(5H)-yl)methanol [Int-9]

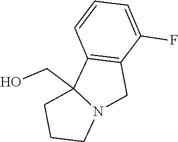 [SMILES: OCC12CCCN1Cc1c(F)cccc12]

The title compound was prepared by proceeding analogously as described in Example 6, Steps 1-3, using 2-bromo-6-fluorobenzaldehyde instead of 2-bromobenzaldehyde in Step 1. MS (ES, m/z): [M+H]⁺=208.1.

### Intermediate 10

### Synthesis of (7-fluoro-2,3-dihydro-1H-pyrrolo[2,1-a]isoindol-9b(5H)-yl)methanol [Int-10]

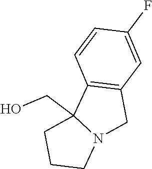 [SMILES: OCC12CCCN1Cc1cc(F)ccc12]

The title compound was prepared by proceeding analogously as described Example 6, Steps 1-3, using 2-bromo-5-fluorobenzaldehyde instead of 2-bromobenzaldehyde in Step 1. MS (ES, m/z): [M+H]⁺=208.1.

### Intermediate 11

### Synthesis of (8-fluoro-2,3-dihydro-1H-pyrrolo[2,1-a]isoindol-9b(5H)-yl)methanol [Int-11]

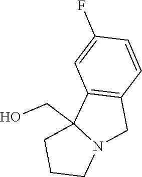 [SMILES: OCC12CCCN1Cc1ccc(F)cc12]

The title compound was prepared by proceeding analogously as described in Example 6, Steps 1-3, using 2-bromo-4-fluorobenzaldehyde instead of 2-bromobenzaldehyde in Step 1. MS (ES, m/z): [M+H]⁺=208.1.

### Intermediate 12

### Synthesis of (9-fluoro-2,3-dihydro-1H-pyrrolo[2,1-a]isoindol-9b(5H)-yl)methanol [Int-12]

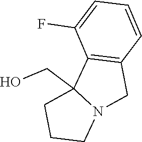 [SMILES: OCC12CCCN1Cc1cccc(F)c12]

The title compound was prepared by proceeding analogously as described in Example 6, Steps 1-3, using 2-bromo-3-fluorobenzaldehyde instead of 2-bromobenzaldehyde in Step 1. MS (ES, m/z): [M+H]⁺=208.2.

### Intermediate 13

### Synthesis of (8,9-dihydro-5H-pyrido[2,3-a]pyrrolizin-9a(7H)-yl)methanol [Int-13]

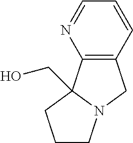 [SMILES: OCC12CCCN1Cc1cccnc12]

The title compound was prepared by proceeding analogously as described in Example 6, Steps 1-3, using 2-bromopyridine-3-carbaldehyde instead of 2-bromobenzaldehyde in Step 1. MS (ES, m/z): [M+H]⁺=191.1.

### Intermediate 14

### Synthesis of (6-methoxy-2,3-dihydro-1H-pyrrolo[2,1-a]isoindol-9b(5H)-yl)methanol [Int-14]

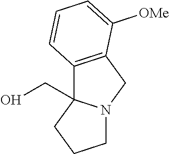 [SMILES: COc1cccc2c1CN1CCCC21CO]

The title compound was prepared by proceeding analogously as described Example 6. Step 1-3, using 2-bromo-6-methoxybenzaldehyde instead of 2-bromobenzaldehyde in in Step 1. MS (ES, m/z): [M+H]⁺=220.1.

### Intermediate 15

### Synthesis of (7-methoxy-2,3-dihydro-1H-pyrrolo[2,1-a]isoindol-9b(5H)-yl)methanol [Int-15]

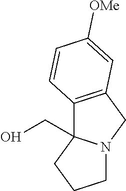 [SMILES: COc1ccc2c(c1)CN1CCCC21CO]

The title compound was prepared by proceeding analogously as described Example 6, Stepsl-3, using 2-bromo-5-methoxybenzaldehyde instead of 2-bromobenzaldehyde in Step 1. MS (ES, m/z): [M+H]⁺=220.1

### Intermediate 16

### Synthesis of (6,7-dihydro-5H-pyrido[4,3-a]pyrrolizin-4b(9H)-yl)methanol [Int-16]

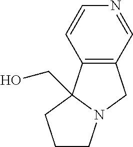 [SMILES: OCC12CCCN1Cc1cnccc12]

The title compound was prepared by proceeding analogously as described Example 6, Steps1-3, using 4-bromopyridine-3-carbaldehyde instead of 2-bromobenzaldehyde in Step 1. MS (ES, m/z): [M+H]⁺=191.0.

### Intermediate 17

### Synthesis of (8,9-dihydro-5H-pyrido[3,4-a]pyrrolizin-9a(7H)-yl)methanol [Int-17]

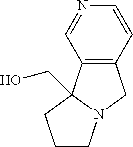 [SMILES: OCC12CCCN1Cc1ccncc12]

The title compound was prepared by proceeding analogously as described Example 6, Steps 1-3, using 3-bromopyridine-4-carbaldehyde instead of 2-bromobenzaldehyde in Step 1. MS (ES, m/z): [M+H]⁺=191.2.

### Intermediate 18

### Synthesis of (6-(methoxymethyl)-2,3-dihydro-1H-pyrrolo[2,1-a]isoindol-9b(5H)-yl)methanol [Int-18]

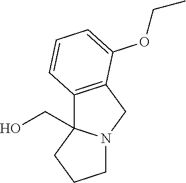 [SMILES: CCOc1cccc2c1CN1CCCC21CO]

The title compound was prepared by proceeding analogously as described Example 6, Steps 1-3, using 2-bromo-6-(methoxymethyl)benzaldehyde instead of 2-bromobenzaldehyde in Step 1. MS (ES, m/z): [M+H]⁺=234.2.

### Intermediate 19

### Synthesis of (6-(trifluoromethyl)-2,3-dihydro-1H-pyrrolo[2,1-a]isoindol-9b(5H)-yl)methanol [Int-19]

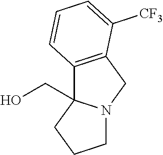 [SMILES: OCC12CCCN1Cc1c(C(F)(F)F)cccc12]

### Step 1: 1-bromo-2-(bromomethyl)-3-(trifluoromethyl)benzene

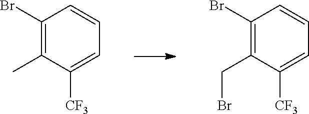

A solution of 1-bromo-2-methyl-3-(trifluoromethyl)benzene (2.0 g, 8.4 mmol, 1.0 eq.) and NBS (1.8 g, 10.1 mmol, 1.2 eq.), benzoyl peroxide (0.21 g, 0.87 mmol, 0.10 eq.) in CCl₄ (20 mL) was stirred for 2 h at 80° C. under nitrogen atmosphere. The resulting mixture was concentrated under reduced pressure. The residue was triturated in petroleum ether and the precipitated solid was collected by filtration to afford the title compound (2.0 g, 75.0%) as a yellow solid.

### Step 2: tert-butyl (2-bromo-6-(trifluoromethyl)benzyl)prolinate

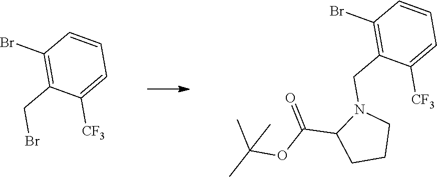 [SMILES: FC(F)(F)c1cccc(Br)c1CBr ; CC(C)(C)OC(=O)C1CCCN1Cc1c(Br)cccc1C(F)(F)F]

A mixture of 1-bromo-2-(bromomethyl)-3-(trifluoromethyl)benzene (2.0 g, 6.3 mmol, 1.0 eq.) and tert-butyl pyrrolidine-2-carboxylate (1.3 g, 7.6 mmol, 1.2 eq.) and K₂CO₃ (1.7 g, 12.6 mmol, 2.0 eq.) in CH₃CN (20 mL) was stirred for 2 h at 80° C. under nitrogen atmosphere. The resulting mixture was concentrated under reduced pressure. The residue was purified by silica gel column chromatography, eluted with EA/PE (0-10%), to afford the title compound (2.1 g, 81.0%) as a yellow oil.

### Step 3: (6-(trifluoromethyl)-2,3-dihydro-1H-pyrrolo[2,1-a]isoindol-9b(5H)-yl)methanol

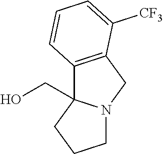 [SMILES: OCC12CCCN1Cc1c(C(F)(F)F)cccc12]

The title compound was prepared by proceeding analogously as described in Example 6, Steps 2-3, using tert-butyl (2-bromo-6-(trifluoromethyl)-benzyl)prolinate instead of methyl (2-bromobenzyl)prolinate in Step 2. MS (ES, m/z): [M+H]⁺=258.1.

### Intermediate 20

### Synthesis of a mixture of (cis-2-fluoro-2,3-dihydro-1H-pyrrolo[2,1-a]isoindol-9b(5H)-yl)methanol and (trans-2-fluoro-2,3-dihydro-1H-pyrrolo[2,1-a]isoindol-9b(5H)-yl)methanol [Int-20]

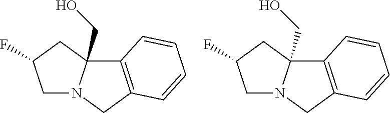

The title compounds were prepared by proceeding analogously as described Example 6, Step 1-3, using methyl cis-4-fluoropyrrolidine-2-carboxylate instead of methyl prolinate in Step 1. The crude product was purified by prep-HPLC to afford the title compounds (200 mg and 220 mg) as brown oil. MS (ES, m/z): [M+H]⁺=208.2.

### Intermediate 21

### Synthesis of (1-methyl-1,5,6,8-tetrahydropyrazolo[4,3-a]pyrrolizin-3b(4H)-yl)methanol [Int-21]

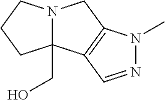

### Step 1: 1-(tert-butyl) 2-methyl (S,E)-3-((dimethylamino)methylene)-4-oxopyrrolidine-1,2-dicarboxylate

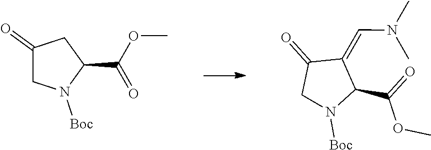 [SMILES: COC(=O)[C@@H]1CC(=O)CN1C(=O)OC(C)(C)C ; COC(=O)[C@@H]1/C(=C\N(C)C)C(=O)CN1C(=O)OC(C)(C)C]

A solution of 1-tert-butyl 2-methyl (2S)-4-oxopyrrolidine-1,2-dicarboxylate (20.0 g, 82.2 mmol, 1.0 eq.) and DMF-DMA (13.9 g, 116.6 mmol, 1.4 eq.) in DMF (100 mL) was stirred overnight at 105° C. The reaction mixture was cooled and then diluted with water. The resulting mixture was extracted with EtOAc. The combined organic layers were washed with water, brine, dried over anhydrous Na₂SO₄, filtered and then concentrated. The residue was purified by silica gel column chromatography, eluted with EA/PE (50%), to afford the title compound (13.2 g, 53.8%) as a brown oil.

### Step 2: 5-(tert-butyl) 4-methyl (S)-2,6-dihydropyrrolo[3,4-c]pyrazole-4,5 (4H)-dicarboxylate

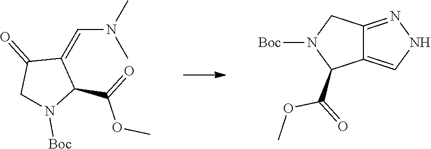 [SMILES: COC(=O)[C@@H]1/C(=C\N(C)C)C(=O)CN1C(=O)OC(C)(C)C ; COC(=O)[C@@H]1c2c[nH]nc2CN1C(=O)OC(C)(C)C]

To a stirred solution of 1-(tert-butyl) 2-methyl (S,E)-3-((dimethylamino)methylene)-4-oxopyrrolidine-1,2-dicarboxylate (13.2 g, 44.2 mmol, 1.0 eq.) in EtOH (60 mL) was added 80% NH₂NH₂·H₂O (3.2 g, 63.9 mmol, 1.4 eq.) dropwise at room temperature. The resulting mixture was stirred for 3 h at room temperature, diluted with water and then extracted with EtOAc. The combined organic layers were washed with water, brine, dried over anhydrous Na₂SO₄, filtered and then concentrated. The residue was dissolved in DCM (100 mL) and then a solution of TsOH (2.0 g, 11.6 mmol, 0.26 eq.) in MeOH (20 mL) was added dropwise at 0-5° C. The resulting mixture was stirred for 2 h at 0-5° C., diluted with water, and then extracted with DCM. The combined organic layers were washed with water, brine, dried over anhydrous Na₂SO₄, filtered and then concentrated. The residue was purified by silica gel column chromatography, eluted with EA/PE (50%), to afford the title compound (6.0 g, 50.7%) as a yellow oil.

### Step 3: 5-(tert-butyl) 4-methyl (S)-1-methyl-4,6-dihydropyrrolo[3,4-c]pyrazole-4,5 (1H)-dicarboxylate

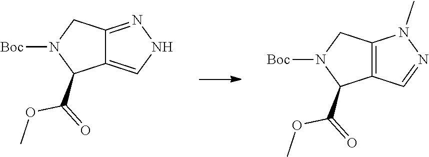 [SMILES: COC(=O)[C@@H]1c2c[nH]nc2CN1C(=O)OC(C)(C)C ; COC(=O)[C@@H]1c2cnn(C)c2CN1C(=O)OC(C)(C)C]

To a stirred mixture of 5-(tert-butyl) 4-methyl (S)-2,6-dihydropyrrolo[3,4-c]pyrazole-4,5 (4H)-dicarboxylate (6.0 g, 22.4 mmol, 1.0 eq.) and K₂CO₃ (4.6 g, 33.3 mmol, 1.5 eq.) in DMF (3 mL) was added CH₃I (9.6 g, 67.6 mmol, 3.0 eq.) dropwise at 0° C. After stirring at ambient temperature overnight, the reaction mixture was diluted with water and then extracted with EtOAc. The combined organic layers were washed with water, brine, dried over anhydrous Na₂SO₄, filtered and then concentrated. The residue was purified by silica gel column chromatography, eluted with EA/PE (0-70%), to afford the title compound (5.5 g, 87.5%) as a yellow solid.

### Step 4: 1-(tert-butyl) 2-methyl (2S,4S)-4-((tert-butyldimethylsilyl)oxy)-2-(2-(chloromethyl)allyl)-pyrrolidine-1,2-dicarboxylate

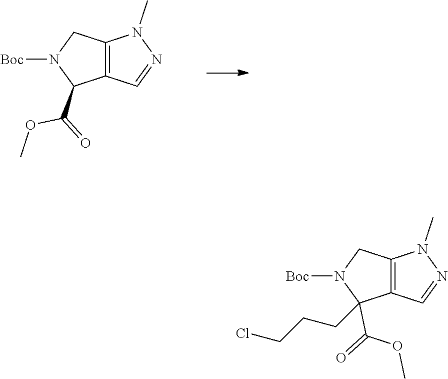 [SMILES: COC(=O)[C@@H]1c2cnn(C)c2CN1C(=O)OC(C)(C)C ; COC(=O)C1(CCCCl)c2cnn(C)c2CN1C(=O)OC(C)(C)C]

To a stirred solution of HMDS (988 mg, 6.1 mmol, 1.1 eq.) in THF (20 mL) was added 2.5 M n-BuLi in n-hexane (2.4 mL, 6.0 mmol, 1.1 eq.) slowly at −78° C. under nitrogen atmosphere. After stirring for 30 min, a solution of 5-(tert-butyl) 4-methyl (S)-1-methyl-4,6-dihydro-pyrrolo[3,4-c]pyrazole-4,5 (1H)-dicarboxylate (1.55 g, 5.5 mmol, 1.00 eq.) in THF (20 mL) was added slowly at −78° C. The resulting mixture was stirred for 15 min at −78° C., and then 1-bromo-3-chloropropane (1.04 g, 6.6 mmol, 1.2 eq.) was added dropwise. The resulting mixture was stirred for additional 12 h at ambient temperature, quenched with NH₄Cl aq. at 5° C. and then extracted with EtOAc. The combined organic layers were washed with brine, dried over anhydrous Na₂SO₄, filtered and then concentrated. The residue was purified by silica gel column chromatography, eluted with EA/PE (30%), to afford the title compound (1.0 g, 50.9%) as a light yellow oil.

### Step 5: (1-methyl-1,5,6,8-tetrahydropyrazolo[4,3-a]pyrrolizin-3b(4H)-yl)methanol

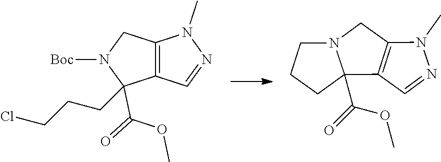 [SMILES: COC(=O)C1(CCCCl)c2cnn(C)c2CN1C(=O)OC(C)(C)C ; COC(=O)C12CCCN1Cc1c2cnn1C]

A solution of 5-(tert-butyl) 4-methyl 4-(3-chloropropyl)-1-methyl-4,6-dihydropyrrolo [3,4-c]pyrazole-4,5 (1H)-dicarboxylate (1.2 g, 3.4 mmol, 1.0 eq.) and TFA (1.0 mL) in THF (10 mL) was stirred at 0° C. for 1 h. The resulting mixture was concentrated. The residue was dissolved in MeOH (10 mL) and then K₂CO₃ (10.0 g, 72.4 mmol, 21.3 eq.) was added at 25° C. The resulting mixture was stirred for additional 1 h at 25° C. and then concentrated. The residue was purified by silica gel column chromatography, eluted with PE/EA (50%), to afford the title compound (300 mg, 41.2%) as a light-yellow oil.

### Step 6: (1-methyl-1,5,6,8-tetrahydropyrazolo[4,3-a]pyrrolizin-3b(4H)-yl)methanol

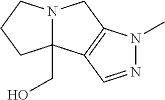

The title compound was prepared by proceeding analogously as described in Intermediate 1, Step 2. MS (ES, m/z): [M+H]⁺=194.2.

### Intermediate 22

### Synthesis of a mixture of ((3bR,7aR,8aR)-6,7,8,8a-tetrahydro-5H,9H-pyrazolo-[1′,5′: 1,5]pyrrolo[3,4-b]-pyrrolizin-7a(3bH)-yl)methanol and ((3bS,7aS,8aS)-6,7,8,8a-tetrahydro-5H,9H-pyrazolo[1′,5′: 1,5]pyrrolo[3,4-b]pyrrolizin-7a(3bH)-yl)methanol [Int-22]

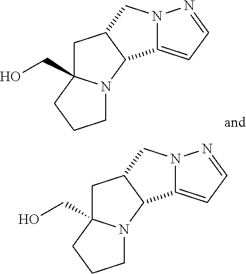

### Step 1: 7a-(tert-butyl) 2-methyl (2S,3R,7aR)-3-(1-(tetrahydro-2H-pyran-2-yl)-1H-pyrazol-3-yl)tetrahydro-1H-pyrrolizine-2,7a(5H)-dicarboxylate and 7a-(tert-butyl) 2-methyl (2R,3S,7aS)-3-(1-(tetrahydro-2H-pyran-2-yl)-1H-pyrazol-3-yl)tetrahydro-1H-pyrrolizine-2,7a(5H)-dicarboxylate

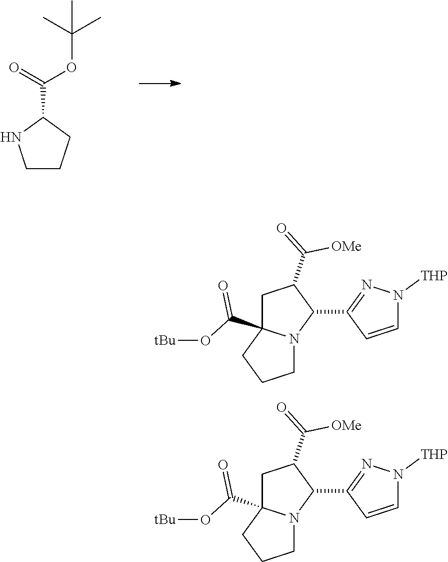

A mixture of tert-butyl L-prolinate (10.0 g, 58.4 mmol, 1.0 eq.), AgOAc (490 mg, 2.9 mmol, 0.050 eq.), 1-(tetrahydro-2H-pyran-2-yl)-1H-pyrazole-3-carbaldehyde (11.0 g, 61.0 mmol, 1.04 eq.), methyl acrylate (5.0 g, 58.1 mmol, 1.0 eq.) and TEA (6.5 g, 64.2 mmol, 1.1 eq.) in toluene (220 mL) was stirred for 16 h at room temperature. The resulting mixture was diluted with water and then extracted with EtOAc. The combined organic layers were washed with brine, dried over anhydrous Na₂SO₄, filtered and then concentrated. The residue was purified by silica gel column chromatography, eluted with EA/PE (0-50%), to afford the title compounds (15.0 g, 61.6%) as a brown solid

### Step 2: tert-butyl (2S,3R,7aR)-2-(hydroxymethyl)-3-(1-(tetrahydro-2H-pyran-2-yl)-1H-pyrazol-3-yl)-tetrahydro-1H-pyrrolizine-7a(5H)-carboxylate and tert-butyl (2R,3S,7aS)-2-(hydroxymethyl)-3-(1-(tetrahydro-2H-pyran-2-yl)-1H-pyrazol-3-yl)tetrahydro-TH-pyrrolizine-7a(5H)-carboxylate

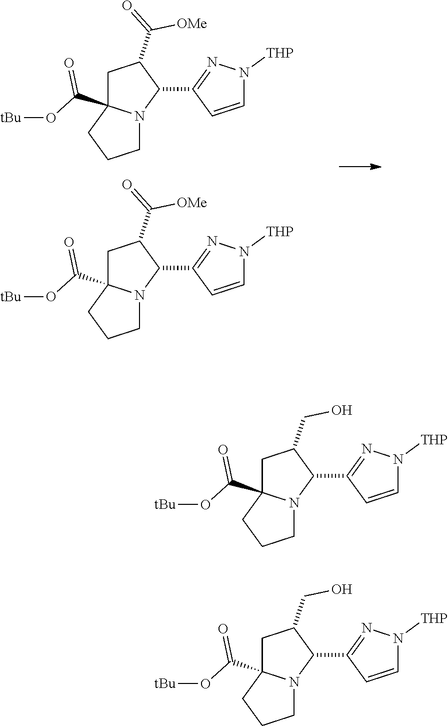

To a stirred solution of 7a-(tert-butyl) 2-methyl (2S,3R,7aR)-3-(1-(tetrahydro-2H-pyran-2-yl)-1H-pyrazol-3-yl)tetrahydro-H-pyrrolizine-2,7a(5H)-dicarboxylate and 7a-(tert-butyl) 2-methyl (2R,3S,7aS)-3-(1-(tetrahydro-2H-pyran-2-yl)-1H-pyrazol-3-yl)tetrahydro-1H-pyrrolizine-2,7a(5H)-dicarboxylate (7.0 g, 16.7 mmol, 1.0 eq. in THF (70 mL) was added DIBAL-H (42.0 mL, 42.0 mmol, 1.0 eq.) dropwise at 0° C. The reaction mixture was stirred for 16 h at room temperature and then quenched with water and Na₂SO₄ at 0° C. The resulting mixture was filtered and the filter cake was washed with THF. The filtrate was concentrated and the residue was purified by silica gel column chromatography, eluted with MeOH/DCM (0-10%), to afford the title compounds (3.5 g 53.3%) as a colorless oil.

### Step 3: tert-butyl (2S,3R,7aR)-2-(hydroxymethyl)-3-(1H-pyrazol-3-yl)tetrahydro-1H-pyrrolizine-7a(5H)-carboxylate and tert-butyl (2R,3S,7aS)-2-(hydroxymethyl)-3-(1H-pyrazol-3-yl)tetrahydro-1H-pyrrolizine-7a(5H)-carboxylate

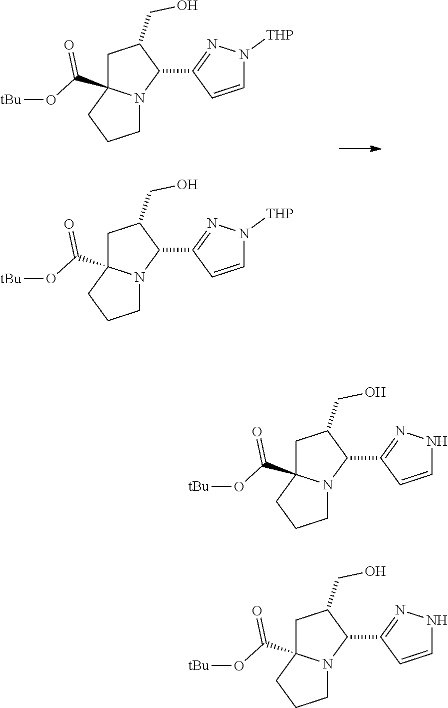

To a stirred solution of tert-butyl (2S,3R,7aR)-2-(hydroxymethyl)-3-(1-(tetrahydro-2H-pyran-2-yl)-1H-pyrazol-3-yl)tetrahydro-1H-pyrrolizine-7a(5H)-carboxylate and tert-butyl (2R,3S,7aS)-2-(hydroxymethyl)-3-(1-(tetrahydro-2H-pyran-2-yl)-1H-pyrazol-3-yl)tetrahydro-H-pyrrolizine-7a(5H)-carboxylate (2.5 g, 6.4 mmol, 1.0 eq.) in DCM (25 mL) was added 4M HCl in MeOH (10.0 mL) dropwise at 0° C. The resulting mixture was stirred for 2 h at room temperature and then concentrated. The residue was purified by silica gel column chromatography, eluted with EA/PE (0-100%), to afford the title compounds (2.4 g, crude) as a yellow oil.

### Step 4: tert-butyl (3bR,7aR,8aR)-6,7,8,8a-tetrahydro-5H,9H-pyrazolo[1′,5′: 1,5]pyrrolo[3,4-b]-pyrrolizine-7a(3bH)-carboxylate and tert-butyl (3bS,7aS,8aS)-6,7,8,8a-tetrahydro-5H,9H-pyrazolo[1′,5′: 1,5]pyrrolo[3,4-b]pyrrolizine-7a(3bH)-carboxylate

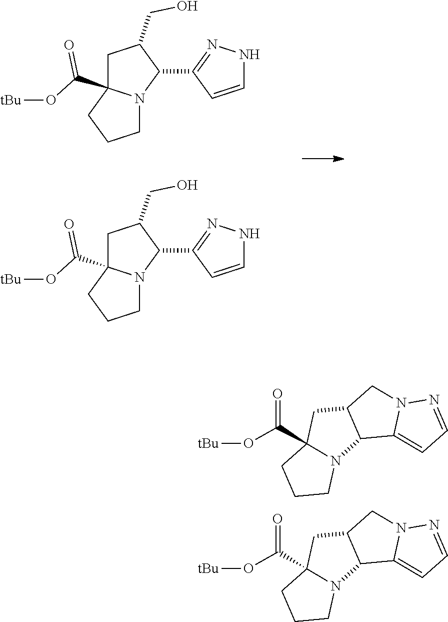

To a stirred solution of tert-butyl (2S,3R,7aR)-2-(hydroxymethyl)-3-(1H-pyrazol-3-yl)tetrahydro-1H-pyrrolizine-7a(5H)-carboxylate and tert-butyl (2R,3S,7aS)-2-(hydroxymethyl)-3-(1H-pyrazol-3-yl)tetrahydro-1H-pyrrolizine-7a(5H)-carboxylate (2.4 g, 7.8 mmol, 1.0 eq.) in THF (24 mL) was added SOCl₂ (2.8 g, 23.5 mmol, 3.0 eq.) dropwise at 0° C. The resulting mixture was stirred for 2 h at room temperature, diluted with water and then extracted with EtOAc. The combined organic layers were washed with brine, dried over anhydrous Na₂SO₄, filtered and then concentrated. The residue was purified by silica gel column chromatography, eluted with EA/PE (0-70%), to afford the title compounds (750 mg, 40.6% over 2 steps) as a yellow oil.

### Step 5: ((3bR,7aR,8aR)-6,7,8,8a-tetrahydro-5H,9H-pyrazolo[1′,5′: 1,5]pyrrolo[3,4-b]pyrrolizin-7a(3bH)-yl)methanol and ((3bS,7aS,8aS)-6,7,8,8a-tetrahydro-5H,9H-pyrazolo[1′,5′: 1,5]-pyrrolo[3,4-b]pyrrolizin-7a(3bH)-yl)methanol

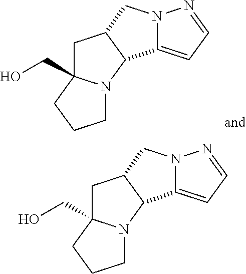

The title compounds were prepared by proceeding analogously as described in Example 1, Step 2. MS (ES, m/z): [M+H]⁺=220.2

### Intermediate 23

### Synthesis of a mixture of ((6aS,7aR,11aR)-6a,9,10,11a-tetrahydro-6H,7H-pyrido[3′,2′: 5,6]pyrano[3,4-b]-pyrrolizin-7a(8H)-yl)methanol and ((6aR,7aS,11aS)-6a,9,10,11a-tetrahydro-6H,7H-pyrido[3′,2′: 5,6]pyrano[3,4-b]pyrrolizin-7a(8H)-yl)methanol [Int-23]

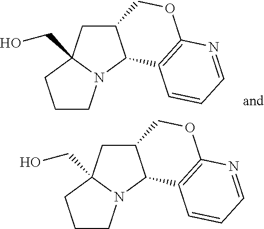

### Step 1: 7a-(tert-butyl) 2-methyl (2S,3R,7aR)-3-(2-chloropyridin-3-yl)tetrahydro-1H-pyrrolizine-2,7a(5H)-dicarboxylate and 7a-(tert-butyl) 2-methyl (2R,3S,7aS)-3-(2-chloropyridin-3-yl)tetrahydro-1H-pyrrolizine-2,7a(5H)-dicarboxylate

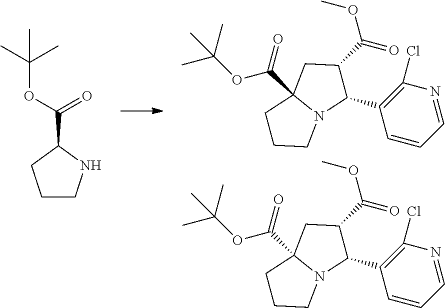

To a stirred mixture of tert-butyl (2S)-pyrrolidine-2-carboxylate (15.0 g, 87.6 mmol, 1.0 eq.) in toluene (150 mL) were added AgOAc (0.73 g, 4.4 mmol, 0.050 eq.), 2-chloropyridine-3-carbaldehyde (12.4 g, 87.6 mmol, 1.0 eq.), TEA (9.75 g, 96.4 mmol, 1.1 eq.) and methyl acrylate (7.54 g, 87.6 mmol, 1.0 eq.) at room temperature. The resulting mixture was stirred for 48 h at room temperature in black box and then concentrated. The residue was purified by silica gel column chromatography, eluted with PE/EA (0-25%), to afford the crude product. The crude product was further purified by Prep-HPLC to afford the title compounds (2.0 g, 6.1%) as a white solid.

### Step 2: tert-butyl (2S,3R,7aR)-3-(2-chloropyridin-3-yl)-2-(hydroxymethyl)tetrahydro-1H-pyrrolizine-7a(5H)-carboxylate and tert-butyl (2R,3S,7aS)-3-(2-chloropyridin-3-yl)-2-(hydroxymethyl)tetrahydro-1H-pyrrolizine-7a(5H)-carboxylate

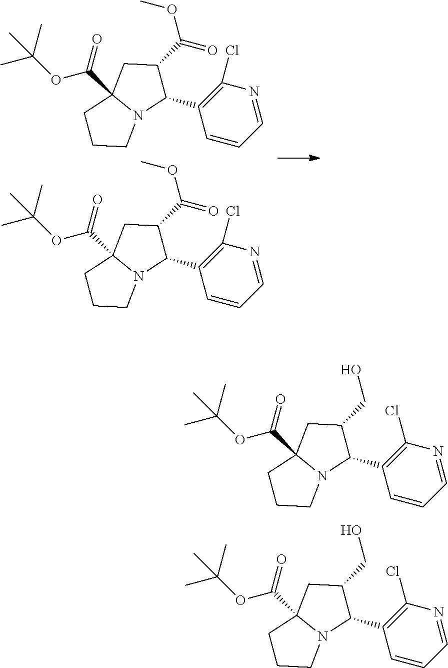

To a stirred solution of 7a-(tert-butyl) 2-methyl (2S,3R,7aR)-3-(2-chloropyridin-3-yl)tetrahydro-1H-pyrrolizine-2,7a(5H)-dicarboxylate and 7a-(tert-butyl) 2-methyl (2R,3S,7aS)-3-(2-chloropyridin-3-yl)tetrahydro-1H-pyrrolizine-2.7a(5H)-dicarboxylate (3.5 g, 9.2 mmol, 1.0 eq.) in THF (5 mL) was added DIBAL-H (18.4 mL, 18.4 mmol, 2.0 eq.,1.0 M in THF) dropwise at 0° C. under nitrogen atmosphere. The resulting mixture was stirred for 2 h, quenched with water at 0° C. and then filtered. The filter cake was washed with THF. The filtrate was concentrated and the residue was purified by silica gel column chromatography, eluted with MeOH/DCM (0-10%), to afford the title compounds (2.2 g, 67.4%) as a white solid.

Step 3: tert-butyl (6aS,7aR,11aR)-6a.9,10,11a-tetrahydro-6H,7H-pyrido[3′,2′: 5,6]pyrano[3,4-b]pyrrolizine-7a(8H)-carboxylate and tert-butyl (6aR,7aS,11aS)-6a,9,10,11a-tetrahydro-6H,7H-pyrido[3′,2′: 5,6]pyrano[3,4-b]pyrrolizine-7a(8H)-carboxylate

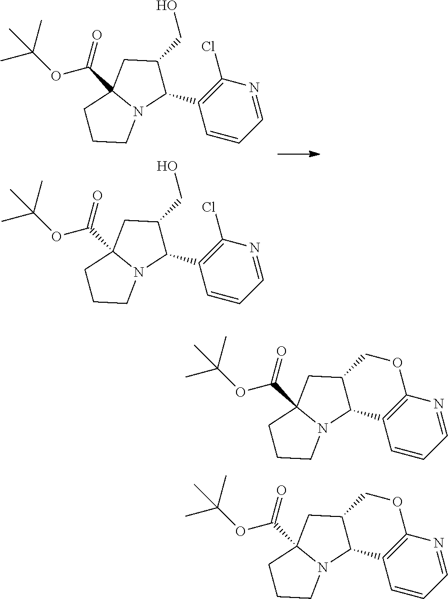

To a stirred solution of tert-butyl (2S,3R,7aR)-3-(2-chloropyridin-3-yl)-2-(hydroxymethyl)tetrahydro-1H-pyrrolizine-7a(5H)-carboxylate and tert-butyl (2R,3S,7aS)-3-(2-chloropyridin-3-yl)-2-(hydroxymethyl)tetrahydro-1H-pyrrolizine-7a(5H)-carboxylate (1.0 g, 2.8 mmol, 1.0 eq.) in t-BuOH (10 mL) was added t-BuOK (0.95 g, 8.5 mmol, 3.0 eq.) in portions at room temperature under nitrogen atmosphere. The reaction mixture was stirred for 3 h at 45° C., diluted with water and then extracted with EtOAc. The combined organic layers were washed with water, brine, dried over anhydrous Na₂SO₄, filtered and then concentrated. The residue was purified by silica gel column chromatography, eluted with EA/PE (0-100%), to afford the title compounds (500 mg, 57.1%) as a brown oil.

### Step 4: ((6aS,7aR,11aR)-6a,9,10,11a-tetrahydro-6H,7H-pyrido[3′,2′: 5,6]pyrano[3,4-b]pyrrolizin-7a(8H)-yl)methanol and ((6aR,7aS,11aS)-6a,9,10,11a-tetrahydro-6H,7H-pyrido[3′,2′: 5,6]-pyrano[3,4-b]pyrrolizin-7a(8H)-yl)methanol

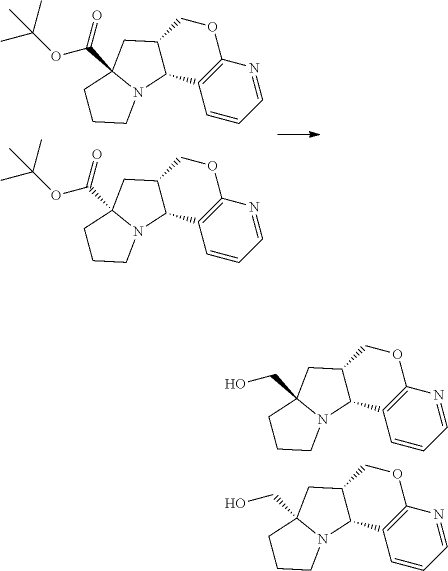

The title compounds were prepared by proceeding analogously as described in Example 1, Step 2. MS (ES, m/z): [M+H]⁺=247.2.

### Intermediate 24

### Synthesis of a mixture of ((6aS,7aR,11aR)-6a,9,10,11a-tetrahydro-6H,7H-chromeno[3,4-b]pyrrolizin-7a(8H)-yl)methanol and ((6aR,7aS,11aS)-6a,9,10,11a-tetrahydro-6H,7H-chromeno[3,4-b]pyrrolizin-7a(8H)-yl)methanol [Int-24]

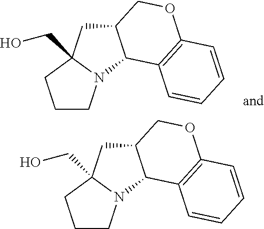

### Step 1: 7a-(tert-butyl) 2-methyl (2S,3R,7aR)-3-(2-(methoxymethoxy)phenyl)tetrahydro-1H-pyrrolizine-2,7a(5H)-dicarboxylate and 7a-(tert-butyl) 2-methyl (2R,3S,7aS)-3-(2-(methoxymethoxy)phenyl)tetrahydro-1H-pyrrolizine-2,7a(5H)-dicarboxylate

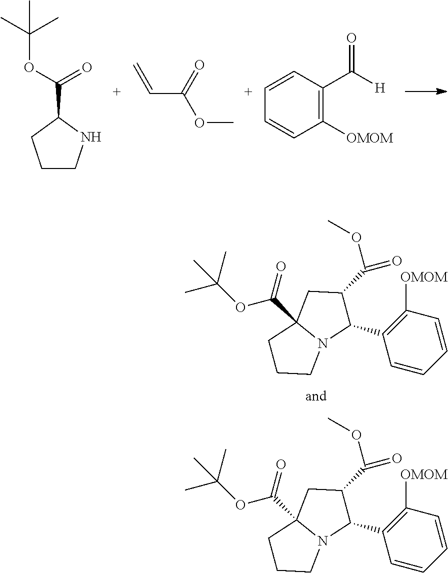

To a stirred mixture of tert-butyl L-prolinate (15.0 g, 87.6 mmol, 1.0 eq.) in toluene (600 mL) were added AgOAc (0.73 g, 4.4 mmol, 0.05 eq.), 2-(methoxymethoxy)benzaldehyde (14.6 g, 87.9 mmol, 1.0 eq.), TEA (9.8 g, 96.8 mmol, 1.1 eq.) and methyl acrylate (7.5 g, 87.1 mmol, 1.0 eq.) at room temperature. The resulting mixture was stirred for 48 h at room temperature under dart and then concentrated. The residue was purified by silica gel column chromatography, eluted with PE/EA (0˜25%), to afford the crude compound. The crude product was further purified by Prep-HPLC to afford the title compound (6.0 g, 17.0%) as a light-yellow oil.

### Step 2: tert-butyl (2S,3R,7aR)-2-(hydroxymethyl)-3-(2-(methoxymethoxy)phenyl)tetrahydro-1H-pyrrolizine-7a(5H)-carboxylate and tert-butyl (2R,3S,7aS)-2-(hydroxymethyl)-3-(2-(methoxymethoxy)phenyl)tetrahydro-1H-pyrrolizine-7a(5H)-carboxylate

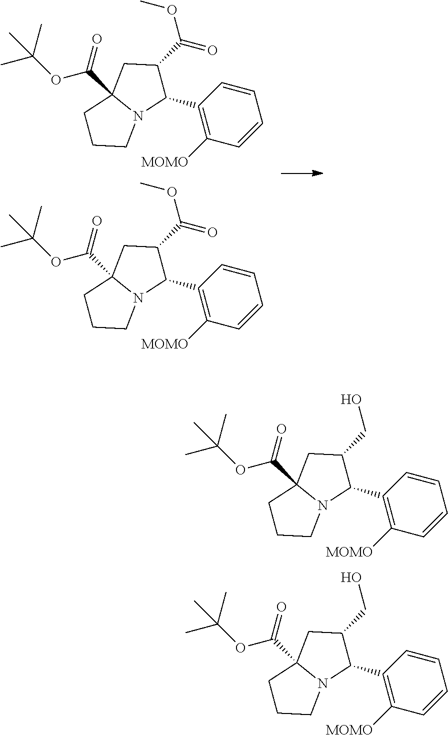

To a stirred solution of 7a-(tert-butyl) 2-methyl (2S,3R,7aR)-3-(2-(methoxymethoxy) phenyl)tetrahydro-1H-pyrrolizine-2,7a(5H)-dicarboxylate and 7a-(tert-butyl) 2-methyl (2R,3S,7aS)-3-(2-(methoxymethoxy) phenyl)tetrahydro-1H-pyrrolizine-2,7a(5H)-dicarboxylate (3.0 g, 7.4 mmol, 1.0 eq.) in THF (30 mL) was added DIBAL-H (22.2 mL, 22.2 mmol, 3.0 eq., 1.0 M in hexane) dropwise at 0° C. The resulting mixture was stirred for 30 min at 0° C., quenched with water and then filtered. The filter cake was washed with EtOAc. The filtrate was concentrated and the residue was purified by silica gel column chromatography, eluted with MeOH/DCM (0˜10%), to afford the title compounds (2.1 g, 75.7%) as a light yellow oil.

### Step 3: tert-butyl (2S,3R,7aR)-2-(hydroxymethyl)-3-(2-hydroxyphenyl)tetrahydro-1H-pyrrolizine-7a(5H)-carboxylate and tert-butyl (2R,3S,7aS)-2-(hydroxymethyl)-3-(2-hydroxyphenyl)-tetrahydro-1H-pyrrolizine-7a(5H)-carboxylate

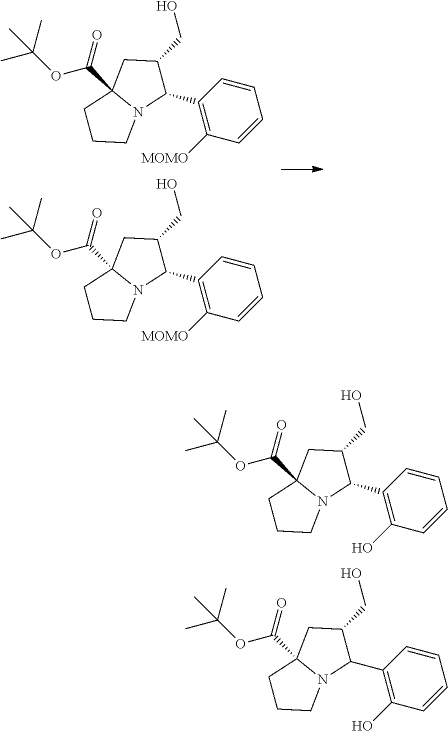

A solution of tert-butyl (2S,3R,7aR)-2-(hydroxymethyl)-3-(2-(methoxymethoxy) phenyl)tetrahydro-1H-pyrrolizine-7a(5H)-carboxylate and tert-butyl (2R,3S,7aS)-2-(hydroxymethyl)-3-(2-(methoxymethoxy) phenyl)tetrahydro-1H-pyrrolizine-7a(5H)-carboxylate (2.0 g, 5.3 mmol, 1.0 eq.) in DCM (20 mL) was added 4.0 M HCl(gas) in 1,4-dioxane (10 me) at 0° C. The reaction mixture was stirred for 2 h at 0° C., basified to pH=7 with NH₃ in MeOH and then concentrated. The residue was purified by silica gel column chromatography, eluted with MeOH/DCM (0˜10%), to afford the title compounds (1.4 g, 79.2%) as a light yellow oil.

### Step 4: tert-butyl (6aS,7aR,11aR)-6a.9,10,11a-tetrahydro-6H,7H-chromeno[3,4-b]pyrrolizine-7a(8H)-carboxylate and tert-butyl (6aR,7aS,11aS)-6a,9,10,11a-tetrahydro-6H,7H-chromeno[3,4-b]pyrrolizine-7a(8H)-carboxylate

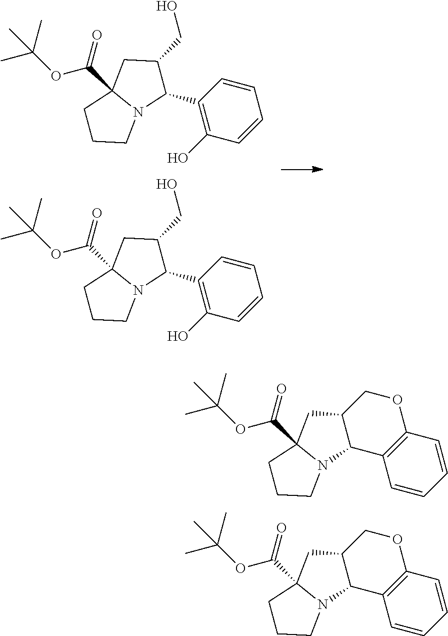

To a stirred solution of tert-butyl (2S,3R,7aR)-2-(hydroxymethyl)-3-(2-hydroxyphenyl) tetrahydro-1H-pyrrolizine-7a(5H)-carboxylate and tert-butyl (2R,3S,7aS)-2-(hydroxymethyl)-3-(2-hydroxyphenyl) tetrahydro-1H-pyrrolizine-7a(5H)-carboxylate (1.3 g, 3.9 mmol, 1.0 eq.) in THF (40 mL) was added 60% NaH (470 mg, 11.8 mmol, 3.0 eq.) at 0° C. under nitrogen. The resulting mixture was stirred for 15 min at 0° C., and a solution of MsCl (450 mg, 3.9 mmol, 1.0 eq.) in THF (0.5 mL) was added slowly. The reaction mixture was allowed to warm to RT and stirred for 16 h, quenched by water and then extracted with EtOAc. The combined organic layers were washed with water, brine, dried over anhydrous Na₂SO₄, filtered and then concentrated. The residue was purified by silica gel column chromatography, eluted with EA/PE (0˜35%), to afford the title compounds (360 mg, 28.2%) as a light yellow oil.

### Step 5: ((6aS,7aR,11aR)-6a,9,10,11a-tetrahydro-6H,7H-chromeno[3,4-b]pyrrolizin-7a(8H)-yl)methanol and ((6aR,7aS,11aS)-6a,9,10,11a-tetrahydro-6H,7H-chromeno[3,4-b]pyrrolizin-7a(8H)-yl)methanol

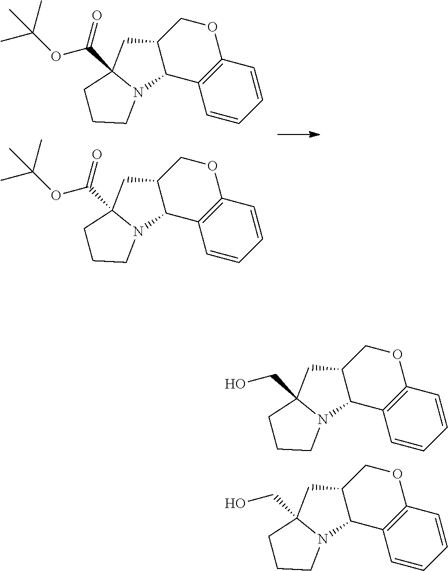

The title compounds were prepared by proceeding analogously as described in Example 1, Step 2. MS (ES, m/z): [M+H]⁺=246.2.

### Intermediate 25

### Synthesis of ((9R)-9-fluoro-6a,9,10,11a-tetrahydro-6H,7H-chromeno[3,4-b]pyrrolizin-7a(8H)-yl)methanol [Int-25]

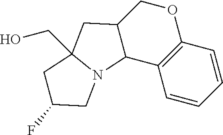 [SMILES: OCC12CC3COc4ccccc4C3N1C[C@H](F)C2]

The title compound was prepared by proceeding analogously as in intermediate Int-23, Step 1-5 using di-tert-butyl (2S,4R)-4-fluoropyrrolidine-1,2-dicarboxylate instead of tert-butyl L-prolinate in Step 1. MS (ES, m/z): [M+H]⁺=264.1.

### Intermediate 26

### Synthesis of ((9S)-9-fluoro-6a,9,10,11a-tetrahydro-6H,7H-chromeno[3,4-b]pyrrolizin-7a(8H)-yl)-methanol [Int-26]

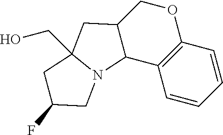 [SMILES: OCC12CC3COc4ccccc4C3N1C[C@@H](F)C2]

The title compound was prepared by proceeding analogously as described in intermediate Int-23, Steps 1-5 using di-tert-butyl (2S,4S)-4-fluoropyrrolidine-1,2-dicarboxylate instead of tert-butyl L-prolinate in step 1. MS (ES, m/z): [M+H]⁺=264.1.

### Intermediate 27

### Synthesis of (7-methylenehexahydroindolizin-8a(1H)-yl)methanol [Int-27]

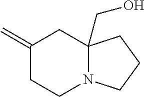

The title compound was prepared by proceeding analogously as described in Example 5, Steps 3-6, using 1-(tert-butyl) 2-methyl 4-methylenepiperidine-1,2-dicarboxylate instead of ethyl 2,3-dihydro-1H-pyrrolo[1,2-a]indole-9a(9H)-carboxylate.

### Intermediate 28

### Synthesis of (2-methylenehexahydroindolizin-8a(1H)-yl)methanol [Int-28]

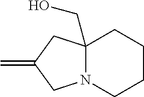

### Step 1: methyl 1-(2-(chloromethyl)allyl)piperidine-2-carboxylate

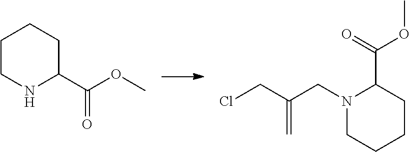

To a stirred mixture of methyl piperidine-2-carboxylate (0.6 g, 4.2 mmol, 1.0 eq.) and K₂CO₃ (848 mg, 6.1 mmol, 1.5 eq.) in DMF (5 mL) was added 3-chloro-2-(chloromethyl)prop-1-ene (756 mg, 6.0 mmol, 1.4 eq.) at 0° C. The result mixture was stirred for 16 h at room temperature, quenched with water and then extracted with EtOAc. The combined organic layers were washed with brine, dried over Na₂SO₄, filtered and then concentrated to afford the title compound as a yellow oil (1.0 g, crude, 100%), which was used for next step without further purification.

### Step 2: methyl 2-methylenehexahydroindolizine-8a(1H)-carboxylate

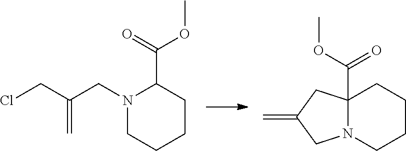

To a stirred solution of methyl 1-(2-(chloromethyl)allyl)piperidine-2-carboxylate (1.0 g, 4.3 mmol, 1.0 eq.) in anhydrous THF (10 mL) was added 1.0 M LiHMDS (8.0 mL, 8.0 mmol, 1.9 eq.) dropwise at −78° C. under nitrogen atmosphere. The resulting mixture was stirred at −78° C. for 2 h, and then at room temperature for 16 h. The reaction mixture was quenched with water and then extracted with EtOAc. The combined organic layers were washed with brine, dried over Na₂SO₄, filtered, and then concentrated. The residue was purification by silica gel chromatogram; y, eluting with EA/PE (0-50%), to afford the title compound (0.60 g, 72.1%) as a yellow oil.

### Step 3: (2-methylenehexahydroindolizin-8a(1H)-yl)methanol

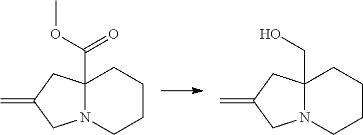

The title compound was prepared by proceeding analogously as described in Example 1, Step 2.

### Intermediate 29

### Synthesis of (2-methylenehexahydro-1H-pyrrolo[1,2-a]azepin-9a(5H)-yl)methanol [Int-29]

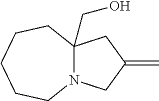

The title compound was prepared by proceeding analogously as described in Example 1, Steps 1-2 using methyl azepane-2-carboxylate instead of ethyl 5-oxopyrrolidine-2-carboxylate in step 1. MS (ES, m/z): [M+H]⁺=182.2.

### Intermediate 30

### Synthesis of (1R,5S)-3,8-diazaspiro[bicyclo[3.2.1]octane-6,1′-cyclopropane] [Int-30]

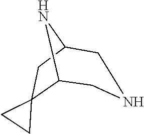

### Step 1: tert-butyl (1R,5S)-8-(2-phenylpropan-2-yl)-3,8-diazaspiro[bicyclo[3.2.1]octane-6,1′-cyclopropane]-3-carboxylate

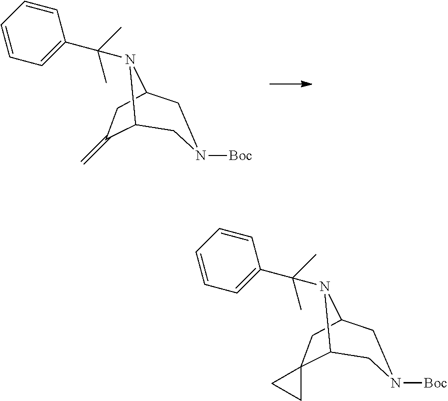 [SMILES: C=C1CC2CN(C(=O)OC(C)(C)C)CC1N2C(C)(C)c1ccccc1 ; CC(C)(C)OC(=O)N1CC2CC3(CC3)C(C1)N2C(C)(C)c1ccccc1]

To a stirred mixture of 40% KOH (30 mL) in Et₂O (15 mL) was added N-Nitroso-N-methylurea (ISOPAC, 2708.8 mg, 26.3 mmol, 30.0 eq.) in portions at 0° C. The resulting mixture was stirred for 10 min at 0° C. and then the layers were separated. The aqueous layer was extracted with Et₂O. The combined Et₂O solution was added slowly to a stirred mixture of tert-butyl 6-methylidene-8-(2-phenylpropan-2-yl)-3,8-diazabicyclo[3.2.1]octane-3-carboxylate (300 mg, 0.88 mmol, 1.0 eq.) and Pd(acac)₂ (30 mg, 0.098 mmol, 0.11 eq.) in DCM (5 mL) at room temperature under nitrogen atmosphere. After stirring for 1 h at room temperature, the reaction mixture was quenched with water at room temperature and then extracted with DCM. The organic layer was concentrated and the residue was purified by silica gel column chromatography, eluted with PE:EA (10:1), to afford the title compound (115 mg, 36.4%) as a white solid.

### Step 2: (1R,5S)-3,8-diazaspiro[bicyclo[3.2.1]octane-6,1′-cyclopropane]

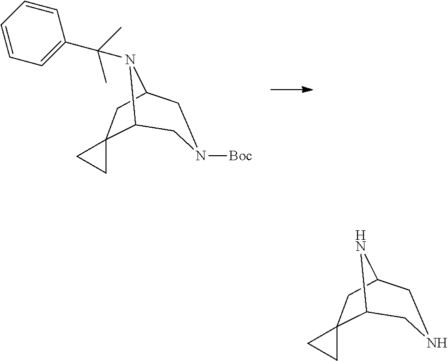 [SMILES: CC(C)(C)OC(=O)N1CC2CC3(CC3)C(C1)N2C(C)(C)c1ccccc1 ; C1NCC2NC1CC21CC1]

A mixture of tert-butyl (1R,5S)-8-(2-phenylpropan-2-yl)-3,8-diazaspiro[bicyclo[3.2.1]octane-6,1′-cyclopropane]-3-carboxylate (290 mg, 0.81 mmol, 1.00 eq.) in TFA (3 mL) was stirred for 2 h at 80° C. under nitrogen atmosphere. The residue was concentrated and purified by trituration with Et₂O to afford the title compound (80 mg, 71.6%) as a white oil. MS (ES, m/z): [M+H]⁺=139.1.

### Intermediate 31

### Synthesis of 3.8-diazaspiro[bicyclo[3.2.1]octane-6,1′-cyclopropane] [Int-31]

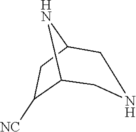

### Step 1: tert-butyl (1R,5S)-6-cyano-8-(2-phenylpropan-2-yl)-3,8-diazabicyclo[3.2.1]octane-3-carboxylate

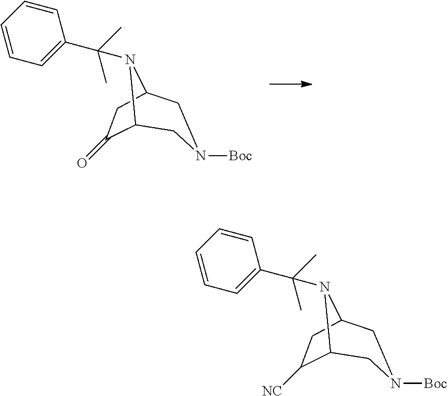 [SMILES: CC(C)(C)OC(=O)N1CC2CC(=O)C(C1)N2C(C)(C)c1ccccc1 ; CC(C)(C)OC(=O)N1CC2CC(C#N)C(C1)N2C(C)(C)c1ccccc1]

To a stirred solution of tert-butyl (1R,5R)-6-oxo-8-(2-phenylpropan-2-yl)-3,8-diazabicyclo[3.2.1]octane-3-carboxylate (300 mg, 0.87 mmol, 1.00 eq.) and 1-((isocyanomethyl) sulfonyl)-4-methylbenzene (221 mg, 1.13 mmol, 1.30 eq.) in DME (2 mL) was added a solution of t-BuOK (215 mg, 1.92 mmol, 2.21 eq.) in DME (1 mL) dropwise, followed by EtOH (3 mL) dropwise at 0° C. under nitrogen atmosphere. The resulting mixture was stirred at 0° C. for 1 h, and then at room temperature for 16 h. The reaction mixture was quenched with water, and then extracted with EtOAc. The combined organic layers were concentrated and the residue was purified by silica gel column chromatography, eluted with PE/EA (9:1), to afford the title compound (75 mg, 24.1%).

### Step 2: (1R,5S)-3,8-diazaspiro[bicyclo[3.2.1]octane-6,1′-cyclopropane]

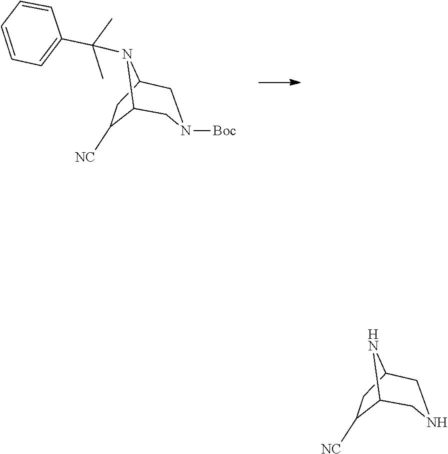 [SMILES: CC(C)(C)OC(=O)N1CC2CC(C#N)C(C1)N2C(C)(C)c1ccccc1 ; N#CC1CC2CNCC1N2]

A solution of tert-butyl (1R,5S)-6-cyano-8-(2-phenylpropan-2-yl)-3,8-diazabicyclo[3.2.1]octane-3-carboxylate (70 mg, 0.20 mmol, 1.00 eq.) and TFA (1.0 mL, 13.07 mmol, 65.35 eq.) in DCM (1 mL) was stirred for 1.5 h at room temperature. The reaction mixture was concentrated to afford the title compound (25 mg, 90.0%) as a white solid, which was used for next step without further purification. MS (ES, m/z): [M+H]⁺=138.2.

### Intermediate 32

### Synthesis of 2,2,2-trifluoro-1-((1S,5R)-2-methyl-3,8-diazabicyclo[3.2.1]octan-8-yl)ethan-1-one [Int-32]

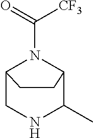

### Step 1: tert-butyl (1S,5R)-8-benzyl-2-methyl-3,8-diazabicyclo[3.2.1]octane-3-carboxylate

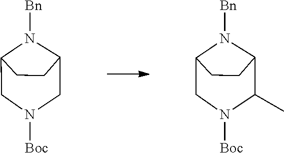 [SMILES: CC(C)(C)OC(=O)N1CC2CCC(C1)N2Cc1ccccc1 ; CC1C2CCC(CN1C(=O)OC(C)(C)C)N2Cc1ccccc1]

To a stirred solution of tert-butyl (1R,5S)-8-benzyl-3,8-diazabicyclo[3.2.1]octane-3-carboxylate (1.1 g, 3.6 mmol, 1.0 eq.) and TMEDA (2.2 g, 18.9 mmol, 5.2 eq.) in THF (5 mL) was added 1.3M s-BuLi in n-hexane (14.5 mL, 18.9 mmol, 5.3 eq.) at −78° C. The resulting mixture was stirred for 1 h at −78° C., and then Mel (1.0 g, 7.0 mmol, 1.9 eq.) was added dropwise. The resulting mixture was stirred for 2 h at room temperature, quenched with water and then extracted with EtOAc. The combined organic layers were washed with water, brine, dried over anhydrous Na₂SO₄, filtered and then concentrated. The residue was purified by silica gel column chromatography, eluted with PE/EA (0˜10%), to afford the title compound (880 mg, 77.8%) as a light yellow oil.

### Step 2: tert-butyl (1S,5R)-2-methyl-3,8-diazabicyclo[3.2.1]octane-3-carboxylate

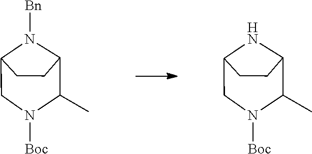 [SMILES: CC1C2CCC(CN1C(=O)OC(C)(C)C)N2Cc1ccccc1 ; CC1C2CCC(CN1C(=O)OC(C)(C)C)N2]

A mixture of tert-butyl (1S,5R)-8-benzyl-2-methyl-3,8-diazabicyclo[3.2.1]octane-3-carboxylate (1.1 g, 3.5 mmol, 1.0 eq.) and 5% Pd/C (185 mg) in MeOH (10 mL) was stirred under hydrogen atmosphere at room temperature for 16 h. The reaction mixture was filtered through a Celite pad and then concentrated to afford the title compound (760 mg, 97.1%) as a light-yellow oil.

### Step 3: tert-butyl (1S,5R)-2-methyl-8-(2,2,2-trifluoroacetyl)-3,8-diazabicyclo[3.2.1]octane-3-carboxylate

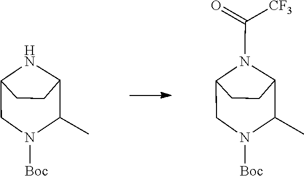 [SMILES: CC1C2CCC(CN1C(=O)OC(C)(C)C)N2 ; CC1C2CCC(CN1C(=O)OC(C)(C)C)N2C(=O)C(F)(F)F]

To a stirred solution of tert-butyl (1S,5R)-2-methyl-3,8-diazabicyclo[3.2.1]octane-3-carboxylate (880 mg, 3.9 mmol, 1.0 eq.) and TEA (1.18 g, 11.7 mmol, 3.0 eq.) in DCM (10 mL) was added TFAA (2.5 g, 11.9 mmol, 3.1 eq.) at 0° C. under nitrogen atmosphere. After stirring for 1 h, the reaction mixture was quenched with water and then extracted with EtOAc. The combined organic layers were washed with water, brine, dried over anhydrous Na₂SO₄, filtered and then concentrated. The residue was purified by silica gel column chromatography, eluted with PE/THF (0˜20%), to afford the title compound (560 mg, 43.6%) as a light-yellow oil.

### Step 4: 2,2,2-trifluoro-1-((1S,5R)-2-methyl-3,8-diazabicyclo[3.2.1]octan-8-yl)ethan-1-one

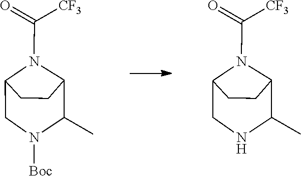 [SMILES: CC1C2CCC(CN1C(=O)OC(C)(C)C)N2C(=O)C(F)(F)F ; CC1NCC2CCC1N2C(=O)C(F)(F)F]

To a stirred solution of tert-butyl (1S,5R)-2-methyl-8-(2,2,2-trifluoroacetyl)-3,8-diazabicyclo[3.2.1]octane-3-carboxylate (500 mg, 1.55 mmol, 1.00 eq.) in DCM (5 mL) was added HCl in 1,4-dioxane (2.5 mL, 4.0 M) at 0° C. The resulting mixture was stirred at room temperature for 1 h, concentrated, diluted with ACN, and then basified to pH=7 with K₂CO₃ (powder). The resulting mixture was filtered and the filter cake was washed with ACN. The filtrate was concentrated to give the title compound (210 mg, 61.3%) as alight-yellow oil. MS (ES, m/z): [M+H]⁺=223.1.

### Intermediate 33

### Synthesis of tert-butyl (1R,5S)-3-(2-chloro-7-(8-ethynyl-7-fluoronaphthalen-1-yl)-8-fluoro-pyrido[4,3-d]pyrimidin-4-yl)-3,8-diazabicyclo[3.2.1]octane-8-carboxylate [Int-33]

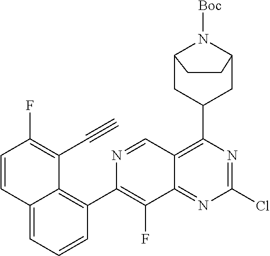 [SMILES: C#Cc1c(F)ccc2cccc(-c3ncc4c(C5CC6CCC(C5)N6C(=O)OC(C)(C)C)nc(Cl)nc4c3F)c12]

### Step 1: 8-fluoro-7-(7-fluoro-8-((triisopropylsilyl)ethynyl)naphthalen-1-yl)pyrido[4,3-d]-pyrimidine-2,4-diol

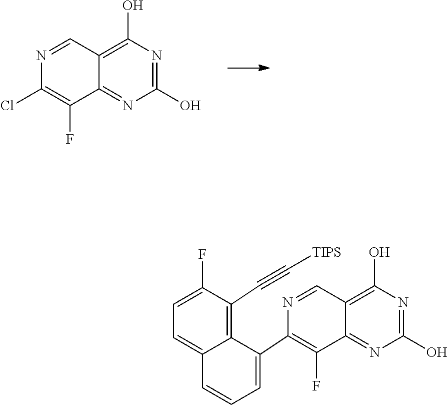

To a stirred solution of 7-chloro-8-fluoropyrido[4,3-d]pyrimidine-2,4-diol (4.0 g, 18.6 mmol, 1.0 eq.) and ((2-fluoro-8-(4,4,5,5-tetramethyl-1,3,2-dioxaborolan-2-yl)naphthalen-1-yl)ethynyl)triisopropylsilane (10.9 g, 24.1 mmol, 1.3 eq.) in EtOH (150 mL) and H₂O (50 mL) were added cataCXium A Pd G3 (2.4 g, 3.3 mmol, 0.18 eq.), K₃PO₄ (11.7 g, 55.1 mmol, 3.0 eq.) at room temperature under nitrogen atmosphere. The resulting mixture was stirred for 16 h at 80° C. under nitrogen atmosphere, cooled, diluted with water, and then extracted with EtOAc. The combined organic layers were washed with water, brine, dried over anhydrous Na₂SO₄, filtered and then concentrated. The residue was purified by silica gel column chromatography, eluted with MeOH/DCM (0-10%), to afford the title compound (5.5 g, 58.6%) as a yellow solid.

### Step 2: 7-(8-ethynyl-7-fluoronaphthalen-1-yl)-8-fluoropyrido[4,3-d]pyrimidine-2,4-diol

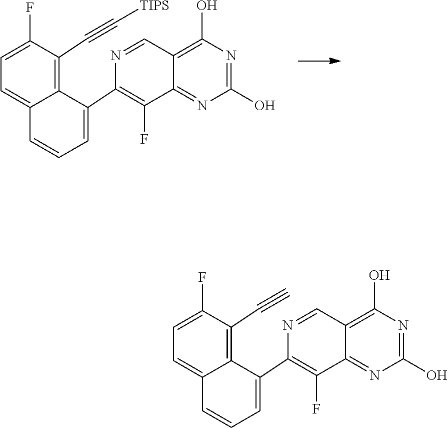

A mixture of 8-fluoro-7-(7-fluoro-8-((triisopropylsilyl)ethynyl)naphthalen-1-yl)pyrido [4,3-d]pyrimidine-2,4-diol (10.0 g, 19.8 mmol, 1.0 eq.) and CsF (12.0 g, 79.0 mmol, 4.0 eq.) in DMF (100 mL) was stirred for 1 h at 50° C. under nitrogen atmosphere. The reaction mixture was concentrated and the residue was purified by silica gel column chromatography, eluted with MeOH/DCM (0-10%), to afford the title compound (6.0 g, 86.9%) as a yellow solid.

### Step 3: 2,4-dichloro-7-(8-ethynyl-7-fluoronaphthalen-1-yl)-8-fluoropyrido[4,3-d]pyrimidine

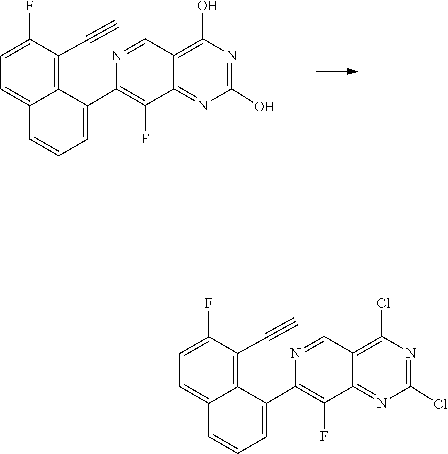 [SMILES: C#Cc1c(F)ccc2cccc(-c3ncc4c(O)nc(O)nc4c3F)c12 ; C#Cc1c(F)ccc2cccc(-c3ncc4c(Cl)nc(Cl)nc4c3F)c12]

To a stirred solution of POCl₃ (15.8 g, 103.0 mmol, 30.3 eq.) and DIPEA (13.4 g, 103.7 mmol, 30.5 eq.) was added 7-(8-ethynyl-7-fluoronaphthalen-1-yl)-8-fluoropyrido[4,3-d]-pyrimidine-2,4-diol (1.2 g, 3.4 mmol, 1.0 eq.) in portions at 0-5° C. The resulting mixture was stirred for 1 h and then concentrated. The residue was diluted with ice water, and then extracted with EtOAc. The combined organic layers were washed with water, brine, dried over anhydrous Na₂SO₄, filtered and then concentrated to afford the title compound (1.5 g, crude) as a brown solid, which was used for next step without further purification.

### Step 4: tert-butyl (1R,5S)-3-(2-chloro-7-(8-ethynyl-7-fluoronaphthalen-1-yl)-8-fluoropyrido[4,3-d]pyrimidin-4-yl)-3,8-diazabicyclo[3.2.1]octane-8-carboxylate

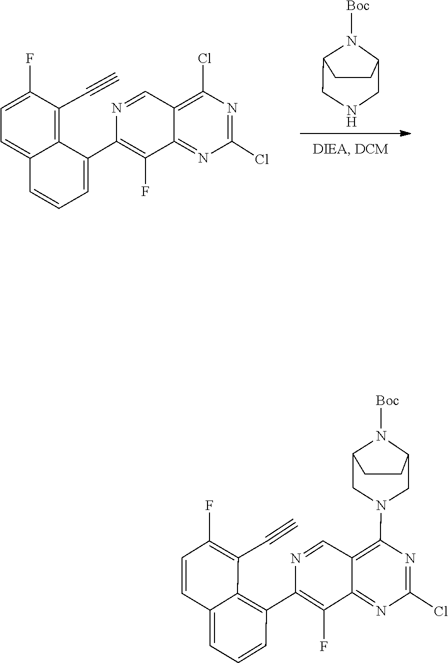 [SMILES: C#Cc1c(F)ccc2cccc(-c3ncc4c(Cl)nc(Cl)nc4c3F)c12 ; CC(C)(C)OC(=O)N1C2CCC1CNC2 ; C#Cc1c(F)ccc2cccc(-c3ncc4c(N5CC6CCC(C5)N6C(=O)OC(C)(C)C)nc(Cl)nc4c3F)c12]

To a stirred solution of 2,4-dichloro-7-(8-ethynyl-7-fluoronaphthalen-1-yl)-8-fluoropyrido-[4,3-d]pyrimidine (1.5 g, 3.9 mmol, 1.0 eq., crude) in DCM (30 mL) was added DIEA (1.1 g, 8.5 mmol, 2.2 eq.) dropwise at −40° C. After stirring for 5 min at −40° C., a solution of tert-butyl (1R,5S)-3,8-diazabicyclo[3.2.1]octane-8-carboxylate (870 mg, 4.1 mmol, 1.1 eq.) in THF (5 mL) was added dropwise. The resulting mixture was stirred for additional 15 min at −40° C., concentrated and the residue was purified by silica gel column chromatography, eluted with EA/PE (0-50%), to afford the title compound (770 mg, 41.2% over 2 steps) as a yellow solid. MS (ES, m/z): [M+H]⁺=562.3.

### Example 1

### Synthesis of 4-(4-((1R,5S)-3,8-diazabicyclo[3.2.1]octan-3-yl)-8-fluoro-2-((2-methylene-tetrahydro-1H-pyrrolizin-7a(5H)-yl)methoxy)pyrido[4,3-d]pyrimidin-7-yl)-5-ethynyl-naphthalen-2-ol

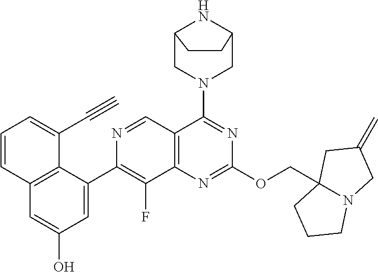 [SMILES: C#Cc1cccc2cc(O)cc(-c3ncc4c(N5CC6CCC(C5)N6)nc(OCC56CCCN5CC(=C)C6)nc4c3F)c12]

### Step 1: ethyl 2-methylene-5-oxotetrahydro-1H-pyrrolizine-7a(5H-carboxylate

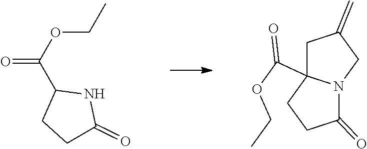

To a stirred mixture of ethyl 5-oxopyrrolidine-2-carboxylate 50.0 g, 318.13 mmol, 1.00 eq.) and 3-chloro-2-(chloromethyl)prop-1-ene (159.0 g, 1272.10 mmol, 4.00 eq.) in THF (300 mL) was added LiHMDS (668 mL, 1.0 M, 668 mmol, 2.10 eq.) dropwise at −40° C. under nitrogen atmosphere. The resulting mixture was stirred overnight at RT, quenched with sat. NH₄Cl (aq.) at 0-5° C., and then neutralized to pH=7 with 1.0 M aqueous HCl. The resulting mixture was extracted with EtOAc. The combined organic layers were washed with water, brine, and then dried over anhydrous Na₂SO₄. After filtration, the filtrate was concentrated and the residue was purified by silica gel column chromatography, eluted with EA/PE (0-70%), to afford the title compound (35.0 g, 52.6%) as a yellow oil.

### Step 2: ethyl 2-methylene-5-oxotetrahydro-1H-pyrrolizine-7a(5H)-carboxylate

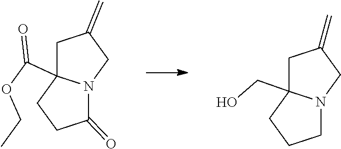

To a stirred solution of ethyl 2-methylidene-5-oxo-tetrahydropyrrolizine-7a-carboxylate (1.0 g, 4.78 mmol, 1.00 eq.) in THF (10 mL) was added 1.0 M LiAlH₄ in THF (14.4 mL, 14.4 mmol, 3.01 eq.) dropwise at RT under nitrogen atmosphere. The resulting mixture was refluxed for 3 h, cooled and then quenched with MeOH and Na₂SO₄·10H₂O. The resulting mixture was filtered through a Celite pad and concentrated. The crude product was purified by Prep-HPLC to afford the title compound (200 mg, 27.3%) as a colorless oil.

### Step 3: tert-butyl (1R,5S)-3-(2,7-dichloro-8-fluoropyrido[4,3-d]pyrimidin-4-yl)-3,8-diazabicyclo-[3.2.1]octane-8-carboxylate

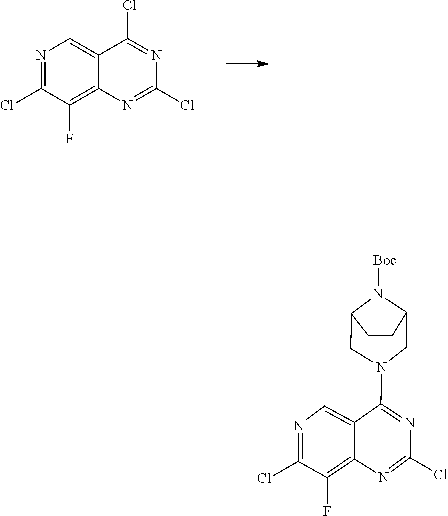 [SMILES: Fc1c(Cl)ncc2c(Cl)nc(Cl)nc12 ; CC(C)(C)OC(=O)N1C2CCC1CN(c1nc(Cl)nc3c(F)c(Cl)ncc13)C2]

To a stirred solution of 2,4,7-trichloro-8-fluoropyrido[4,3-d]pyrimidine (10.0 g, 39.61 mmol, 1.00 eq.) in DCM (160 mL) was added DIPEA (12.8 g, 99.03 mmol, 2.50 eq.) slowly at −40° C. The resulting mixture was stirred at −40° C. for 15 min, a solution of tert-butyl (1R,5S)-3,8-diazabicyclo[3.2.1]octane-8-carboxylate (8.4 g, 39.57 mmol, 1.00 eq.) in DCM (35 mL) then was added dropwise at −40° C. The resulting mixture was stirred for additional 15 min at −40° C., diluted with water, and then extracted with EtOAc. The combined organic layers were washed with brine, dried over anhydrous Na₂SO₄, filtered, and then concentrated. The residue was purified by silica gel column chromatography, eluted with EA/PE (0-50%), to afford the title compound (13.6 g, 80.2%) as a yellow solid.

### Step 4: tert-butyl (1R,5S)-3-(7-chloro-8-fluoro-2-((2-methylenetetrahydro-1H-pyrrolizin-7a(5H)-yl)methoxy) pyrido[4,3-d]pyrimidin-4-yl)-3,8-diazabicyclo[3.2.1]octane-8-carboxylate

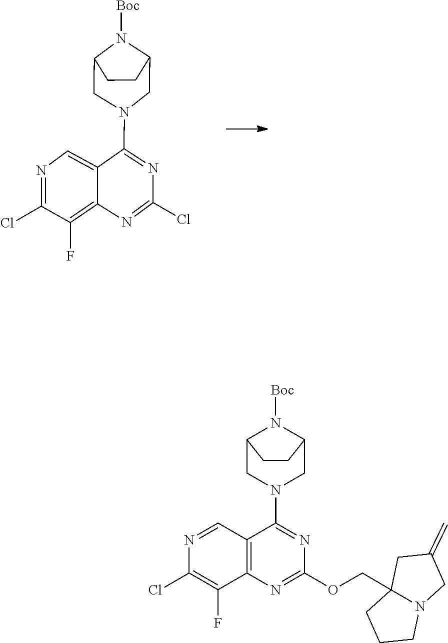 [SMILES: CC(C)(C)OC(=O)N1C2CCC1CN(c1nc(Cl)nc3c(F)c(Cl)ncc13)C2 ; C=C1CN2CCCC2(COc2nc(N3CC4CCC(C3)N4C(=O)OC(C)(C)C)c3cnc(Cl)c(F)c3n2)C1]

A mixture of tert-butyl (1R,5S)-3-(2,7-dichloro-8-fluoropyrido[4,3-d]pyrimidin-4-yl)-3,8-diazabicyclo[3.2.1]octane-8-carboxylate (200 mg, 0.47 mmol, 1.00 eq.), (2-methylidene-tetrahydro-1H-pyrrolizin-7a-yl)methanol (107 mg, 0.70 mmol, 1.49 eq.) and DIPEA (181 mg, 1.40 mmol, 2.98 eq.) in 1,4-dioxane (2 mL) was stirred for 3 h at 80° C. The resulting mixture was cooled and diluted with water. The resulting mixture was extracted with EtOAc. The combined organic layers were washed with water, brine, dried over anhydrous Na₂SO₄, filtered and then concentrated. The residue was purified by silica gel column chromatography, eluted with MeOH/CH₂Cl₂ (0-10%), to afford the title compound (135 mg, 53.2%) as a yellow solid.

### Step 5: tert-butyl (1R,5S)-3-(8-fluoro-7-(3-(methoxymethoxy)-8-((triisopropylsilyl)ethynyl) naphthalen-1-yl)-2-((2-methylenetetrahydro-1H-pyrrolizin-7a(5H)-yl)methoxy)pyrido[4,3-d]pyrimidin-4-yl)-3,8-diazabicyclo[3.2.1] octane-8-carboxylate

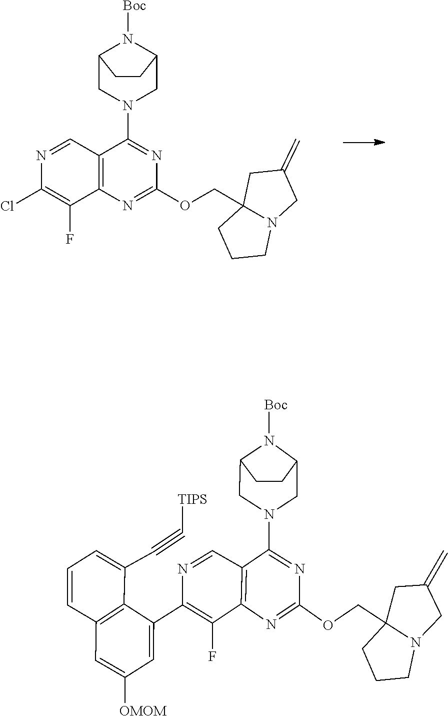

A mixture of tert-butyl (1R,5S)-3-(7-chloro-8-fluoro-2-((2-methylenetetrahydro-1H-pyrrolizin-7a(5H)-yl)methoxy)pyrido[4,3-d]pyrimidin-4-yl)-3,8-diazabicyclo[3.2.1]octane-8-carboxylate (120 mg, 0.22 mmol, 1.00 eq.), triisopropyl((6-(methoxymethoxy)-8-(4,4,5,5-tetramethyl-1,3,2-dioxaborolan-2-yl)naphthalen-1-yl)ethynyl)silane (130 mg, 0.26 mmol, 1.18 eq.), PdCl₂ (dtbpf) (14 mg, 0.021 mmol, 0.095 eq.) and K₂CO₃ (60 mg, 0.43 mmol, 1.95 eq.) in 1,4-dioxane (1.0 mL) and H₂O (0.1 mL) was stirred overnight at 85° C. under nitrogen atmosphere. The resulting mixture was cooled, diluted with water and then extracted with EtOAc. The combined organic layers were washed with water, brine, dried over anhydrous Na₂SO₄, filtered and then concentrated. The residue was purified by silica gel column chromatography, eluted with MeOH/CH₂Cl₂ (0-10%), to afford the title compound (30 mg, 15.5%) as a yellow solid.

### Step 6: tert-butyl (1R,5S)-3-(7-(8-ethynyl-3-(methoxymethoxy)naphthalen-1-yl)-8-fluoro-2-((2-methylene tetrahydro-1H-pyrrolizin-7a(5H)-yl)methoxy)pyrido[4,3-d]pyrimidin-4-yl)-3,8-diazabicyclo[3.2.1]octane-8-carboxylate

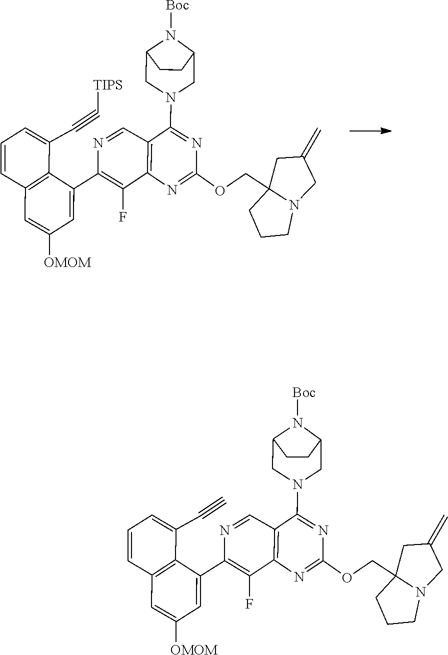

To a stirred solution of tert-butyl (1R,5S)-3-(8-fluoro-7-(3-(methoxymethoxy)-8-((triisopropylsilyl)ethynyl)naphthalen-1-yl)-2-((2-methylenetetrahydro-1H-pyrrolizin-7a(5H)-yl)methoxy)pyrido[4,3-d]pyrimidin-4-yl)-3,8-diazabicyclo[3.2.1]octane-8-carboxylate (33 mg, 0.038 mmol, 1.00 eq.) in DMF (0.6 mL) was added CsF (29 mg, 0.19 mmol, 5.00 eq.) at RT. The resulting mixture was stirred for 3 h at RT, diluted with water, and then extracted with EtOAc. The combined organic layers were washed with water, brine, dried over anhydrous Na₂SO₄, filtered and then concentrated to give the title compound (24 mg, 86.8%) as a light yellow solid.

### Step 7: 4-(4-((1R,5S)-3,8-diazabicyclo[3.2.1]octan-3-yl)-8-fluoro-2-((2-methylenetetrahydro-1H-pyrrolizin-7a(5H)-yl)methoxy)pyrido[4,3-d]pyrimidin-7-yl)-5-ethynylnaphthalene-2-ol

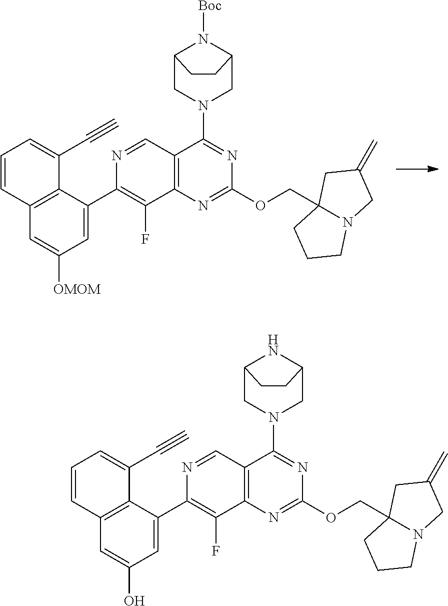

A solution of tert-butyl (1R,5S)-3-(7-(8-ethynyl-3-(methoxymethoxy)naphthalen-1-yl)-8-fluoro-2-((2-methylene tetrahydro-1H-pyrrolizin-7a(5H)-yl)methoxy)pyrido[4,3-d]pyrimidin-4-yl)-3,8-diazabicyclo[3.2.1]octane-8-carboxylate (24 mg, 0.033 mmol, 1.00 eq.) in DCM (0.5 mL) was added HCl in dioxane (0.5 mL, 4.0 M, 2.0 mmol, 60.61 eq.) at 0° C. The reaction mixture was stirred for 30 min at 0° C. basified to pH=8 with NH₃ in MeOH and then concentrated. The crude product was purified by Prep-HPLC to afford the title compound (5.5 mg, 28.8%) as a yellow solid. MS (ES, m/z): [M+1]⁺=577.3.

Table below provides compound of Formula (IIA1′) prepared by proceeding analogously as described in Example 1, Step 4-7, using starting materials indicated therein.

| Cpd. # | | | | |
|---|---|---|---|---|
| From | | | | |
| Table | | Amino | | MS |
| 1 | Compound | alcohol | Boronic ester in | [M + |
| above | Structure | Step 4 | Step 5 | H]⁺ |
| 15 | 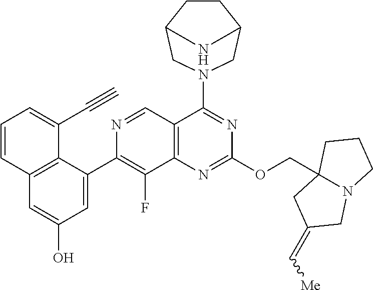 | Int-3 | 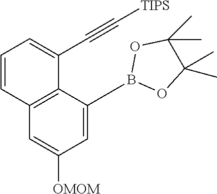 | 591.3 |
| 16 | 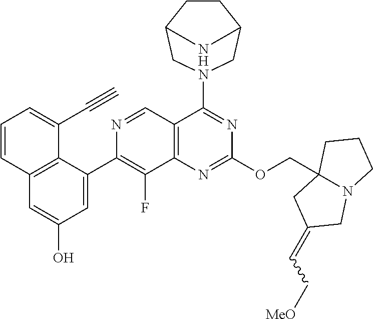 [SMILES: C#Cc1cccc2cc(O)cc(-c3ncc4c(N5CC6CCC(C5)N6)nc(OCC56CCCN5CC(=CCOC)C6)nc4c3F)c12] | Int-4 | 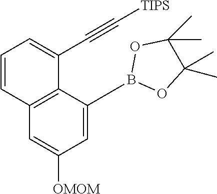 | 621.3 |
| 17 | 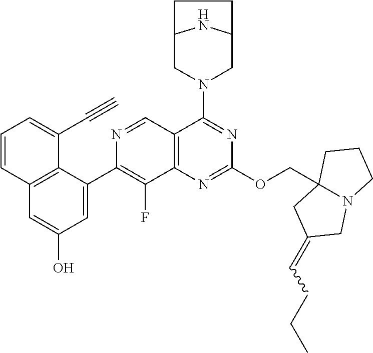 [SMILES: C#Cc1cccc2cc(O)cc(-c3ncc4c(N5CC6CCC(C5)N6)nc(OCC56CCCN5CC(=CCCC)C6)nc4c3F)c12] | Int-5 | 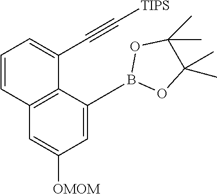 | 619.3 |
| 18 | 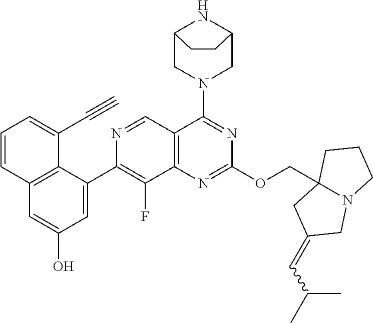 [SMILES: C#Cc1cccc2cc(O)cc(-c3ncc4c(N5CC6CCC(C5)N6)nc(OCC56CCCN5CC(=CC(C)C)C6)nc4c3F)c12] | Int-6 | 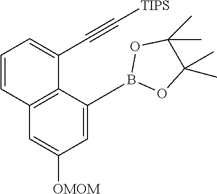 | 619.4 |
| 19 | 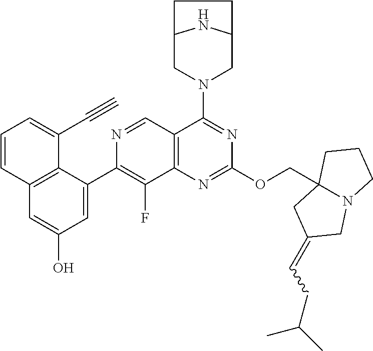 [SMILES: C#Cc1cccc2cc(O)cc(-c3ncc4c(N5CC6CCC(C5)N6)nc(OCC56CCCN5CC(=CCC(C)C)C6)nc4c3F)c12] | Int-7 | 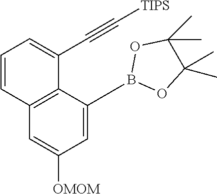 | 633.4 |
| 20 | 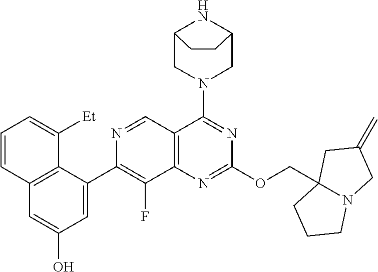 [SMILES: C=C1CN2CCCC2(COc2nc(N3CC4CCC(C3)N4)c3cnc(-c4cc(O)cc5cccc(CC)c45)c(F)c3n2)C1] | Ex. 1 | 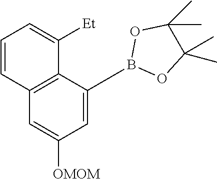 | 581.4 |
| 21 | 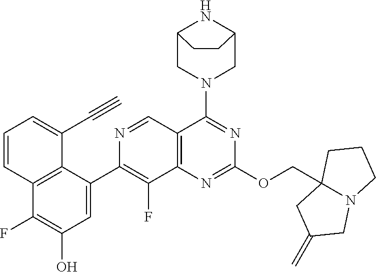 [SMILES: C#Cc1cccc2c(F)c(O)cc(-c3ncc4c(N5CC6CCC(C5)N6)nc(OCC56CCCN5CC(=C)C6)nc4c3F)c12] | 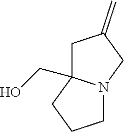 | 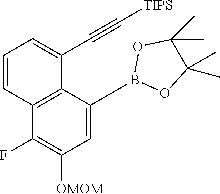 | 595.4 |
| 22 | 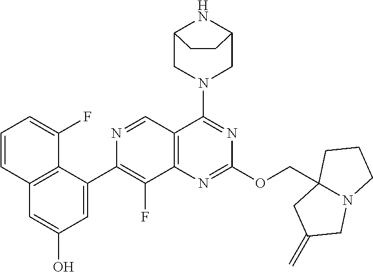 [SMILES: C=C1CN2CCCC2(COc2nc(N3CC4CCC(C3)N4)c3cnc(-c4cc(O)cc5cccc(F)c45)c(F)c3n2)C1] | 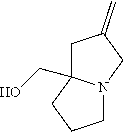 | 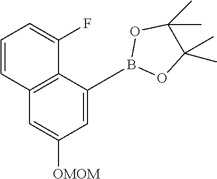 | 571.3 |
| 23 | 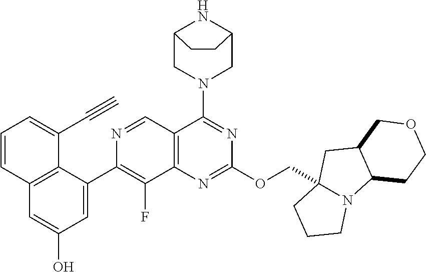 | Int-8 | 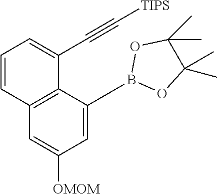 | 621.4 |
| | and | | | |
| | 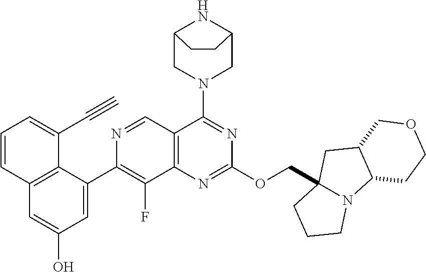 | | | |
| 24 | 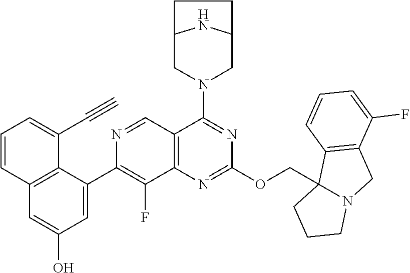 [SMILES: C#Cc1cccc2cc(O)cc(-c3ncc4c(N5CC6CCC(C5)N6)nc(OCC56CCCN5Cc5c(F)cccc56)nc4c3F)c12] | Int-9 | 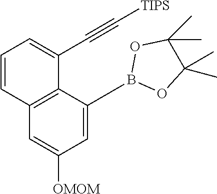 | 631.3 |
| 25 | 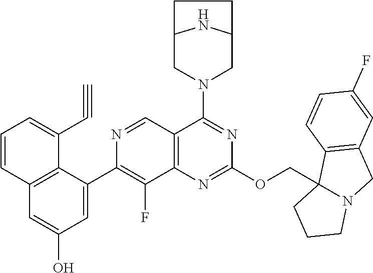 [SMILES: C#Cc1cccc2cc(O)cc(-c3ncc4c(N5CC6CCC(C5)N6)nc(OCC56CCCN5Cc5cc(F)ccc56)nc4c3F)c12] | Int-10 | 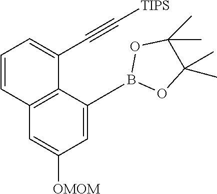 | 631.3 |
| 26 | 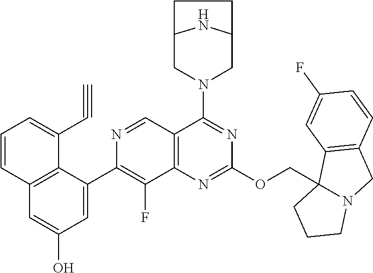 [SMILES: C#Cc1cccc2cc(O)cc(-c3ncc4c(N5CC6CCC(C5)N6)nc(OCC56CCCN5Cc5ccc(F)cc56)nc4c3F)c12] | Int-11 | 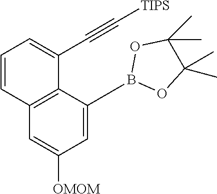 | 631.3 |
| 27 | 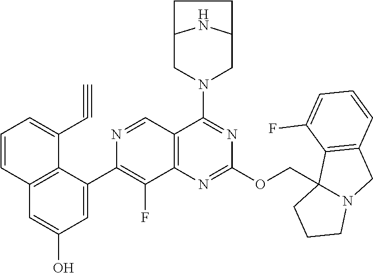 [SMILES: C#Cc1cccc2cc(O)cc(-c3ncc4c(N5CC6CCC(C5)N6)nc(OCC56CCCN5Cc5cccc(F)c56)nc4c3F)c12] | Int-12 | 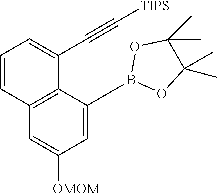 | 631.3 |
| 28 | 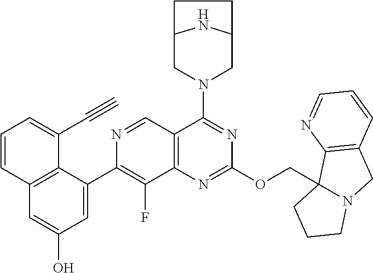 [SMILES: C#Cc1cccc2cc(O)cc(-c3ncc4c(N5CC6CCC(C5)N6)nc(OCC56CCCN5Cc5cccnc56)nc4c3F)c12] | Int-13 | 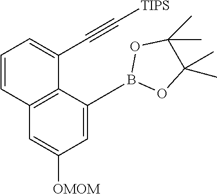 | 614.3 |
| 29 | 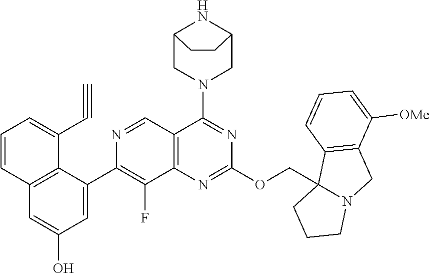 [SMILES: C#Cc1cccc2cc(O)cc(-c3ncc4c(N5CC6CCC(C5)N6)nc(OCC56CCCN5Cc5c(OC)cccc56)nc4c3F)c12] | Int-14 | 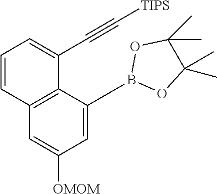 | 643.3 |
| 30 | 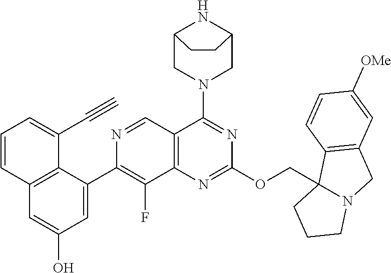 [SMILES: C#Cc1cccc2cc(O)cc(-c3ncc4c(N5CC6CCC(C5)N6)nc(OCC56CCCN5Cc5cc(OC)ccc56)nc4c3F)c12] | Int-15 | 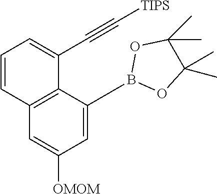 | 643.3 |
| 31 | 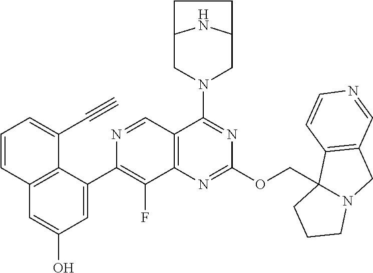 [SMILES: C#Cc1cccc2cc(O)cc(-c3ncc4c(N5CC6CCC(C5)N6)nc(OCC56CCCN5Cc5cnccc56)nc4c3F)c12] | Int-16 | 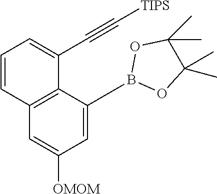 | 614.3 |
| 32 | 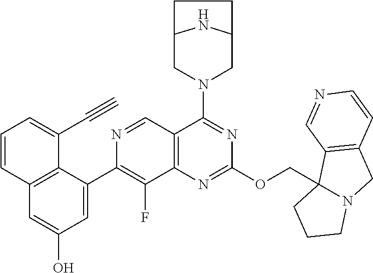 [SMILES: C#Cc1cccc2cc(O)cc(-c3ncc4c(N5CC6CCC(C5)N6)nc(OCC56CCCN5Cc5ccncc56)nc4c3F)c12] | Int-17 | 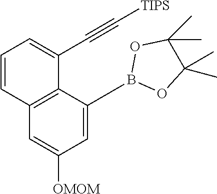 | 614.3 |
| 33 | 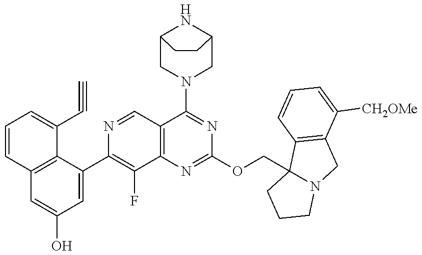 | Int-18 | 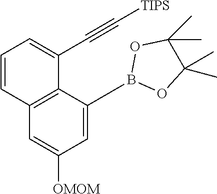 | 657.3 |
| 34 | 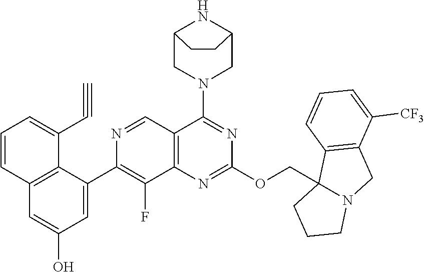 [SMILES: C#Cc1cccc2cc(O)cc(-c3ncc4c(N5CC6CCC(C5)N6)nc(OCC56CCCN5Cc5c(C(F)(F)F)cccc56)nc4c3F)c12] | Int-19 | 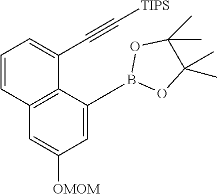 | 681.3 |
| 35a | 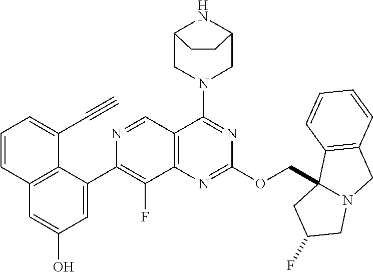 [SMILES: C#Cc1cccc2cc(O)cc(-c3ncc4c(N5CC6CCC(C5)N6)nc(OC[C@]56C[C@@H](F)CN5Cc5ccccc56)nc4c3F)c12] | Int-20a | 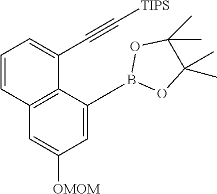 | 631.4 |
| 35b | 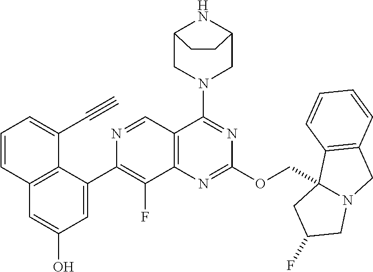 [SMILES: C#Cc1cccc2cc(O)cc(-c3ncc4c(N5CC6CCC(C5)N6)nc(OC[C@@]56C[C@@H](F)CN5Cc5ccccc56)nc4c3F)c12] | Int-20b | | |
| 36 | 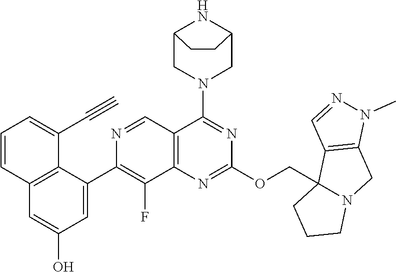 [SMILES: C#Cc1cccc2cc(O)cc(-c3ncc4c(N5CC6CCC(C5)N6)nc(OCC56CCCN5Cc5c6cnn5C)nc4c3F)c12] | 21 | 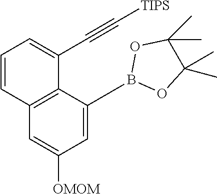 | 617.4 |
| 37 | 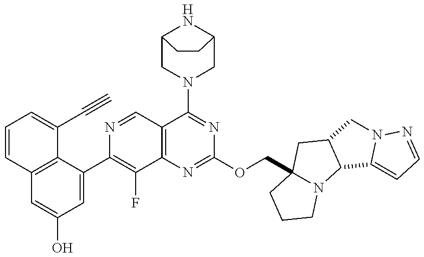 | Int-22 | 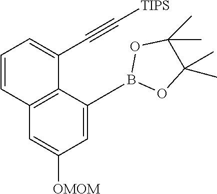 | 643.4 |
| | and | | | |
| | 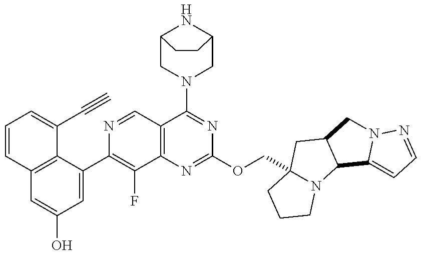 | | | |
| 38 | 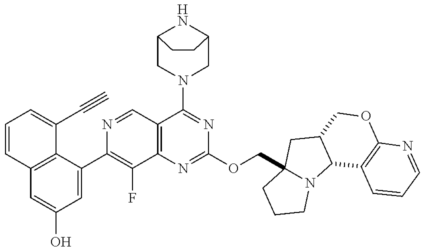 | Int-23 | 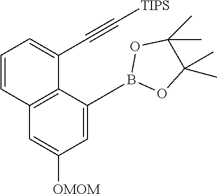 | 670.3 |
| | and | | | |
| | 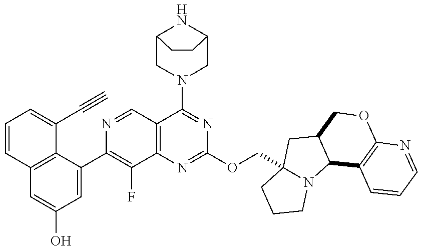 | | | |
| 39 | 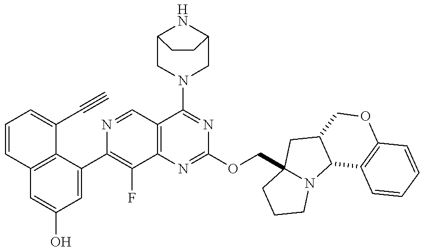 | Int-24 | 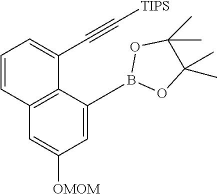 | 669.3 |
| | and | | | |
| | 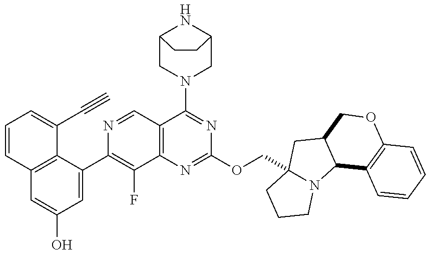 | | | |
| 40 | 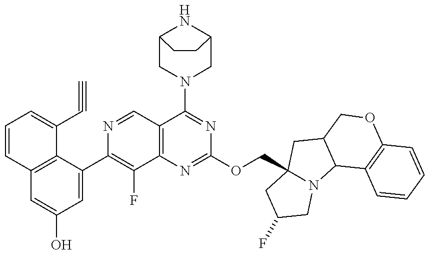 [SMILES: C#Cc1cccc2cc(O)cc(-c3ncc4c(N5CC6CCC(C5)N6)nc(OC[C@@]56CC7COc8ccccc8C7N5C[C@H](F)C6)nc4c3F)c12] | Int-25 | 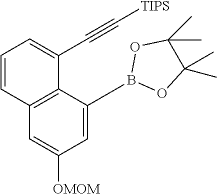 | 687.4 |
| 41 | 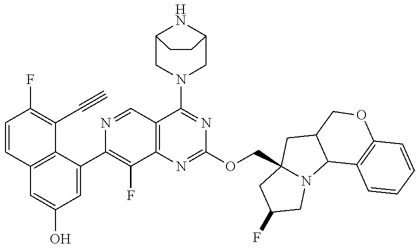 [SMILES: C#Cc1c(F)ccc2cc(O)cc(-c3ncc4c(N5CC6CCC(C5)N6)nc(OC[C@@]56CC7COc8ccccc8C7N5C[C@@H](F)C6)nc4c3F)c12] | Int-26 | 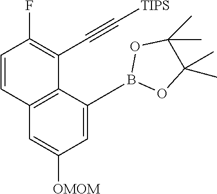 | 705.3 |
| 42 | 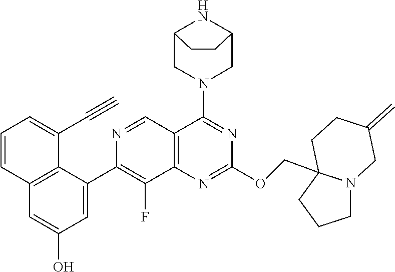 [SMILES: C#Cc1cccc2cc(O)cc(-c3ncc4c(N5CC6CCC(C5)N6)nc(OCC56CCCN5CC(=C)CC6)nc4c3F)c12] | Int-27 | 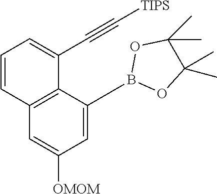 | 591.4 |
| 43 | 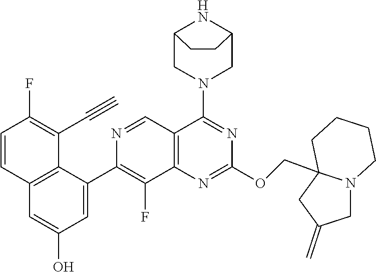 [SMILES: C#Cc1c(F)ccc2cc(O)cc(-c3ncc4c(N5CC6CCC(C5)N6)nc(OCC56CCCCN5CC(=C)C6)nc4c3F)c12] | Int-28 | 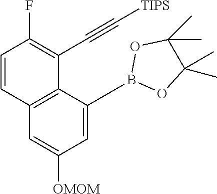 | 609.3 |
| 44 | 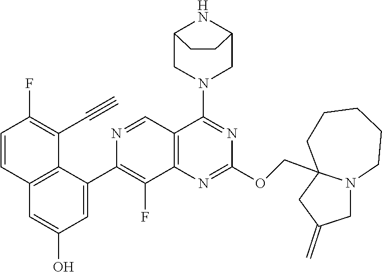 [SMILES: C#Cc1c(F)ccc2cc(O)cc(-c3ncc4c(N5CC6CCC(C5)N6)nc(OCC56CCCCCN5CC(=C)C6)nc4c3F)c12] | Int-29 | 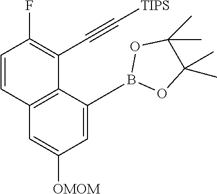 | 623.3 |
| 46 | 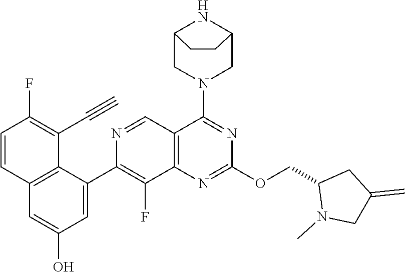 | 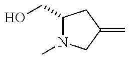 | 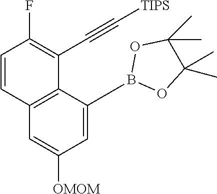 | 569.3 |
| 50 | 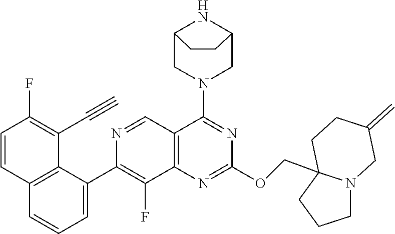 [SMILES: C#Cc1c(F)ccc2cccc(-c3ncc4c(N5CC6CCC(C5)N6)nc(OCC56CCCN5CC(=C)CC6)nc4c3F)c12] | Int-27 | 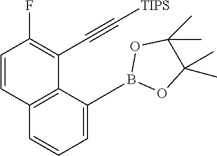 | 593.3 |
| 51 | 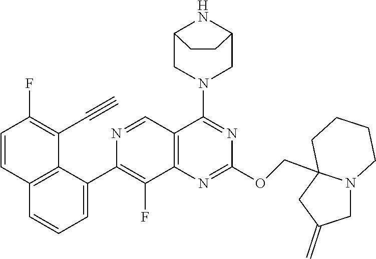 [SMILES: C#Cc1c(F)ccc2cccc(-c3ncc4c(N5CC6CCC(C5)N6)nc(OCC56CCCCN5CC(=C)C6)nc4c3F)c12] | Int-28 | 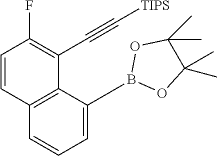 | 593.3 |
| 52 | 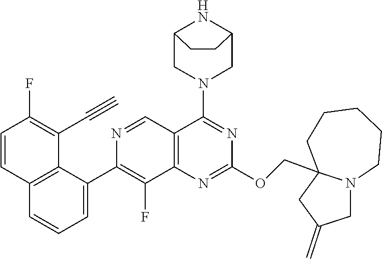 [SMILES: C#Cc1c(F)ccc2cccc(-c3ncc4c(N5CC6CCC(C5)N6)nc(OCC56CCCCCN5CC(=C)C6)nc4c3F)c12] | Int-29 | 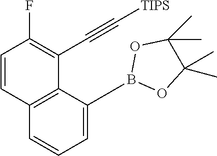 | 608.4 |

### Example 2

### Synthesis of 4-(4-((1R,5S)-3,8-diazabicyclo[3.2.1]octan-3-yl)-8-fluoro-2-((2-(fluoro-methylene)tetrahydro-1H-pyrrolizin-7a(5H)-yl)methoxy)pyrido[4,3-d]pyrimidin-7-yl)-5-ethynylnaphthalen-2-ol

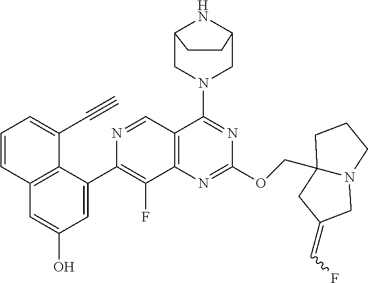 [SMILES: C#Cc1cccc2cc(O)cc(-c3ncc4c(N5CC6CCC(C5)N6)nc(OCC56CCCN5CC(=CF)C6)nc4c3F)c12]

### Step 1: ethyl-2-(fluoromethylene)-5-oxotetrahydro-TH-pyrrolizine-7a(5H)-carboxylate

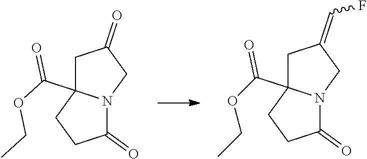 [SMILES: CCOC(=O)C12CCC(=O)N1CC(=O)C2 ; CCOC(=O)C12CCC(=O)N1CC(=CF)C2]

To a stirred mixture of (fluoromethyl)triphenylphosphanium tetrafluoroborate (3.6 g, 9.42 mmol, 2.49 eq.) in THF (26 mL) was added n-BuLi (3.8 mL, 2.5 M, 9.50 mmol, 2.51 eq.) dropwise at −78° C. under nitrogen atmosphere. After stirring for 1 h at −78° C., a solution of ethyl 2,5-dioxo-tetrahydropyrrolizine-7a-carboxylate (800 mg, 3.79 mmol, 1.00 eq.) in THF (30 mL) was added dropwise over 10 min at −78° C. The resulting mixture was warmed and stirred overnight at RT. The reaction mixture was quenched with saturated NH₄Cl (aq.) at RT, diluted with water, and then extracted with EtOAc. The combined organic layer was concentrated and then purified by silica gel column chromatography, eluted with PE/EA (5:2), to afford the title compound (100 mg, 11.6%) as a colorless oil.

### Step 2: (2-(fluoromethylene)tetrahydro-1H-pyrrolizin-7a(5H)-yl)methanol

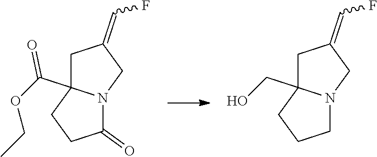 [SMILES: CCOC(=O)C12CCC(=O)N1CC(=CF)C2 ; OCC12CCCN1CC(=CF)C2]

To a stirred solution of ethyl-2-(fluoromethylene)-5-oxotetrahydro-1H-pyrrolizine-7a(5H)-carboxylate (80 mg, 0.35 mmol, 1.00 eq.) in THF (0.80 mL) was added a solution of LiAlH₄ in THF (0.70 mL, 2.0 M, 1.40 mmol, 4.00 eq.) slowly at 0° C. under nitrogen atmosphere. The resulting mixture was refluxed for 3h at 70° C. under nitrogen atmosphere, cooled and then was added Na₂SO₄. The resulting mixture was quenched by addition of water, filtered and the solid cake was washed with THF. The filtrate was concentrated to afford the title compound (70 mg) as a colorless oil, which was used for next step without further purification.

### Step 3: 4-(4-((1R,5S)-3,8-diazabicyclo[3.2.1]octan-3-yl)-8-fluoro-2-((2-(fluoromethylene) tetrahydro-1H-pyrrolizin-7a(5H)-yl)methoxy)pyrido[4,3-d]pyrimidin-7-yl)-5-ethynylnaphthalen-2-ol

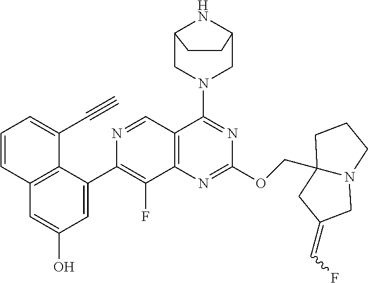 [SMILES: C#Cc1cccc2cc(O)cc(-c3ncc4c(N5CC6CCC(C5)N6)nc(OCC56CCCN5CC(=CF)C6)nc4c3F)c12]

Proceeding analogously as described in Example 1, Steps 4-7, but using (2-(fluoromethylene)tetrahydro-1H-pyrrolizin-7a(5H)-yl)methanol instead of (2-methylidene-tetrahydro-1H-pyrrolizin-7a-yl)methanol in Example 1, Step 4, provided the title compound MS (ES, m/z): [M+1]⁺=595.3.

### Example 3

### Synthesis of 4-(4-((1R,5S)-3,8-diazabicyclo[3.2.1]octan-3-yl)-2-((2-(2,2-difluoroethylidene)-tetrahydro-1H-pyrrolizin-7a(5H)-yl)methoxy)-8-fluoropyrido[4,3-d]pyrimidin-7-yl)-5-ethynylnaphthalen-2-ol

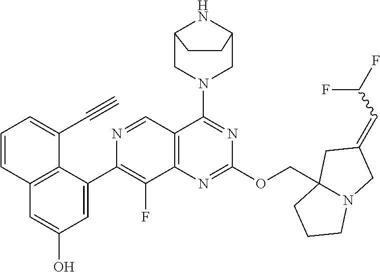 [SMILES: C#Cc1cccc2cc(O)cc(-c3ncc4c(N5CC6CCC(C5)N6)nc(OCC56CCCN5CC(=CC(F)F)C6)nc4c3F)c12]

### Step 1: potassium 2-bromo-2,2-difluoroacetate

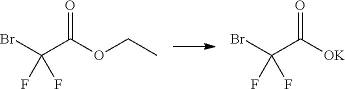

To a stirred mixture of KOH (4.15 g, 73.90 mmol, 1.00 eq.) in MeOH (70 mL) was added ethyl 2-bromo-2,2-difluoroacetate (15.0 g, 73.90 mmol, 1.00 eq.) dropwise at 0° C. The resulting mixture was stirred for 16h at RT and then concentrated. The residue was triturated with DCM to give the title compound (14.0 g, 88.9%) as a white solid.

### Step 2: 2,2-difluoro-2-(triphenylphosphonio)acetate

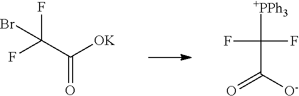

To a stirred mixture of potassium 2-bromo-2,2-difluoroacetate (14.0 g, 65.72 mmol, 1.00 eq.) in DMF (140 mL) was added PPh₃ (34.0 g, 129.63 mmol, 1.97 eq.) at RT under N2 atmosphere. After stirring at RT for 16 h, the reaction mixture was filtered and the solid cake wash washed with cool DMF, water and then Et₂O to give the title compound (12.0 g, 51.2%) as a white solid.

### Step 3: (difluoromethyl)triphenylphosphonium bromide

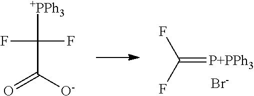

To a stirred mixture of 2,2-difluoro-2-(triphenylphosphonio)acetate (11.0 g, 30.87 mmol, 1.00 eq.) in THF (30 mL) was added 40% HBr aqueous solution (5.0 mL) at RT under N₂ atmosphere. The resulting mixture was refluxed for 1h, cooled, concentrated to remove most THF, and then extracted with DCM. The combined organic layers were washed with water, brine, dried over anhydrous Na₂SO₄, filtered and then concentrated. The residue was purified by trituration with Et₂O to give the title compound (8.0 g, 66.1%) as a white solid.

### Step 4: ethyl 2-(2,2-difluoroethylidene)-5-oxotetrahydro-1H-pyrrolizine-7a(5H)-carboxylate

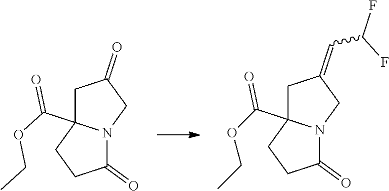 [SMILES: CCOC(=O)C12CCC(=O)N1CC(=O)C2 ; CCOC(=O)C12CCC(=O)N1CC(=CC(F)F)C2]

A mixture of Ir(ppy)₃ (75 mg, 0.115 mmol, 0.03 eq.), CuBr₂ (171 mg, 0.765 mmol, 0.2 equiv), (difluoromethyl)triphenylphosphonium bromide (4.51 g, 11.499 mmol, 3.00 eq.) and ethyl 2-methylidene-5-oxo-tetrahydropyrrolizine-7a-carboxylate (800 mg, 3.823 mmol, 1.00 eq.) in DMF (5 mL) was degassed for three times by the freeze-pump-thaw procedure. The reaction mixture was irradiated with blue LEDs for 10 h, and then was added DBU (3.49 g, 22.924 mmol, 6.00 eq.). After stirring for 10h, the reaction mixture was poured into brine, extracted with EtOAc. The combined organic layers were washed with brine, dried with Na₂SO₄ and then concentrated. The residue was purified by column chromatography on silica gel to give the title compound (380 mg, 38.3%) as a light-yellow solid.

### Step 5: (2-(2,2-difluoroethylidene)tetrahydro-1H-pyrrolizin-7a(5H)-yl)methanol

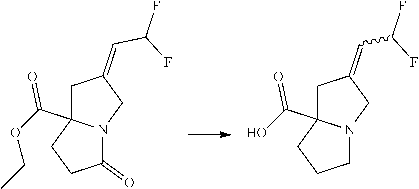 [SMILES: CCOC(=O)C12CCC(=O)N1C/C(=C\C(F)F)C2 ; O=C(O)C12CCCN1CC(=CC(F)F)C2]

To a stirred solution of ethyl 2-(2,2-difluoroethylidene)-5-oxotetrahydro-1H-pyrrolizine-7a(5H)-carboxylate (300 mg, 1.157 mmol, 1.00 eq.) in THF (3.0 mL) was charged with 2.0 M LiAlH₄ in THF (2.3 mL, 4.60 mmol, 3.98 eq.) over 10 min at 0° C. under nitrogen atmosphere. The resulting mixture was stirred for 10 min at 0° C., and then refluxed for 3h. To the cooled reaction mixture was added Na₂SO₄, followed by water slowly at RT. The resulting mixture was filtered and the solid cake was washed with THF. The filtrate was concentrated to afford the title compound (230 mg, 97.8%) as a colorless oil. MS (ES, m/z): [M+1]⁺=228.2.

### Step 6: 4-(4-((1R,5S)-3,8-diazabicyclo[3.2.1]octan-3-yl)-2-(((E)-2-(2,2-difluoroethylidene-)tetrahydro-1H-pyrrolizin-7a(5H)-yl)methoxy)-8-fluoropyrido[4,3-d]pyrimidin-7-yl)-5-ethynylnaphthalen-2-ol

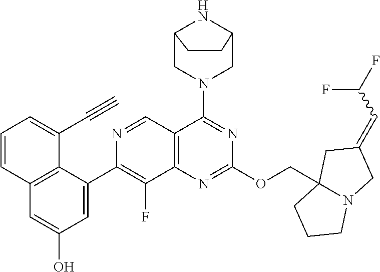 [SMILES: C#Cc1cccc2cc(O)cc(-c3ncc4c(N5CC6CCC(C5)N6)nc(OCC56CCCN5CC(=CC(F)F)C6)nc4c3F)c12]

Proceeding analogously as described in Example 1, Steps 4-7, but using (2-(2,2-difluoroethylidene)tetrahydro-1H-pyrrolizin-7a(5H)-yl)methanol instead of (2-methylidene-tetrahydro-1H-pyrrolizin-7a-yl)methanol, in Example 1, Step 4, provided the the title compound. MS (ES, m/z): [M+1]⁺=627.3.

### Example 4

### Synthesis of a mixture of 4-(4-((1R,5S)-3,8-diazabicyclo[3.2.1]octan-3-yl)-8-fluoro-2-(((3aR,7aR,8aS)-hexahydro-1H-furo[3,4-b]pyrrolizin-7a(5H)-yl)methoxy)pyrido[4,3-d]pyrimidin-7-yl)-5-ethynylnaphthalen-2-ol and 4-(4-((1R,5S)-3,8-diazabicyclo[3.2.1]octan-3-yl)-8-fluoro-2-(((3aS,7aS,8aR)-hexahydro-1H-furo[3,4-b]pyrrolizin-7a(5H)-yl)methoxy)pyrido[4,3-d]pyrimidin-7-yl)-5-ethynylnaphthalen-2-ol

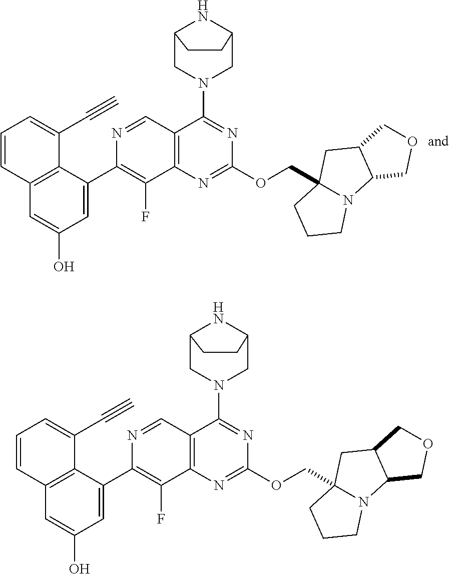

### Step 1: 2-(tert-butyl) 7a-methyl (2S,3R,7aR)-3-((benzyloxy)methyl)tetrahydro-1H-pyrrolizine-2,7a(5H)-dicarboxylate and 2-(tert-butyl) 7a-methyl (2R,3S,7aS)-3-((benzyloxy)methyl)-tetrahydro-1H-pyrrolizine-2,7a(5H)-dicarboxylate

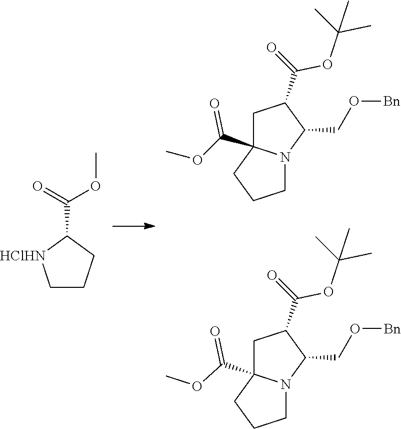

To a stirred solution of methyl L-prolinate hydrochloride (22.0 g, 132.83 mmol, 1.00 eq.) in toluene (800 mL) were added AgOAc (1.11 g, 6.65 mmol, 0.050 eq.), 2-(benzyloxy) acetaldehyde (19.95 g, 132.84 mmol, 1.00 eq.), tert-butyl acrylate (17.00 g, 132.64 mmol, 1.00 eq.) and triethylamine (14.79 g, 146.16 mmol, 1.10 eq.) at RT. After stirring at RT for 16 h under dark condition, the reaction mixture was concentrated and the residue was purified by silica gel column chromatography, eluted with EA/PE (0-50%), to afford the title compounds (2.50 g, 4.8%) as a light-yellow oil.

### Step 2: 2-(tert-butyl) 7a-methyl (2S,3R,7aR)-3-(hydroxymethyl)tetrahydro-1H-pyrrolizine-2,7a(5H)-dicarboxylate and 2-(tert-butyl) 7a-methyl (2R,3S,7aS)-3-(hydroxymethyl)tetrahydro-1H-pyrrolizine-2,7a(5H)-dicarboxylate

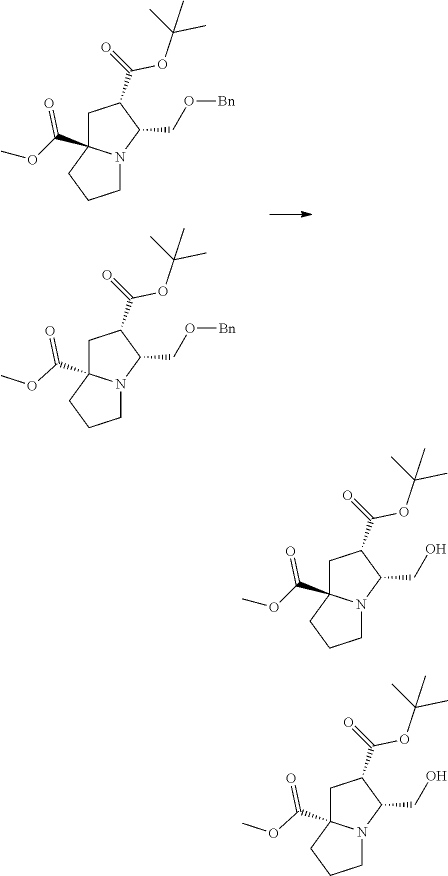 [SMILES: COC(=O)[C@]12CCCN1[C@@H](COCc1ccccc1)[C@@H](C(=O)OC(C)(C)C)C2 ; COC(=O)[C@@]12CCCN1[C@@H](COCc1ccccc1)[C@@H](C(=O)OC(C)(C)C)C2 ; COC(=O)[C@]12CCCN1[C@@H](CO)[C@@H](C(=O)OC(C)(C)C)C2 ; COC(=O)[C@@]12CCCN1[C@@H](CO)[C@@H](C(=O)OC(C)(C)C)C2]

A mixture of 2-(tert-butyl) 7a-methyl (2S,3R,7aR)-3-((benzyloxy)methyl)tetrahydro-1H-pyrrolizine-2,7a(5H)-dicarboxylate and 2-(tert-butyl) 7a-methyl (2R,3S,7aS)-3-((benzyloxy)-methyl)tetrahydro-1H-pyrrolizine-2,7a(5H)-dicarboxylate (2.40 g, 6.16 mmol, 1.00 eq.), HCl (g) in MeOH (1 drop) and 10% Pd/C (7.20 g) in MeOH (240 mL) was stirred for 16 h at 50° C. under hydrogen atmosphere (20 atm). The reaction mixture was basified to pH=9 with ammonia aqueous solution. The resulting mixture was filtered, and the filter cake was washed with MeOH. The filtrate was concentrated, and the residue was purified by silica gel column chromatography, eluted with MeOH/CH₂Cl₂ (0-10%), to afford the title compounds (900 mg, 48.9%) as a yellow oil.

### Step 3: 2-(tert-butyl) 7a-methyl (2S,3R,7aR)-3-((tosyloxy)methyl)tetrahydro-1H-pyrrolizine-2,7a(5H)-dicarboxylate and 2-(tert-butyl) 7a-methyl (2R,3S,7aS)-3-((tosyloxy)methyl)tetrahydro-1H-pyrrolizine-2,7a(5H)-dicarboxylate

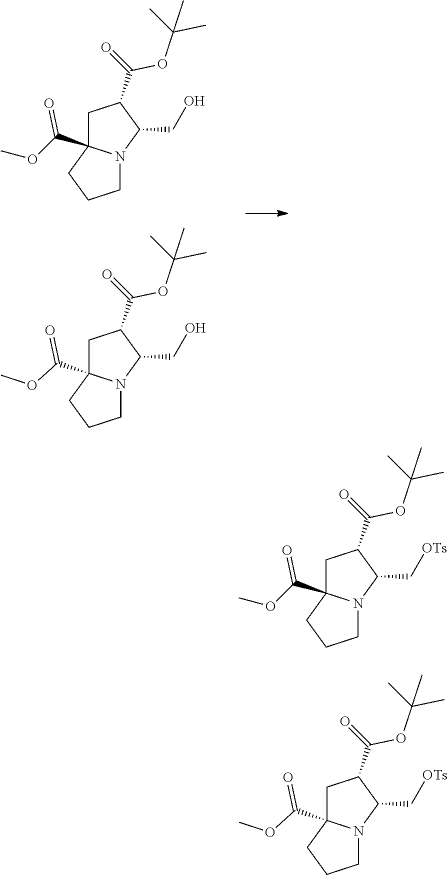 [SMILES: COC(=O)[C@]12CCCN1[C@@H](CO)[C@@H](C(=O)OC(C)(C)C)C2 ; COC(=O)[C@@]12CCCN1[C@@H](CO)[C@@H](C(=O)OC(C)(C)C)C2 ; COC(=O)[C@]12CCCN1[C@@H](COS(=O)(=O)c1ccc(C)cc1)[C@@H](C(=O)OC(C)(C)C)C2 ; COC(=O)[C@@]12CCCN1[C@@H](COS(=O)(=O)c1ccc(C)cc1)[C@@H](C(=O)OC(C)(C)C)C2]

To a stirred solution of 2-(tert-butyl) 7a-methyl (2S,3R,7aR)-3-(hydroxymethyl) tetrahydro-1H-pyrrolizine-2,7a(5H)-dicarboxylate and 2-(tert-butyl) 7a-methyl (2R,3S,7aS)-3-(hydroxymethyl)tetrahydro-1H-pyrrolizine-2,7a(5H)-dicarboxylate (900 mg, 3.01 mmol, 1.00 eq.), DMAP (73 mg, 0.60 mmol, 0.20 eq.) and triethylamine (608 mg, 6.01 mmol, 2.00 eq.) in DCM (18 mL) was added a solution of p-toluenesulfonyl chloride (745 mg, 3.91 mmol, 1.30 eq.) in DCM (3.6 mL) dropwise at 5° C. The resulting mixture was stirred at RT for 2 h, concentrated and then purified by silica gel column chromatography, eluted with EA/PE (0-50%), to afford the title compound (900 mg, 65.8%) as a colorless oil.

### Step 4: ((2S,3R,7aR)-2,7a-bis(hydroxymethyl)hexahydro-1H-pyrrolizin-3-yl)methyl 4-methyl-benzenesulfonate and ((2R,3S,7aS)-2,7a-bis(hydroxymethyl)hexahydro-1H-pyrrolizin-3-yl)methyl 4-methylbenzenesulfonate

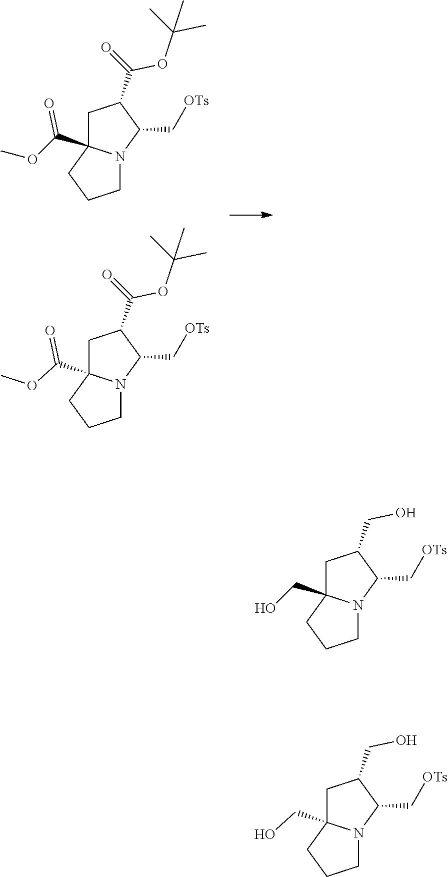 [SMILES: COC(=O)[C@]12CCCN1[C@@H](COS(=O)(=O)c1ccc(C)cc1)[C@@H](C(=O)OC(C)(C)C)C2 ; COC(=O)[C@@]12CCCN1[C@@H](COS(=O)(=O)c1ccc(C)cc1)[C@@H](C(=O)OC(C)(C)C)C2 ; Cc1ccc(S(=O)(=O)OC[C@H]2[C@@H](CO)C[C@@]3(CO)CCCN23)cc1 ; Cc1ccc(S(=O)(=O)OC[C@H]2[C@@H](CO)C[C@]3(CO)CCCN23)cc1]

To a stirred solution of 2-(tert-butyl) 7a-methyl (2S,3R,7aR)-3-((tosyloxy)methyl) tetrahydro-1H-pyrrolizine-2,7a(5H)-dicarboxylate and 2-(tert-butyl) 7a-methyl (2R,3S,7aS)-3-((tosyloxy)methyl)tetrahydro-1H-pyrrolizine-2,7a(5H)-dicarboxylate (760 mg, 1.68 mmol, 1.00 eq.) in THF (5.0 mL) was added 1.0 M LiAlH₄ in THF (5.0 mL, 5.00 mmol, 2.98 eq.) dropwise at 5° C. After stirring at 5° C. for additional 1h, the reaction mixture was diluted with THF, added Na₂SO₄, quenched with water at 5° C. The resulting mixture was filtered, and the filter cake was washed with THF. The filtrate was concentrated to afford the crude title compounds (450 mg, 75.6%) as a colorless oil, which was used for next step without further purification.

### Step 5: ((3aR,7aR,8aS)-hexahydro-1H-furo[3,4-b]pyrrolizin-7a(5H)-yl)methanol and ((3aS,7aS,8aR)-hexahydro-1H-furo[3,4-b]pyrrolizin-7a(5H)-yl)methanol

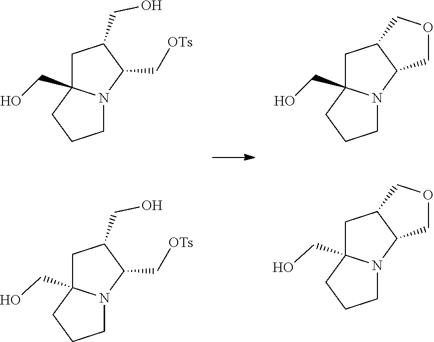 [SMILES: Cc1ccc(S(=O)(=O)OC[C@H]2[C@@H](CO)C[C@@]3(CO)CCCN23)cc1 ; OC[C@]12CCCN1[C@H]1COC[C@H]1C2 ; Cc1ccc(S(=O)(=O)OC[C@H]2[C@@H](CO)C[C@]3(CO)CCCN23)cc1 ; OC[C@@]12CCCN1[C@H]1COC[C@H]1C2]

To a stirred solution of ((2S,3R,7aR)-2,7a-bis(hydroxymethyl)hexahydro-1H-pyrrolizin-3-yl)methyl 4-methylbenzenesulfonate and ((2R,3S,7aR)-2,7a-bis(hydroxymethyl)hexahydro-1H-pyrrolizin-3-yl)methyl 4-methylbenzenesulfonate (450 mg, 1.27 mmol, 1.00 eq.) in THF (12 mL) was added 60% sodium hydride (152 mg, 3.80 mmol, 3.00 eq.) in portions at 5° C. The resulting mixture was stirred at RT for 40 h. This reaction mixture was used for next step directly without further purification.

### Step 6: 4-(4-((1R,5S)-3,8-diazabicyclo[3.2.1]octan-3-yl)-8-fluoro-2-(((3aR,7aR,8aS)-hexahydro-1H-furo[3,4-b]pyrrolizin-7a(5H)-yl)methoxy)pyrido[4,3-d]pyrimidin-7-yl)-5-ethynylnaphthalen-2-ol and 4-(4-((1R,5S)-3,8-diazabicyclo[3.2.1]octan-3-yl)-8-fluoro-2-(((3aS,7aS,8aR)-hexahydro-1H-furo[3,4-b]pyrrolizin-7a(5H)-yl)methoxy)pyrido[4,3-d]pyrimidin-7-yl)-5-ethynylnaphthalen-2-ol

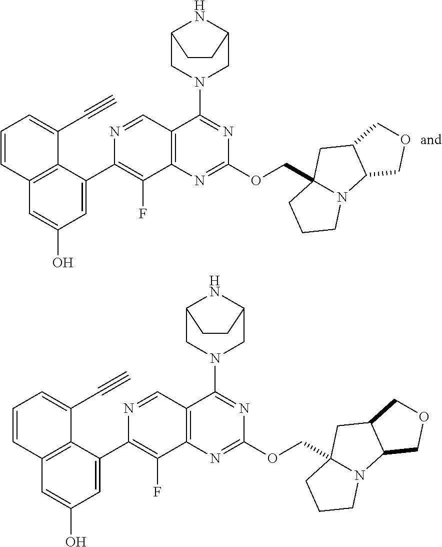

Proceeding analogously as described in Example 1, Steps 4-7 above, but using ((3aR,7aR,8aS)-hexahydro-1H-furo[3,4-b]pyrrolizin-7a(5H)-yl)methanol and ((3aS,7aS,8aR)-hexahydro-1H-furo[3,4-b]pyrrolizin-7a(5H)-yl)methanol instead of (2-methylidene-tetrahydro-1H-pyrrolizin-7a-yl)methanol, in Example 1, Step 4 provided the title compounds. MS (ES, m/z): [M+1]⁺=607.3.

### Example 5

### Synthesis of 4-(4-((1R,5S)-3,8-diazabicyclo[3.2.1]octan-3-yl)-2-((2,3-dihydro-1H-pyrrolo[1,2-a]-indol-9a(9H)-yl)methoxy)-8-fluoropyrido[4,3-d]pyrimidin-7-yl)-5-ethynylnaphthalen-2-ol

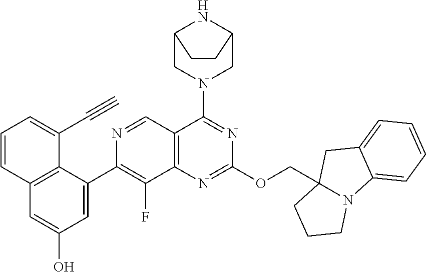 [SMILES: C#Cc1cccc2cc(O)cc(-c3ncc4c(N5CC6CCC(C5)N6)nc(OCC56CCCN5c5ccccc5C6)nc4c3F)c12]

### Step 1: 1-(tert-butyl) 2-ethyl 1H-indole-1,2-dicarboxylate

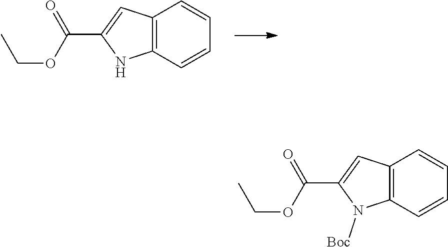 [SMILES: CCOC(=O)c1cc2ccccc2[nH]1 ; CCOC(=O)c1cc2ccccc2n1C(=O)OC(C)(C)C]

To a stirred solution of ethyl 1H-indole-2-carboxylate (30.0 g, 158.66 mmol, 1.00 eq.) and DMAP (3.87 g, 31.68 mmol, 0.20 eq.) in CH₃CN (400 mL) was added a solution of Boc20 (69.2 g, 317.07 mmol, 2.00 eq.) in CH₃CN (200 mL) dropwise at 0° C. under nitrogen atmosphere. The reaction mixture was stirred for 3h at RT, concentrated and then purified by silica gel column chromatography, eluted with PE/EA (7:1), to afford the title compound (38.1 g, 83.0%) as a white solid.

### Step 2: 1-(tert-butyl) 2-ethyl indoline-1,2-dicarboxylate

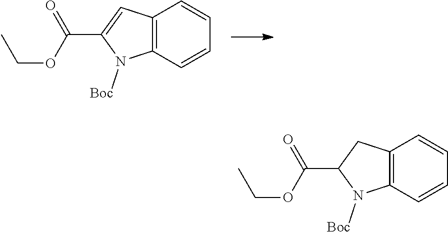 [SMILES: CCOC(=O)c1cc2ccccc2n1C(=O)OC(C)(C)C ; CCOC(=O)C1Cc2ccccc2N1C(=O)OC(C)(C)C]

A mixture of 1-(tert-butyl) 2-ethyl 1H-indole-1,2-dicarboxylate (38.1 g, 131.68 mmol, 1.00 eq.) and 10% Pd/C (3.8 g) in EtOH (380 mL) was stirred for 6 h at RT under hydrogen atmosphere (1 atm). The reaction mixture was filtered and concentrated to give the title compound (37.0 g, 96.4%) as an off-white solid.

### Step 3: 1-(tert-butyl) 2-ethyl 2-(3-chloropropyl)indoline-1,2-dicarboxylate

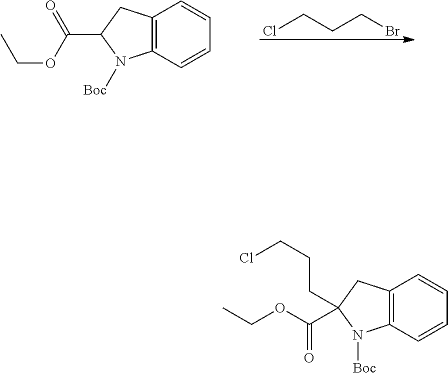 [SMILES: CCOC(=O)C1Cc2ccccc2N1C(=O)OC(C)(C)C ; ClCCCBr ; CCOC(=O)C1(CCCCl)Cc2ccccc2N1C(=O)OC(C)(C)C]

To a stirred solution of 1-(tert-butyl) 2-ethyl indoline-1,2-dicarboxylate (15.0 g, 51.48 mmol, 1.00 eq.) in THF (300 mL) was added 1.0 M LiHMDS in THF (77 mL, 77 mmol, 1.50 eq.) dropwise at −78° C. under nitrogen atmosphere. After stirring for 30 min, 1-chloro-3-bromopropane (40.5 g, 257.24 mmol, 5.00 eq.) was added dropwise at −78° C. The resulting mixture was warm to RT and then stirred overnight. The reaction mixture was quenched with saturated NH₄Cl (aq.) and then extracted with EtOAc. The combined organic layers were washed with water, brine, dried over anhydrous Na₂SO₄, filtered and then concentrated. The residue was purified by silica gel column chromatography, eluted with EtOAc/PE (0-30%), to afford the title compound (10.0 g, 52.8%) as a yellow oil.

### Step 4: ethyl 2-(3-chloropropyl)indoline-2-carboxylate

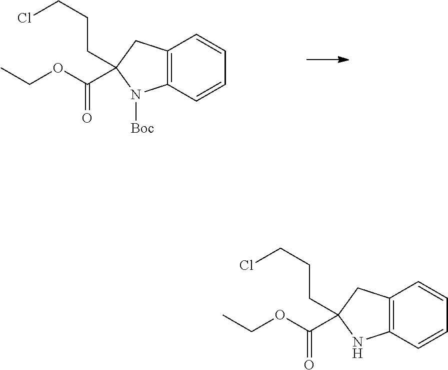 [SMILES: CCOC(=O)C1(CCCCl)Cc2ccccc2N1C(=O)OC(C)(C)C ; CCOC(=O)C1(CCCCl)Cc2ccccc2N1]

A mixture of 1-(tert-butyl) 2-ethyl 2-(3-chloropropyl)indoline-1,2-dicarboxylate (10.0 g, 27.18 mmol, 1.00 eq.) in TFA (100 mL) was stirred for 30 min at RT. The reaction mixture was concentrated and the residue was purified by silica gel column chromatography, eluted with EA/PE (0-70%), to afford the title compound (7.0 g, 96.2%) as a yellow oil.

### Step 5: ethyl 2,3-dihydro-1H-pyrrolo[1,2-a]indole-9a(9H)-carboxylate

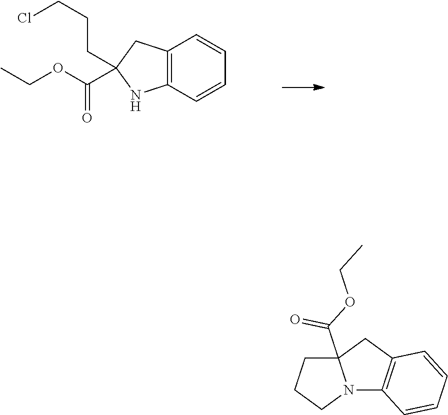 [SMILES: CCOC(=O)C1(CCCCl)Cc2ccccc2N1 ; CCOC(=O)C12CCCN1c1ccccc1C2]

A mixture of ethyl ethyl 2-(3-chloropropyl)indoline-2-carboxylate (1.50 g, 5.60 mmol, 1.00 eq.) and K₂CO₃ (3.87 g, 28.00 mmol, 5.00 eq.) in EtOH (30 mL) was stirred for 2 h at RT. The resulting mixture was diluted with water and then extracted with EtOAc. The combined organic layers were washed with water, brine, dried over anhydrous Na₂SO₄, filtered and then concentrated to give the title compound (1.02 g, 78.8%) as a yellow oil.

### Step 6: (2,3-dihydro-1H-pyrrolo[1,2-a]indol-9a(9H)-yl)methanol

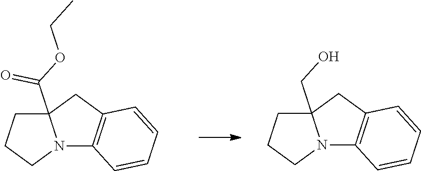 [SMILES: CCOC(=O)C12CCCN1c1ccccc1C2 ; OCC12CCCN1c1ccccc1C2]

To a stirred solution of ethyl 2,3-dihydro-1H-pyrrolo[1,2-a]indole-9a(9H)-carboxylate (1.0 g, 4.32 mmol, 1.00 eq.) in THF (10 mL) was added 1.0 M LiAlH₄ in THF (5.2 mL, 5.2 mmol, 1.20 eq.) dropwise at RT. The resulting mixture was stirred for 2 h at RT, quenched by addition of MeOH and Na₂SO₄·10H₂O. The resulting mixture was filtered and then concentrated. The residue was purified by silica gel column chromatography, eluted with CH₂Cl₂/MeOH (10:1), to afford the title compound (450 mg, 55.1%) as a yellow oil.

### Step 7: 4-(4-((1R,5S)-3,8-diazabicyclo[3.2.1]octan-3-yl)-2-((2,3-dihydro-1H-pyrrolo[1,2-a]indol-9a(9H)-yl)methoxy)-8-fluoropyrido[4,3-d]pyrimidin-7-yl)-5-ethynylnaphthalen-2-ol

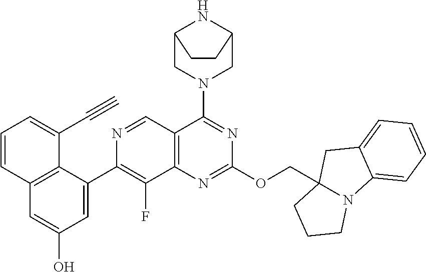 [SMILES: C#Cc1cccc2cc(O)cc(-c3ncc4c(N5CC6CCC(C5)N6)nc(OCC56CCCN5c5ccccc5C6)nc4c3F)c12]

Proceeding analogously as described in Example 1, Steps 4-7 above, but using (2,3-dihydro-1H-pyrrolo[1,2-a]-indol-9a(9H)-yl)methanol instead of (2-methylidene-tetrahydro-1H-pyrrolizin-7a-yl)methanol provided the title compound. MS (ES, m/z): [M+1]⁺=613.3.

### Example 6

### Synthesis of 4-(4-((1R,5S)-3,8-diazabicyclo[3.2.1]octan-3-yl)-2-((2,3-dihydro-1H-pyrrolo[2,1-a]-isoindol-9b(5H)-yl)methoxy)-8-fluoropyrido[4,3-d]pyrimidin-7-yl)-5-ethynylnaphthalen-2-ol

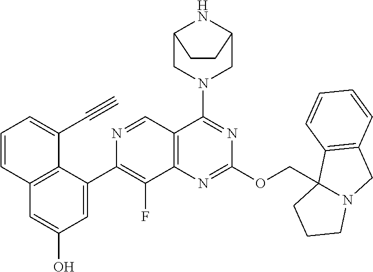 [SMILES: C#Cc1cccc2cc(O)cc(-c3ncc4c(N5CC6CCC(C5)N6)nc(OCC56CCCN5Cc5ccccc56)nc4c3F)c12]

### Step 1: methyl (2-bromobenzyl)prolinate

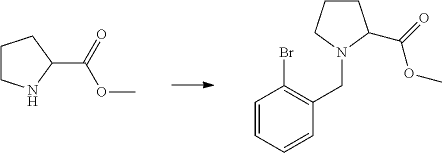

A solution of methyl prolinate (5.0 g, 38.71 mmol, 1.00 eq.) and 2-bromobenzaldehyde (14.32 g, 77.40 mmol, 2.00 eq.) in DCM (50 mL) was stirred at 25° C. for 1 h. To this solution was added NaBH(OAc)₃ (16.41 g, 77.43 mmol, 2.00 eq.) in portions at 5° C. and the resulting mixture was stirred at 25° C. for 5h. The reaction mixture was diluted with water and then extracted with EtOAc. The combined organic layers were washed with brine, dried over anhydrous Na₂SO₄, filtered and then concentrated. The residue was purified by silica gel column chromatography, eluted with PE/EA (9/1), to afford the title compound (4.5 g, 39.0%) as a colorless oil.

### Step 2: tert-butyl 2,3-dihydro-1H-pyrrolo[2,1-a]isoindole-9b(5H)-carboxylate

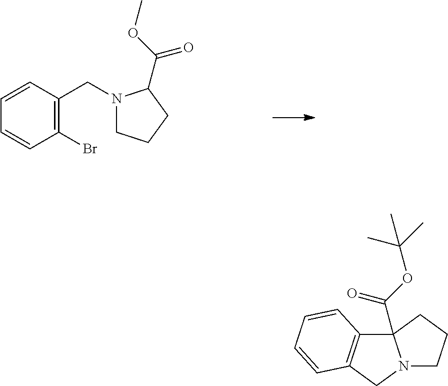 [SMILES: COC(=O)C1CCCN1Cc1ccccc1Br ; CC(C)(C)OC(=O)C12CCCN1Cc1ccccc12]

A mixture of t-BuOLi (2.35 g, 29.34 mmol, 3.50 eq.), DavePhos (0.16 g, 0.42 mmol, 0.05 eq.) and Pd₂(dba)₃ (0.15 g, 0.17 mmol, 0.02 eq.) in 1,4-dioxane (25 mL) was stirred at 25° C. for 5 min under N₂ atmosphere. To this mixture were added dodecane (0.43 g, 2.52 mmol, 0.30 eq.) and methyl (2-bromobenzyl)prolinate (2.5 g, 8.38 mmol, 1.00 eq.) at 25° C. The resulting mixture was stirred at 85° C. for 12 h under N₂ atmosphere, diluted with water and then extracted with DCM. The combined organic layers were washed with brine, dried over anhydrous Na₂SO₄, filtered and then concentrated. The residue was purified by silica gel column chromatography, eluted with PE/EA (1/5), to afford the title compound (1.1 g, 50.6%) as a light-yellow solid.

### Step 3: (2,3-dihydro-1H-pyrrolo[2,1-a]isoindol-9b(5H)-yl)methanol

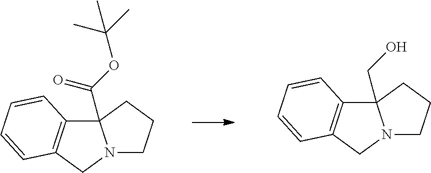 [SMILES: CC(C)(C)OC(=O)C12CCCN1Cc1ccccc12 ; OCC12CCCN1Cc1ccccc12]

To a stirred solution of tert-butyl 2,3-dihydro-1H-pyrrolo[2,1-a]isoindole-9b(5H)-carboxylate (1.0 g, 3.86 mmol, 1.00 eq.) in THF (1.0 mL) was added 1.0 M LiAlH₄ in THF (5.8 mL, 5.8 mmol, 1.50 eq.) dropwise at 0° C. under N₂ atmosphere. The resulting mixture was stirred at 0° C. for 1h, quenched with water, 15% aqueous NaOH and then water in sequence at 0° C. The resulting mixture was filtered, and the filter cake was washed with EtOAc. The filtrate was concentrated to give the title compound (369.7 mg, 50.6%) as a light-yellow solid.

### Step 4: 4-(4-((1R,5S)-3,8-diazabicyclo[3.2.1]octan-3-yl)-2-((2,3-dihydro-1H-pyrrolo[2,1-a]isoindol-9b(5H)-yl)methoxy)-8-fluoropyrido[4,3-d]pyrimidin-7-yl)-5-ethynylnaphthalen-2-ol

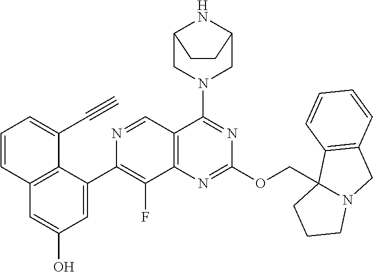 [SMILES: C#Cc1cccc2cc(O)cc(-c3ncc4c(N5CC6CCC(C5)N6)nc(OCC56CCCN5Cc5ccccc56)nc4c3F)c12]

Proceeding as described in Example 1, Steps 4 to 7, using: (2,3-dihydro-1H-pyrrolo[2,1-a]isoindol-9b(5H)-yl)methanol instead of (2-methylidene-tetrahydro-1H-pyrrolizin-7a-yl)methanol provided the title compound. MS (ES, m/z): [M+1]⁺=613.3.

### Example 7

### Synthesis of 3-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]oct-6-ene-6-carbonitrile

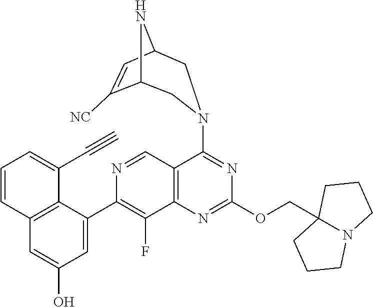 [SMILES: C#Cc1cccc2cc(O)cc(-c3ncc4c(N5CC6C=C(C#N)C(C5)N6)nc(OCC56CCCN5CCC6)nc4c3F)c12]

### Step 1: tert-butyl 3-bromo-9-(2-phenylpropan-2-yl)-3a,4,5,7,8,8a-hexahydro-6H-4,8-epiminoisoxazolo[4,5-d]azepine-6-carboxylate

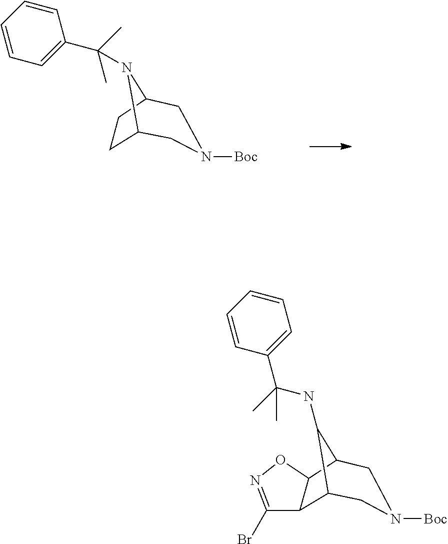 [SMILES: CC(C)(C)OC(=O)N1CC2CCC(C1)N2C(C)(C)c1ccccc1 ; CC(C)(C)OC(=O)N1CC2C(NC(C)(C)c3ccccc3)C(C1)C1C(Br)=NOC21]

A mixture of tert-butyl (1R,5S)-8-(2-phenylpropan-2-yl)-3,8-diazabicyclo[3.2.1]oct-6-ene-3-carboxylate (1.00 g, 3.04 mmol, 1.00 eq.), hydroxycarbonimidic dibromide (1.24 g, 6.11 mmol, 2.00 eq.) and NaHCO₃ (1.28 g, 15.24 mmol, 5.01 eq.) in EtOAc (10 mL) was stirred for 16 h at 75° C. under nitrogen atmosphere. The reaction mixture was cooled, quenched with water, and then extracted with EtOAc. The combined organic layers were washed with brine, dried with Na₂SO₄, filtered and then concentrated. The residue was purified by silica gel column chromatography, eluted with PE/EA (12:1), to afford the title compound (0.95 g, 69.4%) as a white solid.

### Step 2: tert-butyl 6-cyano-7-hydroxy-8-(2-phenylpropan-2-yl)-3,8-diazabicyclo[3.2.1]octane-3-carboxylate

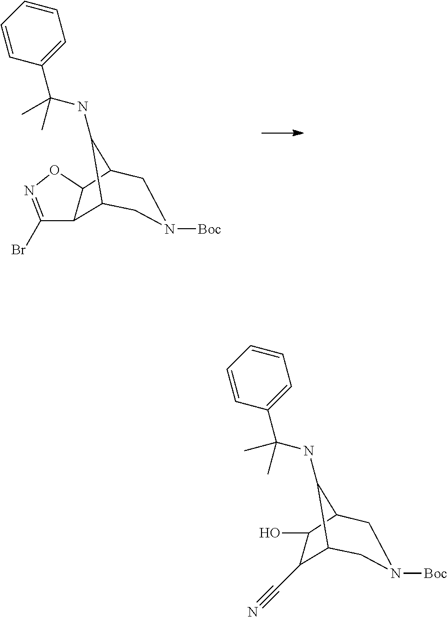 [SMILES: CC(C)(C)OC(=O)N1CC2C(NC(C)(C)c3ccccc3)C(C1)C1C(Br)=NOC21 ; CC(C)(C)OC(=O)N1CC2C(O)C(C#N)C(C1)C2NC(C)(C)c1ccccc1]

A mixture of tert-butyl (3aR,4S,8S,8aR)-3-bromo-9-(2-phenylpropan-2-yl)-3a,4,5,7,8,8a-hexahydro-6H-4,8-epiminoisoxazolo[4,5-d]azepine-6-carboxylate (900 mg, 2.00 mmol, 1.00 eq.), boric acid (371 mg, 6.00 mmol, 3.00 eq.) and Raney Ni (8.56 mg) in MeOH (5 mL) and H₂O (1 mL) was stirred for 2 h at RT under hydrogen atmosphere (2 atm). The resulting mixture was filtered, and the filter cake was washed with MeOH. The filtrate was concentrated under reduced pressure to give the title compound (380 mg, 51.0%) as an off-white oil.

### Step 3: tert-butyl 6-cyano-8-(2-phenylpropan-2-yl)-3,8-diazabicyclo[3.2.1]oct-6-ene-3-carboxylate

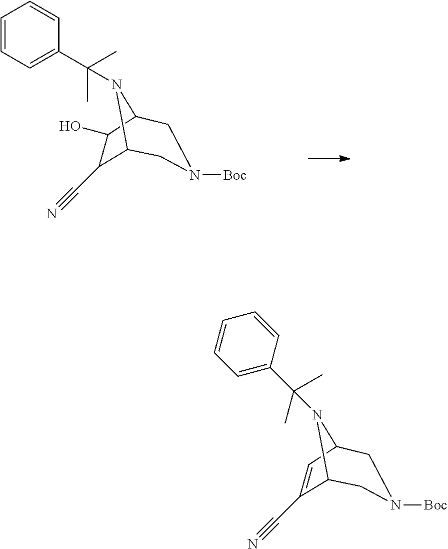 [SMILES: CC(C)(C)OC(=O)N1CC2C(O)C(C#N)C(C1)N2C(C)(C)c1ccccc1 ; CC(C)(C)OC(=O)N1CC2C=C(C#N)C(C1)N2C(C)(C)c1ccccc1]

To a stirred solution of tert-butyl (1S,5S,7R)-6-cyano-7-hydroxy-8-(2-phenylpropan-2-yl)-3,8-diazabicyclo[3.2.1]octane-3-carboxylate (380 mg, 1.02 mmol, 1,00 eq.) and TEA (207.02 mg, 2.05 mmol, 2.00 eq.) in DCM (10 mL) was added MsCl (140.61 mg, 1.23 mmol, 1.20 eq.) dropwise at 0° C. under nitrogen atmosphere. After stirring at RT for 16 h, the reaction mixture was quenched with water and then extracted with DCM. The combined organic layers were concentrated. The residue was dissolved in DCE (10 mL), and to the resulting solution was added DBU (467.18 mg, 3.07 mmol, 3.00 eq.) dropwise at RT. After stirring further at RT for 1h, the reaction mixture was quenched with water and then extracted with EtOAc. The organic layer was dried over Na₂SO₄, filtered and then concentrated. The residue was purified by silica gel column chromatography, eluted with PE/EA (10:1) to afford the title compound (295 mg, 81.4%) as a white solid.

### Step 4: 3,8-diazabicyclo[3.2.1]oct-6-ene-6-carbonitrile bis(2,2,2-trifluoroacetate)

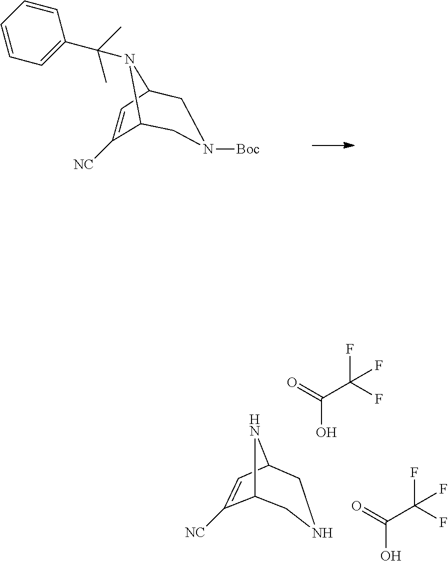 [SMILES: CC(C)(C)OC(=O)N1CC2C=C(C#N)C(C1)N2C(C)(C)c1ccccc1 ; N#CC1=CC2CNCC1N2 ; O=C(O)C(F)(F)F ; O=C(O)C(F)(F)F]

A mixture of tert-butyl (1R,5S)-6-cyano-8-(2-phenylpropan-2-yl)-3,8-diazabicyclo [3.2.1]oct-6-ene-3-carboxylate (290 mg, 0.82 mmol, 1.00 eq.) in TFA (3 mL) was stirred for 2 h at 80° C. under nitrogen atmosphere. The reaction mixture was cooled and then concentrated. The residue was triturated with Et₂O to afford the title compound (215 mg, 72.0%) as a white oil.

### Step 5: 2,7-dichloro-8-fluoro-4-(2-(trimethylsilyl)ethoxy)pyrido[4,3-d]pyrimidine

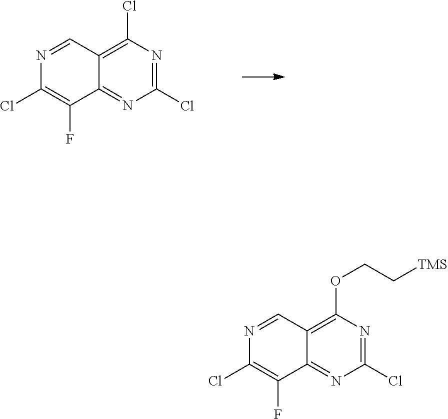 [SMILES: Fc1c(Cl)ncc2c(Cl)nc(Cl)nc12 ; C[Si](C)(C)CCOc1nc(Cl)nc2c(F)c(Cl)ncc12]

To a stirred solution of 2,4,7-trichloro-8-fluoropyrido[4,3-d]pyrimidine (5.6 g, 22.18 mmol, 1.18 eq.) and 2-(trimethylsilyl)ethanol (2.23 g, 18.86 mmol, 1.00 eq.) in THF (28 mL) was added t-BuOK (2.6 g, 23.17 mmol, 1.23 eq.) at −60° C. under N₂ atmosphere. The reaction mixture was stirred for 1h at −60° C., quenched with water and then extracted with EtOAc. The combined organic layers were washed with water, brine, dried over anhydrous Na₂SO₄, filtered and then concentrated. The residue was purified by silica gel column chromatography, eluted with PE/EA (5/1), to afford the title compound (6.5 g, 100%) as a light-yellow solid.

### Step 6: 7-chloro-8-fluoro-2-((tetrahydro-1H-pyrrolizin-7a(5H)-yl)methoxy)-4-(2-(trimethylsilyl)ethoxy)pyrido [4,3-d]pyrimidine

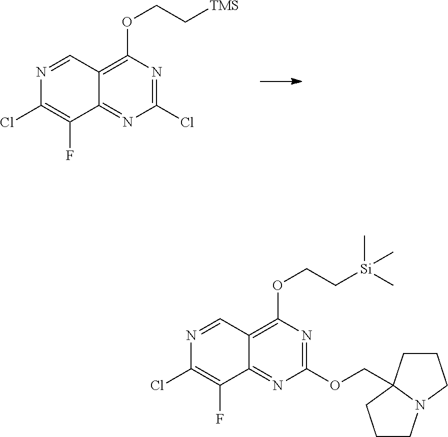 [SMILES: C[Si](C)(C)CCOc1nc(Cl)nc2c(F)c(Cl)ncc12 ; C[Si](C)(C)CCOc1nc(OCC23CCCN2CCC3)nc2c(F)c(Cl)ncc12]

To a stirred solution of 2,7-dichloro-8-fluoro-4-(2-(trimethylsilyl)ethoxy)pyrido[4,3-d]pyrimidine (1.0 g, 2.99 mmol, 1.00 eq.) and hexahydropyrrolizin-7a-ylmethanol (0.84 g, 5.95 mmol, 1.99 eq.) in 1,4-dioxane (10 mL) was added DIPEA (0.77 g, 5.96 mmol, 1.99 eq.) at RT under N₂ atmosphere. The resulting mixture was stirred for 16 h at 80° C., cooled, quenched with water and then extracted with EtOAc. The combined organic layers were washed with water, brine, dried over anhydrous Na₂SO₄, filtered and then concentrated. The residue was purified by silica gel column chromatography, eluted with PE/EA (1/1), to afford the title compound (934 mg, 71.2%) as a light-yellow solid.

### Step 7: 8-fluoro-7-(3-(methoxymethoxy)-8-((triisopropylsilyl)ethynyl)naphthalen-1-yl)-2-((tetrahydro-1H-pyrrolizin-7a(5H)-yl)methoxy)-4-(2-(trimethylsilyl)ethoxy)pyrido[4,3-d]-pyrimidine

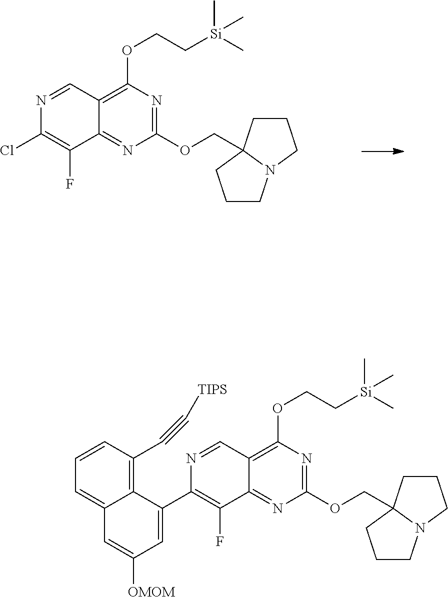

To a stirred solution of 7-chloro-8-fluoro-2-((tetrahydro-1H-pyrrolizin-7a(5H)-yl)methoxy)-4-(2-(trimethylsilyl) ethoxy)pyrido[4,3-d]pyrimidine (934 mg, 2.13 mmol, 1.00 eq.) and triisopropyl((6-(methoxymethoxy)-8-(4,4,5,5-tetramethyl-1,3,2-dioxaborolan-2-yl)naphthalen-1-yl)ethynyl)silane (1683 mg, 3.40 mmol, 1.60 eq.) in 10 mL of DCE/H₂O (10/1) were added K₂CO₃ (882 mg, 6.38 mmol, 3.00 eq.) and cataCXium A Pd G3 (47 mg, 0.064 mmol, 0.030 eq.) under nitrogen atmosphere. After stirring for 4h at 85° C., the reaction mixture was concentrated and the residue was purified by silica gel column chromatography, eluted with PE/EA (1/1), to afford the title compound (910 mg, 55.4%) as a light-yellow solid.

### Step 8: 7-(8-ethynyl-3-(methoxymethoxy)naphthalen-1-yl)-8-fluoro-2-((tetrahydro-1H-pyrrolizin-7a(5H)-yl)methoxy)pyrido[4,3-d]pyrimidin-4-ol

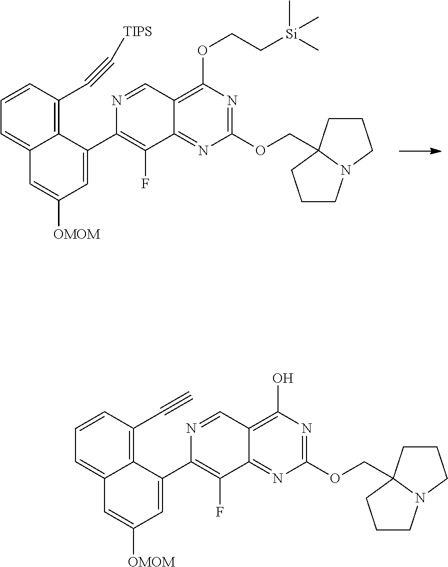

A solution of 8-fluoro-7-(3-(methoxymethoxy)-8-((triisopropylsilyl)ethynyl)naphthalen-1-yl)-2-((tetrahydro-TH-pyrrolizin-7a(5H)-yl)methoxy)-4-(2-(trimethylsilyl)ethoxy)pyrido[4,3-d]pyrimidine (910 mg, 1.18 mmol, 1.00 eq.) and CsF (717 mg, 4.72 mmol, 4.00 eq.) in DMF (5 mL) was stirred for 1 h at 60° C. under N₂ atmosphere. The reaction mixture was cooled, quenched with water and then extracted with EtOAc. The combined organic layers were washed with water, brine, dried over anhydrous Na₂SO₄, filtered and then concentrated. The residue was purified by silica gel column chromatography, eluted with PE/EA (0˜100), to afford the title compound (560 mg, 92.4%) as a yellow solid.

### Step 9: 3-(7-(8-ethynyl-3-(methoxymethoxy)naphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]oct-6-ene-6-carbonitrile

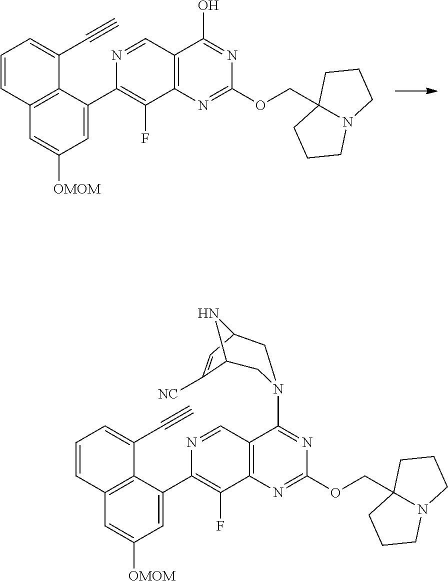

A mixture of 7-(8-ethynyl-3-(methoxymethoxy)naphthalen-1-yl)-8-fluoro-2-((tetrahydro-1H-pyrrolizin-7a(5H)-yl)methoxy)pyrido[4,3-d]pyrimidin-4-ol (100 mg, 0.19 mmol, 1.00 eq.), (1R,5S)-3,8-diazabicyclo [3.2.1]oct-6-ene-6-carbonitrile bis(2,2,2-trifluoroacetate) (84.6 mg, 0.23 mmol, 1.21 eq.), PyBOP (132 mg, 0.25 mmol, 1.32 eq.) and DIPEA (51 mg, 0.39 mmol, 2.05 eq.) in DMF (2 mL) was stirred for 2 h at RT. The reaction mixture was quenched with water and then extracted with EtOAc. The combined organic layers were washed with brine, dried over anhydrous Na₂SO₄, filtered and then concentrated. The residue was purified by silica gel column chromatography, eluted with DCM/MeOH (7:1), to afford the title compound (75 mg, 63.2%) as a yellow solid.

### Step 10: 3-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-3, 8-diazabicyclo[3.2.1]oct-6-ene-6-carbonitrile

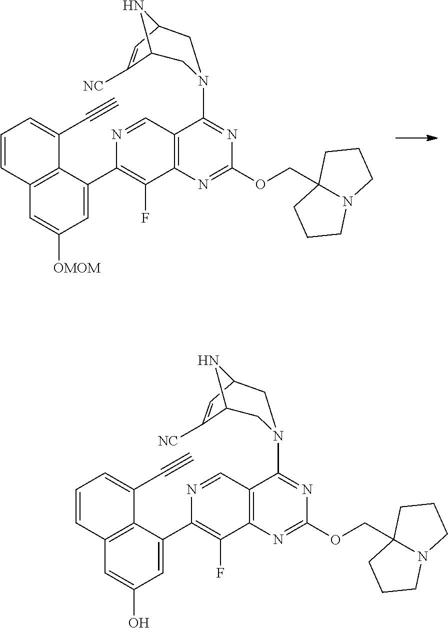

To a stirred solution of (1R,5S)-3-(7-(8-ethynyl-3-(methoxymethoxy)naphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]oct-6-ene-6-carbonitrile (40 mg, 0.063 mmol, 1.00 eq.) in DCM (1.5 mL) was added a combined solution of 4.0 M HCl(gas) in 1,4-dioxane (0.1 mL) and DCM (1.0 mL) dropwise at −30° C. The resulting mixture was stirred for 1h at −30° C., basified to pH=8 with NH₃ in MeOH. The resulting mixture was concentrated and the residue was purified by Prep-HPLC to afford the title compound (10 mg, 27.0%) as a yellow solid. MS (ES, m/z): [M+1]⁺=588.3.

### Example 8

### Synthesis of 4-(4-(3,8-diazabicyclo[3.2.1]oct-6-en-3-yl)-8-fluoro-2-((tetrahydro-1H-pyrrolizin-7a(5H)-yl)methoxy)pyrido[4,3-d]pyrimidin-7-yl)-5-ethynylnaphthalen-2-ol and 4-(4-((1R,5S)-3,8-diazabicyclo[3.2.1]oct-6-en-8-yl)-8-fluoro-2-((tetrahydro-1H-pyrrolizin-7a(5H)-yl)-methoxy)pyrido[4,3-d]pyrimidin-7-yl)-5-ethynylnaphthalen-2-ol

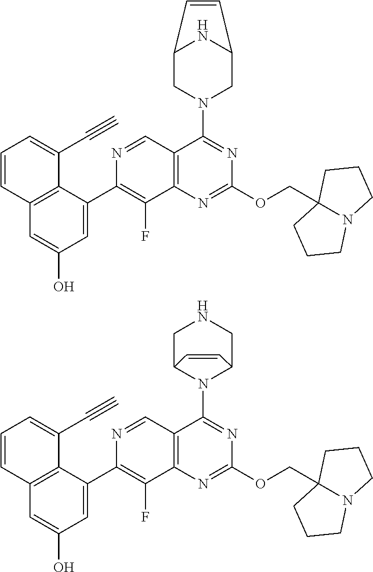 [SMILES: C#Cc1cccc2cc(O)cc(-c3ncc4c(N5CC6C=CC(C5)N6)nc(OCC56CCCN5CCC6)nc4c3F)c12 ; C#Cc1cccc2cc(O)cc(-c3ncc4c(N5C6C=CC5CNC6)nc(OCC56CCCN5CCC6)nc4c3F)c12]

### Step 1: tert-butyl (3S,4R)-3,4-dihydroxypyrrolidine-1-carboxylate

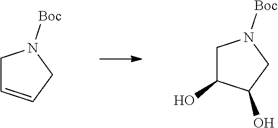 [SMILES: CC(C)(C)OC(=O)N1CC=CC1 ; CC(C)(C)OC(=O)N1C[C@@H](O)[C@@H](O)C1]

To a stirred solution of tert-butyl 2,5-dihydro-1H-pyrrole-1-carboxylate (2.5 g, 14.78 mmol, 1.00 eq.) in acetone (24 mL) and H₂O (6 mL) was added NMO (2.6 g, 22.16 mmol, 1.50 eq.) and K₂O_{S}O₄·2H₂O (163 mg, 0.44 mmol, 0.030 eq.) in portions at RT under nitrogen atmosphere. The resulting mixture was stirred for 3 h at RT, quenched with aqueous Na₂S₂O₃ and then extracted with EtOAc. The combined organic layers were washed with brine, dried over anhydrous Na₂SO₄, filtered and then concentrated. The residue was purified by silica gel column chromatography, eluted with DCM/EA (10:1 to 1:10), to afford the title compound (2.8 g, 93.2%) as a yellow oil.

### Step 2: tert-butyl bis(2-hydroxybut-3-en-1-yl)carbamate

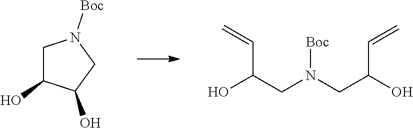 [SMILES: CC(C)(C)OC(=O)N1C[C@@H](O)[C@@H](O)C1 ; C=CC(O)CN(CC(O)C=C)C(=O)OC(C)(C)C]

To stirred solution tert-butyl (3R,4S)-3,4-dihydroxypyrrolidine-1-carboxylate (2.50 g, 12.30 mmol, 1.00 eq.) in DCM (25 mL) was added (diacetoxyiodo)benzene (5.94 g, 18.44 mmol, 1.50 eq.) slowly at 0° C. under nitrogen atmosphere. The resulting mixture was stirred for 1 h at RT, cooled to −78° C. and then added 1.0 M vinylmagnesium bromide in THF (74 mL, 74.0 mmol, 6.02 eq.) slowly over 20 minutes. After stirring for 16 h at RT, the reaction mixture was quenched with 1.0 M aqueous HCl and then extract with DCM. The combined organic layers were washed with water, dried over Na₂SO₄, filtered and then concentrated. The residue was purified by silica gel column chromatography, eluting with PE/EA (1/1), to afford the title compound (2.6 g, 82.1%) as a yellow oil.

### Step 3: ((tert-butoxycarbonyl)azanediyl)bis(but-3-ene-1,2-diyl) bis(2,2,2-trichloroacetimidate)

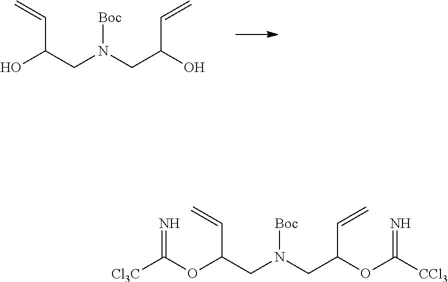

To a stirred solution of tert-butyl bis(2-hydroxybut-3-en-1-yl)carbamate (1.2 g, 4.66 mmol, 1.00 eq.) in DCM (16 mL) were add 2,2,2-trichloroacetonitrile (4.04 g, 27.98 mmol, 6.00 eq.), DBU (0.35 g, 2.30 mmol, 0.49 eq.) dropwise at 0° C. After stirring for 18h at RT, the reaction mixture was concentrated and the residue was purified by silica gel column chromatography, eluted with PE/EA (10:1), to afford title compound (0.8 g, 31.3%) as a yellow oil.

### Step 4: tert-butyl (3S,5R)-4-(2-phenylpropan-2-yl)-3,5-divinylpiperazine-1-carboxylate

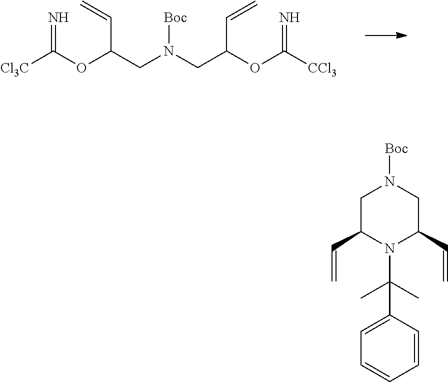

To a stirred solution of 2-phenylpropan-2-amine (238 mg, 1.76 mmol, 1.21 eq.) in DCE (5 mL) was added [Ir(cod)Cl]₂ (49 mg, 0.074 mmol, 0.051 eq.), followed by a solution of ((tert-butoxycarbonyl)azanediyl)bis(but-3-ene-1,2-diyl) bis(2,2,2-trichloroacetimidate) (800 mg, 1.46 mmol, 1.00 eq.) in DCE (4.4 mL) dropwise at 0° C. under nitrogen atmosphere. After stirring overnight at RT, the reaction mixture was concentrated and the residue was purified by silica gel column chromatography, eluted with PE/EA (5:1), to afford the title compound (315 mg, 60.3%) as a yellow oil.

### Step 5: (1R,5S)-8-(2-phenylpropan-2-yl)-3,8-diazabicyclo[3.2.1]oct-6-ene-3-carboxylate

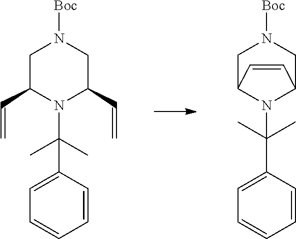 [SMILES: C=C[C@@H]1CN(C(=O)OC(C)(C)C)C[C@H](C=C)N1C(C)(C)c1ccccc1 ; CC(C)(C)OC(=O)N1CC2C=CC(C1)N2C(C)(C)c1ccccc1]

To a stirred solution of tert-butyl (3S,5R)-4-(2-phenylpropan-2-yl)-3,5-divinylpiperazine-1-carboxylate (315 mg, 0.88 mmol, 1.00 eq.) in toluene (10 mL) was added Grubbs 2nd generation catalyst (37.51 mg, 0.044 mmol, 0.050 eq.) in one portion at RT under nitrogen atmosphere. The reaction mixture was stirred for 12 h at 120° C., cooled and then concentrated. The residue was purified by silica gel column chromatography, eluted with PE/EA (10:1), to afford the title compound (70 mg, 23.9%) as a light-yellow oil.

### Step 6: (1R,5S)-3,8-diazabicyclo[3.2.1]oct-6-ene bis(2,2,2-trifluoroacetate)

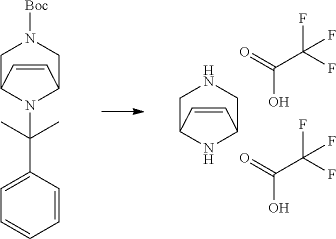

A mixture of tert-butyl (1R,5S)-8-(2-phenylpropan-2-yl)-3,8-diazabicyclo[3.2.1]oct-6-ene-3-carboxylate (200 mg, 0.61 mmol, 1.00 eq.) in TFA (3 mL) was refluxed for 2 h under nitrogen atmosphere. The reaction mixture was concentrated and the residue was triturated with Et₂O to afford the title compound (172.3 mg, 83.6%) as a white oil.

### Step 7: 4-(3,8-diazabicyclo[3.2.1]oct-6-en-3-yl)-7-(8-ethynyl-3-(methoxymethoxy)naphthalen-1-yl)-8-fluoro-2-((tetrahydro-1H-pyrrolizin-7a(5H)-yl)methoxy)pyrido[4,3-d]pyrimidine and 4-((1R,5S)-3,8-diazabicyclo[3.2.1]oct-6-en-8-yl)-7-(8-ethynyl-3-(methoxymethoxy)naphthalen-1-yl)-8-fluoro-2-((tetrahydro-1H-pyrrolizin-7a(5H)-yl)methoxy)pyrido[4,3-d]pyrimidine

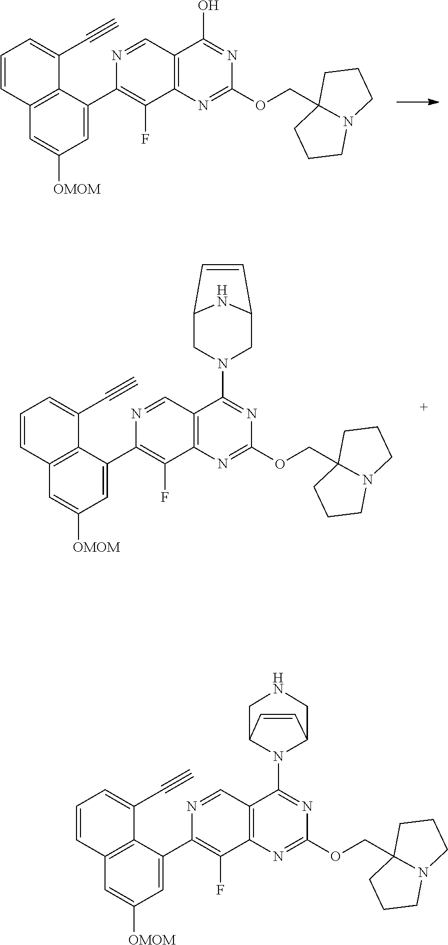

A mixture of 7-(8-ethynyl-3-(methoxymethoxy)naphthalen-1-yl)-8-fluoro-2-((tetrahydro-1H-pyrrolizin-7a(5H)-yl)methoxy)pyrido[4,3-d]pyrimidin-4-ol (100 mg, 0.19 mmol, 1.00 eq.), (1R,5S)-3,8-diazabicyclo[3.2.1]oct-6-ene bis(2,2,2-trifluoroacetate) (79 mg, 0.23 mmol, 1.21 eq.), PyBOP (132 mg, 0.25 mmol, 1.32 eq.) and DIPEA (51 mg, 0.39 mmol, 2.05 eq.) in DMF (2 mL) was stirred for 2 h at RT under nitrogen atmosphere. The reaction mixture was quenched with water and then extracted with EtOAc. The combined organic layers were washed with brine, dried over anhydrous Na₂SO₄, filtered and then concentrated. The residue was purified by silica gel column chromatography, eluted with DCM/MeOH (7:1), to afford the title compounds (50 mg, 43.2%) as a yellow solid.

### Step 8: 4-(4-(3,8-diazabicyclo[3.2.1]oct-6-en-3-yl)-8-fluoro-2-((tetrahydro-1H-pyrrolizin-7a(5H)-yl)methoxy)pyrido[4,3-d]pyrimidin-7-yl)-5-ethynylnaphthalen-2-ol and 4-(4-((1R,5S)-3,8-diazabicyclo[3.2.1]oct-6-en-8-yl)-8-fluoro-2-((tetrahydro-1H-pyrrolizin-7a(5H)-yl)methoxy)pyrido[4,3-d]pyrimidin-7-yl)-5-ethynylnaphthalen-2-ol

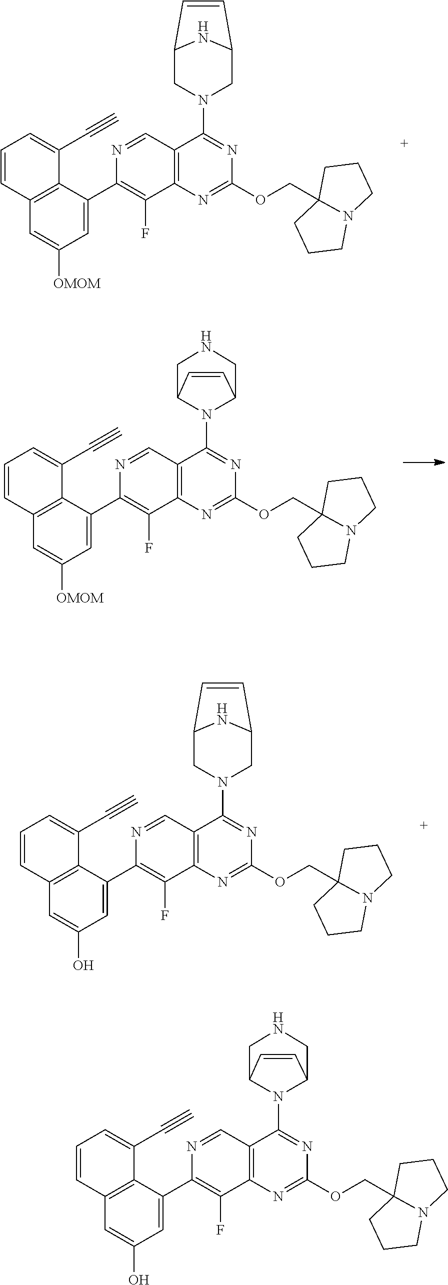

To a stirred solution of the mixture of 4-(3,8-diazabicyclo[3.2.1]oct-6-en-3-yl)-7-(8-ethynyl-3-(methoxymethoxy)naphthalen-1-yl)-8-fluoro-2-((tetrahydro-1H-pyrrolizin-7a(5H)-yl)methoxy)pyrido[4,3-d]pyrimidine and 4-((1R,5S)-3,8-diazabicyclo[3.2.1]oct-6-en-8-yl)-7-(8-ethynyl-3-(methoxymethoxy)naphthalen-1-yl)-8-fluoro-2-((tetrahydro-1H-pyrrolizin-7a(5H)-yl)methoxy)pyrido[4,3-d]pyrimidine (50 mg, 0.082 mmol, 1.00 eq.) in DCM (0.5 mL) was added a combined solution of HCl (gas) in 1,4-dioxane (4.0 M, 0.1 mL) and DCM (1 mL) dropwise at −30° C. The resulting mixture was stirred for 1h at −30° C., basified to pH=8 with NH₃ in MeOH and then concentrated. The residue was purified by Prep-HPLC to afford 4-(4-(3,8-diazabicyclo[3.2.1]oct-6-en-3-yl)-8-fluoro-2-((tetrahydro-1H-pyrrolizin-7a(5H)-yl)methoxy)pyrido[4,3-d]pyrimidin-7-yl)-5-ethynylnaphthalen-2-ol MS (ES, m/z): [M+1]⁺=563.3; and 4-(4-((1R,5S)-3,8-diazabicyclo[3.2.1]oct-6-en-8-yl)-8-fluoro-2-((tetrahydro-1H-pyrrolizin-7a(5H)-yl)methoxy)pyrido[4,3-d]pyrimidin-7-yl)-5-ethynylnaphthalen-2-ol, MS (ES, m/z): [M+1]⁺=563.3.

### Example 9

### Synthesis of 5-ethynyl-4-(8-fluoro-2-((tetrahydro-1H-pyrrolizin-7a(5H)-yl)methoxy)-4-(7,9-diazatricyclo[3.3.1.02,4]nonan-7-yl)pyrido[4,3-d]pyrimidin-7-yl)naphthalen-2-ol

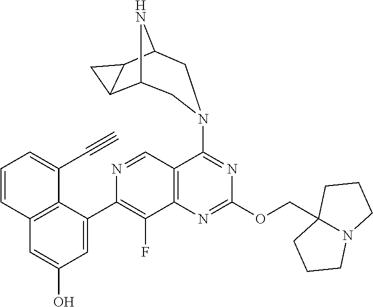 [SMILES: C#Cc1cccc2cc(O)cc(-c3ncc4c(N5CC6NC(C5)C5CC65)nc(OCC56CCCN5CCC6)nc4c3F)c12]

### Step 1: tert-butyl 9-(2-phenylpropan-2-yl)-7,9-diazatricyclo[3.3.1.02,4]nonane-7-carboxylate

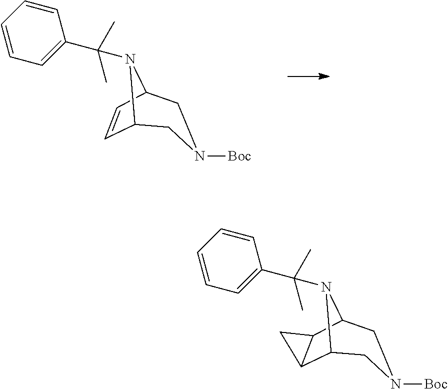 [SMILES: CC(C)(C)OC(=O)N1CC2C=CC(C1)N2C(C)(C)c1ccccc1 ; CC(C)(C)OC(=O)N1CC2C3CC3C(C1)N2C(C)(C)c1ccccc1]

To a stirred mixture of KOH (3.0 g, 21.39 mmol, 14.07 eq., 40% aqueous solution) and ether (10 mL) was added 1-methyl-1-nitrosourea (1.0 g, 9.70 mmol, 6.38 eq.) in portions at 0-5° C. The resulting mixture was stirred for 5 min at the same temperature and the ether layer was separated. To a stirred solution of tert-butyl (1R,5S)-8-(2-phenylpropan-2-yl)-3,8-diazabicyclo[3.2.1]oct-6-ene-3-carboxylate (500 mg, 1.52 mmol, 1.00 eq.) and Pd(acac)₂ (46 mg, 0.15 mmol, 0.10 eq.) in DCM (10 mL) was added the above ether solution dropwise at RT. The resulting mixture was stirred for 30 min at RT, diluted with water and then extracted with DCM. The combined organic layers were washed with water, brine, dried over anhydrous Na₂SO₄, filtered and then concentrated. The residue was purified by silica gel column chromatography, eluted with EA/PE (0-20%), to afford the title compound (350 mg, 67.1%) as a yellow oil.

### Step 2: 7,9-diazatricyclo[3.3.1.0^{2,4}]nonane bis(2,2,2-trifluoroacetate)

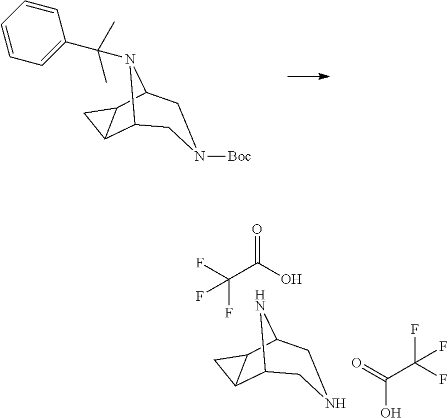

Proceeding analogously as described in Example 7, Step 4 above, but using tert-butyl (1R,2R,4S,5S)-9-(2-phenylpropan-2-yl)-7,9-diazatricyclo[3.3.1.02,4]nonane-7-carboxylate and TFA provided the title compound.

### Step 3: 5-ethynyl-4-(8-fluoro-2-((tetrahydro-1H-pyrrolizin-7a(5H)-yl)methoxy)-4-(7,9-diazatricyclo[3.3.1.02,4]nonan-7-yl)pyrido[4,3-d]pyrimidin-7-yl)naphthalen-2-ol

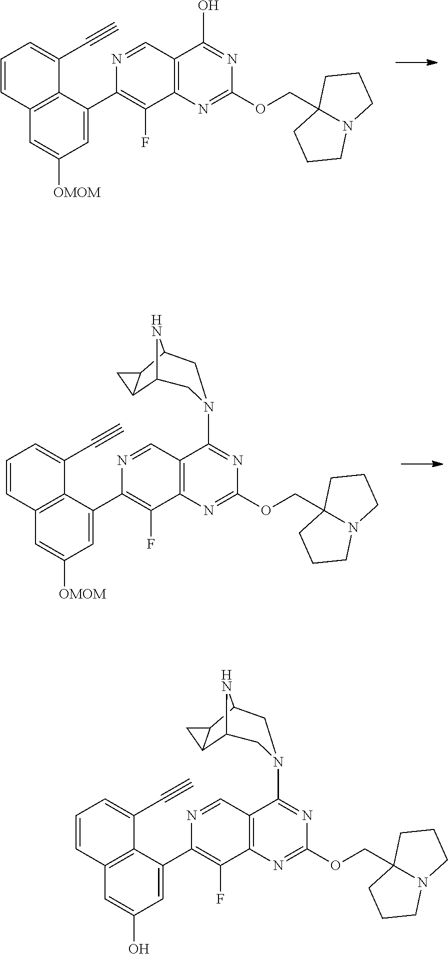

Proceeding analogously as described in Example 7, Steps 9-10 above, but using diazatricyclo[3.3.1.0^{2,4}]nonane bis(2,2,2-trifluoroacetate) instead of (1R,5S)-3,8-diazabicyclo [3.2.1]oct-6-ene-6-carbonitrile bis(2,2,2-trifluoroacetate) provided the title compound. MS (ES, m/z): [M+1]⁺=577.3.

### Example 10

### Synthesis of 5-ethynyl-4-(8-fluoro-4-(6-methylene-3,8-diazabicyclo[3.2.1]octan-3-yl)-2-((tetrahydro-1H-pyrrolizin-7a(5H)-yl)methoxy)pyrido[4,3-d]pyrimidin-7-yl)naphthalen-2-ol

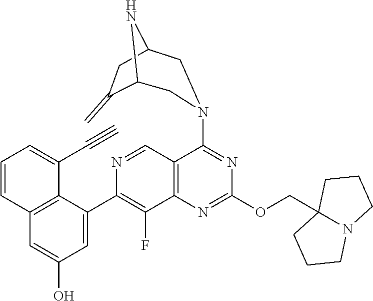 [SMILES: C#Cc1cccc2cc(O)cc(-c3ncc4c(N5CC6CC(=C)C(C5)N6)nc(OCC56CCCN5CCC6)nc4c3F)c12]

### Step 1: tert-butyl 6-hydroxy-8-(2-phenylpropan-2-yl)-3,8-diazabicyclo[3.2.1]octane-3-carboxylate

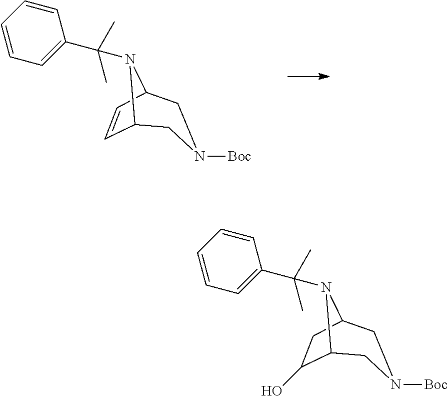 [SMILES: CC(C)(C)OC(=O)N1CC2C=CC(C1)N2C(C)(C)c1ccccc1 ; CC(C)(C)OC(=O)N1CC2CC(O)C(C1)N2C(C)(C)c1ccccc1]

To a stirred solution of tert-butyl 8-(2-phenylpropan-2-yl)-3,8-diazabicyclo[3.2.1]oct-6-ene-3-carboxylate (374 mg, 1.14 mmol, 1.00 eq.) in THF (12 mL) was added 1.0 M BH₃·THF (4.5 15 mL, 4.5 mmol, 3.95 eq.) dropwise at 0° C. under nitrogen atmosphere. After stirring overnight at RT, 5% NaOH (aq.) (4.5 mL) was added over 10 min, followed by 30% H₂O₂ (390 mg, 3.44 mmol, 3.02 eq.) at 0° C. The resulting mixture was stirred for 1h at RT and then extracted with DCM. The organic layer was concentrated and the residue was purified by trituration with Et₂O to afford he title compound (300 mg, 76.3%) as a white oil.

### Step 2: tert-butyl 6-oxo-8-(2-phenylpropan-2-yl)-3,8-diazabicyclo[3.2.1]octane-3-carboxylate

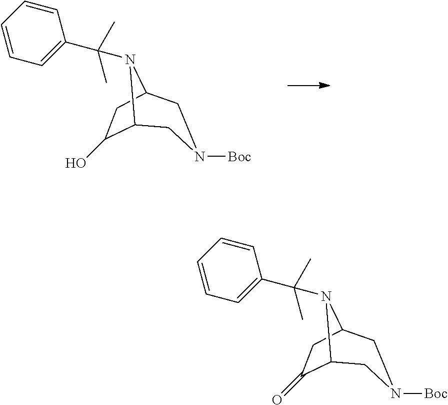 [SMILES: CC(C)(C)OC(=O)N1CC2CC(O)C(C1)N2C(C)(C)c1ccccc1 ; CC(C)(C)OC(=O)N1CC2CC(=O)C(C1)N2C(C)(C)c1ccccc1]

To a stirred solution of tert-butyl 6-hydroxy-8-(2-phenylpropan-2-yl)-3,8-diazabicyclo[3.2.1] octane-3-carboxylate (300 mg, 0.87 mmol, 1.00 eq.) in DCM (10 mL) was added Dess-Martin reagent (551 mg, 1.30 mmol, 1.49 eq.) in portions at 0° C. After stirring at 0° C. for 1h, the reaction mixture was diluted with water and then extracted with EtOAc. The organic layer was washed with water, NaHCO₃(aq.), dried over Na₂SO₄, filtered and then concentrated. The residue was purified by silica gel column chromatography, eluted with PE/EA (10:1), to afford the title compound (210 mg, 70.1%) as a yellow oil.

### Step 3: tert-butyl 6-methylene-8-(2-phenylpropan-2-yl)-3,8-diazabicyclo[3.2.1]octane-3-carboxylate

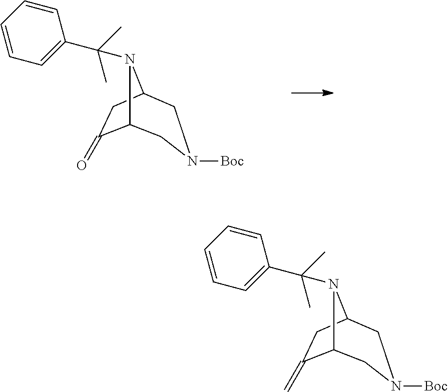 [SMILES: CC(C)(C)OC(=O)N1CC2CC(=O)C(C1)N2C(C)(C)c1ccccc1 ; C=C1CC2CN(C(=O)OC(C)(C)C)CC1N2C(C)(C)c1ccccc1]

A solution of methyltriphenylphosphanium iodide (528 mg, 1.31 mmol, 3.00 eq.) and t-BuOK (147 mg, 1.31 mmol, 3.00 eq.) in DMSO (3 mL) was stirred for 10 min at 10° C. under nitrogen atmosphere. To this stirred mixture was added a solution of tert-butyl (1R,5R)-6-oxo-8-(2-phenylpropan-2-yl)-3,8-diazabicyclo[3.2.1]octane-3-carboxylate (150 mg, 0.44 mmol, 1.00 eq.) in DMSO (1 mL) dropwise at 10° C. After stirring at RT for 3 h, the reaction mixture was quenched with water and then extracted with EtOAc. The combined organic layers were washed with brine, dried over anhydrous Na₂SO₄, filtered and then concentrated. The residue was purified by silica gel column chromatography, eluted with PE/EA (10:1), to afford the title compound (99 mg, 65.9%) as a yellow oil.

### Step 4: 6-methylene-3,8-diazabicyclo[3.2.1]octane bis(2,2,2-trifluoroacetate)

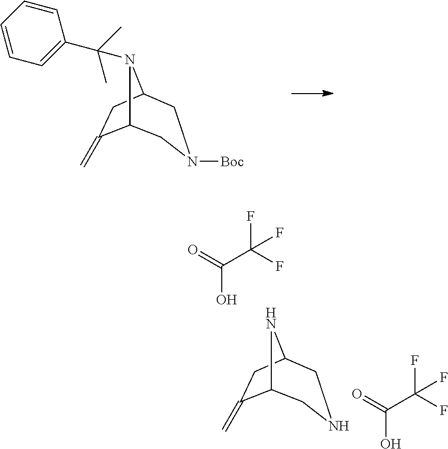

Proceeding analogously as described in Example 7, Step 4 above, but using tert-butyl 6-methylene-8-(2-phenylpropan-2-yl)-3,8-diazabicyclo[3.2.1]octane-3-carboxylate and TFA provided the title compound.

### Step 5: 5-ethynyl-4-(8-fluoro-4-(6-methylene-3,8-diazabicyclo[3.2.1]octan-3-yl)-2-((tetrahydro1H-pyrrolizin-7a(5H)-yl)methoxy)pyrido[4,3-d]pyrimidin-7-yl)naphthalen-2-ol

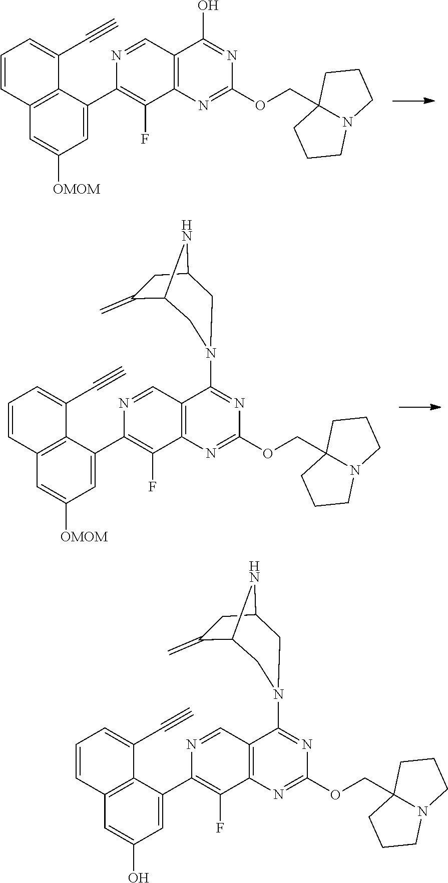

Proceeding analogously as described in Example 7, Steps 9-10 above, using 6-methylene-3,8-diazabicyclo[3.2.1]octane bis(2,2,2-trifluoroacetate) instead of (1R,5S)-3,8-diazabicyclo [3.2.1]oct-6-ene-6-carbonitrile bis(2,2,2-trifluoroacetate), provided the title compound. MS (ES, m/z): [M+1]⁺=577.3.

### Example 11

### Synthesis of 4-((1R,5S)-3,8-diazabicyclo[3.2.1]octan-3-yl)-7-(8-ethynyl-7-fluoronaphthalen-1-yl)-8-fluoro-2-((2-methylenetetrahydro-TH-pyrrolizin-7a(5H)-yl)methoxy)pyrido[4,3-d]pyrimidine

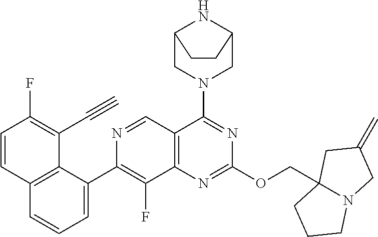 [SMILES: C#Cc1c(F)ccc2cccc(-c3ncc4c(N5CC6CCC(C5)N6)nc(OCC56CCCN5CC(=C)C6)nc4c3F)c12]

Proceeding as described in Example 1, Steps 5 to 7, using ((2-fluoro-8-(4,4,5,5-tetramethyl-1,3,2-dioxaborolan-2-yl)naphthalen-1-yl)ethynyl)triisopropylsilane instead of triisopropyl((6-(methoxymethoxy)-8-(4,4,5,5-tetramethyl-1,3,2-dioxaborolan-2-yl)naphthalen-1-yl)ethynyl)silane in Step 5, provided the title compound. MS (ES, m/z): [M+1]⁺=579.4.

### Example 12

### Synthesis of 9b-(((4-(3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-2,3,5,9b-tetrahydro-1H-pyrrolo[2,1-a]isoindole

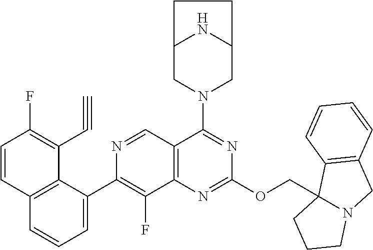 [SMILES: C#Cc1c(F)ccc2cccc(-c3ncc4c(N5CC6CCC(C5)N6)nc(OCC56CCCN5Cc5ccccc56)nc4c3F)c12]

Proceeding as described in Example 1, Steps 4 to 7, using (2,3-dihydro-1H-pyrrolo[2,1-a]isoindol-9b(5H)-yl)methanol instead of (2-methylidene-tetrahydro-1H-pyrrolizin-7a-yl)methanol in Step 4 and ((2-fluoro-8-(4,4,5,5-tetramethyl-1,3,2-dioxaborolan-2-yl)naphthalen-1-yl)ethynyl)triisopropylsilane instead of triisopropyl((6-(methoxymethoxy)-8-(4,4,5,5-tetramethyl-1,3,2-dioxaborolan-2-yl)naphthalen-1-yl)ethynyl)silane in Step 5, gave the title compound. MS (ES, m/z): [M+1]⁺=615.3.

### Example 13

### Synthesis of 4-((1R,5S)-3,8-diazabicyclo[3.2.1]octan-3-yl)-2-((2,6-dimethylenetetrahydro-1H-pyrrolizin-7a(5H)-yl)methoxy)-7-(8-ethynyl-7-fluoronaphthalen-1-yl)-8-fluoropyrido[4,3-d]-pyrimidine

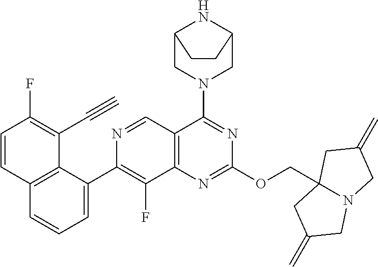 [SMILES: C#Cc1c(F)ccc2cccc(-c3ncc4c(N5CC6CCC(C5)N6)nc(OCC56CC(=C)CN5CC(=C)C6)nc4c3F)c12]

### Step 1: tert-butyl (1R,5S)-3-(2-((2,6-dimethylenetetrahydro-1H-pyrrolizin-7a(5H)-yl)methoxy)-7-(8-ethynyl-7-fluoronaphthalen-1-yl)-8-fluoropyrido[4,3-d]pyrimidin-4-yl)-3,8-diazabicyclo[3.2.1]octane-8-carboxylate

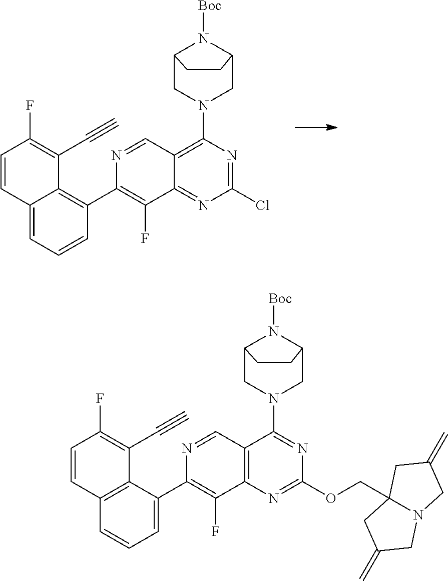 [SMILES: C#Cc1c(F)ccc2cccc(-c3ncc4c(N5CC6CCC(C5)N6C(=O)OC(C)(C)C)nc(Cl)nc4c3F)c12 ; C#Cc1c(F)ccc2cccc(-c3ncc4c(N5CC6CCC(C5)N6C(=O)OC(C)(C)C)nc(OCC56CC(=C)CN5CC(=C)C6)nc4c3F)c12]

To a stirred solution of (2,6-dimethylenetetrahydro-1H-pyrrolizin-7a(5H)-yl)methanol (22 mg, 0.13 mmol, 1.46 eq.) in THF (0.50 mL) was added 60% NaH (7.0 mg, 0.18 mmol, 2.02 eq.) at 0° C. under nitrogen atmosphere. After stirring at 0° C. for 5 min, a solution of tert-butyl (1R,5S)-3-(2-chloro-7-(8-ethynyl-7-fluoronaphthalen-1-yl)-8-fluoropyrido[4,3-d]pyrimidin-4-yl)-3,8-diazabicyclo[3.2.1]octane-8-carboxylate (50 mg, 0.089 mmol, 1.00 eq.) in THF (0.50 mL) was added dropwise at 0° C. The resulting mixture was stirred for 30 min, quenched with saturated NH₄Cl (aq.), and then extracted with EtOAc. The combined organic layers were washed with brine, dried over anhydrous MgSO₄, filtered and then concentrated. The residue was purified by silica gel column chromatography, eluted with PE/EA (0˜100%), to afford the title compound (48 mg, 77.5%) as a light yellow solid.

### Step 2: 4-((1R,5S)-3,8-diazabicyclo[3.2.1]octan-3-yl)-2-((2,6-dimethylenetetrahydro-1H-pyrrolizin-7a(5H)-yl)methoxy)-7-(8-ethynyl-7-fluoronaphthalen-1-yl)-8-fluoropyrido[4,3-d]-pyrimidine

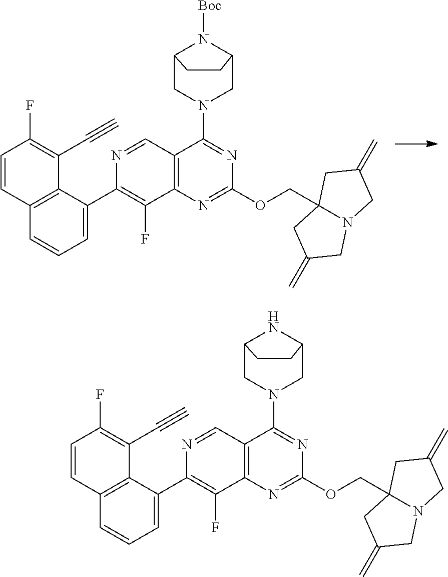

To a stirred solution of tert-butyl (1R,5S)-3-(2-((2,6-dimethylenetetrahydro-1H-pyrrolizin-7a(5H)-yl)methoxy)-7-(8-ethynyl-7-fluoronaphthalen-1-yl)-8-fluoropyrido[4,3-d]pyrimidin-4-yl)-3,8-diazabicyclo[3.2.1]octane-8-carboxylate (48 mg, 0.069 mmol, 1.00 eq.) in DCM (0.50 mL) was added 4.0 M HCl in 1,4-dioxane (0.25 mL) dropwise at 5° C. The resulting mixture was stirred for 1 h at 0° C., basified to pH=9 with 7.0 M NH₃ in MeOH. The resulting mixture was concentrated and the residue was purified by Prep-HPLC to afford the title compound (13.3 mg, 33.3%) as a light yellow solid. MS (ES, m/z): [M+H]⁺=591.3.

### Example 14

### Synthesis of 4-((1R,5S)-3,8-diazabicyclo[3.2.1]octan-3-yl)-2-((2-ethylidenetetrahydro-1H-pyrrolizin-7a(5H)-yl)methoxy)-7-(8-ethynyl-7-fluoronaphthalen-1-yl)-8-fluoropyrido[4,3-d]-pyrimidine

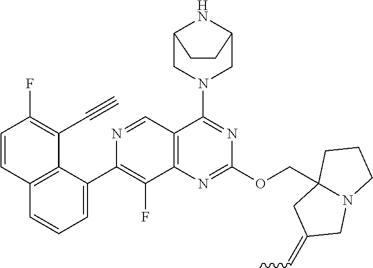

Following compounds were prepared analogously as described in synthesis of Example 13, Step 1-2, using (2-ethylidenetetrahydro-1H-pyrrolizin-7a(5H)-yl)methanol instead of (2-methylidene-tetrahydro-1H-pyrrolizin-7a-yl)methanol in step 1, provided the title compound. MS (ES, m/z): [M+H]⁺=593.4.

### Example 15

### Synthesis of a mixture (6aS,7aR,11aR)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6a,7a,8,9,10,11a-hexahydro-6H,7H-chromeno[3,4-b]pyrrolizine and (6aR,7aS,11aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]-pyrimidin-2-yl)oxy)methyl)-6a,7a,8,9,10,11a-hexahydro-6H,7H-chromeno[3,4-b]pyrrolizine

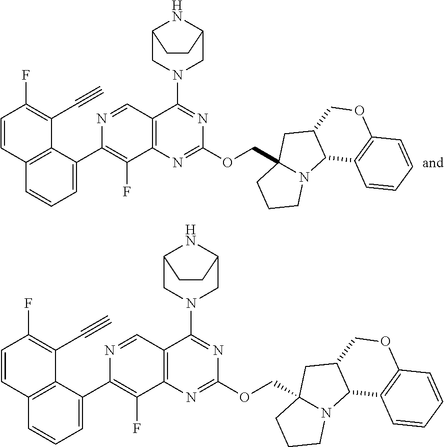

The title compounds were prepared analogously as described in synthesis of Example 13, Step 1-2, using ((6aS,7aR,11aR)-6a,9,10,11a-tetrahydro-6H,7H-chromeno[3,4-b]pyrrolizin-7a(8H)-yl)methanol and ((6aR,7aS,11aS)-6a,9,10,11a-tetrahydro-6H,7H-chromeno[3,4-b]pyrrolizin-7a(8H)-yl)methanol instead of (2-methylidene-tetrahydro-1H-pyrrolizin-7a-yl)methanol in step 1, provided the title compound. MS (ES, m/z): [M+H]⁺=671.4.

### Example 16

### Synthesis of 5-ethynyl-4-(8-fluoro-4-(3,8-diazaspiro[bicyclo[3.2.1]octane-6,1′-cyclopropan]-3-yl)-2-((tetrahydro-1H-pyrrolizin-7a(5H)-yl)methoxy)pyrido[4,3-d]pyrimidin-7-yl)naphthalen-2-ol

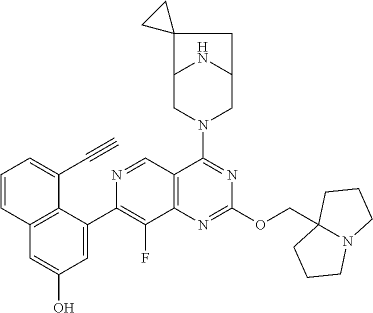 [SMILES: C#Cc1cccc2cc(O)cc(-c3ncc4c(N5CC6CC7(CC7)C(C5)N6)nc(OCC56CCCN5CCC6)nc4c3F)c12]

Proceeding analogously as described in Example 7, Step 1-10 using 5-ethynyl-4-(8-fluoro-4-(3,8-diazaspiro[bicyclo[3.2.1]octane-6,1′-cyclopropan]-3-yl)-2-((tetrahydro-1H-pyrrolizin-7a(5H)-yl)methoxy)pyrido[4,3-d]pyrimidin-7-yl)naphthalen-2-ol instead of (1R,5S)-3,8-diazabicyclo [3.2.1]oct-6-ene-6-carbonitrile bis(2,2,2-trifluoroacetate) in Step 9, provided the title compound. MS (ES, m/z): [M+H]⁺=591.3.

### Example 17

### Synthesis of 3-(7-(8-ethynyl-3-hydroxynaphthalen-1-yl)-8-fluoro-2-((tetrahydro-1H-pyrrolizin-7a(5H)-yl)methoxy)pyrido[4,3-d]pyrimidin-4-yl)-3,8-diazabicyclo[3.2.1]octane-6-carbonitrile

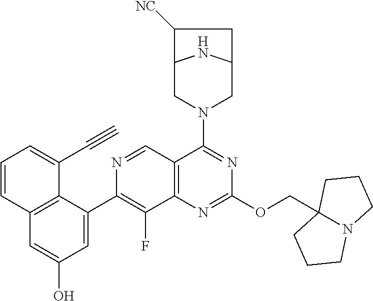 [SMILES: C#Cc1cccc2cc(O)cc(-c3ncc4c(N5CC6CC(C#N)C(C5)N6)nc(OCC56CCCN5CCC6)nc4c3F)c12]

Proceeding analogously as described in Example 7, Step 1-10 using 3,8-diazabicyclo-[3.2.1]octane-6-carbonitrile instead of (1R,5S)-3,8-diazabicyclo [3.2.1]oct-6-ene-6-carbonitrile bis(2,2,2-trifluoroacetate) in Step 9, provided the title compound. MS (ES, m/z): [M+H]⁺=590.3.

### Example 18

### Synthesis of 4-(4-(3,8-diazabicyclo[3.2.1]oct-6-en-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethynylnaphthalen-2-ol

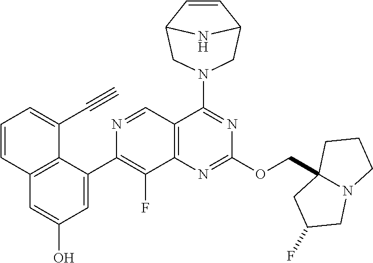

Proceeding analogously as described in Example 7, Step 1-10 using 3,8-diazabicyclo-[3.2.1]oct-6-ene instead of (1R,5S)-3,8-diazabicyclo [3.2.1]oct-6-ene-6-carbonitrile bis(2,2,2-trifluoroacetate) in Step 9, provided the title compound. MS (ES, m/z): [M+H]⁺=581.3.

### Example 19

### Synthesis of 5-ethynyl-6-fluoro-4-(8-fluoro-4-((1S,5R)-2-methyl-3,8-diazabicyclo[3.2.1]-octan-3-yl)-2-((2-methylenetetrahydro-1H-pyrrolizin-7a(5H)-yl)methoxy)pyrido[4,3-d]pyrimidin-7-yl)naphthalen-2-ol

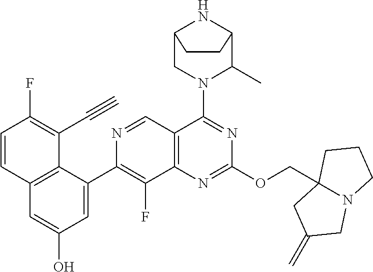 [SMILES: C#Cc1c(F)ccc2cc(O)cc(-c3ncc4c(N5CC6CCC(N6)C5C)nc(OCC56CCCN5CC(=C)C6)nc4c3F)c12]

### Step 1: 8-fluoro-7-(7-fluoro-3-(methoxymethoxy)-8-((triisopropylsilyl)ethynyl)naphthalen-1-yl)-2-((2-methylenetetrahydro-1H-pyrrolizin-7a(5H)-yl)methoxy)-4-(2-(trimethylsilyl)ethoxy)pyrido-[4,3-d]pyrimidine

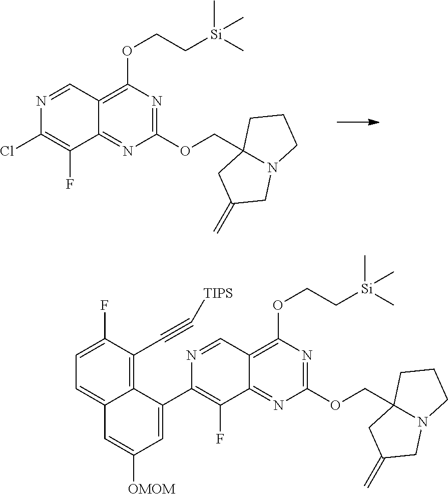

To a stirred mixture of 7-chloro-8-fluoro-2-((2-methylenetetrahydro-1H-pyrrolizin-7a(5H)-yl)methoxy)-4-(2-(trimethylsilyl)ethoxy)pyrido[4,3-d]pyrimidine (160 mg, 0.35 mmol, 1.00 eq.) and ((2-fluoro-6-(methoxymethoxy)-8-(4,4,5,5-tetramethyl-1,3,2-dioxaborolan-2-yl)naphthalen-1-yl)ethynyl)triisopropylsilane (200 mg, 0.39 mmol, 1.11 eq.) in DME (1.5 mL) and H₂O (0.15 mL) were added K₂CO₃ (147 mg, 1.06 mmol, 3.03 eq.) and cataCXium A Pd G3 (9.0 mg, 0.012 mmol, 0.034 eq.). After stirring at 85° C. overnight under nitrogen atmosphere, the reaction mixture was concentrated and the residue was purified by silica gel column chromatography, eluted with PE/EA (0˜50%), to afford the title compound (135 mg, 48.6%) as a light-yellow solid.

### Step 6: 7-(8-ethynyl-7-fluoro-3-(methoxymethoxy)naphthalen-1-yl)-8-fluoro-2-((2-methylenetetrahydro-1H-pyrrolizin-7a(5H)-yl)methoxy)pyrido[4,3-d]pyrimidin-4-ol

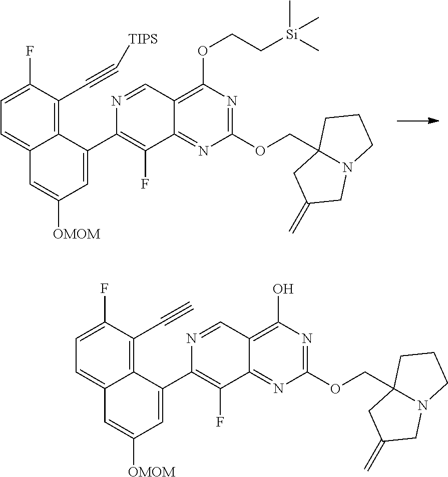

A mixture of 8-fluoro-7-(7-fluoro-3-(methoxymethoxy)-8-((triisopropylsilyl)ethynyl) naphthalen-1-yl)-2-((2-methylenetetrahydro-1H-pyrrolizin-7a(5H)-yl)methoxy)-4-(2-(trimethyl-silyl)ethoxy)pyrido[4,3-d]pyrimidine (140 mg, 0.175 mmol, 1.00 eq.) and CsF (265 mg, 1.75 mmol, 10.00 eq.) in DMF (1.4 mL) was stirred for 1 h at room temperature. The resulting mixture was diluted with water and then extracted with EtOAc. The combined organic layers were washed with water, brine, dried over anhydrous Na₂SO₄, filtered and then concentrated. The residue was purified by silica gel column chromatography, eluted with EA/PE (0˜50%), to afford the title compound (80 mg, 84.0%) as a light-yellow solid.

### Step 7: 1-((1S,5R)-3-(7-(8-ethynyl-7-fluoro-3-(methoxymethoxy)naphthalen-1-yl)-8-fluoro-2-((2-methylenetetrahydro-1H-pyrrolizin-7a(5H)-yl)methoxy)pyrido[4,3-d]pyrimidin-4-yl)-2-methyl-3,8-diazabicyclo[3.2.1]octan-8-yl)-2,2,2-trifluoroethan-1-one

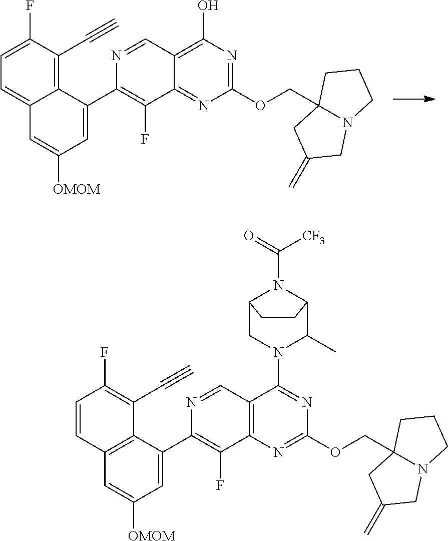

To a stirred solution of 7-(8-ethynyl-7-fluoro-3-(methoxymethoxy)naphthalen-1-yl)-8-fluoro-2-((2-methylenetetrahydro-1H-pyrrolizin-7a(5H)-yl)methoxy)pyrido[4,3-d]pyrimidin-4-ol (150 mg, 0.275 mmol, 1.00 eq.), 2,2,2-trifluoro-1-((1S,5R)-2-methyl-3,8-diazabicyclo [3.2.1]octan-8-yl)ethan-1-one (92 mg, 0.414 mmol, 1.51 eq.) and PyBOP (215 mg, 0.413 mmol, 1.50 eq.) in DMF (1.5 mL) was added DIEA (107 mg, 0.828 mmol, 3.01 eq.) dropwise at room temperature. After stirring for 36 h at 37° C., the reaction mixture was quenched with water at room temperature, and then extracted with EtOAc. The combined organic layers were washed with water, brine, dried over anhydrous Na₂SO₄, filtered and then concentrated. The residue was purified by silica gel column chromatography, eluted with DCM/MeOH (0˜10%), to afford the title compound (180 mg, 87.3%) as alight brown oil.

### Step 8: 7-(8-ethynyl-7-fluoro-3-(methoxymethoxy)naphthalen-1-yl)-8-fluoro-4-((1S,5R)-2-methyl-3,8-diazabicyclo[3.2.1]octan-3-yl)-2-((2-methylenetetrahydro-1H-pyrrolizin-7a(5H)-yl)methoxy)pyrido[4,3-d]pyrimidine

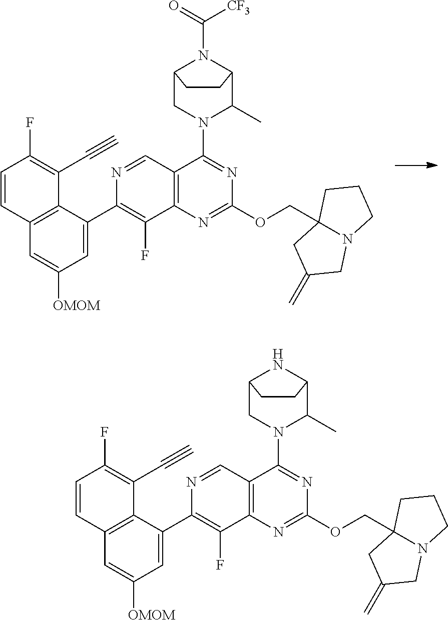

A mixture of 1-((1S,5R)-3-(7-(8-ethynyl-7-fluoro-3-(methoxymethoxy)naphthalen-1-yl)-8-fluoro-2-((2-methylenetetrahydro-1H-pyrrolizin-7a(5H)-yl)methoxy)pyrido[4,3-d]pyrimidin-4-yl)-2-methyl-3,8-diazabicyclo [3.2.1]octan-8-yl)-2,2,2-trifluoroethan-1-one (180 mg, 0.24 mmol, 1.00 eq.) and K₂CO₃ (332 mg, 2.40 mmol, 10.00 eq.) in EtOH (2 mL) and H₂O (0.2 mL) was stirred for 2 h at 70° C. The reaction mixture was cooled to room temperature, quenched with water and then extracted with EtOAc. The combined organic layers were washed with water, brine, dried over anhydrous Na₂SO₄, filtered and then concentrated. The residue was purified by silica gel column chromatography, eluted with MeOH/DCM (0˜5%), to afford the title compound (90 mg, 58.3%) as a light brown solid.

### Step 9: 5-ethynyl-6-fluoro-4-(8-fluoro-4-((1S,5R)-2-methyl-3,8-diazabicyclo[3.2.1]octan-3-yl)-2-((2-methylenetetrahydro-1H-pyrrolizin-7a(5H)-yl)methoxy)pyrido[4,3-d]pyrimidin-7-yl)naphthalen-2-ol

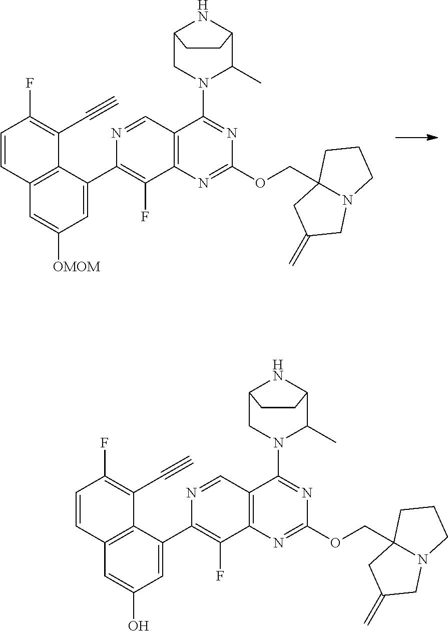

To a stirred solution of 7-(8-ethynyl-7-fluoro-3-(methoxymethoxy)naphthalen-1-yl)-8-fluoro-4-((1S,5R)-2-methyl-3,8-diazabicyclo[3.2.1]octan-3-yl)-2-((2-methylenetetrahydro-1H-pyrrolizin-7a(5H)-yl)methoxy)pyrido[4,3-d]pyrimidine (25 mg, 0.038 mmol, 1.00 eq.) in DCM (0.4 mL) was added 4.0 M HCl in 1,4-dioxane (0.2 mL) dropwise at 0° C. The resulting mixture was stirred for 1 h, concentrated and then neutralized to pH=7 with NH₃ in MeOH. After concentration, the crude product was purified by Prep-HPLC to afford the title compound (2.5 mg, 10.8%) as a light brown solid. MS (ES, m/z): [M+H]⁺=609.4.

### Example 20

### Synthesis of 4-(4-((1R,5S)-3,8-diazabicyclo[3.2.1]octan-3-yl)-6-chloro-8-fluoro-2-((2-methylenetetrahydro-1H-pyrrolizin-7a(5H)-yl)methoxy)quinazolin-7-yl)-5-ethynylnaphthalen-2-ol

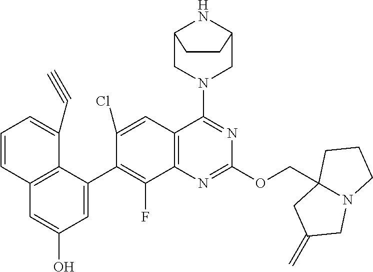 [SMILES: C#Cc1cccc2cc(O)cc(-c3c(Cl)cc4c(N5CC6CCC(C5)N6)nc(OCC56CCCN5CC(=C)C6)nc4c3F)c12]

### Step 1: tert-butyl (1R,5S)-3-(7-bromo-6-chloro-8-fluoro-2-((2-methylenetetrahydro-1H-pyrrolizin-7a(5H)-yl)methoxy)quinazolin-4-yl)-3,8-diazabicyclo[3.2.1]octane-8-carboxylate

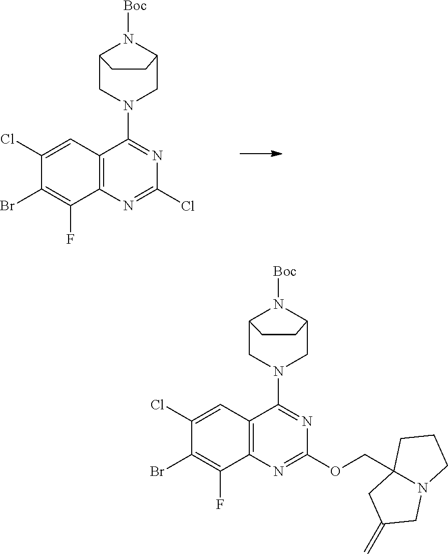 [SMILES: CC(C)(C)OC(=O)N1C2CCC1CN(c1nc(Cl)nc3c(F)c(Br)c(Cl)cc13)C2 ; C=C1CN2CCCC2(COc2nc(N3CC4CCC(C3)N4C(=O)OC(C)(C)C)c3cc(Cl)c(Br)c(F)c3n2)C1]

A vial charged with tert-butyl 3-(7-bromo-2,6-dichloro-8-fluoroquinazolin-4-yl)-3,8-diazabicyclo[3.2.1]octane-8-carboxylate (1.0 g, 1.98 mmol, 1.0 equiv), (2-methylidene-tetrahydro-1H-pyrrolizin-7a-yl)methanol (0.36 g, 2.37 mmol, 1.2 equiv) and DIEA (10 mL) was stirred for 6 h at 150° C. under nitrogen atmosphere. After cooling the reaction mixture at room temperature, the reaction mixture was quenched with water/ice and then extracted with EtOAc. The combined organic layers were washed with brine, dried over anhydrous Na₂SO₄ and concentrated. The residue was purified by silica gel column chromatography, eluted with THF/PE (0-50%) to afford the title compound (485 mg, 39.41%) as a light brown solid.

### Step 2: tert-butyl (1R,5S)-3-(6-chloro-8-fluoro-7-(3-(methoxymethoxy)-8-((triisopropylsilyl)-ethynyl)naphthalen-1-yl)-2-((2-methylenetetrahydro-1H-pyrrolizin-7a(5H)-yl)methoxy)quinazolin-4-yl)-3,8-diazabicyclo[3.2.1]octane-8-carboxylate

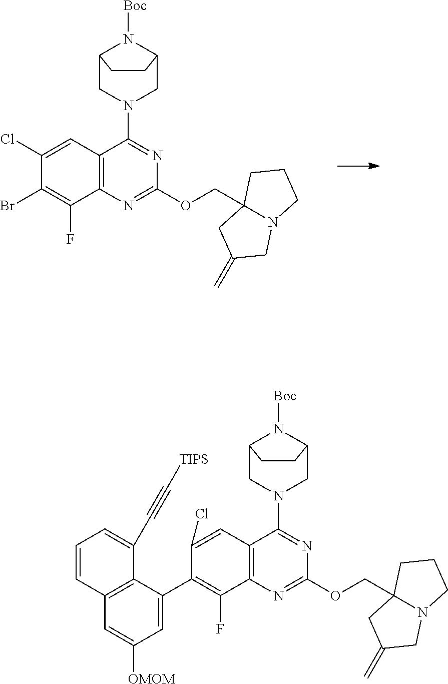

A 20 mL vial was charged with tert-butyl (1R,5S)-3-(7-bromo-6-chloro-8-fluoro-2-((2-methylene-tetrahydro-1H-pyrrolizin-7a(5H)-yl)methoxy)quinazolin-4-yl)-3,8-diazabicyclo[3.2.1]octane-8-carboxylate (250 mg, 0.40 mmol, 1.0 equiv), triisopropyl((6-(methoxymethoxy)-8-(4,4,5,5-tetramethyl-1,3,2-dioxaborolan-2-yl)naphthalen-1-yl)ethynyl)silane (238 mg, 0.48 mmol, 1.2 equiv), K₂CO₃ (166 mg, 1.20 mmol, 3.0 equiv), CATACXIUM A Pd G3 (29 mg, 0.04 mmol, 0.10 equiv) and DME (5 mL), water (0.5 mL). The resulting mixture was stirred for 16 h at 55° C. under nitrogen atmosphere and then cooled to room temperature. The reaction mixture was then concentrated under reduced pressure and the residue was purified by Prep-TLC MeOH/CH₂Cl₂=0.05 to afford the title compound (40 mg, 10.95%) as a light brown solid.

### Step 3: tert-butyl (1R,5S)-3-(6-chloro-7-(8-ethynyl-3-(methoxymethoxy)naphthalen-1-yl)-8-fluoro-2-((2-methylenetetrahydro-1H-pyrrolizin-7a(5H)-yl)methoxy)quinazolin-4-yl)-3,8-diazabicyclo[3.2.1]octane-8-carboxylate

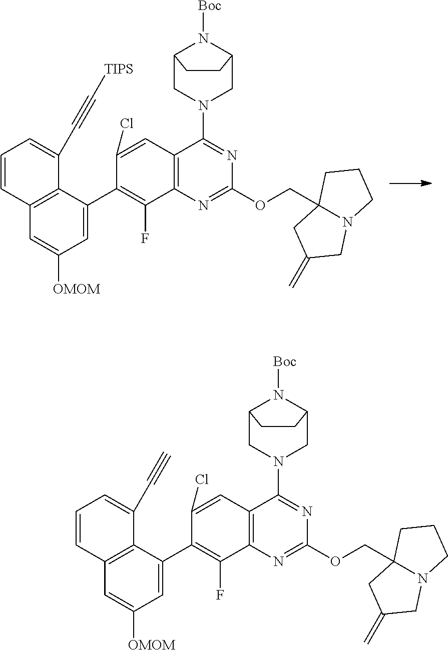

An 8 mL vial was charged with tert-butyl (1R,5S)-3-(6-chloro-8-fluoro-7-(3-(methoxymethoxy)-8-((triisopropylsilyl)ethynyl)naphthalen-1-yl)-2-((2-methylenetetrahydro-1H-pyrrolizin-7a(5H)-yl)methoxy)quinazolin-4-yl)-3,8-diazabicyclo[3.2.1]octane-8-carboxylate (40 mg, 0.044 mmol, 1 equiv), DMF (2 mL) and caesium fluoride (53 mg, 0.35 mmol, 8.0 equiv) at room temperature. The resulting mixture was stirred for 2 h at room temperature under nitrogen atmosphere. The reaction mixture was then quenched with water/ice and then extracted with EtOAc. The combined organic layers were washed with brine, dried over anhydrous Na₂SO₄ and concentrated to afford the title compound (30 mg, 90.54%) as a light brown solid.

### Step 4: 4-(4-((1R,5S)-3,8-diazabicyclo[3.2.1]octan-3-yl)-6-chloro-8-fluoro-2-((2-methylenetetrahydro-1H-pyrrolizin-7a(5H)-yl)methoxy)quinazolin-7-yl)-5-ethynylnaphthalen-2-ol

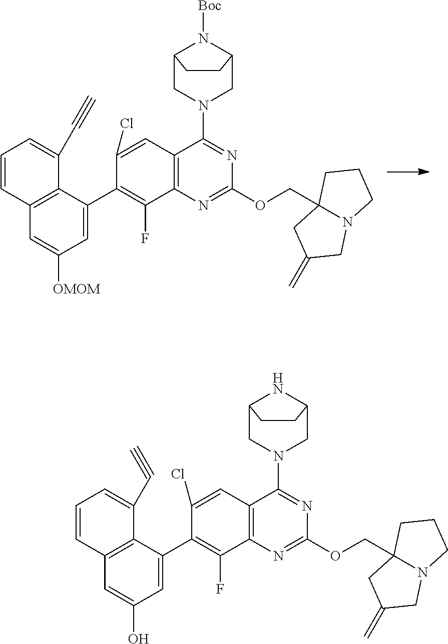

To a solution of tert-butyl (1R,5S)-3-(6-chloro-7-(8-ethynyl-3-(methoxymethoxy)naphthalen-1-yl)-8-fluoro-2-((2-methylenetetrahydro-1H-pyrrolizin-7a(5H)-yl)methoxy)quinazolin-4-yl)-3,8-diazabicyclo[3.2.1]octane-8-carboxylate (30 mg, 0.040 mmol, 1.0 equiv) and methanol (3.0 mL) was added conc. HCl (0.3 mL, 37% in water) dropwise at 0° C. under nitrogen atmosphere and the resulting mixture was stirred for 16 h at room temperature under nitrogen atmosphere. The reaction was basified to pH 8 with a solution of NH₃ in MeOH. The resulting mixture was concentrated under reduced pressure and the residue was purified by Prep-HPLC to afford the title compound (10 mg, 41.21%) as an off-white solid. MS (ES, m/z): [M+H]⁺=610.3.

### Example 21

### Synthesis of 4-((1R,5S)-3,8-diazabicyclo[3.2.1]octan-3-yl)-6-chloro-7-(8-ethynyl-7-fluoronaphthalen-1-yl)-8-fluoro-2-((2-methylenetetrahydro-1H-pyrrolizin-7a(5H)-yl)methoxy)quinazoline

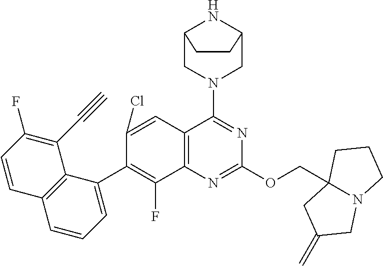 [SMILES: C#Cc1c(F)ccc2cccc(-c3c(Cl)cc4c(N5CC6CCC(C5)N6)nc(OCC56CCCN5CC(=C)C6)nc4c3F)c12]

Proceeding analogously as described in Example 1, Step 1-4 using ((2-fluoro-8-(4,4,5,5-tetramethyl-1,3,2-dioxaborolan-2-yl)naphthalen-1-yl)ethynyl)triisopropylsilane instead of triisopropyl((6-(methoxymethoxy)-8-(4,4,5,5-tetramethyl-1,3,2-dioxaborolan-2-yl)naphthalen-1-yl)ethynyl)silane in Step 2, provided the title compound. MS (ES, m/z): [M+H]⁺=612.3.

### BIOLOGICAL EXAMPLES

### Example 1

### p-ERK Cellular Assay

The ability of the compound of Formula (IIA1′), (IIA′), (II′), or (II) or a pharmaceutically acceptable salt thereof (test compound) to inhibit K-Ras 12D activity was tested using PANC-1 (ATCC, CRL-1469) and A-427 (ATCC, HTB-53) cell lines which harbor KRAS G12D mutation as described below.

PANC-1 (ATCC, CRL-1469) or A-427 (ATCC, HTB-53) were seeded in 96-well plates and cultured overnight (20,000 cells per well, 200 μl total volume). The following morning, cells were treated with test compound, with starting concentration at 10 μM and 2 log dilution down to 1 nM for 3 hours at 37° C. DMSO treatment served as control. p-ERK was then measured using Advanced Phospho-ERK 1/2 (Thr202/Tyr204) Assay Kit (Cisbio, Cat #64AERPET) following the manufacturer's instruction.

Briefly, medium was removed and 40 μl 1× lysis buffer was added to each well followed by 30 min. incubation on a plate shaker at room temperature. Then 8 μl of lysate was transferred to a white low volume 384-well plate. Acceptor d2 antibody and Crsptate antibody (Advanced Phospho-ERK 1/2 (Thr202/Tyr204) Assay Kit (Cisbio, Cat #64AERPET)) were diluted (1:20) with detection buffer and gently mixed (1:1) according to manufacturer's instruction, 2 μl antibodies mixture was added to the cell lysate and the plate was wrapped with foil, shaken for 1-2 minutes on a plate leader, and incubated for >4 hours at room temperature. Signal was then measured on a CLARIOstar® plate reader. Percentage inhibition was calculated with DMSO treatment as 100% of signal, and IC₅₀ was calculated by Graphpad Prism 7.

### Example 2

### p-ERK Cellular 2-Plate Assay

The ability of the compound of Formula (IIA′), (II′), or (II) or a pharmaceutically acceptable salt thereof (test compound) to inhibit K-Ras G12D activity was tested using AGS (Cobioer, CBP60476) cell lines which harbor KRAS G12D mutation as described below.

AGS (Cobioer, CBP60476) were seeded in 384-well plates and cultured overnight (10.000 cells per well 40 μl total volume). The following morning, cells were treated with test compound, with staring concentration at 10 μM and 3-fold dilution down to 0.5 nM for 3 h at 37° C., DMSO treatment served as control. p-ERK was then measured using AlphaLISA SureFire Ultra p-ERK1/2 (Thr202/Tyr204) Assay Kit (Perkin Elmer, cat #ALSU-PERK) following the manufacturer's instruction as follows.

Briefly, the culture medium was removed and 20 μl 1× lysis buffer was added to each well, followed by 30 minutes incubation on a plate shaker at room temperature. Then 10 μl of lysate was transferred to a white 384-well plate. Acceptor mixture was prepared according to manufacturer's instruction. 5 μl acceptor mixture was added to the cell lysate and the plate was wrapped with foil, spun at 500 rpm for 10 s, and incubated at RT for 60 min. Donor mixture was prepared under subdued light. 5 μl donor mixture was added to the cell lysate and the plate was spun at 500 rpm for 10s and incubated at RT for another 60 min. in the dark. Signal was then measured on a EnVision 2105 multimode plate reader. Percentage inhibition was calculated with DMSO treatment as 100% of signal, and IC50 was calculated by XLfit 5.5.x.

### Example 3

### p-ERK Cellular 1-Plate Assay

The ability of the compound of Formula (IIA′), (II′), or (II) or a pharmaceutically acceptable salt thereof (test compound) to inhibit K-Ras G12D activity was tested using AGS (Cobioer, CBP60476) cell lines which harbor KRAS G12D mutation as described below.

AGS (Cobioer, CBP60476) were seeded in 384-well plates and cultured overnight (5,000 cells per well, 40 μl total volume). The following morning, cells were treated with test compound, with starting concentration at 10 μM and 3-fold dilution down to 0.5 nM for 3 h at 3° C. DMSO treatment served as control. pERK was then measured using AlphaLISA SureFire Ultra p-ERK/2 (Thr202/Tyr204) Assay Kit (Perkin Elmer, cat #ALSU-PERK) following the manufacturer's instruction as follows.

Briefly, the culture medium was removed and 10 μl 1× lysis buffer was added to each well, followed by 10 minutes incubation on a plate shaker at room temperature. Acceptor mixture was prepared according to manufacturer's instruction. 5 μl acceptor mixture was added to the cell lysate and the plate was wrapped with foil, spun at 500 rpm for 10s and incubated at RT for 60 min. Donor mixture was prepared under subdued light. 5 μl donor mixture was added to the cell lysate and the plate was spun at 500 rpm for 10s and incubated at RT for another 60 min. in the dark. Signal was then measured on a EnVision 2105 multimode plate reader. Percentage inhibition was calculated with DMSO treatment as 100% of signal, and IC₅₀ was calculated by XLfit 5.5.x.

TABLE 1

| Cpd | AGS |
|---|---|
| # | PERK |
| (from | EC₅₀ |
| Cpd | (nM) |
| 1 | 0.2 |
| 2 | 0.2 |
| 3 | 1.5 |
| 4 | 5^{#} |
| 5 | 320 |
| 6 | 0.6 |
| 7 | 260 |
| 8 | One of 8 and 14 is |
| and | 27nm and the other |
| 14 | of 8 and 14 602 |
| 9 | 253 |
| 10 | 128 |
| 11 | 7 |
| 12 | 105 |
| 13 | 0.1 |
| 15 | 0.1 |
| 16 | 0.9 |
| 17 | 1.0 |
| 18 | 0.37 |
| 19 | 4.5 |
| 20 | 0.1 |
| 21 | 0.7 |
| 22 | 1.0 |
| 23 | 0.48 |
| 24 | 3.7 |
| 25 | 3.0 |
| 26 | 7.0 |
| 27 | 20 |
| 28 | 46 |
| 29 | 2.0 |
| 30 | 4.0 |
| 31 | 4.1 |
| 32 | 6.0 |
| 33 | 1.4 |
| 34 | 25 |
| 35a | one of 35a and 35b is |
| 35 b | 55 and the other of |
| | 35a and 35b is 2.5 |
| 36 | 5.2 |
| 37 | 6.6 |
| 38 | 1.1 |
| 39 | 0.25 |
| 40 | 3.6 |
| 41 | >100 |
| 42 | 36* |
| 43 | 18* |
| 44 | >100* |
| 46 | 5.3* |
| 47 | 52* |
| 48 | 33 |
| 49 | 19 |
| 50 | 213* |
| 51 | 836* |
| 52 | >100* |
| 53 | 266 |
| 54 | 1.4* |
| 55 | >100 |
| 56 | 1.3 |

^{#}EC₅₀ was measured as described in Biological Example 1;
*EC₅₀ was measured as described in Biological Example 3; and
EC₅₀ for remaining compounds was measured as described in Biological Example 2.

### EXAMPLES

The following are representative pharmaceutical formulations containing a compound of the present disclosure.

### Tablet Formulation

The following ingredients are mixed intimately and pressed into single scored tablets.

| Ingredient | Quantity per tablet (mg) |
|---|---|
| compound Formula (IIA’), (II’), or (II) | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| Ingredient | Quantity per capsule (mg) |
|---|---|
| compound Formula (IIA’), (II’), or (II) | 200 |
| lactose spray dried | 148 |
| magnesium stearate | 2 |

### Injectable Formulation

Compound of the disclosure (e.g., compound 1) in 2% HPMC, 1% Tween 80 in DI water, pH 2.2 with MSA, q.s. to at least 20 mg/mL

### Inhalation Composition

To prepare a pharmaceutical composition for inhalation delivery, 20 mg of a compound disclosed herein is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound disclosed herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound disclosed herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 10 g of a compound disclosed herein is mixed with 30 mL of a 0.05M phosphate buffer solution (pH 4.4). The solution is placed in a nasal administrator designed to deliver 100 ul of spray for each application.

What is claimed:

1. A compound of Formula (IIA′): 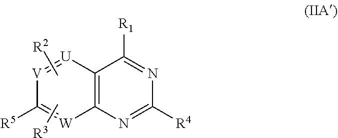
wherein:
U, V, and W are CH; or one or two of U, V, and W are N and the other of U, V, and W are CH;
R¹ is a ring of formula: 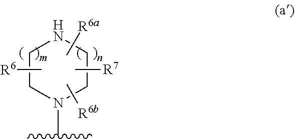
where
m and n are independently 0, 1, or 2;
R⁶ is hydrogen, deuterium, alkyl, alkoxy, halo, haloalkyl, hydroxy, hydroxylalkyl, alkoxyalkyl, cyano, or cyanomethyl, provided R⁶ is not attached to the ring —NH—;
R⁷ is hydrogen, deuterium, alkyl, alkoxy, halo, haloalkyl, hydroxy, hydroxylalkyl, or alkoxyalkyl, provided R⁷ is not attached to the ring —NH—; or
when R⁶ and R⁷ are attached to the carbon atoms of the ring that are opposite or diagonal to each other, then R⁶ and R⁷ can combine to form —(CH₂)_{z}— where (z is 1, 2, or 3), or —CH═CH—;
R^{6a} is hydrogen, deuterium, alkyl, alkylidienyl, alkoxy, halo, haloalkyl, hydroxy, hydroxyalkyl, alkoxyalkyl, cyano, or cyanomethyl, provided R^{6a} is not attached to the ring —NH—;
R^{6b} is hydrogen or alkyl, provided R^{6b} is not attached to the ring —NH—, or
when R^{6a} and R^{6b} are attached to the same carbon of ring (a′), they can combine to form alkylidienyl or cycloalkylene;
R² is hydrogen, deuterium, alkyl, halo, haloalkyl, alkoxy, hydroxy, or cyano, provided that R² is absent when two of U, V, and W are N;
R³ is hydrogen, deuterium, alkyl, halo, haloalkyl, alkoxy, cycloalkyloxy, hydroxy, or cyano;
R⁴ is:
(i) —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, heterocyclyl fused bicyclic heterocyclyl, heterocyclyl fused bicyclic heterocyclylalkyl, tricyclic heterocyclyl, tricyclic heterocyclylalkyl, fused tricyclic heterocyclyl, fused tricyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, heterocyclyl fused bicyclic heterocyclyl, by itself of as part of heterocyclyl fused bicyclic heterocyclylalkyl, tricyclic heterocyclyl, by itself or as part of tricyclic heterocyclylalkyl, fused tricyclic heterocyclyl, by itself or as part of fused tricyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, alkenyl, haloalkenyl, cycloalkyl, cycloalkyloxy, cycloalkylalkyl, bridged cycloalkyl, halo, haloalkyl, haloalkoxy, hydroxy, alkoxy, alkylidienyl, haloalkylidienyl, alkoxvalkylidienyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is aryl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, then at least one of R^{d}, R^{e}, and R^{f} is alkylidenyl, haloalkylidenyl, alkoxyalkylidenyl, alkylsulfonyl, alkylsulfonylalkyl, dialkyl(oxo)phosphinyl, or dialkyl(oxo)phosphinylalkyl; and
R⁵ is -Q-R³⁶ where Q is bond, alkylene, or —C(═O)—; and R³⁶ is hydrogen, cycloalkyl, fused cycloalkyl, aryl, aralkyl, heteroaryl, or heteroaralkyl wherein aryl, aryl in aralkyl, heteroaryl, and heteroaryl in heteroaralkyl are independently substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl; or
a pharmaceutically acceptable salt thereof.

2. The compound of claim 1, or a pharmaceutically acceptable salt thereof wherein:
R¹ is a ring of formula: 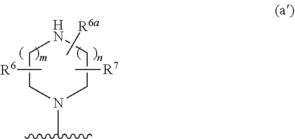
where:
R^{6a} is hydrogen, deuterium, alkyl, alkoxy, halo, haloalkyl, hydroxy, hydroxylalkyl, alkoxyalkyl, cyano, or cyanomethyl, provided R^{6a} is not attached to the ring —NH—;
R⁴ is:
(i) —Z—R³⁰ where Z is a bond, O, NH, N(alkyl), or S; and R³⁰ is aryl, heteroaryl, heteroaralkyl, heterocyclyl, heterocyclylalkyl, bicyclic heterocyclyl, bicyclic heterocyclylalkyl, phosphinyl bicyclic heterocyclyl, phosphinyl bicyclic heterocyclylalkyl, fused bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein aryl, heteroaryl, by itself or as part of heteroaralkyl, heterocyclyl, by itself or as part of heterocyclylalkyl, bicyclic heterocyclyl, by itself or as part of bicyclic heterocyclylalkyl, phosphinyl bicyclic heterocyclyl, by itself or as part of phosphinyl bicyclic heterocyclylalkyl, fused bicyclic heterocyclyl as part of fused bicyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that; when R³⁰ is aryl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

3. The compound of claim 1 or 2, or a pharmaceutically acceptable salt thereof, wherein R⁴ is —Z—R³⁰ where Z is O; and R³⁰ is phosphinyl bicyclic heterocyclylalkyl or bicyclic heterocyclylalkyl, wherein bicyclic heterocyclyl as part of bicyclic heterocyclylalkyl and phosphinyl bicyclic heterocyclyl as part of phosphinyl bicyclic heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is bicyclic heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

4. The compound of claim 2, or a pharmaceutically acceptable salt thereof, wherein R⁴ is —Z—R³⁰ where Z is O; and R³⁰ is heterocyclylalkyl, bicyclic heterocyclylalkyl, phosphinyl bicyclic heterocyclyl, phosphinyl bicyclic heterocyclylalkyl, or fused bicyclic heterocyclylalkyl, wherein heterocyclyl as part of heterocyclylalkyl, bicyclic heterocyclyl as part of bicyclic heterocyclylalkyl, phosphinyl bicyclic heterocyclyl, by itself or as part of phosphinyl bicyclic heterocyclylalkyl, or fused bicyclic heterocyclyl as part of fused bicyclic heterocyclylalkyl are substituted with R^{d}, R^{e}, and R^{f}; provided that; when R³⁰ is heterocyclylalkyl or bicyclic heterocyclylalkyl, then at least one of R^{d}, R^{e}, and R^{f} is alkylidenyl, alkoxyalkylidenyl, alkylsulfonyl, alkylsulfonylalkyl, dialkyl(oxo)phosphinyl, or dialkyl(oxo)phosphinylalkyl.

5. The compound of claim 1, or a pharmaceutically acceptable salt thereof, wherein R⁴ is —Z—R³⁰ where Z is O; and R³⁰ is tricyclic heterocyclyl, tricyclic heterocyclylalkyl, fused tricyclic heterocyclyl, fused tricyclic heterocyclylalkyl, bicyclic heterocyclyl, or bicyclic heterocyclylalkyl wherein tricyclic heterocyclyl, by itself or as part of tricyclic heterocyclylalkyl and fused tricyclic heterocyclyl, by itself or as part of fused tricyclic heterocyclylalkyl, are substituted with R^{d}, R^{e}, and R^{f} independently selected from hydrogen, alkyl, alkenyl, haloalkenyl, cycloalkyl, cycloalkyloxy, cycloalkylalkyl, bridged cycloalkyl, halo, haloalkyl, haloalkoxy, hydroxy, alkoxy, alkylidenyl, haloalkylidenyl, alkoxyalkylidenyl, alkoxyalkyl, alkoxyalkyloxy, alkylsulfonyl, alkylsulfonylalkyl, dialkyl(oxo)phosphinyl, dialkyl(oxo)phosphinylalkyl, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; and bicyclic heterocyclyl and bicyclic heterocyclyl as part of bicyclic heterocyclylalkyl are substituted with alkylidene or haloalkylidene.

6. The compound of claim 1, or a pharmaceutically acceptable salt thereof, wherein R⁴ is —Z—R³⁰ where Z is O; and R³⁰ is tricyclic heterocyclylalkyl, fused tricyclic heterocyclylalkyl, or bicyclic heterocyclylalkyl wherein R³⁰ is tricyclic heterocyclylalkyl, fused tricyclic heterocyclylalkyl, or bicyclic heterocyclylalkyl wherein the tricyclic heterocyclyl as part of tricyclic heterocyclylalkyl, and fused tricyclic heterocyclyl as part of fused tricyclic heterocyclylalkyl, are substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkoxyalkyl, alkoxyalkyloxy, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; and the bicyclic heterocyclyl as part of bicyclic heterocyclylalkyl is substituted with R^{d}, R^{e}, and R^{f} independently selected from hydrogen, alkyl, cycloalkyl, cycloalkyloxy, cycloalkylalkyl, bridged cycloalkyl, halo, haloalkyl, haloalkoxy, hydroxy, alkoxy, alkylidenyl, haloalkylidenyl, alkoxyalkylidenyl, alkoxyalkyl, alkoxyalkyloxy, acyl, cyano, oxo, hydroxyalkyl, alkylamino, dialkylamino, dialkylaminocarbonylalkyl, optionally substituted aryl, optionally substituted aralkyl, optionally substituted heteroaryl, and optionally substituted heterocyclyl; provided that when R³⁰ is bicyclic heterocyclyalkyl, then at least one of R^{d}, R^{e}, and R^{f} alkylidene, alkoxyalkylidene, or haloalkylidene.

7. The compound of claim 6, or a pharmaceutically acceptable salt thereof, wherein R³⁰ is tricyclic heterocyclylmethylene, fused tricyclic heterocyclylmethylene, or bicyclic heterocyclylmethylene independently substituted with R^{d}, R^{e}, and R^{f}.

8. The compound of claim 1, 6, or 7, or a pharmaceutically acceptable salt thereof, wherein R³⁰ is tricyclic heterocyclylmethylene wherein tricyclic heterocyclyl as part of tricyclic heterocyclylmethylene is substituted with R^{d}, R^{e}, and R^{f}.

9. The compound of claim 1, 6, or 7, or a pharmaceutically acceptable salt thereof, wherein R³⁰ is fused tricyclic heterocyclylmethylene wherein fused tricyclic heterocyclyl as part of fused tricyclic heterocyclylmethylene is substituted with R^{d}, R^{e}, and R^{f}.

10. The compound of claim 1, 6, or 7, or a pharmaceutically acceptable salt thereof, wherein R³⁰ is bicyclic heterocyclylmethylene wherein bicyclic heterocyclyl as part of bicyclic heterocyclylmethylene is substituted with R^{d}, R^{e}, and R^{f}.

11. The compound of any one of claims 1 to 10, or a pharmaceutically acceptable salt thereof, wherein R^{d} is hydrogen.

12. The compound of any one of claims 1 to 7 and 10, or a pharmaceutically acceptable salt thereof, wherein R⁴ is: 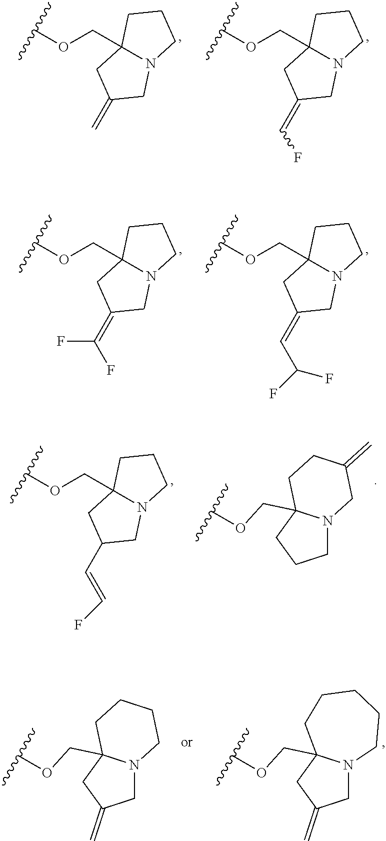
each ring optionally substituted with R^{e} selected from hydrogen, methyl, ethyl, isopropyl, cyclopropyl, cyclopropyloxy, fluoro, chloro, difluoromethyl, trifluoromethyl, difluoromethyloxy, trifluoromethoxy, methoxy, ethoxy, methoxymethyl, methoxymethyloxy, cyano, methylamino, dimethylamino, diethylamino, hydroxymethyl, phenyl, and benzyl.

13. The compound of any one of claims 1 to 7 and 8, or a pharmaceutically acceptable salt thereof, wherein R⁴ is. 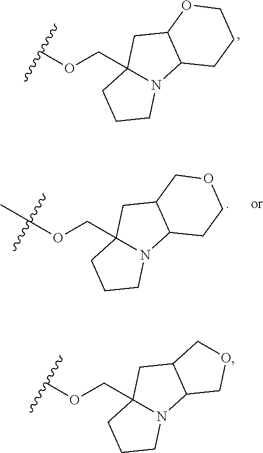 [SMILES: *OCC12CCCN1C1CCCOC1C2 ; *OCC12CCCN1C1CCOCC1C2 ; *OCC12CCCN1C1COCC1C2]
each ring optionally substituted with R^{e} selected from hydrogen, hydrogen, methyl, ethyl, isopropyl, cyclopropyl, cyclopropyloxy, fluoro, chloro, difluoromethyl, trifluoromethyl, difluoromethyloxy, trifluoromethoxy, methoxy, ethoxy, methoxymethyl, methoxymethyloxy, cyano, methylamino, dimethylamino, diethylamino, hydroxymethyl, phenyl, and benzyl.

14. The compound of any one of claims 1 to 13, or a pharmaceutically acceptable salt thereof, wherein R^{e} is hydrogen.

15. The compound of any of one of claims 1 to 14, or a pharmaceutically acceptable salt thereof, wherein:
R¹ is:

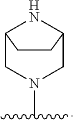 [SMILES: *N1CC2CCC(C1)N2]

16. The compound of any of one of claims 1 to 14, or a pharmaceutically acceptable salt thereof, wherein:
R¹ is: 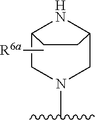
where R^{6a} is other than hydrogen.

17. The compound of any of one of claims 1 to 14, or a pharmaceutically acceptable salt thereof, wherein:
R¹ is: 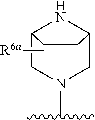
where R^{6a} is alkyl.

18. The compound of any one of claims 1 to 17, or a pharmaceutically acceptable salt thereof, having a structure of formula (II′c) as follows:

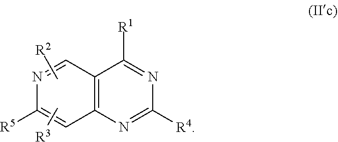

19. The compound of any one of claims 1 to 18, or a pharmaceutically acceptable salt thereof, having a structure of formula (II′d) as follows:

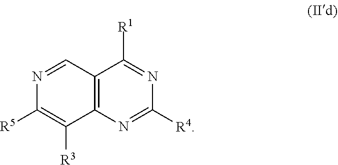

20. The compound of any one of claims 1 to 19, or a pharmaceutically acceptable salt thereof, wherein R⁵ is -Q-R³⁶ where Q is bond and R³⁶ is cycloalkyl, fused cycloalkyl, aryl, aralkyl, heteroaryl, or heteroaralkyl wherein aryl, aryl in aralkyl, heteroaryl, and heteroaryl in heteroaralkyl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.

21. The compound of any one of claims 1 to 20, or a pharmaceutically acceptable salt thereof, wherein R⁵ is -Q-R³⁶ where Q is bond and R³⁶ is phenyl or naphthyl substituted with R^{aa}R^{bb}, R^{cc} and R^{dd}.

22. The compound of any one of claims 1 to 21, or a pharmaceutically acceptable salt thereof, wherein R^{aa} and R^{bb} independently selected from hydrogen, methyl, ethyl, fluoro, chloro, trifluoromethyl, difluoromethyl, trifluoromethoxy, hydroxy, methyl, ethoxy, cyclopropyl, amino, cyano, and hydroxymethyl, R^{cc} is hydrogen, ethynyl, 2-cyanoethyn-1-yl, or fluoro, and R^{dd} is hydrogen, methyl, fluoro, amino, or cyclopropyl.

23. The compound of any one of claims 1 to 22, or a pharmaceutically acceptable salt thereof, wherein R⁵ is -Q-R³⁶ where Q is bond and R³⁶ is:

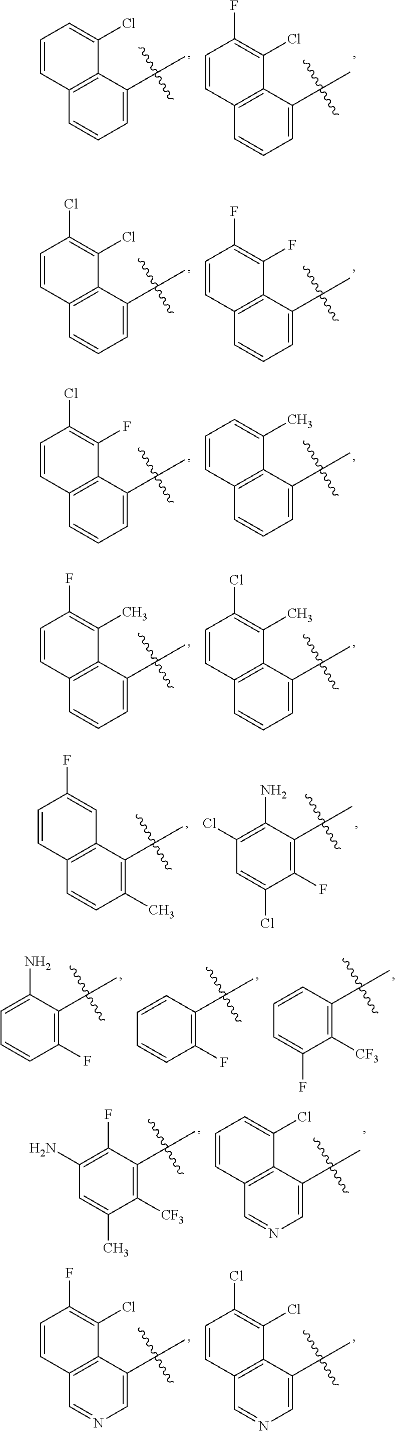

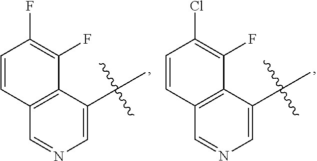 [SMILES: *c1cncc2ccc(F)c(F)c12 ; *c1cncc2ccc(Cl)c(F)c12]

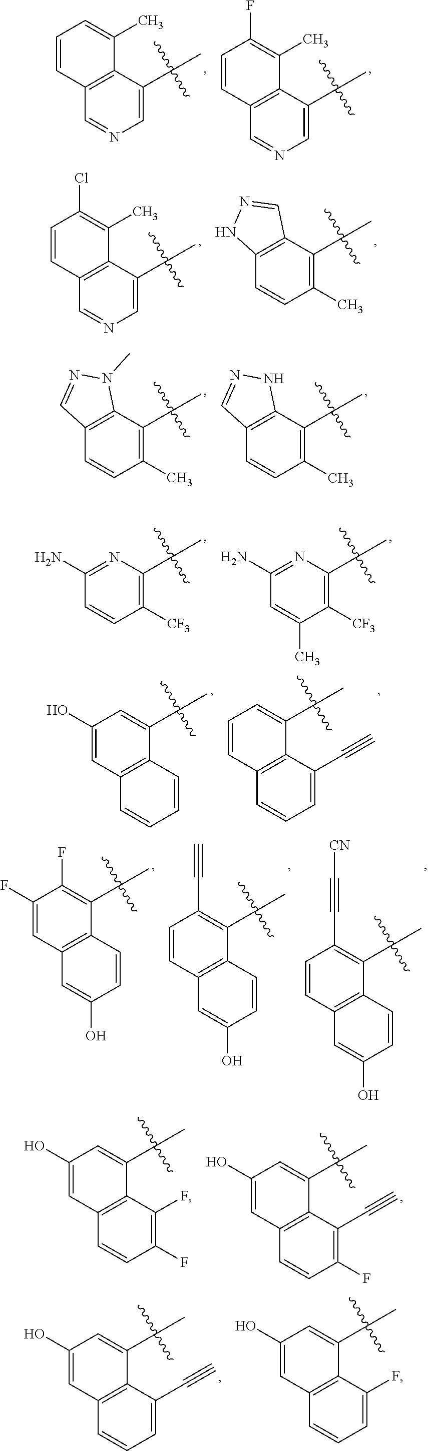

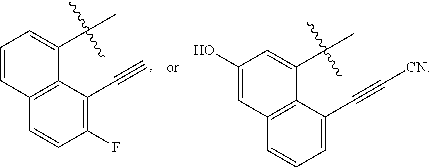 [SMILES: *c1cccc2ccc(F)c(C#C)c12 ; *c1cc(O)cc2cccc(C#CC#N)c12]

24. The compound of any one of claims 1 to 23, or a pharmaceutically acceptable salt thereof, wherein R⁵ is -Q-R³⁶ where Q is bond and R³⁶ is:

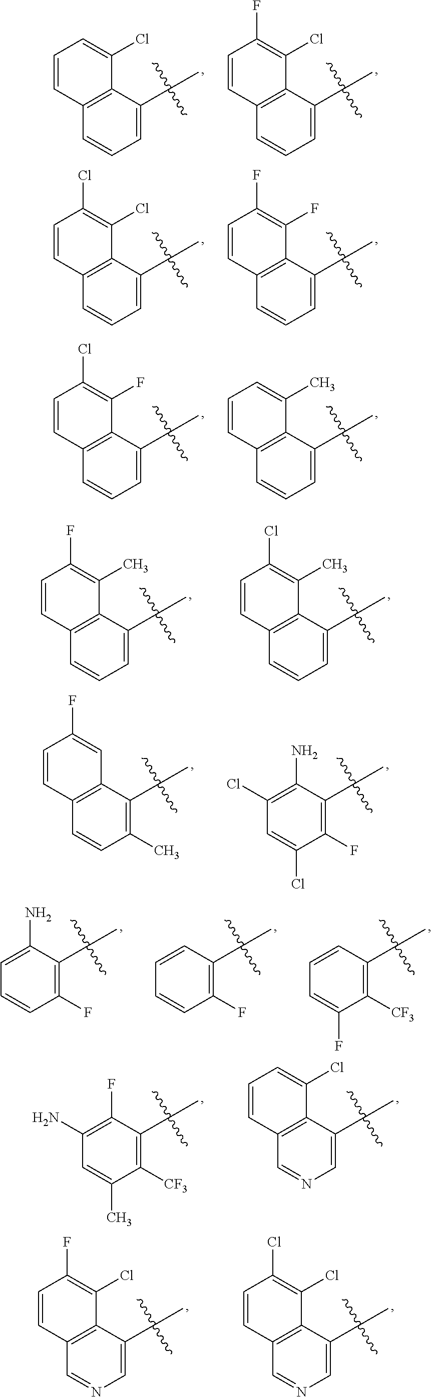

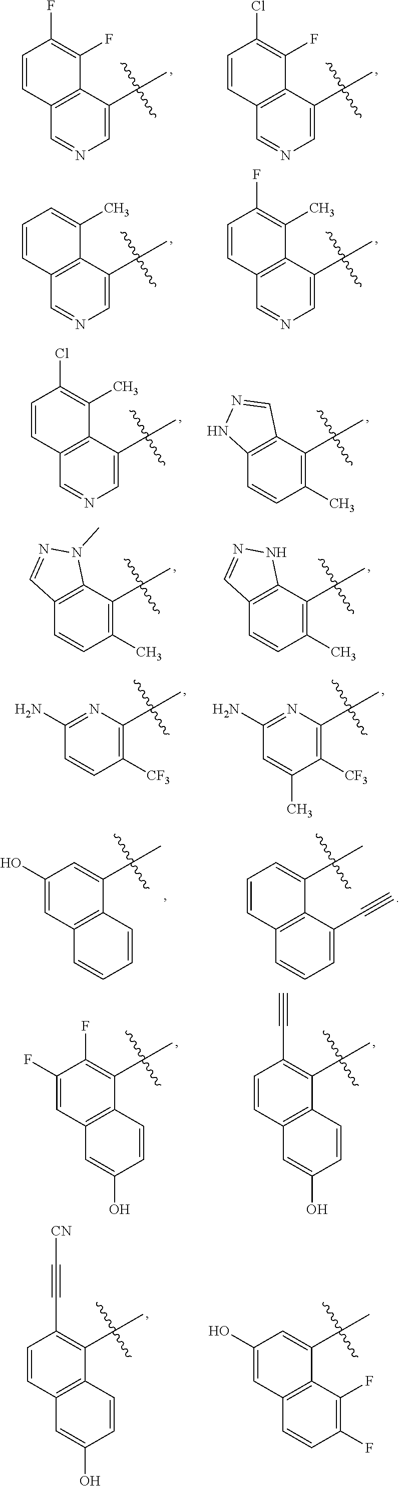

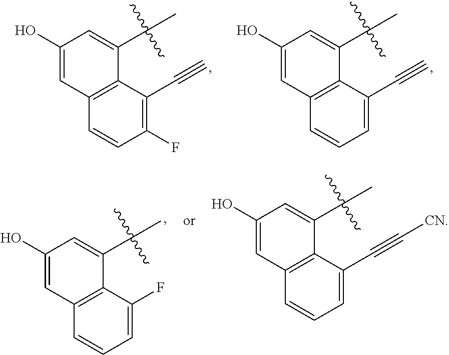 [SMILES: *c1cc(O)cc2ccc(F)c(C#C)c12 ; *c1cc(O)cc2cccc(C#C)c12 ; *c1cc(O)cc2cccc(F)c12 ; *c1cc(O)cc2cccc(C#CC#N)c12]

25. The compound of any one of claims 1 to 24, or a pharmaceutically acceptable salt thereof, wherein, R² is hydrogen, halo, or alkyl, and R³ hydrogen, halo, cycloalkyloxy, or alkyl.

26. The compound of any one of claims 1 to 25, or a pharmaceutically acceptable salt thereof, wherein R² is hydrogen or chloro and R³ is hydrogen, fluoro, or cyclopropyloxy.

27. The compound of any one of claims 1 to 26, or a pharmaceutically acceptable salt thereof, wherein R² is hydrogen and R³ is fluoro.

28. A pharmaceutical composition comprising a compound of any one of claims 1 to 27 or a pharmaceutically acceptable salt thereof and a pharmaceutically acceptable excipient.

29. A method of treating cancer in a patient, which method comprises administering to the patient, a therapeutically effective amount of a compound of any one of claims 1 to 27 or a pharmaceutically acceptable salt thereof in a pharmaceutical composition comprising a compound of any one of claims 1 to 27, or a pharmaceutically acceptable salt thereof, and a pharmaceutically acceptable excipient.

30. The compound of claim 29, wherein the cancer is non-small cell lung cancer, colorectal cancer, or pancreatic cancer.
